(19) United States
(12) Patent Application Publication
(10) Pub. No.: US 2024/0153587 A1
(43) Pub. Date: May 9, 2024

(54) WORKFLOW TO ASSIGN PUTATIVE SOURCE TO DE NOVO PEPTIDE SEQUENCE

(71) Applicant: REGENERON PHARMACEUTICALS, INC., Tarrytown, NY (US)

(72) Inventors: Lili Blumenberg, Mount Kisco, NY (US); Kamil Cygan, Des Plaines, IL (US); Ankur Dhanik, Pleasantville, NY (US); Robert Salzler, Ossining, NY (US)

(73) Assignee: REGENERON PHARMACEUTICALS, INC., Tarrytown, NY (US)

(21) Appl. No.: 18/549,621

(22) PCT Filed: Mar. 11, 2022
(86) PCT No.: PCT/US2022/020049

§ 371 Date: Sep. 8, 2023

Related U.S. Application Data
(60) Provisional application No. 63/159,879, filed on Mar. 11, 2021.
(60) Provisional application No. 63/159,880, filed on Mar. 11, 2021.

(51) Int. Cl. G16B 30/10; G16B 15/30; G16B 40/10

(52) U.S. Cl. CPC G16B 30/10; G16B 15/30; G16B 40/10

(57) ABSTRACT

Methods and systems are described for optimizing search results through querying a plurality of databases according to false discovery, random hit rates are presented herein. Methods and systems adapted to assigning a putative source to a de novo peptide sequence and/or creating a workflow for performing said assignment are presented herein.

### CROSS-REFERENCE TO RELATED APPLICATIONS

This application claims priority to U.S. Provisional Application Ser. No. 63/159,879 filed on Mar. 11, 2021, and U.S. Provisional Application Ser. No. 63/159,880 filed Mar. 11, 2021, the contents of each of which are incorporated herein by reference in their entirety.

### TECHNICAL FIELD

In some embodiments, the present invention is related to computer methods/systems for optimize search results through querying a plurality of databases according to false discovery rates, random hit rates.

### BACKGROUND

Numerous data sources are being created and maintained all over the world. The number of data sources almost guarantees that part or all of a query can be answered using one of these data sources. The mere task of executing a query can be daunting regardless of whether the scope of the query is within the confines of a local computing system, a private network, a local area network, or the World Wide Web. The process of querying these data sources is made further difficult as users must decide which data sources are sufficiently reliable in order to obtain a meaningful search result. For example, the user must consider both the relative accuracy of the sources and the timeliness of the data contained within the sources. These and other shortcomings of are addressed herein.

In the field of bioinformatics, attempts are made to assign a putative source (e.g. translation from RNA, synthesis from DNA, etc.) to de novo peptide sequences. By definition, the putative sources are not fully experimentally confirmed and are, thus, flawed.

### BRIEF SUMMARY

Described herein are embodiments of methods, systems, and devices generally directed to assigning a putative source to a de novo peptide sequence and/or creating a workflow for performing said assignment. In some embodiments, the present invention includes workflows that have increased confidence in assigned putative source in the absence of experimental confirmation of the source assignment.

In one embodiment, a putative source of a peptide sequence can be determined based at least in part on a one or more searches of the peptide sequence within one or more databases such that the one or more searches are performed in order of increasing random hit rate until the putative source is determined. The random hit rate for each respective search can be determined based at least in part on a number of random peptide sequences that are found by the respective search. The one or more databases can include, but are not limited to: an expanded human proteome database, a human genome database, a non-endogenous proteome database, additional databases, and combinations thereof. The one or more searches can include, but are not limited to: a linear human proteome search for the peptide sequence within the expanded human proteome database, a linear human genome search of translations of the human genome database, a linear mismatch search for peptides having a mismatch to the peptide sequence within the expanded human proteome database, a linear non-endogenous search for the peptide sequence within the non-endogenous proteome database, a cis-spliced search within the expanded human proteome database, and a trans-spliced search within the expanded human proteome database. Each of the searches can indicate a respective potential source of the searched peptide sequence when the respective source finds a match. The putative source determined for the searched peptide sequence can be the potential source identified by the search step having the lowest random hit rate which found a match for the search peptide.

In one embodiment, peptide source search steps can be ordered to generate a peptide source assignment workflow. A plurality of random peptide sequences can be generated and each of the random peptide sequences can be searched by each peptide source search step. A random hit rate can be determined for each peptide source search step based at least in part on a number of the plurality of random peptide sequences found by the peptide source search step. The peptide source search steps can be ordered in the workflow from lowest random hit rate to highest random hit rate. The random hit rate can increase as the number of found random peptide sequences increases. The peptide source search steps can include, but are not limited to: a linear human proteome search for the peptide sequence within the expanded human proteome database, a linear human genome search of translations of the human genome database, a linear mismatch search for peptides having a mismatch to the peptide sequence within the, a linear non-endogenous search for the peptide sequence within the non-endogenous proteome database, a cis-spliced search within the expanded human proteome database, and a trans-spliced search within the expanded human proteome database.

Described herein are embodiments of methods of an invention comprising generating a plurality of simulated random queries; determining, based on applying the plurality of simulated random queries to each source of a plurality of sources, a number of matches associated with each source; determining, based on the numbers of matches associated with each source, a false discovery rate associated with each source; and generating, based on the false discovery rates, a query support data structure configured to facilitate application of a new query to the plurality of sources.

Also described are embodiments of the methods comprising receiving a query; applying, based on a query support data structure, the query to one or more sources of a plurality of sources; determining, based on a query result, a label associated with a source of the plurality of sources associated with the query result; and applying the label to the query.

The various steps of the methods disclosed herein, or steps carried out by the systems disclosed herein, may be carried out at the same or different times, in the same or different geographical locations, e.g., countries, and/or by the same or different people.

Additional advantages of the embodiments of the methods and systems will be set forth in part in the description which follows, and in part will be understood from the description, or may be learned by practice of the embodiments of the methods and systems. The advantages of the disclosed embodiments of the methods and systems will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and systems and together with the description, serve to explain the principles of the disclosed methods and systems.

FIG. 1 shows an exemplary embodiment of the system.

FIG. 2 shows an exemplary embodiment of a query support data structure.

FIG. 3 shows an exemplary embodiment of a method.

FIG. 4 is a schematic of linear, cis- and trans-spliced peptides made by proteasome-catalyzed peptide splicing/ligating.

FIG. 5 shows an exemplary embodiment of the system.

FIG. 6 shows an exemplary embodiment of the query support data structure.

FIG. 7 shows an exemplary embodiment of the method.

FIG. 8 shows an example of estimating the random hit rate of each putative source of peptides via a bar plot showing the percent of 5,000 randomly generated peptide sequences that could be matched at each step individually.

FIG. 9 shows an exemplary embodiment of a schematic of a hybridfinder workflow.

FIG. 10 shows exemplary illustrations depicting random hit rate estimation.

FIG. 11 shows an exemplary embodiment of a pattern of potential peptide sources identified for randomly generated peptides.

FIG. 12 shows an exemplary embodiment of a proportion of agreement between search of an embodiment of a putative peptide source assignment workflow and average local confidence score given during de novo sequencing.

FIG. 13 shows an exemplary embodiment using the subject system to provide peptide source identification on the HLA-A02:04-expressing the cell line by hybridfinder (left) and which peptides switched annotations (center) using the disclosed methods (right).

FIG. 14 shows an exemplary embodiment using the subject system to provide peptide source identification on all the HLA-monoallelic cell lines by hybridfinder (left) and which peptides switched annotations (center) using the disclosed methods (right).

FIG. 15 shows an exemplary embodiment using the subject system to provide the Fisher's exact test p-values measuring the enrichment of how many peptides were able to be assigned to a source at each step, compared to how many would be expected based on how many were assigned of the simulated random sequences.

FIG. 16 shows an exemplary embodiment using the subject system to provide stacked bar plots showing the proportion of peptides that mapped to different genomic regions in step 1 of the peptide source assignment workflow applied to the HLA monoallelic cell lines.

FIG. 17 shows heatmaps showing enrichment of genomic annotations for the locations of mapped peptides in step 1 of the exemplary embodiment of the peptide source assignment workflow. signed log 10 Fisher's exact test p-values of the enrichment of peptides identified exclusively in the OpenProt database for the HLA monoallelic cell lines, versus the distribution of all proteins in the OpenProt database.

FIG. 18 shows results of using the exemplary embodiment of the system, showing stacked bar plots showing the proportion of peptides that mapped to different genomic regions in step 2 of the peptide source assignment workflow applied to the HLA monoallelic cell lines.

FIG. 19 shows heatmaps showing enrichment of genomic annotations for the locations of mapped peptides in step 2 of the exemplary embodiment of the peptide source assignment workflow. signed log 10 p-values calculated by HOMER, calculating enrichment of assigned peptide locations.

FIG. 20 shows results of using the exemplary embodiments of the system, showing stacked bar plots showing the proportion of peptides that mapped to different genomic regions in step 3 of the peptide source assignment workflow applied to the HLA monoallelic cell lines.

FIG. 21 shows heatmaps showing enrichment of genomic annotations for the locations of mapped peptides in step 3 of the exemplary embodiment of the workflow. signed log 10 p-values calculated by HOMER, calculating enrichment of assigned peptide locations.

FIG. 22 shows a block diagram of an exemplary embodiment of a computing device for implementing the example methods described herein.

FIGS. 23 and 24 show flowcharts of exemplary embodiments of the method.

### DETAILED DESCRIPTION

The disclosed methods and systems may be understood more readily by reference to the following detailed description of particular embodiments and the Example included therein and to the Figures and their previous and following description.

It is understood that the disclosed methods and systems are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

The term “peptide” can be used interchangeably with “polypeptide” and refers to a polymeric form of amino acids of any length, which can include genetically coded and non-genetically coded amino acids, chemically or biochemically modified or derivatized amino acids, and peptides having modified peptide backbones. In some aspects, the term peptide refers to a string of two or more naturally occurring amino acids.

“Optional” or “optionally” means that the subsequently described event, circumstance, or material may or may not occur or be present, and that the description includes instances where the event, circumstance, or material occurs or is present and instances where it does not occur or is not present.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, the term “computer-readable representation of protein sequence” can include a sequence listing of a protein itself, a genetic sequence (e.g. DNA, RNA) from which a protein sequence can be derived through a process (e.g. transcription, translation) understood to a person skilled in the pertinent art, and/or portions thereof. Similarly, as used herein, the term “computer-readable representations of translations from ribonucleic acids (RNAs) can include a sequence listing of a protein or peptide that can be translated (at least in theory) from the RNAs as understood to a person skilled in the pertinent art, a genetic sequence of the RNA, a genetic sequence of DNA from which the RNAs can (at least in theory) be transcribed as understood to a person skilled in the pertinent art, and/or portions thereof. As used herein, the term “computer-readable representations of translations from RNAs” can refer to specific types of RNA including messenger RNAs (mRNAs), non-coding RNAs, long non-coding RNAs, micro RNAs, and other types of RNAs as understood by a person skilled in the pertinent art. Computer-readable representations of translations from a specific type of RNA can include a sequence listing of a protein or peptide that can be translated (at least in theory) from the specific type of RNAs as understood to a person skilled in the pertinent art, a genetic sequence of the specific type of RNA, a genetic sequence of DNA from which the specific type of RNA can (at least in theory) be transcribed as understood to a person skilled in the pertinent art, and/or portions thereof.

The terms “random hit rate” and “false discovery rate” are used interchangeably herein and are understood to mean a frequency at which randomly generated inputs are found by a search of a database.

An “individual” or “subject” or “animal” refers to humans, veterinary animals (e.g., cats, dogs, cows, horses, sheep, pigs, etc.) and experimental animal models of diseases (e.g., mice, rats). In some embodiments, the subject is a human.

Unless defined otherwise, all technical and scientific terms used herein have the same meanings as commonly understood by one of skill in the art to which the disclosed methods belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

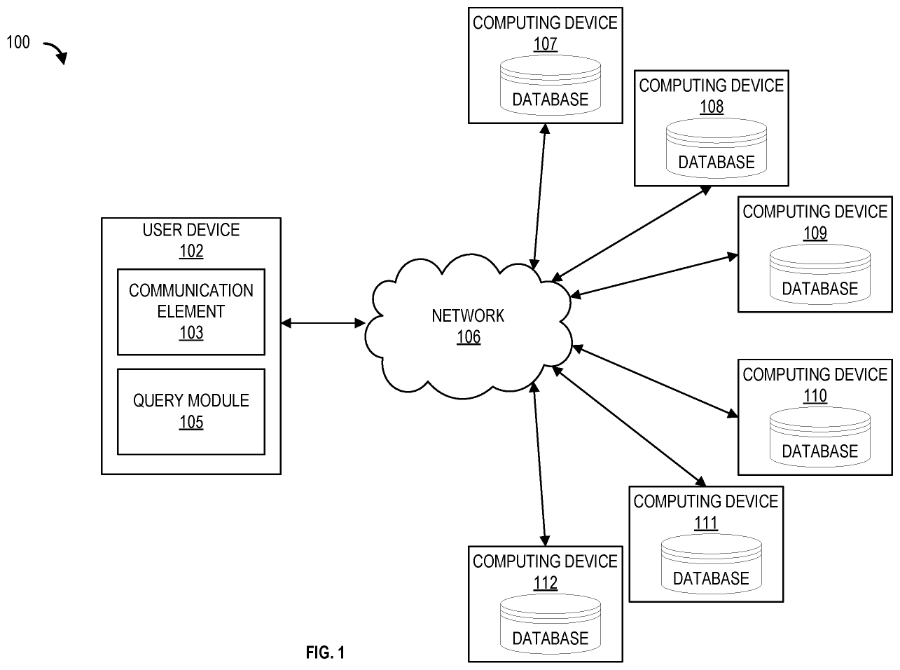

FIG. 1 shows an example system 100. The system 100 may be used to analyze one or more portions of data/information, such as query information and/or the like, and determine/identify a data source, such as an optimal data source and/or device, for analyzing the complete data/information and/or receiving/obtaining additional data/information associated with the data/information.

Devices and/or components of the system 100 may connect to and/or communicate with each other via a network 106. The network 106 may be a public network, a private network, and/or a combination thereof. The network 106 may support any wired and/or wireless communication technology and/or technique. For example, the network 106 may include a and/or support a cellular network, a data network, a content delivery network, a fiber-optic network, and/or any other type of network.

The system 100 may include a user device 102 (e.g., a computing device, a client device, a smart device, etc.). The user device 102 may comprise a communication element 103 for providing an interface to a user to interact with the user device 102 and/or any other device/component of the system 100. The communication element 103 may be any interface for presenting and/or receiving information to/from the user, such as user feedback. An interface may include a display and/or interactive interface (e.g., a keyboard, a touchscreen, a mouse, a/audio controller, etc.). An interface may include a communication interface such as a web browser (e.g., Internet Explorer®, Mozilla Firefox®, Google Chrome®, Safari®, or the like). Other software, hardware, and/or interfaces may be used to provide communication between the user and one or more of the user device 102 and/or any other device/component of the system 100. The communication element 103 may request or query various files from a local source and/or a remote source, such as computing devices 107-112, and/or any other device/component of the system 100. The computing devices 107-112 may be disposed locally or remotely relative to the user device 102.

The communication element 103 may transmit/send data to a local or remote device, such as the computing devices 107-112, and/or any other device/component of the system 100 via wired and/or wireless communication techniques. For example, the communication element 103 may utilize any suitable wired communication technique, such as Ethernet, coaxial cable, fiber optics, and/or the like. The communication element 103 may utilize any suitable long-range communication technique, such as Wi-Fi (IEEE 802.11), BLUETOOTH®, cellular, satellite, infrared, and/or the like. The communication element 103 may utilize any suitable short-range communication technique, such as BLUETOOTH®, near-field communication, infrared, and the like.

The user device 102 may receive and/or analyze data/information, such as query information and/or the like. For example, the user device 102 may receive data/information, query information, and/or the like via the communication element 103. The data/information, query information, and/or the like may include any type of information, such as statistical queries, analytical queries, industry-specific queries (e.g., immunopeptidomics-related queries, bioinformatic-related queries, biotechnology-related queries, healthcare-related queries, business-related queries, chemistry-based queries, mathematical-based queries, etc.).

The user device 102 may include a query module 105 that may analyze data/information, such as query information and/or the like. The query module 105 may be software, hardware, and/or a combination of software and hardware. The query module 105 may be configured for natural language processing, syntax determination/analysis, query language (coding) processing/analysis, and/or the like.

The user device 102 (e.g., the query module 105) may receive and/or generate a query. For example, the user device may receive and/or generate a query such as “Was the health inspection score for XYZ restaurant the same in 2020 as it was in 2019?” In another example, the user device may receive and/or generate a query such as “What was the health inspection score for XYZ restaurant in 2020?” The query module 105 may use, for example, natural language processing, syntax determination/analysis, query language (coding) processing/analysis, and/or the like to determine/identify portions/components of the query. The portions/components of the query may include one or more data constraints, predicates, text strings, syntax elements, semantic components, and/or the like. The query module 105 may combine portions/components of the query to, for example, determine/generate a set expression.

Query-based set expression(s) may be applied to a data/information source and/or system to determine a result and/or the accuracy of results. A result may be an indication of an aggregate value/amount of data records, for example, a number/quantity of matches, hits, correspondences, and/or the like between portions/components of the query and one or more data records stored by and/or associated with the source and/or system. The number/quantity of matches, hits, correspondences, and/or the like may be evaluated and/or compared against a threshold, such as a data discovery threshold. If the number/quantity of matches, hits, correspondences, and/or the like satisfy and/or exceed the discovery threshold, the query module 105 may create a data record, provide an indication of, and/or assign a label to the source and/or system. The label may indicate, for example, the type and/or quantity of matches, hits, correspondences, and/or the like associated with the source and/or system. The label may indicate any data/information relevant to queries applied to the source and/or system and/or a corresponding result.

The user device 102 may evaluate the efficacy of any source and/or system for outputting a result of a query. For example, the user device 102 (e.g., the query module 105, etc.) may send queries to and/or process queries based on one or more data sources. For simplicity and example, the computing devices 107-112 may represent one or more data sources and/or one or more search engines. Although not shown, the computing devices 107-112 may each represent a plurality of associated data sources, systems, devices, repositories, and/or the like. For example, the computing devices 107-112 may each include and/or be associated with a database (e.g., a data store, a data repository, etc.). The databases may include any type of databases, such as the Internet, in-memory/centralized databases, distributed databases, operational databases, relational databases, cloud-based databases, object-oriented databases, query language-based databases (e.g., NoSQL, etc.), graph databases, and/or the like. The databases may include any data/information. In an embodiment, each of the computing devices 107-112 may represent a different search engine configured to search the same database (e.g., the Internet).

To evaluate the efficacy of the computing devices 107-112 for outputting a result of a query, the user device 102 (e.g., the query module 105, etc.) may apply one or more queries to one or more of the computing devices 107-112 and determine false discovery rates (FDRs) associated with the computing devices 107-112. For example, the user device 102 (e.g., the query module 105, etc.) may determine/generate a plurality of random queries. The plurality of random queries may be, for example, uniform random queries, weighted random queries, and/or any other type of query. The plurality of simulated queries may be, for example, immunopeptidomics-related queries and/or bioinformatics/biotechnology-related queries, such as queries associated with a plurality of simulated random peptide sequences. The plurality of simulated random queries may be generated by any known technique. For example, a random number/letter/word generator may be used to generate a plurality of simulated, random queries, and/or test queries/cases. The quantity of simulated random queries may vary based upon the type of query which may impact, for example, a number of combinations and/or permutations of the simulated queries. For example, a number of simulated queries for restaurants, airfare and the like may vary from a number of simulated queries for DNA, RNA, and/or amino acid sequences. In an embodiment, the number of simulated queries may be restrained by a specified length of the simulated queries. For example, the simulated queries may be limited to a number of characters and/or words. In some embodiments, the number of simulated queries may range anywhere from, and including, 10 queries to 10,000,000 of queries. In some embodiments, the number of simulated queries can be, but is not limited to, 10 queries to 1,000 queries. In some embodiments, the number of simulated queries can be, but is not limited to, 10 queries to 10,000 queries. In some embodiments, the number of simulated queries can be, but is not limited to, 10 queries to 100,000 queries. In some embodiments, the number of simulated queries can be, but is not limited to, 10 queries to 1,000,000 queries. In some embodiments, the number of simulated queries can be, but is not limited to, 100 queries to 1,000 queries. In some embodiments, the number of simulated queries can be, but is not limited to, 100 queries to 10,000 queries. In some embodiments, the number of simulated queries can be, but is not limited to, 100 queries to 100,000 queries. In some embodiments, the number of simulated queries can be, but is not limited to, 100 queries to 1,000,000 queries. In some embodiments, the number of simulated queries can be, but is not limited to, 1,000 queries to 100,000 queries. In some embodiments, the number of simulated queries can be, but is not limited to, 10,000 queries to 100,000 queries. In some embodiments, the number of simulated queries can be, but is not limited to, 100,000 or more queries. In some embodiments, the number of simulated queries can be, but is not limited to, 1,000,000 or more queries. In some embodiments, the number of simulated queries can be at least 100,000 queries. In some embodiments, the number of simulated queries can be at least 1,000,000 queries.

For example, the query module 105 may use an application such as MySQL and/or the like to generate a plurality (e.g., tens, hundreds, millions, etc.) of simulated random queries, and/or test queries/cases via a suitable grammar/format. A suitable grammar may be any grammar, language, syntax, encoding, and/or the like understood/executable by the query module 105. The query module 105 may use query templates to generate queries of any suitable grammar/format. Query templates may be generated according to a scripting language. A query template may map and/or correspond to a particular test case. The query module 105 may determine a result and/or expected result for a query determined from a query template by applying the query to a source and/or system, such as the computing devices 107-112.

The query module 105 may generate/determine random queries based on a query determined, for example, from a query template. The query module 105 may apply the random queries to each of the computing devices 107-112 and determine which of the computing devices 107-112 output a positive and/or expected result. The output and/or expected result may be, for example, based on the ability of the computing devices 107-112 to process any given semantic and/or syntax of a query and retrieve data/information associated with the semantic and/or syntax. The user device 102 may determine/generate, for example, based on the output of each of the computing devices 107-112 a false discovery rate associated with each of the computing devices 107-112.

In an aspect, randomly generated queries may be incorrect, nonsensical, and/or illogical queries designed to evaluate the false discovery rate of any source and/or system, such as the computing devices 107-112. For example, a query template may be used to generate a query such as “What is the price for an airplane ticket to Dubai?” The query module 105 may determine/generate incorrect, nonsensical, and/or illogical versions and/or permutations of the query, such as: “what is the price for an apple to daylight,” “when is the price of an airplane to develop,” ‘Dubai airplane ticket currency,” “airflow ticket when the price is low, etc. Incorrect, nonsensical, and/or illogical versions and/or permutations of a query may be determined based on, for example, synonyms, phonetic relationships, and/or the like of elements (e.g., predicates, constraints, conditions, indicators, portions, etc.) of the query. Incorrect, nonsensical, and/or illogical versions and/or permutations of a query may be determined by rearranging elements of a query. Incorrect, nonsensical, and/or illogical versions and/or permutations of a query may be determined by any method.

The query module 105 may determine how frequently the computing devices 107-112 output results for incorrect, nonsensical, and/or illogical versions and/or permutations of a query, such as a plurality of random queries. How frequently the computing devices 107-112 output the results to incorrect, nonsensical, and/or illogical versions and/or permutations of a query may be indicated and/or correspond to the number of matches associated with each of the computing devices 107-112. The false discovery rate (FDR) for any given computing device 107-112 may be determined as a function of the number of matches and the number of the plurality of random queries. Determining the FDR for the computing device 107-112 based on the number of matches associated with each computing device 107-112 may include dividing the number of matches by a number of the plurality of random queries. In an embodiment, determining the FDR may take into account a relevancy score associated with a match provided by the computing device 107-112. For example, a search engine may identify a match and assign a relevancy score to the match indicating how relevant the match is to the query. Each search engine may use a proprietary relevancy scoring technique. A match may count towards an FDR determination if a simulated query returns a match with a relevancy score exceeding a threshold.

The user device 102 may, based on the false discovery rates associated with each of the computing devices 107-112, determine/generate a query support data structure configured to facilitate the application of a new query to the computing devices 107-112. For example, in an embodiment, the computing devices 107-112 may be, include, and/or be associated with search engines (e.g., Google®, Yahoo®, Bing®, Firefox®, etc.) and/or a similar data source, data repository, and/or data access system.

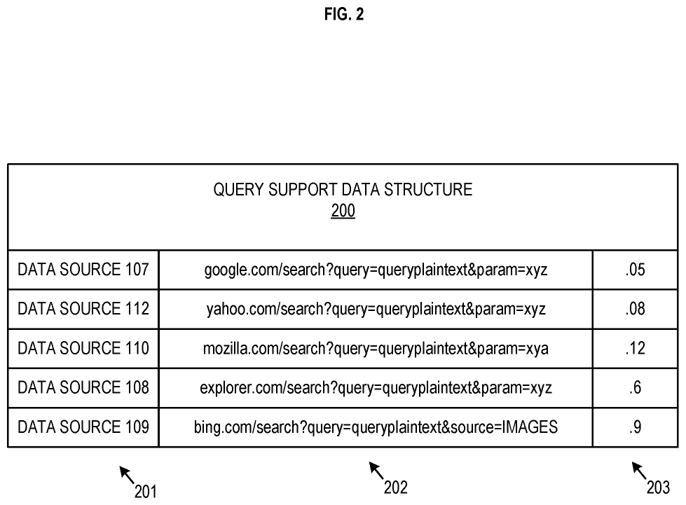

FIG. 2 shows an example data structure 200 that may be used to facilitate the application of a query to the computing devices 107-112. The query support data structure 200 may indicate an order of the computing devices 107-112 (e.g., data sources and/or search engines).

The order of the data sources may be based on a false discovery rate associated with each source. The query support data structure 200 may indicate one or more search techniques for one or more of the data sources 107-112. The query support data structure 200 may, for example, in column 202, indicate a plurality of search techniques for a single data source (e.g., the data source 107, etc.), the query support data structure 200 may indicate a single search technique for the data sources 107-112, and combinations thereof. The query support data structure 200 may comprise an identifier, in column 201, of a data source of the data sources 107-112, indicated in an order according to a false discovery rate. The false discovery rate may optionally be indicated, for example, in column 203. Data sources associated with a lower false discovery rate may be searched before data sources with a higher false discovery rate are searched. For each data source indicated in the query support data structure 200, additional data may be included. The additional data may comprise one or more of, a location of the data source, a query syntax, one or more query parameters, combinations thereof, and/or the like.

The query may be labeled based on which data source returns a query result. The label may be indicative of a source data/information associated with the query. For example, the label may indicate one or more levels of accuracy of results returned by a source based on the query. As another example, the label may indicate one or more of: text data, multimedia data, statistical data, historical data, private/secured data, public data, and/or any other label of the type of data returned by a source based on the query.

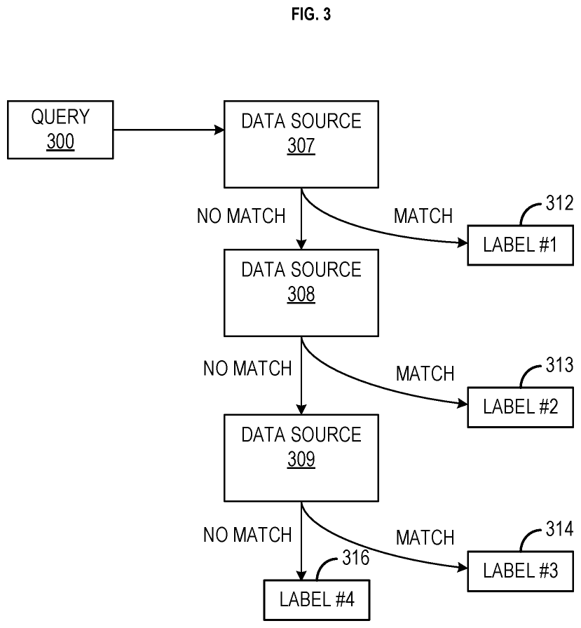

By way of example, shown in FIG. 3, a query 300 may be applied to one or more of a plurality of data sources 307-309 (e.g., search engines, the data sources 107-112, the computing devices 107-112, etc.). Permutations and/or versions of the query 300 may also be applied to the plurality of data sources 307-309. The query 300 may be, for example, “What is the price for an airplane ticket to Dubai?” The permutations and/or versions of the query 300 may be, for example: “what is airfare to Dubai,” “how much for a flight to Dubai,” “Dubai airfare,” and/or the like. The order in which the query 300 is applied to the plurality of data sources 307-309 may be indicated by a query support data structure based on false discovery rates associated with each of the plurality of data sources 307-309, as described herein. In an embodiment, as shown in FIG. 3, the data sources may be ordered according to FDR. For example, the FDR for the data source 307 may be about 1%, the FDR for the data source 308 may be about 10% and the FDR for the data source 309 may be about 68%. The data source with the lowest false discovery rate may be searched first and the data source with the highest false discovery rate may be searched last. In an embodiment, the query 300 may be discontinued at any point upon returning a search result. In an embodiment, the query 300 may be applied to each of the plurality of data sources 307-309 and, after the query 300 is completed, search results may be presented along with an indication of the associated data source and FDR. In this fashion, a user may decide with search result to have greater confidence in and whether the user wishes to apply any FDR-based filters (e.g. remove search results associated with data sources having a high FDR value).

Additionally, each data source of the plurality of data sources 307-309 may be associated with a threshold, such as a data discovery threshold applied to relevancy scores of matches. A data discovery threshold may be a system-defined threshold and/or a user-defined threshold. In an embodiment, a data source associated with a low false discovery rate may be associated with a low data discovery threshold as the data source is generally associated with “good” results and any matches from the data source should be subject to less strict relevancy requirements. A data source associated with a high false discovery rate may be associated with a high data discovery threshold as the data source is associated with less “good” results and any matches from the data source should be subject to stricter relevancy requirements. In another embodiment, a data source associated with a low false discovery rate may be associated with a high data discovery threshold as the data source is generally associated with “good” results and is more likely to contain a relevant result. A data source associated with a high false discovery rate may be associated with a low data discovery threshold as the data source is associated with less “good” results and a low data discovery threshold may be necessary in order to determine a relevant result. In an embodiment, a data discovery threshold may be determined and/or set by a user, for example, via a user interface.

In an embodiment, each data source of the plurality of data sources 307-309 may be associated with the same or a different data discovery threshold. For example, when a query is applied to a first data source a first data discovery threshold may dictate that a match exists only if the match has a relevancy score greater than the first data discovery threshold (e.g., 85%), if no match satisfies the first data discovery threshold, the query may be applied to a second data source associated with a second data discovery threshold that dictates that a match exists only if the match has a relevancy score greater than the second data discovery threshold (e.g., 90%). If no match satisfies the second data discovery threshold, then the query may be applied to a third data source associated with a third data discovery threshold that dictates that a match exists only if the match has a relevancy score greater than the third data discovery threshold (e.g., 95%), if no match satisfies the third data discovery threshold, then no results are output.

When applying the query 300 to the data sources, if a match is found that satisfies a data discovery threshold (e.g., a system-determined threshold, a user-configurable threshold, etc.) for the query 300 in and/or via the data source 307 the result may receive a first label (Highly Accurate Results) at 312 and all relevant and/or possible results may be included in the output. Otherwise, the query 300 may be applied to a next data source 308. If a match is found that satisfies a data discovery threshold (e.g., a system-determined threshold, a user-configurable threshold, etc.), the result may receive a second label (Likely Accurate Results) at 313 and all relevant and/or possible results may be included in the output. Otherwise, the query 300 may be applied to a next data source 309. If a match is found that satisfies a data discovery threshold (e.g., a system-determined threshold, a user-configurable threshold, etc.), the result may receive a third label (Accurate Results) at 314 and all relevant and/or possible results may be included in the output. If no matches are determined/identified, the non-result may receive a fourth label (No Results) at 316.

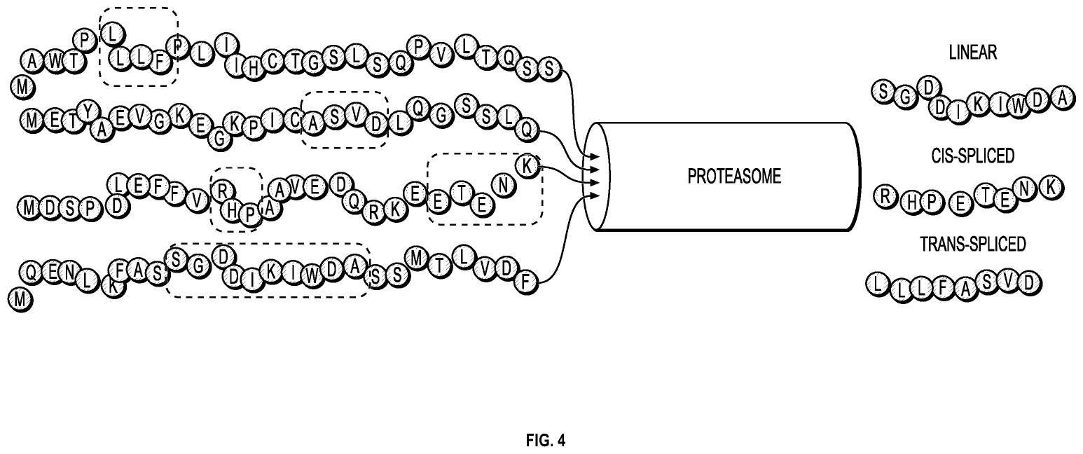

Turning now to an exemplary embodiment of the disclosed methods and systems to de novo peptide sequencing, FIG. 4 shows a schematic of how linear, cis-, and trans-spliced peptides are produced. For example, a linear peptide sequence matches identically to its parental protein, fragments of cis-spliced peptides are from the same protein, and trans-spliced peptide fragments are from different proteins.

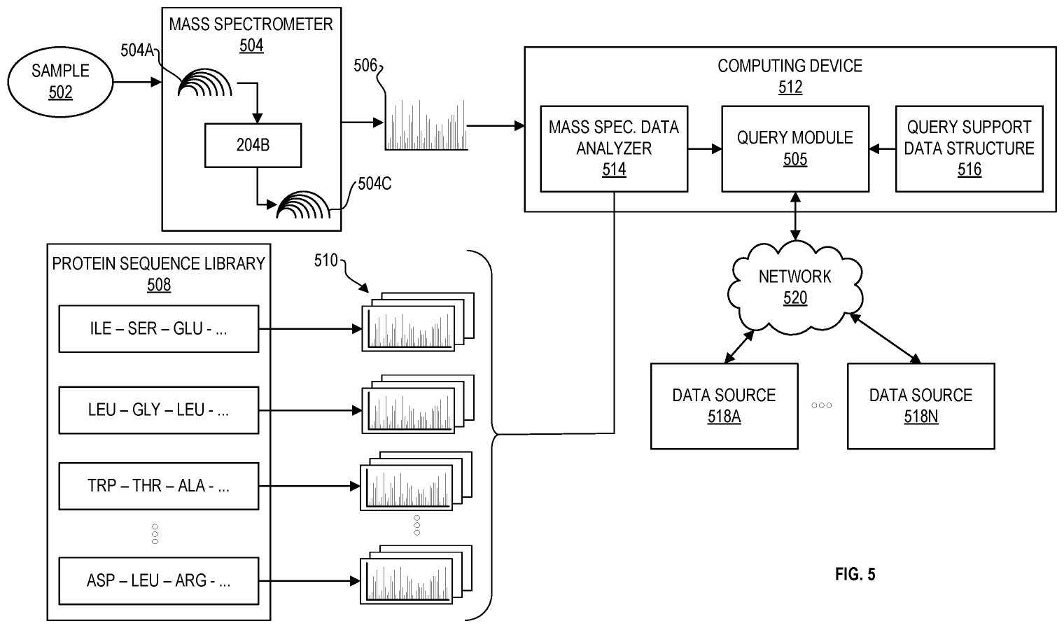

FIG. 5 shows an example system 500. The example system 500 may be configured for mass spectrometry. A mass spectrometer 504 enables precise determination of the molecular mass of peptides as well as their sequences. For example, the mass spectrometer 504 may output data/information, such as mass spectrometry data, that may be used for protein identification, de novo sequencing, and identification of post-translational modifications. The system 500 may be configured to assign a source of de novo sequenced peptides.

Tandem mass spectrometry (MS/MS) has become a leading high-throughput technology for protein identification. A tandem mass spectrometer 504 may be configured for ionizing a mixture of peptides in a sample 502 with different peptide sequences and measuring their respective parent mass/charge ratios, selectively fragmenting each peptide into pieces and measuring the mass/charge ratios of the fragment ions. The tandem mass spectrometer 504 may be, as non-limiting examples, a Linear Ion Trap Mass spectrometer (LTQ) combined with a Fourier Transform Ion Cyclotron Resonance Mass Spectrometer (LTQ-FT). Thus, a tandem mass spectrum can be viewed as a collection of fragment masses from a single peptide. This collection, or set, of fragment masses, or fragment mass values, is a “fingerprint” that identifies the peptide. The peptide sequencing problem is then to derive the sequence of the peptides given their MS/MS spectra. For an ideal fragmentation process and an ideal mass spectrometer, the sequence of a peptide could be easily determined by converting the mass differences of the consecutive ions in a spectrum to the corresponding amino acids. This ideal situation would occur if the fragmentation process could be controlled so that each peptide was cleaved between every two consecutive amino acids and a single charge was retained on only the N-terminal piece. In practice, however, the fragmentation processes in mass spectrometers are not ideal.

The problem for tandem mass spectrometry peptide sequencing is, given a spectrum S, the ion types Δ, and the mass m, finding a peptide of mass m with the maximal match to spectrum S. Peptide fragmentation in a tandem mass spectrometer can be characterized by a set of numbers Δ={δ₁, . . . , δₖ} representing ion types. A δ-ion of a partial peptide P′⊂P is a modification of P′ that has mass m(P′)−δ. For tandem mass spectrometry, the theoretical spectrum of peptide P can be calculated by subtracting all possible ion types {δ₁, . . . , δₖ} from the masses of all partial peptides of P (i.e., every partial peptide generates k masses in the theoretical spectrum). An (experimental) spectrum S={s₁, . . . , sₘ} is a set of masses of fragment ions. A match between spectrum S and peptide P is the number of masses that experimental and theoretical spectra have in common.

LTQ-FT mass spectrometers can generate on the order of 100,000 spectra per day per machine. Software is a significant and limiting factor in mass spectrometry proteomics analysis—typical large datasets may require days or weeks of computational time on expensive computers or grids. Most peptide identification algorithms use database search methods that match the spectra against a protein database. FIG. 5 illustrates an exemplary process for spectrum matching techniques for peptide identification. Specifically, the sample 502 is provided to the mass spectrometer 504. The mass spectrometer 504 may comprise any number of mass spectrometers, for example, two mass spectrometers in a tandem arrangement. A two-step process is illustrated, however, single-step processes are also known. In a first mass spectrometer 504A, a peptide ion is selected, so that a targeted component of a specific mass is separated from the rest of the sample. The targeted component is then activated or decomposed at 504B. In the case of a peptide, the result will be a mixture of the ionized parent peptide (“precursor ion”) and component peptides of lower mass which are ionized to various states. A number of activation methods can be used including collisions with neutral gases (also referred to as collision induced dissolution). The parent peptide and its fragments are then provided to a second mass spectrometer 504C, which outputs an intensity and m/z for each of the plurality of fragments in the fragment mixture. This information can be output as a fragment mass spectrum 506. In the fragment mass spectrum 506, each fragment ion is represented as a bar graph whose abscissa value indicates the mass-to-charge ratio (m/z) and whose ordinate value represents intensity. The fragment mass spectrum 506 may take the form of mass spectrometry data.

A computing device 512 may be configured to analyze the mass spectrometry data (e.g., the fragment mass spectrum 506) generated by the mass spectrometer 504 to identify one or more amino acids based upon a comparison of information derived from the mass spectrometry data to information contained within a protein sequence library 508. In some implementations, a user operating the computing device 512 may access a mass spectrometry data analyzer 514 executing upon the computing device 512. In some implementations, the user supplies the mass spectrometry data generated by the mass spectrometer 504 to the mass spectrometry data analyzer 514. The user, in other implementations, selects the mass spectrometry data from available mass spectrometry data (e.g., previously downloaded, transferred, or otherwise made available to the computing device 512 by the mass spectrometer 504). In some implementations, the mass spectrometer 504 includes the computing device 512. For example, the computing device 512 may be implemented as one or more computer processors functioning within a mass spectrometer system. Each implementation is understood to describe additional embodiments of the method and system described herein.

In some implementations, the mass spectrometry data analyzer 514 calculates additional data from the mass spectrometry data. For example, based upon the experimental information contained within the mass spectrometry data, a mass-charge ratio of ions (e.g., calculated as centroids of the peaks in the so-called “profile” spectra), the relative intensities of the peaks, and/or electric charge.

In an embodiment, sub-sequences contained in the protein sequence library 508 are used as a basis for predicting a plurality of mass spectra 510. The predicted mass spectra 510 of the sub-sequences may be compared, using the mass spectrometry data analyzer 514 of the computing device 512, to the experimentally-derived fragment spectrum 506 to identify one or more of the predicted mass spectra which most closely match the experimentally-derived fragment spectrum 506.

In an embodiment, de novo peptide sequencing may be implemented using, for example, a spectrum graph approach, wherein a spectrum is represented as a graph with peaks as vertices that are connected by edges if their mass difference corresponds to the mass of an amino acid. The vertices of the spectrum graph are further scored based on peak intensities and neutral losses, and a peptide sequence is obtained by finding a longest path in the graph. De novo peptide sequencing can be viewed as a search in the database of all possible peptides. For a typical spectrum identified in a database search, there may be hundreds, and even thousands, of very different peptide sequences that match the spectrum. As a result, de novo peptide sequencing algorithms output multiple peptide reconstructions rather than a single reconstruction.

In an embodiment, the protein sequence library 508 may comprise a spectral dictionary that may be used to generate a full length peptide reconstruction with a high probability of containing the correct peptides. However, an unsolved problem is how many reconstructions must be generated to avoid losing the correct peptide. Generating too few peptides will lead to false negative errors while generating too many peptides will lead to false positive errors. Some de novo algorithms output a single or a fixed number (decided before the search) of peptides. For some spectra, generating only one reconstruction may be enough to guarantee finding the correct peptide while in other cases (even with the same parent mass), a thousand reconstructions may be insufficient. The problem of generating varying numbers of reconstructions for each spectrum becomes particularly important for long peptides with the increasing complexity of the search space.

Predicted peptide sequences resulting from the comparison of the mass spectrometry data to the protein sequence library 508 by the mass spectrometry data analyzer 514 may be provided to a query module 505. The query module 505 may be configured for identifying a source of a peptide sequence using a plurality of data sources 518A-518N in communication with the query module via a network 520. The plurality of data sources 518A-518N may comprise any number and any type of data source. The plurality of data sources 518A-518N may each include and/or be associated with a database (e.g., a data store, a data repository, etc.). The databases may include any type of databases, such as in-memory/centralized databases, distributed databases, operational databases, relational databases, cloud-based databases, object-oriented databases, query language-based databases (e.g., NoSQL, etc.), graph databases, and/or the like. The databases may include any data/information, such as data/information associated with peptides and/or the like.

In an embodiment, the data sources 518A-518N may comprise an expanded human proteome database. The expanded human proteome database can include computer-readable representations of protein sequences. The expanded human proteome database can include computer-readable representations of translations of non-coding RNAs. The expanded human proteome database can include long non-coding RNAs (lncRNAs). The expanded human proteome database can include micro RNAs (miRNAs), which is a type of non-coding RNA. The expanded human proteome database can include RNA transcribed from human endogenous retroviruses (HERVs). The expanded human proteome database can further include messenger RNAs (mRNAs), which canonically code for proteins. In some embodiments, at least a portion of the computer-readable representations of protein sequences of the expanded human proteom database can be associated with a specific subject so the workflow can assign a subject-specific putative source to de-novo peptide sequences derived from the subject.

The expanded human proteome database can include peptides from non-canonically translated regions of the human genome, i.e. peptides from regions annotated as non-coding. The expanded human proteome database can include a portion or all of OpenProt, and/or one or more databases including similar data as a portion or all of OpenProt as understood by a person skilled in the pertinent art. OpenProt is disclosed, for example, in Brunet M. A., Brunelle M., Lucier J.-F., Delcourt V., Levesque M., Grenier F., et al. (2019). OpenProt: A More Comprehensive Guide to Explore Eukaryotic Coding Potential and Proteomes. Nucleic Acids Res. 47, D403-D410. 10.1093/nar/gky936, which is incorporated herein by reference in its entirety. The expanded human proteome database can include computer-readable representations of protein sequences representing translations of non-coding RNA by virtue of including a portion or all of OpenProt and/or one or more databases including non-coding RNA sequences and/or translations thereof. OpenProt a polycistronic model of eukaryotic genomes and includes all open reading frames (ORFs) at least 30 codons long.

The expanded human proteome database can include translations of lncRNAs, i.e. from non-canonically translated regions of the human genome. LncRNAs were first characterized as mRNA-like non-coding RNAs in that they undergo splicing and have features such as a poly(A) signal/tail, while an arbitrary criterion of ‘transcripts longer than 200 nucleotides’ has later been added to its ‘definition’. The expanded human proteome database can include a portion or all of NONCODE, and/or one or more databases including similar data as a portion or all of NONCODE as understood by a person skilled in the pertinent art. NONCODE is disclosed, for example, in Bu, D. et al. NONCODE v3.0: Integrative annotation of long noncoding RNAs. Nucleic Acids Res. 40, D210-5 (2012), which is incorporated herein by reference in its entirety. The expanded human proteome database can include computer-readable representations of protein sequences representing translations of lncRNA by virtue of including a portion or all of NONCODE and/or one or more databases including lncRNA sequences and/or translations thereof.

The expanded human proteome database can include translations of miRNAs, a type of non-coding RNA with a length of about 22 base. Typically miRNAs regulate gene expression by blocking translation of specific mRNAs and cause their degradation. The expanded human proteome database can include a portion or all of miRBase, and/or one or more databases including similar data as a portion or all of miRBase as understood by a person skilled in the pertinent art. miRBase is disclosed, for example, in Kozomara, A., Birgaoanu, M. & Griffiths-Jones, S. miRBase: from microRNA sequences to function. Nucleic Acids Res. 47, D155-D162 (2019), which is incorporated herein by reference in its entirety. The expanded human proteome database can include computer-readable representations of protein sequences representing translations of miRNA by virtue of including a portion or all of miRNA and/or one or more databases including miRNA sequences and/or translations thereof.

The expanded human proteome database can include transcriptions of HERVs, human genome sequences corresponding to endogenous viral elements. The expanded human proteome database can include a portion or all of gEVE, and/or one or more databases including similar data as a portion or all of gEVE as understood by a person skilled in the pertinent art. gEVE is disclosed, for example, in Nakagawa, S. & Takahashi, M. U. gEVE: a genome-based endogenous viral element database provides comprehensive viral protein-coding sequences in mammalian genomes. Database (Oxford). (2016) doi:10.1093/database/baw087, which is incorporated herein by reference in its entirety. The expanded human proteome database can include computer-readable representations of protein sequences representing translations of HERVs by virtue of including a portion or all of gEVE and/or one or more databases including HERV sequences and/or translations thereof.

The expanded human proteome database can include mRNAs by virtue of including a portion or all of UniProt and/or one or more databases including similar data as a portion or all of UniProt as understood by a person skilled in the pertinent art. The expanded human proteome database can include UniProt, to the extent that OpenProt utilizes UniProt, by virtue of the expanded human proteome database including OpenProt. Additionally, or alternatively, UniProt or a portion thereof can be included separately from OpenProt within the expanded human proteome database. In a preferred embodiment, the expanded human proteome database includes UniProt reviewed and/or one or more databases including similar data as a portion or all of UniProt reviewed as understood by a person skilled in the pertinent art. In some embodiments, the expanded human proteome database includes UniProt unreviewed and/or one or more databases including similar data as a portion or all of UniProt unreviewed as understood by a person skilled in the pertinent art.

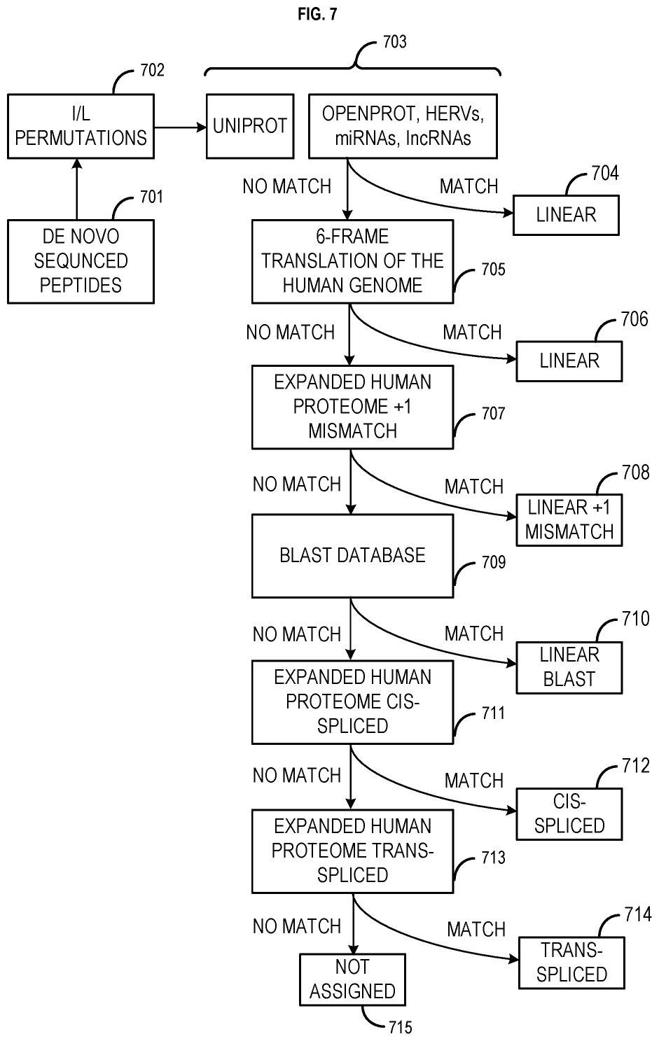

The expanded proteome database can be stored in a single memory or distributed across multiple memories. The expanded proteome database can include multiple disparate databases that can be queried as one database through a single query of a workflow such as, but not limited to the workflow illustrated in FIG. 7 and modifications thereof as well as other workflow embodiments disclosed herein.

In an embodiment, the data sources 518A-518N can include a human genome database including all or a portion of the human genome, from which computer-readable representations of proteins can be computationally synthesized. The human genome includes approximately three billion base pairs of deoxyribonucleic acid (DNA) that make up the entire set of chromosomes of the human organism. The human genome includes the coding regions of DNA, which encode all the genes (between 20,000 and 25,000) of the human organism, as well as the non-coding regions of DNA, which do not encode any genes. In some embodiments, the human genome database can include the entirety of the human genome including coding and non-coding regions of DNA. In some embodiments, the human genome database can include a non-coding portions and/or frame reads of the human genome, excluding portions and/or frame reads of the human genome from which the mRNA and non-coding RNA of the expanded human proteome database are transcribed. In some embodiments, proteins can be computationally synthesized based on one, two, three, four, five, and/or six frame translations of all or a portion of the human genome; such that some portions of the human genome may or may not be translated using the same number of frame reads as other portions of the human genome.

In an embodiment, the data sources 518A-518N can include a non-endogenous proteome database including computer-readable representations of proteins and/or peptides originating from sources non-endogenous to humans including, but not limited to, bacterial sources, viral sources, and other organisms. In an embodiment, the non-endogenous proteome database can include the NCBI BLAST database, and/or one or more databases including similar data as a portion or all of NCBI BLAST as understood by a person skilled in the pertinent art. NCBI BLAST is disclosed, for example, in Johnson, M. et al. NCBI BLAST: a better web interface. Nucleic Acids Res. 36, W5-9 (2008), which is incorporated herein by reference in its entirety. The data sources 518A-518N can include computer-readable representations of protein sequences representing translations of sources non-endogenous to humans by virtue of including a portion or all of NCBI BLAST and/or one or more databases including such sequences and/or translations thereof.

In an embodiment, the data sources 518A-518N can include computer-readable representations of proteins and/or peptides that are subject-specific, associated with an individual subject. These subject-specific data can be incorporated into one or more databases disclosed herein (e.g. expanded human proteome database, human genome database, non-endogenous proteome database, etc.) and/or included in a separate subject-specific database.

The query module 505 may utilize a query support data structure 516 to guide the identification process. The query support data structure 516 may indicate an order of search steps of the plurality of data sources to apply the query. The order may be based on a random hit rate associated with each search step. The query support data structure 516 may indicate one or more search techniques for one or more of the plurality of data sources 518A-518N. The query support data structure 516 may indicate a plurality of search techniques for a single data source, the query support data structure 516 may indicate a single search technique for a plurality of data source 518A-518N, and combinations thereof.

The query support data structure 516 can include a peptide source assignment workflow for assigning a putative source to a peptide sequence input to the workflow, wherein the putative source indicates a mostly likely origin of the peptide sequence. Each search step of the query support data structure 516 can include a peptide source search step indicating a respective potential source of the peptide sequence when the peptide source search step finds a match. A linear expanded human proteome source can be indicated by a linear human proteome search for the peptide sequence within the expanded human proteome database. A linear genome source can be indicated by a linear human genome search of translations of the human genome database. A linear mismatch can be indicated by a linear mismatch search for peptides having a mismatch to the peptide sequence within the expanded human proteome database, a linear mismatch search for peptides having a mismatch to a peptide derived from a translation of the human genome, and/or a linear mismatch search of a subject-specific database. A linear non-endogenous proteome source can be indicated by a linear non-endogenous search for the peptide sequence within the non-endogenous proteome database. A cis-spliced human proteome source can be identified by a cis-spliced search of the expanded human proteome database. A trans-spliced human proteome source can be indicated by a trans-splice search of the expanded human proteome database. The putative source assigned to the peptide sequence can be the potential source found earliest in the workflow, i.e. the search step having the lowest random hit rate.

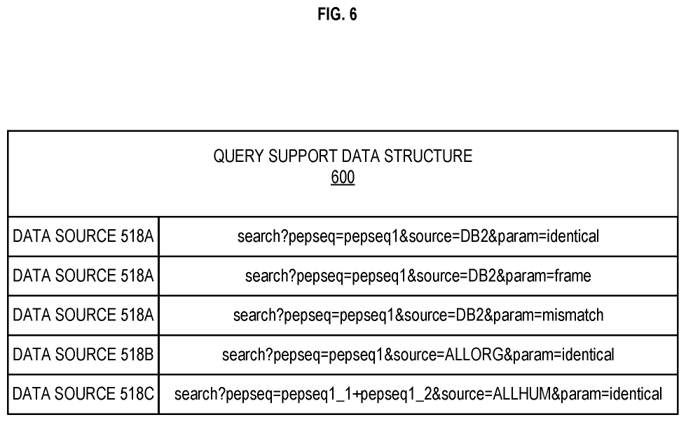

FIG. 6 shows an example of the query support data structure 600. The query support data structure 600 may comprise a search steps for searching the data sources of the plurality of data sources 518A-518N, indicated in an order according to a random hit rate. Search steps associated with a lower random hit rate may be searched prior to performing search steps with a higher random hit rate. For each search step indicated in the query support data structure 600 additional search steps may be included and search steps can be omitted.

In an embodiment, the query support data structure 600 may have been previously generated or may be generated as needed. The query support data structure 600 may be generated by, for example, generating a plurality of simulated random queries, determining, based on applying the plurality of simulated random queries to each search step, a number of matches associated with each search step, determining, based on the numbers of matches associated with each search step, a random hit rate associated with each search step, and generating, based on the random hit rates, the query support data structure configured to facilitate application of a new query to the plurality of sources. The plurality of simulated random queries may comprise at least one of a plurality of uniform random queries or a plurality of weighted random queries. Uniform random queries (e.g., peptide sequences) may be generated by randomly sampling all amino acids uniformly. Weighted random queries (e.g., peptide sequences) may be generated by randomly sampling amino acids with frequencies of amino acids matching those found in vertebrates. Determining, based on the numbers of matches associated with each source, the random hit rate associated with each search step may comprise a function of the number of matches and a number of the plurality of simulated random queries. As a non-limiting example, the random hit rate associated with each source may be determined by dividing the number of matches by a number of the plurality of simulated random queries. The random hit rate may further be dependent upon the size and/or complexity of the data source being searched.

In an embodiment, the mass spectrometry data may be used, or processed and then used, as a query to be applied to one or more of the plurality of data sources 518A-518N according to the query support data structure 600. The query may be further processed prior to being applied to applied to one or more of the plurality of data sources 518A-518N. In an embodiment, one or more permutations of the query may be determined. For example, one or more permutations of a peptide sequence may be determined and the one or more permutations used as queries in addition to the original query. For example, a peptide sequence provided as the query to the workflow of the query support data structure 600 can include one or more ambiguous residues. For example, leucine (L) and isoleucine (I) have the same mass; therefore it is impossible to differentiate them in de novo search sequencing. To account for this, for a given peptide containing I/L, all permutations of I and L residues may be considered such that the associated permutated peptide sequences are provided as queries to the workflow of the query support data structure 600. For example, for the peptide “ATTSLLHN (SEQ ID NO:1” four possible permutations exist: ATTSLLHN (SEQ ID NO:1), ATTSLIHN (SEQ ID NO:2), ATTSILHN (SEQ ID NO:3), and ATTSIIHN (SEQ ID NO:4). Each permutated peptide sequence may be used as a query. Each permutated peptide sequence can be assigned a respective putative source according to the peptide source assignment workflow of the query module 505. The assigned putative sources of the permutations are, in turn, potential sources for the provided peptide sequence having ambiguous residue(s). The potential source indicated by the peptide source step having the lowest random hit rate can be assigned as the putative source of the provided peptide sequence having ambiguous residue(s). Further, the permutations of the provided peptide can be filtered to remove those permutations not assigned the putative source.

FIG. 7 is a flow diagram outlining steps of an example peptide assignment workflow. De novo sequenced peptide sequences 701 may be used to generate one or more permutations 702 of the de novo sequenced peptide sequences.

At a first peptide source search step 703, a query (701 and 702) may be applied to an expanded human proteome database to identify an identical match. If an identical match is found for any permutation, the peptide sequence may be labeled as “Linear,” at 704 and all possible protein sources of the peptide may be included in the output of the workflow. The peptide sequence 701 and permutations 702 found by the linear human proteome search for the peptide sequence within the expanded human proteome database 703 can be assigned a linear expanded human proteome source. The assigned source can be included in the output of the workflow. The permutations found by the linear human proteome search within the expanded human proteome database 703 can be included in the output of the workflow.

At a second peptide source search step 705, BLAT, or a similar alignment tool, may be used to apply the query (701 and 702) to the frames of the translated human genome. BLAT is disclosed, for example, in Genome Res. 2002 April; 12(4): 656-664. BLAT—The BLAST-Like Alignment Tool, which is incorporated herein by reference in its entirety. An example BLAT command may be, as a non-limiting example, “blat -t=dnax -q=prot -minScore=7 -stepSize=1 hg38.2 bit Fasta_query output.psl psl2bed<output.psl>perfect_match.bed”. If an identical match is found, the peptide sequence may be labeled as “Linear,” at 706 and possible source sequences may be included in the output. The peptide sequence 701 and permutations 702 thereof found by the linear human genome search 705 can be assigned a linear genome source. The assigned source can be included in the output of the workflow.

At a third peptide source search step 707, the peptide sequences of the query (701 and 702) may be mapped to the expanded human proteome database 703, permitting a number of mismatches (as a non-limiting example, 1, 2, 3, 4, 5, 6, 7, 8, 9, 10, and the like mismatches. In an embodiment, the number of mismatches may be 1. An example BLAT command may be, for example, “blat -t=prot -q=prot -minScore=7 -stepSize=1 combined DB.processed.fasta Fasta_query output_blat_hits.psl”. If a peptide sequence with a mismatch is found (by way of example, 1 mismatch), the peptide sequence may be labeled as “one mismatch” at 708. The peptide sequence 701 and permutations thereof 702 found by the linear mismatch search for peptides having a mismatch to the peptide sequence within the expanded human proteome database 707 are assigned, as a source, the linear mismatch of the expanded human proteome. The assigned source can be included in the output of the workflow.

At a fourth peptide source search step 709, the peptide sequences of the query (701 and 702) may be mapped to other organisms at 709, for example by using the BLAST NCBI tool. If any identical matches (e.g., a homologous match) are found the results may be annotated as “LINEAR BLAST” at 710. The peptide sequence 701 and permutations thereof 702 found by the linear non-endogenous search for the peptide sequence within the non-endogenous proteome database 709 are assigned the linear non-endogenous proteome source. The assigned source can be included in the output of the workflow. In some embodiments, the fourth peptide source search step 709 can be omitted, and the workflow illustrated in FIG. 7 can be modified to omit block 709 and output 710 associated with this step. In such embodiments, the workflow can proceed from the third peptide source search step 707 to the fifth peptide source search step 711.

At a fifth peptide source search step 711, the peptide sequences of the query (701 and 702) may be fragmented into 2 or more fragments (where each fragment is greater than 1 amino acid). The fragments may be used as a query applied to the expanded human proteome database. If there is a match for both fragments in the same protein, the peptide sequence may be labeled as “cis-spliced” at 712. The peptide sequence 701 and permutations thereof 702 found by the cis-spliced search of the expanded human proteome database 711 are assigned the cis-spliced human proteome source. The assigned source can be included in the output of the workflow.

At a sixth peptide source search step 713, if there are hits for both fragments in two different proteins, the peptide sequence may be labeled as “trans-spliced” at 714. The peptide sequence 701 and permutations thereof 702 found by the trans-spliced search of the expanded human proteome database 711 are assigned the trans-spliced human proteome source. The assigned source can be included in the output of the workflow. In some embodiments, the sixth peptide source search step 713 can be omitted, and the workflow illustrated in FIG. 7 can be modified to omit block 713 and output 714 associated with this step. In such embodiments, the workflow can proceed from the fifth peptide source search step 711 to block 715.

Any remaining peptide sequences may be labeled as not assigned (N/A) at 715. The workflow can halt advancement to a subsequent peptide source search step upon assigning a putative source to a peptide sequence of the query 701, 702.

Returning to FIG. 5, in an embodiment, the computing device 512 may validate data/information received from the mass spectrometer 504 based on a label of a peptide sequence determined according to the query support data structure 600.

### Examples

Examples presented herein generally include a peptide source assignment workflow having search steps sequenced in order of increasing random hit rates and methods and systems for using and generating the peptide source assignment workflow. Examples presented infra are specific to labeling of peptides, although other applications, including those disclosed supra, can be performed following a similar methodology. The examples presented infra can reduce false labeling of peptides as cis-spliced and trans-spliced compared to previous systems and methodologies.

Antigen presenting cells use major histocompatibility (MHC) complexes I or II to present peptides to CD8+ or CD4+ T cells, respectively. Characterization of the peptides presented to T cells, known as the immunopeptidome, is being studied in the fields of infectious disease, autoimmunity as well as cancer immunotherapy. Cancer-associated MHC-presented peptides that illicit an immune response are possible safe and effective targets for cancer immunotherapy. The discovery and characterization of the immunopeptidome can be achieved using a multitude of technologies such as whole-exome sequencing, RNA sequencing, ribosome profiling and tandem mass spectrometry (MS/MS) based peptide sequencing. While next-generation sequencing approaches can characterize the potential endogenous immunopeptidome, only direct detection of peptides, like by MS/MS, can provide experimental evidence for the existence of peptides presented by MHC complexes. Notably, besides using peptides bound to MHC complexes for characterization of an immunopeptidome, peptides can also originate from multiple other genetic and transcription-based aberrations. Examples of additional means for identifying aberrant peptides include cancer specific gene and transposon overexpression (e.g., but not limited to, cancer-testis genes, transposons, and human endogenous retroviruses (HERVs)), alternative splicing, stop codon readthrough or alternative open-reading frame translation.

Immunopeptidomics using peptide-MHC elution followed by MS/MS traditionally requires a reference database of potential peptides that might be detected. Recent advances in peptide spectra matching software allow omitting reference database searches to perform de novo sequencing, whereby the software identifies the sequences of unknown peptides, post-translational modifications (PTMs) and amino acid substitutions directly from MS/MS spectra. Using these methods, the diversity of peptides that may be bound to the MHC complex can be understood, but not their protein sources. For MHC I, the canonical mechanism of presenting peptides starts with proteasomal cleavage of proteins within the cytoplasm, generating fragments between 8 and 12 amino acids in length. Those peptides are then bound to the MHC I complex before its translocation to the cell membrane. However, some studies have suggested that, in addition to cleavage, proteasomes can catalyze the reverse reaction, ligating small peptides together in a process called proteasome-catalyzed peptide splicing (PCPS). While canonical cleavage generates peptides whose sequences are identical to the parental protein (herein called linear), pieces of spliced peptides can be from the same protein (herein called cis-spliced) or, theoretically, from different proteins (herein called trans-spliced).

Prior attempts to use a de novo sequencing approach to identify peptides of unknown origin (“cryptic peptides”), have identified many of these cryptic peptides as likely being generated through post-translational splicing. However, the abundance and even the existence of spliced peptides is a matter of controversy in the field. A strategy was previously developed to identify spliced peptides in the MHC-I immunopeptidome by mass spectrometry. A database was generated containing all possible cis-spliced peptides, allowing for MS/MS spectra to be queried for cis-spliced peptides. It was reported that about 30% of p-HLA are short-distance cis-spliced peptides. The same group also developed a pipeline for mapping the MHC class I spliced immunopeptidome of cancer cells. The study suggested a substantial (˜25%) portion of peptides can be mapped to cis-spliced sequences in HCT116 and HCC1143 cell lines derived from colon and breast carcinomas, respectively. Trans-spliced peptides were excluded from analysis since their occurrence in vivo is controversial, and their addition to a database would massively increase its complexity. Later, a bioinformatics workflow was developed to identify linear, cis-, and trans-spliced peptides called hybridfinder. Hybridfinder first searches for exact matches of peptides in the UniProt human protein sequence database, then it searches for all possible cis- then trans-spliced forms of that peptide in the human proteome. Hybridfinder was used to analyze MS/MS data containing peptides eluted from MHC I complexes purified from seventeen HLA-monoallelic cell lines. Cis- and trans-spliced peptides were found to represent up to 45% of MHC-bound peptides.

### A. Results

1. Expanding the Search for Sources of Non-Canonical Human Peptides

A strategy is disclosed herein for determining the order of putative sources when assigning sources to de novo sequence peptides. The strategy is used to develop a peptide source assignment workflow that searches for the sources of peptides amongst multiple sources in a specific order, with the order optimized to minimize assignment of peptides to incorrect sources. For example, assignment of de novo peptides to post-translational cis- or trans-splicing occurs by chance extremely often and most peptides can be attributed to other sources which are less likely to occur by chance. As disclosed herein, a rigorous derivation of the optimal order of a peptide source assignment is presented and the workflow's utility in identifying the most plausible sources of de novo peptides is presenting, thus furthering the understanding of the immunopeptidome.

Previous studies have shown that up to 45% of MHC-bound peptides that do not map identically to the UniProt human proteome. The workflow disclosed herein includes databases developed of several other potential sources from which unmapped peptides may also stem. Peptides from non-canonically translated regions of the human genome, e.g., peptides from regions annotated as non-coding, were searched. For this source, OpenProt was used which includes all open reading frames (ORFs) at least 30 codons long, which was supplemented with the rest of the human genome translated into six frames. Translations of known transcribed elements were also included, including long non-coding RNAs (lncRNAs), micro RNAs (miRNAs), and HERVs which may be spliced and therefore contain sequences not found via translating genomic DNA. Below, this combination of sources is referred to as the expanded human proteome database. In addition, unknown SNPs, missense mutations, or recurrent errors in either transcription, translation, or MS amino acid identification could generate peptides with a single mismatch to a sequence encoded in the human proteome. Mismatched peptides were searched for using BLAT to align de novo peptides sequences to the expanded human proteome database with a single mismatch allowed. Finally, some peptides may originate from other organisms, especially bacterial or viral sources. For these sources de novo peptide sequences were searched for in the BLAST database (see Methods).

2. Optimal Ordering of Putative Sources Through Estimation of Random Hit Rate

For each potential source (e.g., the computing devices 107-112 of FIG. 1, the peptide sources identified by search steps 703, 705, 707, 709, 711, 713 of FIG. 7, etc.) of peptides described above (e.g., FIG. 3, databases, queries 701, 703 of FIG. 7, etc.), including cis- and trans-splicing of peptides, the chances of finding a match randomly were determined. To estimate the random hit rate associated with each potential putative source of a peptide, it was determined how many randomly generated sequences could be found in each source (e.g. number of randomly generated sequences found in each search step 703, 705, 707, 709, 711, 713 of FIG. 7). The random hit rate associated with a source and/or search step can further depend on database size and/or complexity. 8-12mer peptide sequences (1,000 per length) were generated in two ways: random sequences uniformly sampling all amino acids (referred to below as uniform random) or sequences with frequencies of amino acids matching those found in vertebrates (referred to below as weighted random); see Table 1. However, peptides 8-14 amino acids in length could have been used (e.g., but not limited to, 8-14 amino acids, 9-14 amino acids, 10-14 amino acids, 11-14 amino acids, 12-14 amino acids, 13-14 amino acids, 8-13 amino acids, 8-12 amino acids, 8-11 amino acids, 8-10 amino acids, 8-9 amino acids, 9-13 amino acids, 9-12 amino acids, 9-11 amino acids, 9-10 amino acids, 10-13 amino acids, 10-12 amino acids, 10-11 amino acids, 11-13 amino acids, 11-12 amino acids, 12-13 amino acids).

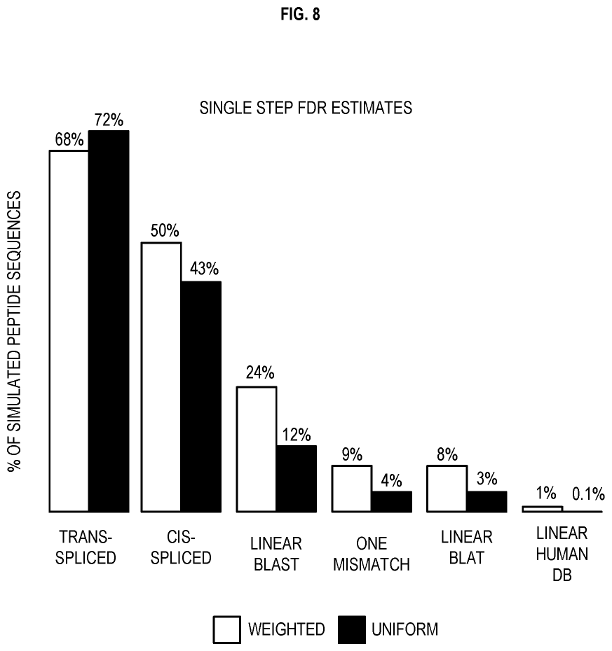

The random sequences were used to estimate the random hit rate of each potential source of peptides (FIG. 8). Using the uniform random sequences, fourteen out of 5,000 peptides (0.28%) were found in the expanded human proteome (e.g., but not limited to, UniProt, OpenProt, lncRNAs, miRNAs and HERVs). When searching in canonically non-coding regions of the human genome using BLAT, 178 out of 5,000 (3.3%) of random peptides could be mapped. Estimating the random hit rate when searching for peptides mapping to the human proteome with a single mismatch was also determined; it was found that 192 out of 5,000 (3.9%) peptides could be mapped. When searching for peptides that may come from non-human organisms in the BLAST database, 604 out of 5,000 (12.1%) sequences could be mapped. Finally, when searching for cis-spliced peptides in the expanded human proteome, 1,936/5,000 (38%) could be mapped; for trans-spliced peptides, 3,598/5,000 (71%) could be mapped (FIG. 8). For weighted random peptides, 50% and 68% of peptides could be assigned as cis- or trans-spliced, respectively (FIG. 8).

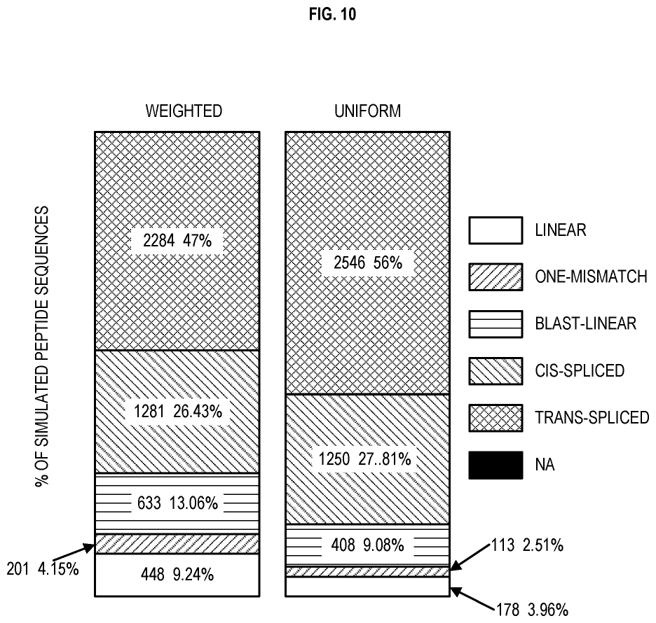

An enhanced peptide mapping pipeline to assign sources for peptides in order of decreasing random hit rate was designed. When using either set of simulated data to order peptide sources by random hit rates, the enhanced pipeline searches for peptide sources in the following order: 1) the expanded human proteome database (assigned as linear), 2) the non-coding regions of the human genome using BLAT (assigned as linear), 3) single mismatch peptides in the expanded human proteome (assigned as linear), 4) the BLAST database (assigned as linear), 5) cis-spliced and 6) trans-spliced peptides in the expanded human proteome. When ordered in series, 4,495/5,000 (90%) of uniform random sequences and 4,847/5,000 (97%) of weighted random sequences are found with this pipeline (FIG. 10); while this random hit rate is high, researchers can choose an appropriate threshold and exclude mapped peptides from high-random hit rate sources. Indeed, previous studies have excluded searches for trans-spliced peptides due to the presumed high random hit rate and assumed rarity of occurrence.

3. Peptide Whose Sequences are Assigned with Higher Confidence During De Novo Sequencing are Identified in Earlier Parts of the Assignment Workflow

A test was performed to determine whether the proportion of peptides found in real experiments are consistent with the final order. The peptides source assignment workflow as applied to six novel immunopeptidomics data sets from IM9 and Raji cell lines (see Methods). During de novo sequencing, amino acid calls are given local confidence scores; the quality of the sequencing across the peptide can be quantified by the average local confidence (ALC %) score. The ALC % score is generated by MS/MS and associated with each de novo peptide sequence 701 (FIG. 7).

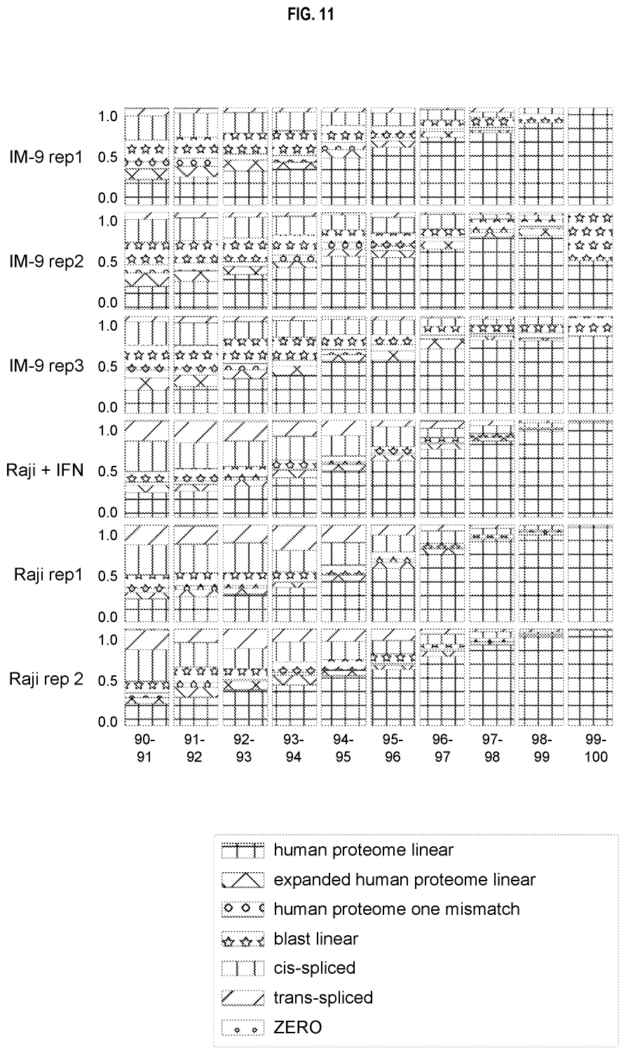

It was hypothesized that peptides with higher ALC % are more likely to be assigned to more reliable sources with lower random hit rates, i.e. sources earlier in our workflow. Indeed, across six experiments the majority of peptides with the highest ALC % were found in the first source of the pipeline (linear expanded human proteome source), in stark contrast to the pattern of sources found for randomly generated peptides (FIG. 11). With decreasing ALC %, more peptides can be found in later sources in the pipeline with the most striking increases for cis-spliced peptides in both cell lines, and blast- and trans-spliced peptides for samples from the IM9 and Raji cell lines, respectively (FIG. 11). Depending on the true make up of a particular sample, different sources of the pipeline are likely to be differentially enriched in the final calls.

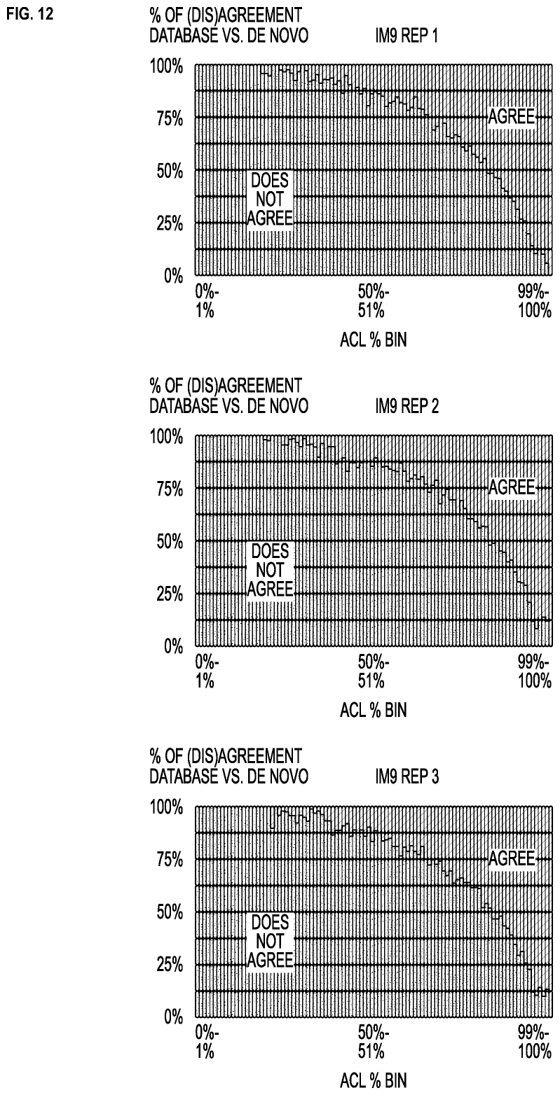

The second fragmentation in MS/MS experiments (MS2 scans) can be inherently noisy due to poor fragmentation or ionization of certain peptides. To evaluate the proportion of ambiguous de novo calls as a function of ALC %, a set of MS2 scans was taken from IM9 cell lines for which both de novo identified peptides as well as conventional database calls were available. As hypothesized, the de novo ALC % goes down so does the proportion of peptide calls that agrees between de novo and conventional database searches (FIG. 12). Taken together, this shows that de novo peptide sequences with low ALC % and their sources should be placed under additional scrutiny.

4. Re-Analysis of Monoallelic Cell Line Data Using the Peptide Source Assignment Workflow

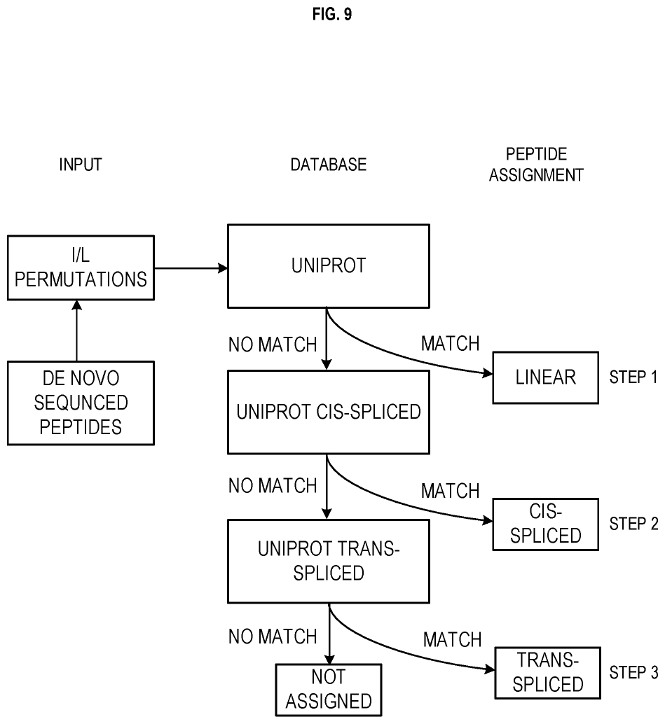

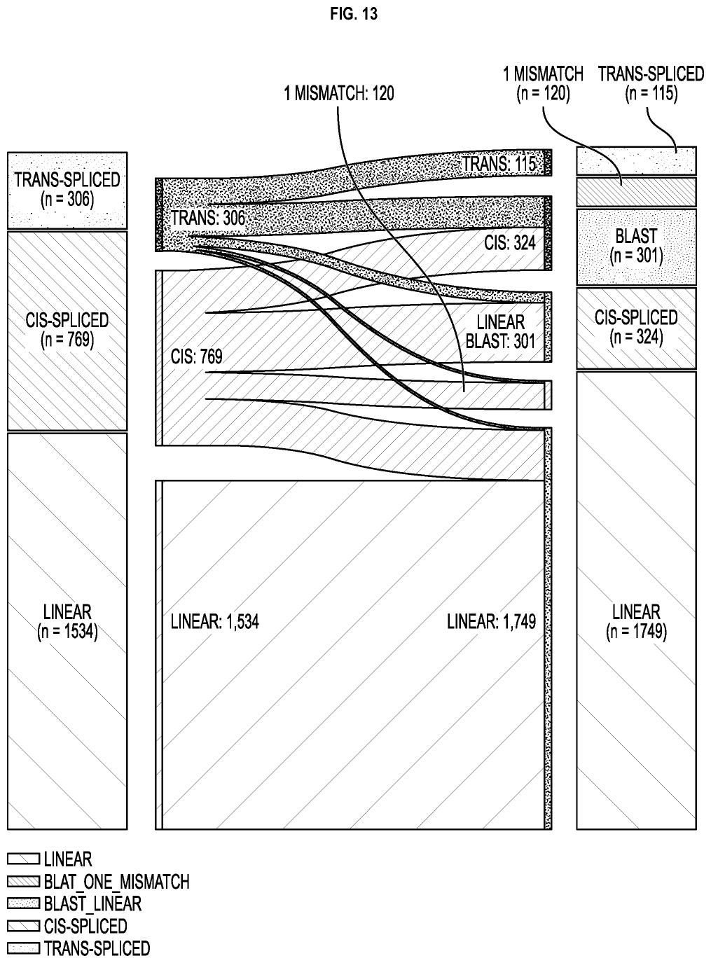

Peptide identification by the peptide source assignment workflow was compared versus hybridfinder on peptides eluted from MHC complexes on the data set from the hybridfinder publication: immunopeptidomics from a collection of cell lines engineered to express a single HLA allele. See FIG. 9 for hybridfinder workflow. It was found that a large fraction of peptides that hybridfinder identifies as cis- or trans-spliced can also be mapped to sources with much lower random hit rates. For example, it was found that for the cell line expressing HLA-A*02:04, of the 1,075 peptides classified as spliced by hybridfinder, 215 could be classified as linear from the expanded human database, 120 could be classified as linear with one mismatch, and 301 could be classified as linear from the BLAST database; overall 636/1,075 (60%) of putatively spliced peptides were reclassified as linear. Additionally, 133 of the peptides that were classified as trans-spliced could be reclassified as cis-spliced using the expanded human proteome (FIG. 10). Across all cell lines, 36% of putative cis-spliced peptides can be reclassified as linear, and 45.9% of putative trans-spliced peptides are reclassified as linear or cis-spliced (FIG. 13).

The peptide source assignment workflow presented here shows that putative spliced peptides are likely peptides stemming from mutated DNA sequences, non-canonically spliced RNA sequences, non-canonically translated regions of the human genome, mismatched human sequences or bacterial proteins. Altogether, 20% of peptides are assigned as spliced peptides with the workflow presented here, down from 29% using hybridfinder (FIG. 13). This overall reduction in identification of putative spliced peptides is notable, as it is part of the subject exemplary method which provides a workflow which results in a higher confidence of peptide assignment due to assigning peptides to a putative source with lowest random hit rate. Because spliced peptides have the highest random hit rate compared to other potential putative sources presented herein, it is likely that a significant portion of peptides assigned as spliced by hybridfinder are improperly assigned. The workflow presented herein is therefore an improvement over hybridfinder because of the overall reduction in identification of putative spliced peptides compared to hybridfinder. In some embodiments, the method of the present invention reduces identification of spliced peptides by 5-60%. In some embodiments, the method of the present invention reduces identification of spliced peptides by 5-50%. In some embodiments, the method of the present invention reduces identification of spliced peptides by 5-40%. In some embodiments, the method of the present invention reduces identification of spliced peptides by 5-30%. In some embodiments, the method of the present invention reduces identification of spliced peptides by 5-20%. In some embodiments, the method of the present invention reduces identification of spliced peptides by 5-10%. In some embodiments, the method of the present invention reduces identification of spliced peptides by 10-60%. In some embodiments, the method of the present invention reduces identification of spliced peptides by 10-50%. In some embodiments, the method of the present invention reduces identification of spliced peptides by 10-40%. In some embodiments, the method of the present invention reduces identification of spliced peptides by 10-30%. In some embodiments, the method of the present invention reduces identification of spliced peptides by 10-20%.

In some embodiments, the method of the present invention reduces identification of spliced peptides by 20-60%. In some embodiments, the method of the present invention reduces identification of spliced peptides by 30-60%. In some embodiments, the method of the present invention reduces identification of spliced peptides by 40-60%. In some embodiments, the method of the present invention reduces identification of spliced peptides by 50-60%. In some embodiments, the method of the present invention reduces identification of spliced peptides by 20-50%. In some embodiments, the method of the present invention reduces identification of spliced peptides by 30-40%.

In some embodiments, the method of the present invention reduces identification of spliced peptides by 5-70%. In some embodiments, the method of the present invention reduces identification of spliced peptides by 14-60%.

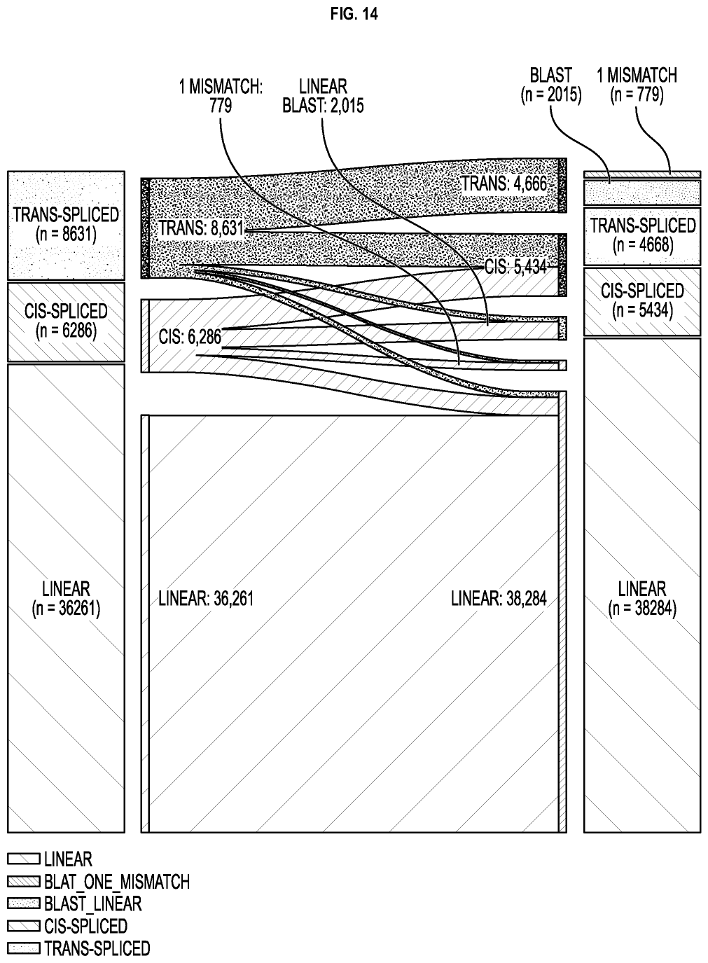

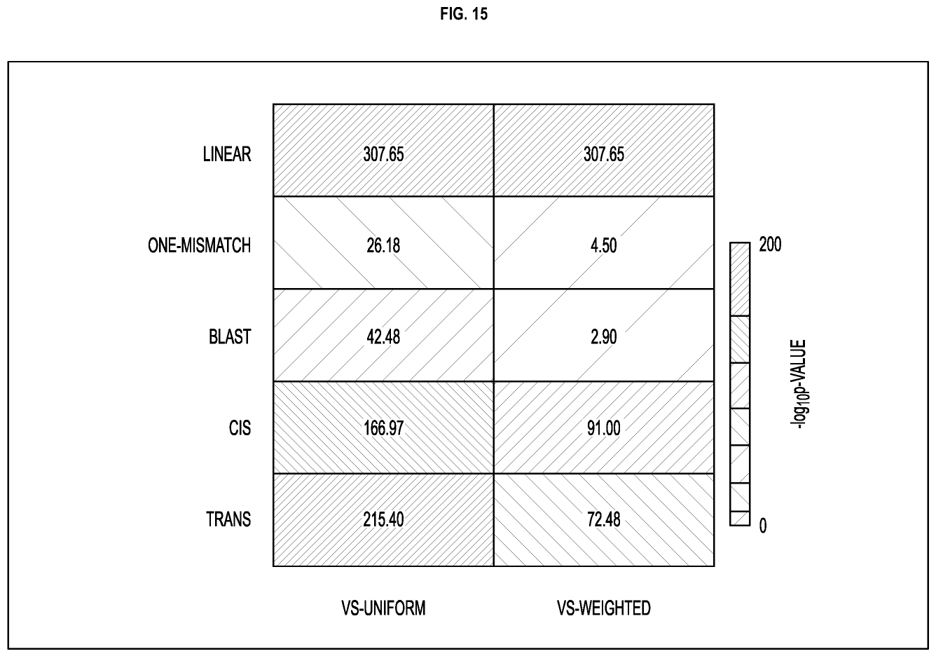

At each step, the random peptides mapping results were used to estimate how many peptides were likely found by chance. When compared to weighted random peptides, more peptides detected in cell lines were assigned as linear (P<1e-308, two-sided Fisher's exact test), linear with a single mismatch (P=3.16e-05), linear from the BLAST database (P=0.00126), cis-spliced (P=9.9e-92) and trans-spliced (P=3.29e-73) (FIG. 14). More peptides were found that would be expected by searching for random peptides, this indicates that, due to optimal ordering of assignment of sources, each source is contributing to the immunopeptidome found in each cell line.

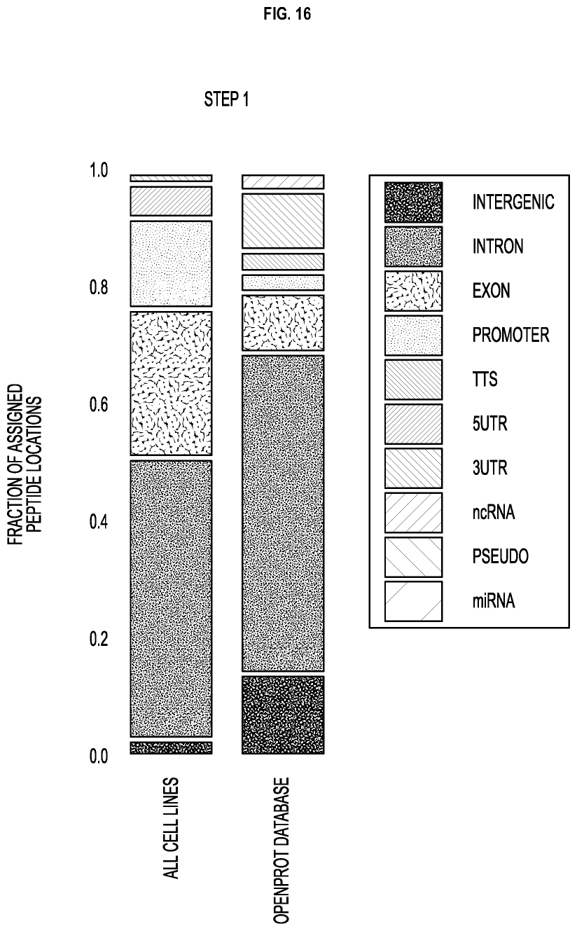

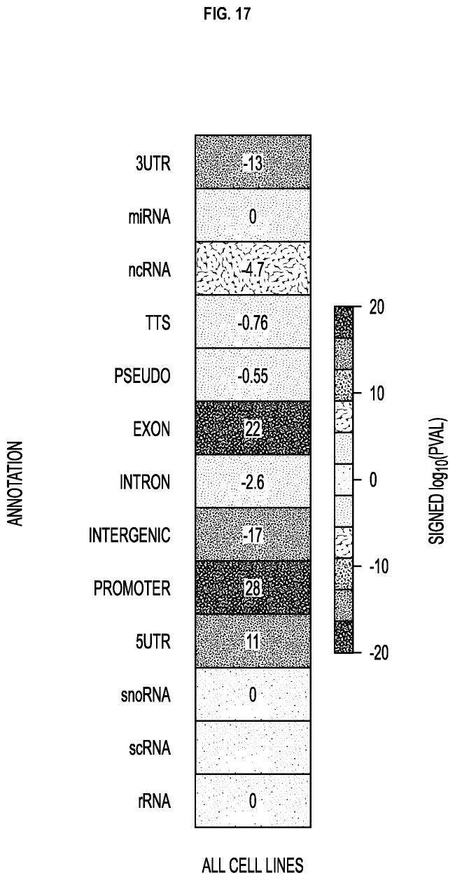

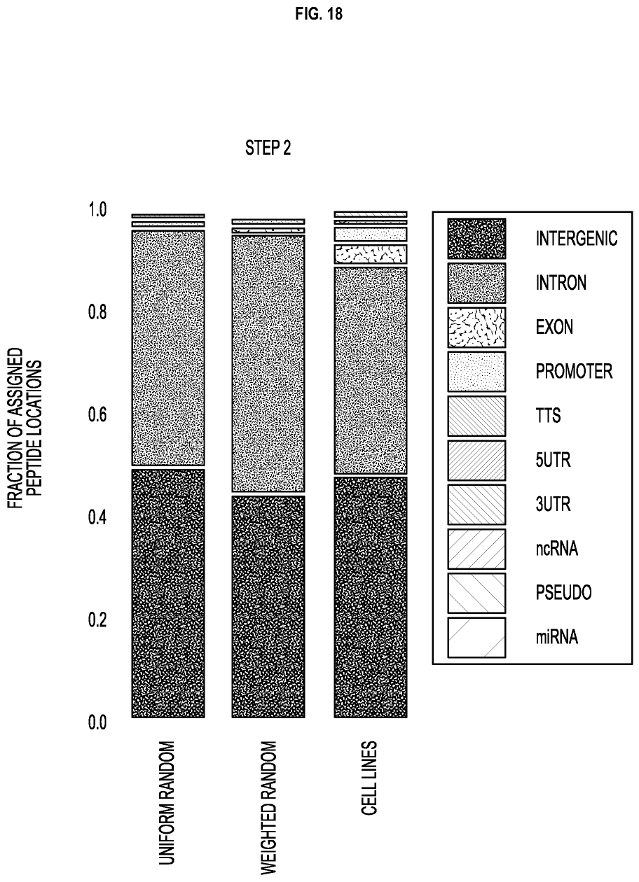

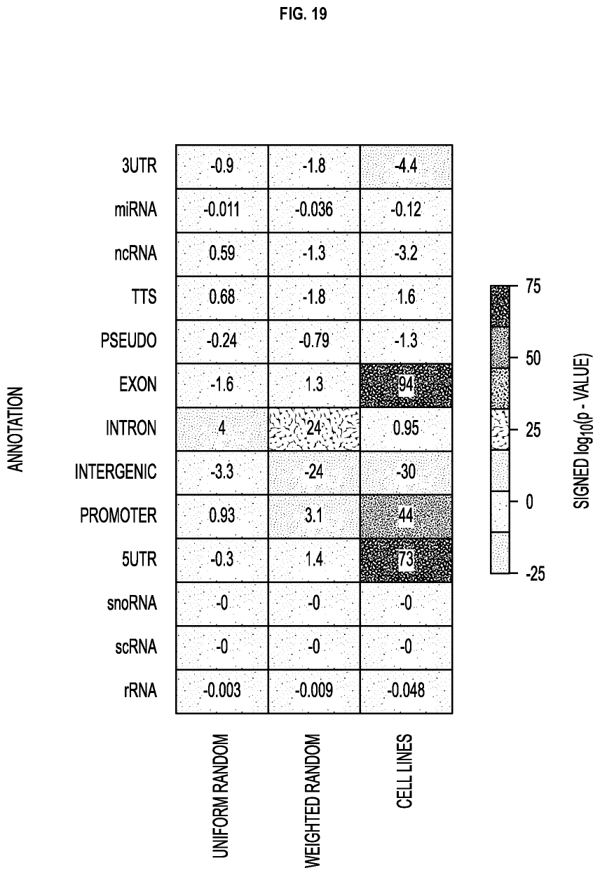

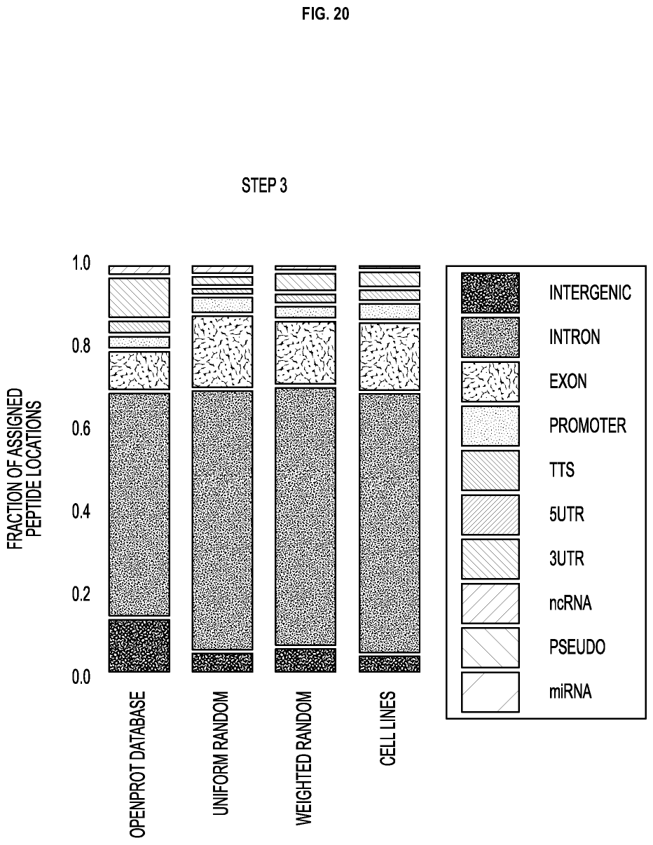

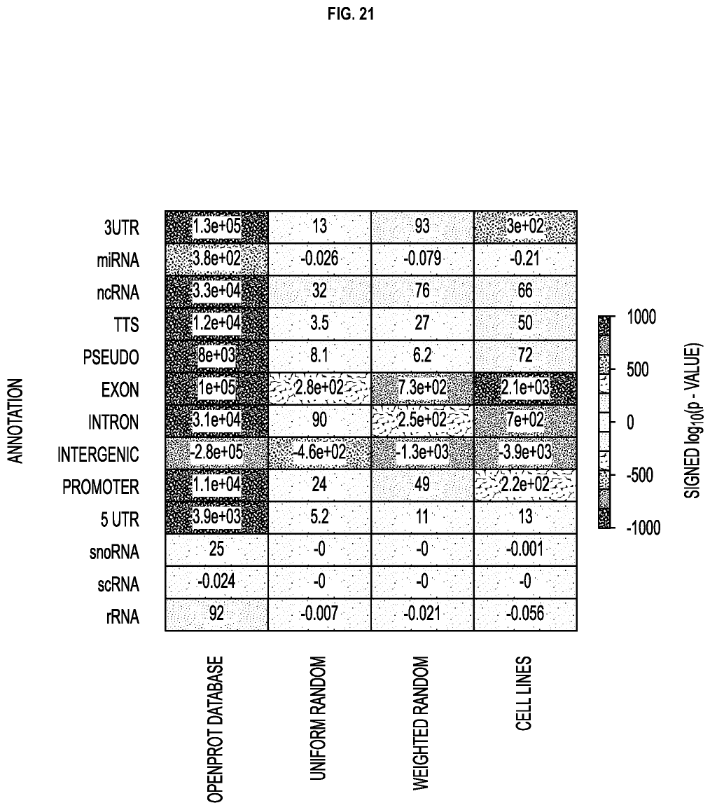

5. Peptides that Map Throughout the Human Genome are Enriched for Expressed Regions

Where peptides that map within the human genome, but outside of the UniProt proteome, land in terms of genomic annotations were examined. The first three steps of the pipeline can map a peptide to regions of the human genome. In the first step, peptides that map exclusively in the OpenProt database land in ORFs that are not in the UniProt human proteome. The location of these peptides in the human genome was analyzed (FIG. 16) and, compared to the locations of all proteins in OpenProt, these peptides are enriched in exons, promoters and 5′UTRs (FIG. 17). The exonic enrichment is likely due to out-of-frame translation. In the subsequent step, peptides are mapped to a 6-frame translation of the human genome, though since OpenProt includes all proteins originating from ORFs longer than 30 amino acids, these peptides must come from ORFs shorter than 30 amino acids. The genomic annotation distribution of peptides mapped at this step is closer to that of the human genome, i.e. the majority of peptides map to intergenic or intronic regions (FIG. 18), indicating these peptides assignments are contaminated with random matching. However, peptides from proteomic data sets were more enriched for exons, promoters and 5′UTRs than peptides from the random uniform or weighted simulations (FIG. 19). In the third step of the enhanced pipeline, peptides can be mapped to the expanded human proteome with a single mismatch (FIG. 20). Peptides mapped at this step show stronger enrichment in exons, introns and promoters than the enrichment found from peptides in the weighted or uniform simulated data sets (FIG. 21). At each step, there is consistent depletion of intergenic regions and enrichment of transcribed regions, as has been found in other studies focused on unidentified peptides in the immunopeptidome. The enrichment of transcribed sequences supports the idea that the peptides assigned in these steps of the pipeline are correctly assigned, even though they do not map to proteins in the UniProt database.

6. Peptides Identified by BLAST are not Enriched for any Microbial Genus

The search within the BLAST database has the highest random hit rate for linear peptides in the peptide source assignment workflow. While peptides from cell lines had modestly more matches in the BLAST database than would be expected based on the uniform or weighted random data (FIG. 14), it was determined if the BLAST assignments show enrichment of specific microorganisms which could be contaminants. To calculate enrichment, peptides that could not be uniquely mapped to a single species were removed; the Fisher's exact test was then applied to the counts of peptides mapping to each genus in each cell line as well as all cell lines. After correcting for multiple hypothesis testing, there were no significantly enriched genera in any cell line, or when considering all cell lines together. There were three possible, non-mutually exclusive causes for observing a lack of enrichment. First, there are no contaminating organisms in the immunopeptidomics preparation. Second, the organisms present are not represented in the BLAST database. Third, the peptides stemming from contaminating organisms cannot be uniquely mapped to a single organism, and therefore were excluded from the above analysis. In the first two possibilities, it is not clear why the cell lines have more BLAST matches than expected. Taken together, these results do not support a biological underpinning for the peptides assigned at the BLAST step; rather, it is likely that the matches found at this step are random and spurious.

7. Recurrent Novel Peptide

To identify reclassified common peptides across multiple datasets peptides shared by more than three cell lines were selected. For example, QSPVALRPL (SEQ ID NO:5) is highly recurrent, and was identified as trans-spliced by the hybridfinder algorithm, but was reclassified as linear using the disclosed pipeline. The same peptide is listed in the Immune Epitope Database as being a part of an unidentified protein. Upon further inspection, this is an out-of-frame peptide in the FAM96A gene, a pro-apoptotic tumor suppressor in gastrointestinal stromal tumors (see, e.g., Schwamb et al. Int. J. Cancer (2015) September 15; 137(6):1318-29 incorporated by reference herein in its entirety). If out-of-frame translation is specific to cancer samples, this peptide could be a cancer immunotherapy target.

### B. Discussion

With the increasing number of peptides identified in immunopeptidomics experiments using de novo sequencing, the need for better characterization of the immunopeptidome is more pressing than ever. Previous studies attributed PCPS as the primary source of peptides of unknown protein identity. Described herein is a peptide source assignment workflow that assigns parental proteins of de novo sequenced peptides from several sources with lower random hit rate than the set of all possible PCPS peptides. It was found that 32% of putative PCPS peptides can be explained by single mismatches with known proteins, translational of supposedly untranslated parts of the human genome, or bacterial and viral peptides. Not surprisingly, the majority of peptides are encoded by known expressed regions. Finally, a recurrent out-of-frame peptide was identified in the tumor suppressor gene FAM96A that could be of interest as a cancer immunotherapy target.

### C. Methods

1. Datasets

i. Simulated Random Peptides

Two sets of peptide sequences were simulated for random hit rate estimation. The “random” built-in python library was used to produce sets of 8-12 length amino acid sequences, 1,000 peptides for each length, a total of 5,000 random peptides in each set. For the first simulated peptide sequence set, all amino acids have an equal probability of being incorporated into a sequence; this set is referred to as “uniform random”. In the second set, the amino acids have a probability of being incorporated that matches their frequency in vertebrates; this set is referred to as “weighted random”. The two sets of peptide sequences are included in Table 1.

ii. IM9 and Raji Cell Line Immunopeptidomics

Three replicates of IM9 and Raji cell line were processed through MS/MS:
210210_IM-9_1_IFN_cl1,
210210_IM-9_2_IFN_cl1,
210210_IM-9_3_IFN_cl1,
180316 RAJI NoIFN,
180323_Raji_IFN, and
180323_Raji. All replicates of IM9 cell line were simulated with IFNγ while only two replicates of Raji cell line received the same treatment.

IFNγ can enhance expression of surface major histocompatibility complex (HLA) molecules and increase the processing and presentation of tumor-specific antigens, facilitating T-cell recognition and cytotoxicity. IFNγ also up-regulates many components of the antigen presenting pathway, as well as induces a shift between the constitutive to immunoproteasome subunits which have different catalytic activity in the proteosome, generating a different population of HLA-associated peptides. We use IFNγ treatment with cell lines to increase the chances to expand our detectable immunopeptidome by mass spectrometry.

a. Immunoprecipitation

HLA-Pan Class I (W6/32) columns were prepared using NHS-activated Sepharose 4 beads (GE Healthcare 17090601) and a coupling buffer of 0.2M sodium bicarbonate and 0.5M sodium chloride; they were washed with 0.1M Tris hydrochloride with a pH of 8.5, and 0.1M acetate buffer. Affinity purification was performed under gravity and the flow-through was captured for further analysis. 0.1M glycine (Sigma) pH 2.7 was used to elute bound HLA molecules under gravity (FIG. 1). 0.1% trifluoroacetic acid (Cat no: LC485-1 Honeywell) was added to the glycine elute. The HLA-associated-peptides were eluted using Sep-Pak (Cat no: WAT054960 Waters) with two-step elution. HLA-specific peptides were eluted using 30% acetonitrile (Cat no: LC34967 Honeywell)/0.1% trifluoroacetic acid and the HLA molecules were eluted using 70% acetonitrile/0.1% trifluoroacetic acid. Aliquots of the lysate, flow-through, glycine, 30% acetonitrile/0.1% trifluoroacetic acid, and 70% acetonitrile/0.1% trifluoroacetic acid eluates were collected throughout the process.

Peptides and HLAs fractions were placed on SpeedVac Vacuum Concentrator (Thermo) for 2 hours. Each sample, after SpeedVac, was resuspended in 0.10% trifluoroacetic acid. The peptide fractions were purified further using a C-18 ZipTip® (Cat no: ZTC185096 Millipore). All samples were then analyzed with the Orbitrap Fusion™ Lumos™ Tribrid™ Mass Spectrometer (Thermo) for peptide sequencing.

b. Data Analysis

Raw data files from the Orbitrap Fusion™ Lumos™ (Thermo) LC/MS were searched with the PEAKS® Studio X (BSI) proteomics software against Human Uniprot Database, custom databases for proteins of interest, and de novo.

iii. HLA-Monoallelic Immunopeptidomics

For the MS/MS data from HLA-I monoallelic cell lines, the peptides were downloaded from the supplementary table of Faridi, P. et al. Sci. Immunol. Vol 3, issue 28, pg 3947, October 12 (2018), incorporated herein by reference in its entirety. The data includes the expression of eight HLA-A alleles (A0101, A0203, A0204, A0207, A0301, A3101, A6802, A2402) and nine different HLA-B alleles (B5801, B5703, B5701, B4402, B5101, B0801, B1502, B2705, B0702). In total, there were more than 51,000 unique peptides.

2. Recapitulation of Hybridfinder

For ease of comparison to the described peptide source assignment workflow, the workflow was recapitulated from hybridfinder as described in Faridi, P. et al. Sci. Immunol. Vol 3, issue 28, pg 3947, October 12 (2018), incorporated herein by reference in its entirety. First, each peptide is sought in the UniProt human reference proteome database. Peptides with identical matches are annotated as linear. For peptides with no linear matches, all possible splits of that peptide where the length of the smaller piece is longer than 1 amino acid were generated. Then, potential matches for each fragment were searched through the database. The peptide was annotated as cis-spliced if identical matches of both fragments were detected in a single protein. The matches can be reverse-ordered. Otherwise, if the matches are available in two distinct proteins, the peptide was annotated as trans-spliced. Peptides for which no split pairs match to any protein sequences are annotated as not available (N/A).

3. The Expanded Human Proteome Database

FASTA files of OpenProt (www.openprot.org), UniProt (www.uniprot.org) reviewed and unreviewed human sequences, which also includes protein sequences from some viruses that use humans as hosts (UniProt proteome version UP0000056430, downloaded in May 2020) were combined. This database was expanded to include translated proteins sequences from lncRNAs (NONCODE Version v5.19, downloaded in May 2020), miRNAs (last modified Mar. 10, 2018, downloaded in May 2020), and endogenous viral elements (gEVE database ORFs21, downloaded in May 2020). This database is used when the workflow searches for linear human peptides and single-mismatched human peptides (steps 1 and 3), as well as in the search for cis- and trans-spliced peptides.

4. The Peptide Source Assignment Workflow

The random hit rate inherent was measured in each source from which peptides in immunopeptidomics experiments can be found using the simulated random datasets described above. The steps of the workflow were ordered in order of ascending random hit rate to construct the workflow. The steps applied to each de novo-sequenced peptide are as follows:

Step 1: Search for identical sequence matches in the expanded human proteome database (described above). Leucine (L) and isoleucine (I) have the same mass; therefore it is impossible to differentiate them in de novo search sequencing. To account for this, for a given peptide containing I/L all permutations of I and L residues are considered. For example, for the peptide “ATTSLLHN (SEQ ID NO:1)” there are four possible permutations: ATTSLLHN (SEQ ID NO:1), ATTSLIHN (SEQ ID NO:2), ATTSILHN (SEQ ID NO:3), and ATTSIIHN (SEQ ID NO:4). If the algorithm finds an identical match (e.g., 100% identical) for any permutation, the peptides are annotated as “Linear”, and all possible protein sources of the peptide are included in the output. The algorithm need not progress to additional steps, e.g., continuing with step 2, since the match has been identified. Otherwise, if a match is not identified, the algorithm progresses to step 2.

Step 2: Search for an identical match in any of the six frames of the translated human genome using BLAT32. The following commands are used:
blat -t=dnax -q=prot -minScore=7 -stepSize=1 hg38.2 bit Fasta_query output.psl
psl2bed<output.psl>perfect_match.bed

If an identical match is found, that peptide is annotated as “Linear” and possible source sequences are included in the output. Otherwise the peptide is passed to the step 3.

Step 3: Peptides are mapped to the expanded human proteome database, this time allowing one mismatch using this code: “blat -t=prot -q=prot -minScore=7 -stepSize=1 combined DB.processed.fasta Fasta_query output_blat_hits.psl” in a genomic location of BLAT hits analysis.

If a sequence with a single mismatch is found, the peptide is annotated as “one mismatch”. Otherwise the peptide is passed to Step 4.

Step 4: Sequences are mapped to other organisms using the BLAST NCBI tool. If any identical matches are found the results are annotated as “LINEAR BLAST”.

Step 5: For the remaining peptides, the algorithm generates all possible splits of the peptide where the length of the smaller piece is larger than 1. Then it looks for matches of both fragments in all human sequence databases. If there is a match for both chunks in the same protein, the tool annotates the peptide as “cis-spliced”. Otherwise, if there are hits for both fragments in two different proteins, the tool annotates the peptide as “trans-spliced”. The rest of peptides that do not have any matches are assigned as not available (N/A).

5. Genomic Location of BLAT Hits Analysis

Analysis of the genomic locations of BLAT hits was performed using the annotatepeaks.pl script from the HOMER suite. Specifically:
annotatePeaks.pl ${file} hg38 -annStats ${file}.summary.txt

Only basic annotations were considered for further analysis. To calculate the enrichment of genomic locations of peptides found in the OpenProt database with either an identical match (step 1) or with a single mismatch (step 3), a fisher's exact test was performed to compare the number of peptides in each genomic annotation in the sample versus in the whole OpenProt database. For peptides that mapped to any translated region in the human genome (step 2), the p-value enrichment calculated by HOMER was used for over or underrepresentation of each genomic annotation.

6. Tools

Python, bedops, psl2bed, BLAT, BLAST, HOMER.

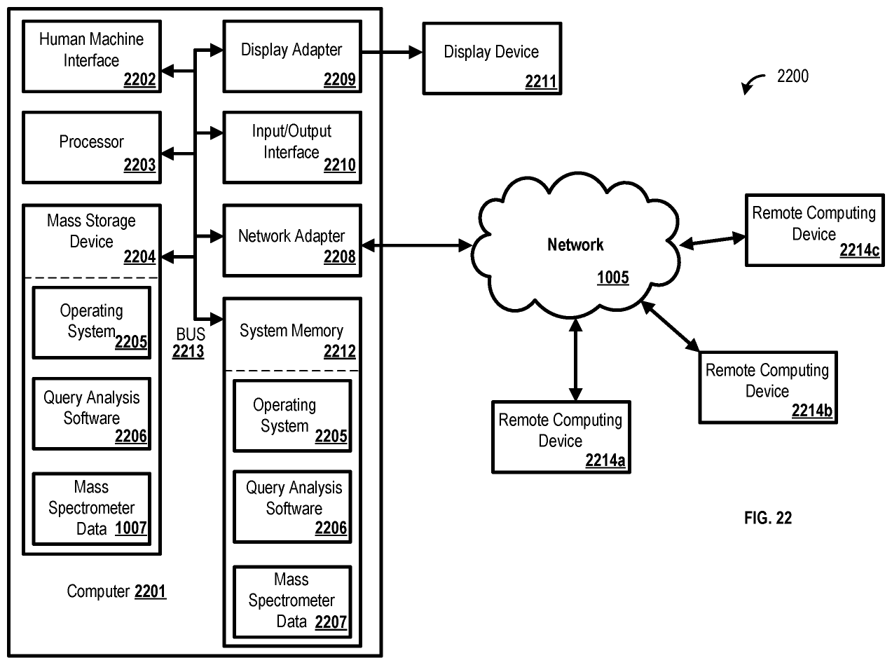

FIG. 22 shows a system 2200 for performing the methods described herein. In an embodiment, the system 2200 can be configured to execute the workflow illustrated in FIG. 7. In an embodiment, the system 2200 can include some or all of the databases utilized by the workflow illustrated in FIG. 7. In an embodiment, the system 2200 can be configured to communicate to one or more of the databases utilized by the workflow illustrated in FIG. 7. In an embodiment, the system 2200 can include some or all of the data sources 518A-518N illustrated in FIG. 5. In an embodiment, the system 2200 can be configured to communicate with one or more of the data sources 518A-518N illustrated in FIG. 5.

Any device/component described herein may include a computer 2201 as shown in FIG. 22. The computer 2201 may comprise one or more processors 2203, a system memory 2212, and a bus 2213 that couples various components of the computer 2201 including the one or more processors 2203 to the system memory 2212. In the case of multiple processors 2203, the computer 2201 may utilize parallel computing.

The bus 2213 may comprise one or more of several possible types of bus structures, such as a memory bus, memory controller, a peripheral bus, an accelerated graphics port, and a processor or local bus using any of a variety of bus architectures.

The computer 2201 may operate on and/or comprise a variety of computer-readable media (e.g., non-transitory). Computer-readable media may be any available media that is accessible by the computer 2201 and comprises, non-transitory, volatile and/or non-volatile media, removable and non-removable media. The system memory 2212 has computer-readable media in the form of volatile memory, such as random access memory (RAM), and/or non-volatile memory, such as read-only memory (ROM). The system memory 2212 may store data such as mass spectrometry data 2207 and/or program modules such as operating system 2205 and query analysis software 2206 that are accessible to and/or are operated on by the one or more processors 2203. The system memory 2212 can further include some or all of the databases utilized by the workflow illustrated in FIG. 7 and/or some or all of the data sources 518A-518N illustrated in FIG. 5.

The computer 2201 may also comprise other removable/non-removable, volatile/non-volatile computer storage media. The mass storage device 2204 may provide non-volatile storage of computer code, computer-readable instructions, data structures, program modules, and other data for the computer 2201. The mass storage device 2204 may be, but is not limited to, a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Any number of program modules may be stored on the mass storage device 2204. An operating system 2205 and query analysis software 2206 may be stored on the mass storage device 2204. One or more of the operating system 2205 and query analysis software 2206 (or some combination thereof) may comprise program modules and the query analysis software 2206. Mass spectrometry data 2207 may also be stored on the mass storage device 2204. Mass spectrometry data 2207 may be stored in any of one or more databases known in the art. The databases may be centralized or distributed across multiple locations within the network 2215. The mass storage device 2204 can further include some or all of the databases utilized by the workflow illustrated in FIG. 7 and/or some or all of the data sources 518A-518N illustrated in FIG. 5.

A user may enter commands and information into the computer 2201 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 2203 via a human-machine interface 2202 that is coupled to the bus 2213, but may be connected by other interface and bus structures, such as a parallel port, game port, an IEEE 1394 Port (also known as a Firewire port), a serial port, network adapter 2208, and/or a universal serial bus (USB).

A display device 2211 may also be connected to the bus 2213 via an interface, such as a display adapter 2209. It is contemplated that the computer 2201 may have more than one display adapter 2209 and the computer 2201 may have more than one display device 2211. A display device 2211 may be a monitor, an LCD (Liquid Crystal Display), a light-emitting diode (LED) display, a television, a smart lens, smart glass, and/or a projector. In addition to the display device 2211, other output peripheral devices may comprise components such as speakers (not shown) and a printer (not shown) which may be connected to the computer 2201 via Input/Output Interface 2210. Any step and/or result of the methods may be output (or caused to be output) in any form to an output device. Such output may be any form of visual representation, including, but not limited to, textual, graphical, animation, audio, tactile, and the like. The display 2211 and computer 2201 may be part of one device, or separate devices.

The computer 2201 may operate in a networked environment using logical connections to one or more remote computing devices 2214a,b,c. A remote computing device 2214a,b,c may be a personal computer, computing station (e.g., workstation), portable computer (e.g., laptop, mobile phone, tablet device), smart device (e.g., smartphone, smartwatch, activity tracker, smart apparel, smart accessory), security and/or monitoring device, a server, a router, a network computer, a peer device, edge device or other common network nodes, and so on. Logical connections between the computer 2201 and a remote computing device 2214a,b,c may be made via a network 2215, such as a local area network (LAN) and/or a general wide area network (WAN). Such network connections may be through a network adapter 2208. A network adapter 2208 may be implemented in both wired and wireless environments. Such networking environments are conventional and commonplace in dwellings, offices, enterprise-wide computer networks, intranets, and the Internet.

Application programs and other executable program components such as the operating system 2205 are shown herein as discrete blocks, although it is recognized that such programs and components may reside at various times in different storage components of the computing device 2201, and are executed by the one or more processors 2203 of the computer 2201. An implementation of query analysis software 2206 may be stored on or sent across some form of computer-readable media. Any of the disclosed methods may be performed by processor-executable instructions embodied on computer-readable media.

In an embodiment, the query analysis software 2206 may be configured to execute some or all of the search steps 703, 705, 707, 709, 711, 713 illustrated in FIG. 7.

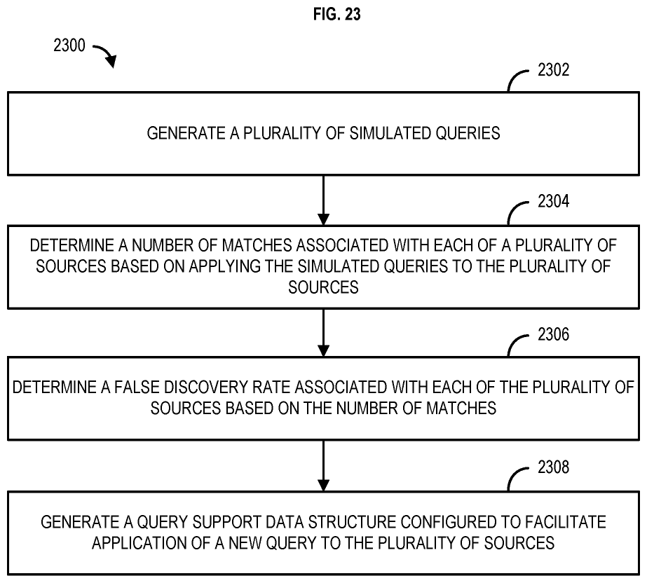

In an embodiment, the query analysis software 2206 may be configured to perform a method 2300, shown in FIG. 23. The method 2300 may be performed in whole or in part by a single computing device, a plurality of electronic devices, and the like. The method 2300 may comprise, at 2302, generating a plurality of simulated random queries. Generating the plurality of simulated random queries may include at least one of: generating a plurality of uniform random queries; or generating a plurality of weighted random queries. The plurality of simulated random queries may include a plurality of simulated random text strings. The plurality of simulated random queries may include a plurality of simulated random peptide sequences.

The method 2300 may comprise, at 2304, determining, based on applying the plurality of simulated random queries to each source of a plurality of sources, a number of matches associated with each source.

In an embodiment, the method 2300 may comprise, at 2306, determining, based on the numbers of matches associated with each source, a false discovery rate associated with each source. In an embodiment, a function of the number of matches and a number of the plurality of simulated random queries may be determined. In an embodiment, a determination may be made by dividing the number of matches by a number of the plurality of simulated random queries. In an embodiment, determining, based on the numbers of matches associated with each source, the false discovery rate associated with each source may include a function of the number of matches and a number of the plurality of simulated random queries. In an embodiment, determining, based on the numbers of matches associated with each source, the false discovery rate associated with each source may include dividing the number of matches by a number of the plurality of simulated random queries.

The method 2300 may comprise, at 2308, generating, based on the false discovery rates, a query support data structure configured to facilitate application of a new query to the plurality of sources.

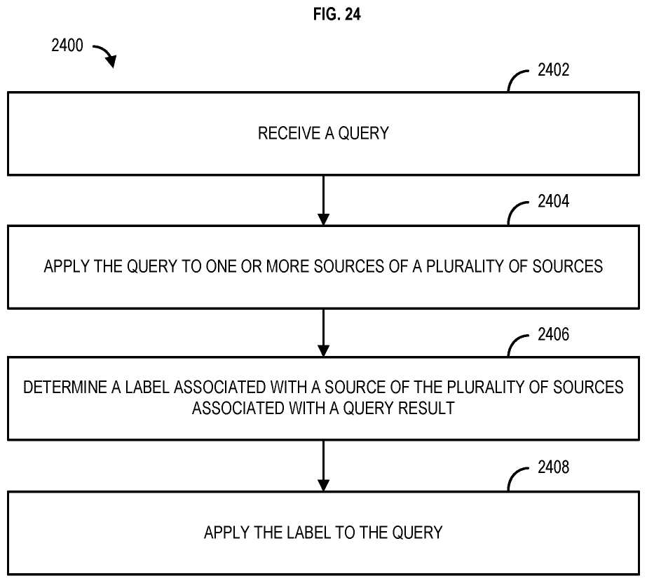

In an embodiment, the query analysis software 2206 may be configured to perform a method 2400, shown in FIG. 24. The method 2400 may be performed in whole or in part by a single computing device, a plurality of electronic devices, and the like. The method 2400 may comprise, at 2402, receiving a query. The query may include a text string. The query may include a peptide sequence. Receiving the query may include receiving the peptide sequence from a mass spectrometer system. The method 2400 may include determining, via the mass spectrometer system, one or more amino acids of the peptide sequence.

The method 2400 may comprise, at 2404, applying, based on a query support data structure, the query to one or more sources of a plurality of sources. The query support data structure may indicate an order of the plurality of sources to apply the query. The order may be based on a false discovery rate associated with each source of the plurality of sources. The method 2400 may also include comprising determining one or more permutations of the query. Applying, based on the query support data structure, the query to the one or more sources of the plurality of sources may include: applying each permutation of the one or more permutations of the query to the one or more sources of the plurality of sources; if an identical match to the one or more permutations of the query is found in a first source of the plurality of sources, discontinuing additional searches and applying a linear label to the one or more permutations of the query associated with the identical match; and assigning the one or more permutations of the query associated with the identical match as a correct query.

Applying the query to one or more sources of a plurality of sources may include: searching for an identical match to the query in a first source of the plurality of sources; and if an identical match to the query is found in the first source of the plurality of sources, discontinuing additional searches. The query result may include the identical match and the label associated with a source of the plurality of sources associated with the query result may include a linear label.

Applying the query to one or more sources of a plurality of sources may include: searching for an identical match to the query in a first source of the plurality of sources; and if an identical match to the one or more permutations of the query is found in the first source of the plurality of sources, discontinuing additional searches. The query result may include the identical match and the label associated with a source of the plurality of sources associated with the query result may include a linear label.

Applying the query to one or more sources of a plurality of sources may include: searching for an identical match to the query in any frame of a plurality of frames of a second source of the plurality of sources; and if an identical match to the query is found in any frame of a plurality of frames of the second source of the plurality of sources, discontinuing additional searches. The query result may include the identical match and the label associated with a source of the plurality of sources associated with the query result may include a linear label.

Applying the query to one or more sources of a plurality of sources may include: searching for a non-identical match to the query in a third source of the plurality of sources; and if a non-identical match to the query is found in the third source of the plurality of sources, discontinuing additional searches. The query result may include the non-identical match and the label associated with a source of the plurality of sources associated with the query result may include a mismatch label.

Applying the query to one or more sources of a plurality of sources may include: searching for a homologous match to the query in a fourth source of the plurality of sources; and if a homologous match to the query is found in the fourth source of the plurality of sources, discontinuing additional searches. The query result may include the homologous match and the label associated with a source of the plurality of sources associated with the query result may include a homologous label.

Applying the query to one or more sources of a plurality of sources may include: splitting the query into a plurality of sets of fragments; searching for each set of fragments in a fifth source of the plurality of sources; if a match for a set of fragments is found in the fifth source of the plurality of sources, discontinuing additional searches; and if a first match for a first fragment of the set of fragments and a second match for a second fragment of the set of fragments is found in the fifth source of the plurality of sources, discontinuing additional searches. The query result may include the match for the set of fragments and the label associated with a source of the plurality of sources associated with the query result may include a cis-spliced label. The query result may include the first match for the first fragment of the set of fragments and the second match for the second fragment of the set of fragments and the label associated with a source of the plurality of sources associated with the query result may include a trans-spliced label.

The method 2400 may comprise, at 2406, determining, based on a query result, a label associated with a source of the plurality of sources associated with the query result.

The method 2400 may comprise, at 2408, applying the label to the query. The method 2400 may also include determining, based on the label, a source of the query. The method 2400 may also include validating an output of a mass spectrometer system based on the source of the query.

In view of the described apparatuses, systems, and methods and variations thereof, herein below are described certain more particularly described embodiments of the invention. These particularly recited embodiments should not however be interpreted to have any limiting effect on any different claims containing different or more general teachings described herein, or that the “particular” embodiments are somehow limited in some way other than the inherent meanings of the language literally used therein.

Embodiment 1: A method of determining a putative source of a peptide sequence of a peptide, the method comprising: receiving the peptide sequence; and determining, based at least in part on one or more searches of the peptide sequence within one or more databases, the putative source associated with the peptide sequence, wherein each respective search of the one or more searches has a random hit rate that is based at least in part on a number of random sequences found by the respective search, and wherein the one or more searches are performed in order of increasing random hit rates until the putative source is determined.

Embodiment 2: The embodiment as in the embodiment 1, wherein the one or more databases comprises an expanded human proteome database, and wherein the expanded human proteome database comprises computer-readable representations of translations from messenger ribonucleic acids (RNAs) and non-coding RNAs.

Embodiment 3: The embodiment as in the embodiment 2, wherein the expanded human proteome database comprises computer-readable representations of translations from micro RNAs.

Embodiment 4: The embodiment of any of embodiments 2-3, wherein the expanded human proteome database comprises computer-readable representations of translations from long non-coding RNAs.

Embodiment 5: The embodiment of any of embodiments 2-4, wherein the expanded human proteome database comprises computer-readable representations of translations of human endogenous retroviruses.

Embodiment 6: The embodiment of any of embodiments 2-5, wherein the one or more searches comprises a linear human proteome search for the peptide sequence within the expanded human proteome database, and wherein the putative source is a linear expanded human proteome source when the linear human proteome search for the peptide sequence within the expanded human proteome database finds the peptide sequence within the expanded human proteome database.

Embodiment 7: The embodiment as in the embodiment 6, further comprising: identifying, when the source is the linear expanded human proteome source, whether the peptide is putatively translated from messenger RNA or non-coding RNA.

Embodiment 8: The embodiment of any of embodiments 2-7, wherein the one or more databases comprises a human genome database, wherein the one or more searches comprises a linear human genome search of translations of the human genome database, and wherein the putative source is a linear genome source when the linear human genome search finds human genome sequence from which the peptide is putatively synthesized.

Embodiment 9: The embodiment as in the embodiment 8, wherein the linear human genome search excludes portions of the human genome from which the messenger RNA and the non-coding RNA of the expanded human proteome database are transcribed and includes remaining portions of the human genome, and wherein the linear human genome search comprises a search of six frame translations of the human genome.

Embodiment 10: The embodiment of any of embodiments 2-9, wherein the one or more searches comprises a linear mismatch search for peptides having a mismatch to the peptide sequence within the expanded human proteome database, and wherein the putative source is a linear mismatch of the expanded human proteome when the linear mismatch search finds a peptide sequence having a mismatch to the peptide sequence within expanded human proteome database.

Embodiment 11: The embodiment as in the embodiment 10, wherein the linear mismatch search is a search for peptide sequences having only a single mismatch to the peptide sequence.

Embodiment 12: The embodiment of any of embodiments 1-11, wherein the one or more databases comprises a non-endogenous proteome database comprising computer-readable representations of proteins translated from RNA from non-endogenous organisms and/or proteins synthesized by non-endogenous organisms, wherein the one or more searches comprises a linear non-endogenous search for the peptide sequence within the non-endogenous proteome database, and wherein the putative source is a linear non-endogenous proteome source when the linear non-endogenous search finds the peptide sequence within the non-endogenous proteome database.

Embodiment 13: The embodiment as in the embodiment 12, wherein the non-endogenous proteome database comprises a Basic Local Alignment Search Tool (BLAST) database.

Embodiment 14: The embodiment of any of embodiments 2-13, wherein the one or more searches comprises a cis-spliced search, within the expanded human proteome database, for peptide fragments that can be cis-spliced to match the peptide sequence, and wherein the source is a cis-spliced human proteome source when the cis-spliced search finds, within the expanded human proteome database, peptide fragments that can be cis-spliced to match the peptide sequence.

Embodiment 15: The embodiment of any of embodiments 2-14, wherein the one or more searches comprises a trans-spliced search, within the expanded human proteome database, for computer-readable representations of peptide fragments that can be trans-spliced to match the peptide sequence, and wherein the source is a trans-spliced human proteome source when the trans-spliced search finds, within the expanded human proteome database, computer-readable representations of peptide fragments that can be trans-spliced to match the peptide sequence.

Embodiment 16: The embodiment as in the embodiment 15, wherein the putative source is determined to be unidentified when the trans-spiced search does not find computer-readable representations of peptide fragments that can be trans-spliced to match the peptide sequence.

Embodiment 17: The embodiment of any of embodiments 2-16, wherein the one or more databases comprises a human genome database, and wherein the one or more searches comprise the following searches ordered sequentially in a workflow as follows: a linear human proteome search for the peptide sequence within the expanded human proteome database; a linear human genome search of translations of the human genome database; a linear mismatch search for peptides having a mismatch to the peptide sequence within the expanded human proteome database; and a cis-spliced search, within the expanded human proteome database, for peptide fragments that can be cis-spliced to match the peptide sequence.

Embodiment 18: The embodiment as in the embodiment 17, wherein the one or more databases comprises a non-endogenous proteome database comprising computer-readable representations of proteins translated from RNA from non-endogenous organisms and/or proteins synthesized by non-endogenous organisms, wherein the one or more searches further comprises a linear non-endogenous search for the peptide sequence within the non-endogenous proteome database, and wherein the linear non-endogenous search is ordered sequentially in the workflow after the linear mismatch search and before the cis-spliced search.

Embodiment 19: The embodiment of any of embodiments 17-18, wherein the one or more searches further comprises a trans-spliced search, within the expanded human proteome database, for peptide fragments that can be trans-spliced to match the peptide sequence, and wherein the trans-spliced search is ordered sequentially in the workflow after the cis-spliced search.

Embodiment 20: The embodiment of any of embodiments 17-19, further comprising: halting advancement of the workflow to a subsequent search of the one or more searches when the putative source is determined for the peptide sequence.

Embodiment 21: The embodiment of any of embodiments 1-20, wherein the peptide sequence comprises at least one ambiguous residue, the method further comprising: generating a plurality of permutated peptide sequences each comprising a potential residue for each of the at least one ambiguous residue; determining, for each of the plurality of permutated peptide sequences, a respective potential source; and determining the putative source of the peptide sequence such that the putative source is a respective potential source.

Embodiment 22: The embodiment as in embodiment 21, wherein the potential residue for each of the at least one ambiguous residue comprises leucine and isoleucine.

Embodiment 23: The embodiment of any of embodiments 21-22, further comprising: determining a respective random hit rate for each of the respective potential sources such that the random hit rate increases as a number of random sequences are found by a respective search of the one or more searches; and determining the putative source such that the respective random hit rate of the putative source is the lowest of the respective random hit rates for each of the potential sources.

Embodiment 24: The embodiment of any of embodiments 21-23, further comprising: identifying one or more likely permutated peptide sequences of the plurality of permutated peptide sequences such that each of the one or more likely permutated peptide sequences are associated with the putative source.

Embodiment 25: The embodiment of any of embodiments 1-24, wherein the peptide sequence is a de novo peptide sequence determined via mass spectrometry.

Embodiment 26: Non-transitory computer-readable medium configured to communicate with one or more processor(s) of a computational device, the non-transitory computer-readable medium including instructions thereon, that when executed by the processor(s), cause the computational device to: receive, as an input, a peptide sequence; determine, based at least in part on one or more searches of the peptide sequence within one or more databases, a putative source associated with the peptide sequence, wherein each respective search of the one or more searches has a random hit rate that is based at least in part on a number of random sequences found by the respective search, and wherein the one or more searches are performed in order of increasing random hit rates until the putative source is determined; and provide, as an output, the putative source.

Embodiment 27: The embodiment as in the embodiment 26, wherein the one or more databases comprises an expanded human proteome database, and wherein the expanded human proteome database comprises computer-readable representations of translations from messenger ribonucleic acids (RNAs) and non-coding RNAs.

Embodiment 28: The embodiment as in the embodiment 27, wherein the expanded human proteome database comprises computer-readable representations of translations from micro RNAs.

Embodiment 29: The embodiment of any of embodiments 27-28, wherein the expanded human proteome database comprises computer-readable representations of translations from long non-coding RNAs.

Embodiment 30: The embodiment of any of embodiments 27-29, wherein the expanded human proteome database comprises computer-readable representations of translations of human endogenous retroviruses.

Embodiment 31: The embodiment of any of embodiments 27-29, wherein the one or more searches comprises a linear human proteome search for the peptide sequence within the expanded human proteome database, and wherein the putative source is a linear expanded human proteome source when the linear human proteome search for the peptide sequence within the expanded human proteome database finds the peptide sequence within the expanded human proteome database.

Embodiment 32: The embodiment as in the embodiment 31, wherein, the instructions, when executed by the processor(s), cause the computational device to: identify, when the source is the linear expanded human proteome source, whether the peptide is putatively translated from messenger RNA or non-coding RNA.

Embodiment 33: The embodiment of any of embodiments 27-32, wherein the one or more databases comprises a human genome database, wherein the one or more searches comprises a linear human genome search of translations of the human genome database, and wherein the putative source is a linear genome source when the linear human genome search finds human genome sequence from which the peptide is putatively synthesized.

Embodiment 34: The embodiment as in the embodiment 33, wherein the linear human genome search excludes portions of the human genome from which the messenger RNA and the non-coding RNA of the expanded human proteome database are transcribed and includes remaining portions of the human genome.

Embodiment 35: The embodiment of any of embodiments 27-34, wherein the one or more searches comprises a linear mismatch search for peptides having a mismatch to the peptide sequence within the expanded human proteome database, and wherein the putative source is a linear mismatch of the expanded human proteome when the linear mismatch search finds a peptide sequence having a mismatch to the peptide sequence within expanded human proteome database.

Embodiment 36: The embodiment as in the embodiment 35, wherein the linear mismatch search is a search for peptide sequences having only a single mismatch to the peptide sequence.

Embodiment 37: The embodiment of any of embodiments 26-36, wherein the one or more databases comprises a non-endogenous proteome database comprising computer-readable representations of proteins translated from RNA from non-endogenous organisms and/or proteins synthesized by non-endogenous organisms, wherein the one or more searches comprises a linear non-endogenous search for the peptide sequence within the non-endogenous proteome database, and wherein the putative source is a linear non-endogenous proteome source when the linear non-endogenous search finds the peptide sequence within the non-endogenous proteome database.

Embodiment 38: The embodiment as in the embodiment 37, wherein the non-endogenous proteome database comprises a Basic Local Alignment Search Tool (BLAST) database.

Embodiment 39: The embodiment of any of embodiments 27-38, wherein the one or more searches comprises a cis-spliced search, within the expanded human proteome database, for peptide fragments that can be cis-spliced to match the peptide sequence, and wherein the source is a cis-spliced human proteome source when the cis-spliced search finds, within the expanded human proteome database, peptide fragments that can be cis-spliced to match the peptide sequence.

Embodiment 40: The embodiment of any of embodiments 27-39, wherein the one or more searches comprises a trans-spliced search, within the expanded human proteome database, for computer-readable representations of peptide fragments that can be trans-spliced to match the peptide sequence, and wherein the source is a trans-spliced human proteome source when the trans-spliced search finds, within the expanded human proteome database, computer-readable representations of peptide fragments that can be trans-spliced to match the peptide sequence.

Embodiment 41: The embodiment as in the embodiment 40, wherein the putative source is determined to be unidentified when the trans-spiced search does not find computer-readable representations of peptide fragments that can be trans-spliced to match the peptide sequence.

Embodiment 42: The embodiment of any of embodiments 27-41, wherein the one or more databases comprises a human genome, and wherein the one or more searches comprise the following searches ordered sequentially in a workflow as follows: a linear human proteome search for the peptide sequence within the expanded human proteome database; a linear human genome search of translations of the human genome database; a linear mismatch search for peptides having a mismatch to the peptide sequence within the expanded human proteome database; and a cis-spliced search, within the expanded human proteome database, for peptide fragments that can be cis-spliced to match the peptide sequence.

Embodiment 43: The embodiment as in the embodiment 42, wherein the one or more databases comprises a non-endogenous proteome database comprising computer-readable representations of proteins translated from RNA from non-endogenous organisms and/or proteins synthesized by non-endogenous organisms, wherein the one or more searches further comprises a linear non-endogenous search for the peptide sequence within the non-endogenous proteome database, and wherein the linear non-endogenous search is ordered sequentially in the workflow after the linear mismatch search and before the cis-spliced search.

Embodiment 44: The embodiment of any of embodiments 42-43, wherein the one or more searches further comprises a trans-spliced search, within the expanded human proteome database, for peptide fragments that can be trans-spliced to match the peptide sequence, and wherein the trans-spliced search is ordered sequentially in the workflow after the cis-spliced search.

Embodiment 45: The embodiment of any of embodiments 42-44, wherein, the instructions, when executed by the processor(s), cause the computational device to: halt advancement of the workflow to a subsequent search of the one or more searches when the putative source is determined for the peptide sequence.

Embodiment 46: The embodiment of any of embodiments 26-45, wherein the peptide sequence comprises at least one ambiguous residue, and wherein, the instructions, when executed by the processor(s), cause the computational device to: generate a plurality of permutated peptide sequences each comprising a potential residue for each of the at least one ambiguous residue; determine, for each of the plurality of permutated peptide sequences, a respective potential source; and determine the putative source of the peptide sequence such that the putative source is a respective potential source.

Embodiment 47: The embodiment as in the embodiment 46, wherein the potential residue for each of the at least one ambiguous residue comprises leucine and isoleucine.

Embodiment 48: The embodiment of any of embodiments 46-47, wherein, the instructions, when executed by the processor(s), cause the computational device to: determine a respective random hit rate for each of the respective potential sources such that the random hit rate increases as a number of random sequences are found by a respective search of the one or more searches; and determine the putative source such that the respective random hit rate of the putative source is the lowest of the respective random hit rates for each of the potential sources.

Embodiment 49: The embodiment of any of embodiments 46-48, wherein, the instructions, when executed by the processor(s), cause the computational device to: identify one or more likely permutated peptide sequences of the plurality of permutated peptide sequences such that each of the one or more likely permutated peptide sequences are associated with the putative source.

Embodiment 50: The embodiment of any of embodiments 26-49, wherein the peptide sequence is a de novo peptide sequence determined via mass spectrometry.

Embodiment 51: A method of ordering a peptide source assignment workflow, the method comprising: generating a plurality of random peptide sequences; determining a plurality of peptide source search steps; searching for each of the plurality of random peptide sequences by each of the plurality of peptide source search steps; determining, for each of the plurality of peptide source search steps, a random hit rate for a respective search step of the plurality of peptide source search steps based at least in part on a number of the plurality of random peptide sequences found by the respective search step; and ordering the peptide source search steps in the peptide source assignment workflow from lowest random hit rate to highest random hit rate.

Embodiment 52: The embodiment as in the embodiment 51, wherein the random peptide sequences comprise random sequences uniformly sampling all amino acids.

Embodiment 53: The embodiment of any of embodiments 51-52, wherein the random peptide sequences comprise sequences with frequencies of amino acids matching those found in vertebrates.

Embodiment 54: The embodiment of any of embodiments 51-53, wherein each peptide of the random peptide sequences comprises a length of eight to fourteen amino acids.

Embodiment 55: The embodiment of any of embodiments 51-54, wherein each peptide of the random peptide sequences comprises a length of nine to fourteen amino acids, ten to fourteen amino acids, eleven to fourteen amino acids, twelve to fourteen amino acids, thirteen to fourteen amino acids, eight to thirteen amino acids, eight to twelve amino acids, eight to eleven amino acids, eight to ten amino acids, eight to nine amino acids, nine to thirteen amino acids, nine to twelve amino acids, nine to eleven amino acids, nine to ten amino acids, ten to thirteen amino acids, ten to twelve amino acids, ten to eleven amino acids, eleven to thirteen amino acids, elven to twelve amino acids, or twelve to thirteen amino acids.

Embodiment 56: The embodiment of any of embodiments 51-55, wherein the plurality of peptide source search steps comprises a linear human proteome search for a peptide sequence within an expanded human proteome database, and wherein the expanded human proteome database comprises computer-readable representations of translations from messenger ribonucleic acids (RNAs) and non-coding RNAs.

Embodiment 57: The embodiment as in the embodiment 56, wherein the expanded human proteome database comprises computer-readable representations of translations from micro RNAs.

Embodiment 58: The embodiment of any of embodiments 56-57, wherein the expanded human proteome database comprises computer-readable representations of translations from long non-coding RNAs.

Embodiment 59: The embodiment of any of embodiments 56-58, wherein the expanded human proteome database comprises computer-readable representations of translations of human endogenous retroviruses.

Embodiment 60: The embodiment of any of embodiments 56-59, wherein the plurality of peptide source search steps comprises a linear human genome search of translations of a human genome database.

Embodiment 61: The embodiment as in the embodiment 60, wherein the linear human genome search excludes portions of the human genome from which the messenger RNA and the non-coding RNA of the expanded human proteome database are transcribed and includes remaining portions of the human genome.

Embodiment 62: The embodiment of any of embodiments 51-61, wherein the plurality of peptide source search steps comprises a linear mismatch search for peptides having a mismatch to the peptide sequence within an expanded human proteome database, and wherein the expanded human proteome database comprises computer-readable representations of translations from messenger ribonucleic acids (RNAs) and non-coding RNAs.

Embodiment 63: The embodiment of any of embodiments 51-62, wherein the plurality of peptide source search steps comprises a linear non-endogenous search for the peptide sequence within a non-endogenous proteome database, and wherein the non-endogenous proteome database comprises computer-readable representations of proteins translated from RNA from non-endogenous organisms and/or proteins synthesized by non-endogenous organisms.

Embodiment 64: The embodiment as in the embodiment 63, wherein the non-endogenous proteome database comprises a Basic Local Alignment Search Tool (BLAST) database.

Embodiment 65: The embodiment of any of embodiments 51-64, wherein the plurality of peptide source search steps comprises a cis-spliced search, within an expanded human proteome database, for peptide fragments that can be cis-spliced to match the peptide sequence, and wherein the expanded human proteome database comprises computer-readable representations of translations from messenger ribonucleic acids (RNAs) and non-coding RNAs.

Embodiment 66: The embodiment of any of embodiments 51-65, wherein the plurality of peptide source search steps comprises a trans-spliced search, within an expanded human proteome database, for peptide fragments that can be trans-spliced to match the peptide sequence, and wherein the expanded human proteome database comprises computer-readable representations of translations from messenger ribonucleic acids (RNAs) and non-coding RNAs.

Embodiment 67: The embodiment of any of embodiments 51-66, wherein the peptide source assignment workflow terminates with a peptide being not assigned when the peptide is not assigned a peptide source by any of the plurality of peptide source search steps.

Embodiment 68: The embodiment of any of embodiments 51-67, wherein the peptide source assignment workflow comprises the following searches ordered sequentially as follows: a linear human proteome search for the peptide sequence within the expanded human proteome database; a linear human genome search of translations of a human genome database; a linear mismatch search for peptides having a mismatch to the peptide sequence within the expanded human proteome database; and a cis-spliced search, within the expanded human proteome database, for peptide fragments that can be cis-spliced to match the peptide sequence.

Embodiment 69: The embodiment as in the embodiment 68, wherein the peptide source assignment workflow comprises a linear non-endogenous search for the peptide sequence within a non-endogenous proteome database, and wherein the linear non-endogenous search is ordered sequentially within the peptide assignment workflow after the linear mismatch search and before the cis-spliced search.

Embodiment 70: The embodiment of any of the embodiments 68-69, wherein the peptide source assignment workflow comprises a trans-spliced search, within the expanded human proteome database, for peptide fragments that can be trans-spliced to match the peptide sequence, and wherein the trans-spliced search is ordered sequentially within the peptide assignment workflow after the cis-spliced search.

Embodiment 71: Non-transitory computer-readable medium configured to communicate with one or more processor(s) of a computational device, the non-transitory computer-readable medium including instructions thereon, that when executed by the processor(s), cause the computational device to: receive, as an input, a plurality of peptide source search steps; generate a plurality of random peptide sequences; search for each of the plurality of random peptide sequences by each of the plurality of peptide source search steps; determine, for each of the plurality of peptide source search steps, a random hit rate for a respective search step of the plurality of peptide source search steps based at least in part on a number of the plurality of random peptide sequences found by the respective search step; order the peptide source search steps in a peptide source assignment workflow from lowest random hit rate to highest random hit rate; and provide, as an output, the peptide source assignment workflow.

Embodiment 72: The embodiment as in the embodiment 71, wherein the random peptide sequences comprise random sequences uniformly sampling all amino acids.

Embodiment 73: The embodiment of any of embodiments 71-72, wherein the random peptide sequences comprise sequences with frequencies of amino acids matching those found in vertebrates.

Embodiment 74: The embodiment of any of embodiments 71-73, wherein each peptide of the random peptide sequences comprises a length of eight to fourteen amino acids.

Embodiment 75: The embodiment of any of embodiments 71-74, wherein each peptide of the random peptide sequences comprises a length of nine to fourteen amino acids, ten to fourteen amino acids, eleven to fourteen amino acids, twelve to fourteen amino acids, thirteen to fourteen amino acids, eight to thirteen amino acids, eight to twelve amino acids, eight to eleven amino acids, eight to ten amino acids, eight to nine amino acids, nine to thirteen amino acids, nine to twelve amino acids, nine to eleven amino acids, nine to ten amino acids, ten to thirteen amino acids, ten to twelve amino acids, ten to eleven amino acids, eleven to thirteen amino acids, elven to twelve amino acids, or twelve to thirteen amino acids.

Embodiment 76: The embodiment of any of embodiments 71-75, wherein the plurality of peptide source search steps comprises a linear human proteome search for a peptide sequence within an expanded human proteome database, and wherein the expanded human proteome database comprises computer-readable representations of translations from messenger ribonucleic acids (RNAs) and non-coding RNAs.

Embodiment 77: The embodiment as in the embodiment 76, wherein the expanded human proteome database comprises computer-readable representations of translations from micro RNAs.

Embodiment 78: The embodiment of any of embodiments 76-77, wherein the expanded human proteome database comprises computer-readable representations of translations from long non-coding RNAs.

Embodiment 79: The embodiment of any of embodiments 76-78, wherein the expanded human proteome database comprises computer-readable representations of translations of human endogenous retroviruses.

Embodiment 80: The embodiment of any of embodiments 76-79, wherein the plurality of peptide source search steps comprises a linear human genome search of translations of a human genome database.

Embodiment 81: The embodiment as in the embodiment 80, wherein the linear human genome search excludes portions of the human genome from which the messenger RNA and the non-coding RNA of the expanded human proteome database are transcribed and includes remaining portions of the human genome.

Embodiment 82: The embodiment of any of embodiments 71-81, wherein the plurality of peptide source search steps comprises a linear mismatch search for peptides having a mismatch to the peptide sequence within an expanded human proteome database, and wherein the expanded human proteome database comprises computer-readable representations of translations from messenger ribonucleic acids (RNAs) and non-coding RNAs.

Embodiment 83: The embodiment of any of embodiments 71-82, wherein the plurality of peptide source search steps comprises a linear non-endogenous search for the peptide sequence within a non-endogenous proteome database, and wherein the non-endogenous proteome database comprises computer-readable representations of proteins translated from RNA from non-endogenous organisms and/or proteins synthesized by non-endogenous organisms.

Embodiment 84: The embodiment as in the embodiment 83, wherein the non-endogenous proteome database comprises a Basic Local Alignment Search Tool (BLAST) database.

Embodiment 85: The embodiment of any of embodiments 71-84, wherein the plurality of peptide source search steps comprises a cis-spliced search, within an expanded human proteome database, for peptide fragments that can be cis-spliced to match the peptide sequence, and wherein the expanded human proteome database comprises computer-readable representations of translations from messenger ribonucleic acids (RNAs) and non-coding RNAs.

Embodiment 86: The embodiment of any of embodiments 71-85, wherein the plurality of peptide source search steps comprises a trans-spliced search, within an expanded human proteome database, for peptide fragments that can be trans-spliced to match the peptide sequence, and wherein the expanded human proteome database comprises computer-readable representations of translations from messenger ribonucleic acids (RNAs) and non-coding RNAs.

Embodiment 87: The embodiment of any of embodiments 71-86, wherein the peptide source assignment workflow terminates with a peptide being not assigned when the peptide is not assigned a peptide source by any of the plurality of peptide source search steps.

Embodiment 88: The embodiment of any of embodiments 71-87, wherein the peptide source assignment workflow comprises the following searches ordered sequentially as follows: a linear human proteome search for the peptide sequence within the expanded human proteome database; a linear human genome search of translations of a human genome database; a linear mismatch search for peptides having a mismatch to the peptide sequence within the expanded human proteome database; a linear non-endogenous search for the peptide sequence within a non-endogenous proteome database; a cis-spliced search, within the expanded human proteome database, for peptide fragments that can be cis-spliced to match the peptide sequence; and a trans-spliced search, within the expanded human proteome database, for peptide fragments that can be trans-spliced to match the peptide sequence.

Embodiment 89: The embodiment as in the embodiment 88, wherein the peptide source assignment workflow comprises a linear non-endogenous search for the peptide sequence within a non-endogenous proteome database, and wherein the linear non-endogenous search is ordered sequentially within the peptide assignment workflow after the linear mismatch search and before the cis-spliced search.

Embodiment 90: The embodiment of any of the embodiments 88-89, wherein the peptide source assignment workflow comprises a trans-spliced search, within the expanded human proteome database, for peptide fragments that can be trans-spliced to match the peptide sequence, and wherein the trans-spliced search is ordered sequentially within the peptide assignment workflow after the cis-spliced search.

Embodiment 91: A method comprising: generating a plurality of simulated random queries; determining, based on applying the plurality of simulated random queries to each source of a plurality of sources, a number of matches associated with each source; determining, based on the numbers of matches associated with each source, a false discovery rate associated with each source; and generating, based on the false discovery rates, a query support data structure configured to facilitate application of a new query to the plurality of sources.

Embodiment 92: The embodiment as in the embodiment 91, wherein generating the plurality of simulated random queries comprises at least one of: generating a plurality of uniform random queries; or generating a plurality of weighted random queries.

Embodiment 93: The embodiment of any of embodiments 91-92, wherein the plurality of simulated random queries comprises a plurality of simulated random text strings.

Embodiment 94: The embodiment of any of embodiments 91-93, wherein the plurality of simulated random queries comprises a plurality of simulated random peptide sequences.

Embodiment 95: The embodiment of any of embodiments 91-94, wherein determining, based on the numbers of matches associated with each source, the false discovery rate associated with each source comprises a function of the number of matches and a number of the plurality of simulated random queries.

Embodiment 96: The embodiment of any of embodiments 91-95, wherein determining, based on the numbers of matches associated with each source, the false discovery rate associated with each source comprises dividing the number of matches by a number of the plurality of simulated random queries.

Embodiment 97: A method comprising: receiving a query; applying, based on a query support data structure, the query to one or more sources of a plurality of sources; determining, based on a query result, a label associated with a source of the plurality of sources associated with the query result; and applying the label to the query.

Embodiment 98: The embodiment as in the embodiment 97, wherein the query comprises a text string.

Embodiment 99: The embodiment of any of embodiments 97-98, wherein the query comprises a peptide sequence.

Embodiment 100: The embodiment as in the embodiment 99, wherein receiving the query comprises receiving the peptide sequence from a mass spectrometer system.

Embodiment 101: The embodiment of any of embodiments 97-100, further comprising determining, via the mass spectrometer system, one or more amino acids of the peptide sequence.

Embodiment 102: The embodiment of any of embodiments 97-101, wherein the query support data structure indicates an order of the plurality of sources to apply the query, wherein the order is based on a false discovery rate associated with each source of the plurality of sources.

Embodiment 103: The embodiment of any of embodiments 97-102, further comprising determining one or more permutations of the query.

Embodiment 104: The embodiment as in the embodiment 103, wherein applying, based on the query support data structure, the query to the one or more sources of the plurality of sources comprises: applying each permutation of the one or more permutations of the query to the one or more sources of the plurality of sources; if an identical match to the one or more permutations of the query is found in a first source of the plurality of sources, discontinuing additional searches and applying a linear label to the one or more permutations of the query associated with the identical match; and assigning the one or more permutations of the query associated with the identical match as a correct query.

Embodiment 105: The embodiment of any of embodiments 97-104, wherein applying the query to one or more sources of a plurality of sources comprises: searching for an identical match to the query in a first source of the plurality of sources; and if an identical match to the query is found in the first source of the plurality of sources, discontinuing additional searches.

Embodiment 106: The embodiment as in the embodiment 105, wherein the query result comprises the identical match and wherein the label associated with a source of the plurality of sources associated with the query result comprises a linear label.

Embodiment 107: The embodiment of any of embodiments 97-106, wherein applying the query to one or more sources of a plurality of sources comprises: searching for an identical match to the query in a first source of the plurality of sources; and if an identical match to the one or more permutations of the query is found in the first source of the plurality of sources, discontinuing additional searches.

Embodiment 108: The embodiment as in the embodiment 107, wherein the query result comprises the identical match and wherein the label associated with a source of the plurality of sources associated with the query result comprises a linear label.

Embodiment 109: The embodiment as in the embodiment 107, wherein applying the query to one or more sources of a plurality of sources comprises: searching for an identical match to the query in any frame of a plurality of frames of a second source of the plurality of sources; and if an identical match to the query is found in any frame of a plurality of frames of the second source of the plurality of sources, discontinuing additional searches.

Embodiment 110: The embodiment as in the embodiment 109, wherein the query result comprises the identical match and wherein the label associated with a source of the plurality of sources associated with the query result comprises a linear label.

Embodiment 111: The embodiment as in the embodiment 109, wherein applying the query to one or more sources of a plurality of sources comprises: searching for a non-identical match to the query in a third source of the plurality of sources; and if a non-identical match to the query is found in the third source of the plurality of sources, discontinuing additional searches.

Embodiment 112: The embodiment as in the embodiment 111, wherein the query result comprises the non-identical match and wherein the label associated with a source of the plurality of sources associated with the query result comprises a mismatch label.

Embodiment 113: The embodiment as in the embodiment 111, wherein applying the query to one or more sources of a plurality of sources comprises: searching for a homologous match to the query in a fourth source of the plurality of sources; and if a homologous match to the query is found in the fourth source of the plurality of sources, discontinuing additional searches.

Embodiment 114: The embodiment as in the embodiment 113, wherein the query result comprises the homologous match and wherein the label associated with a source of the plurality of sources associated with the query result comprises a homologous label.

Embodiment 115: The embodiment as in the embodiment 113, wherein applying the query to one or more sources of a plurality of sources comprises: splitting the query into a plurality of sets of fragments; searching for each set of fragments in a fifth source of the plurality of sources; if a match for a set of fragments is found in the fifth source of the plurality of sources, discontinuing additional searches; and if a first match for a first fragment of the set of fragments and a second match for a second fragment of the set of fragments is found in the fifth source of the plurality of sources, discontinuing additional searches.

Embodiment 116: The embodiment as in the embodiment 115, wherein the query result comprises the match for the set of fragments and wherein the label associated with a source of the plurality of sources associated with the query result comprises a cis-spliced label.

Embodiment 117: The embodiment as in the embodiment 115, wherein the query result comprises the first match for the first fragment of the set of fragments and the second match for the second fragment of the set of fragments and wherein the label associated with a source of the plurality of sources associated with the query result comprises a trans-spliced label.

Embodiment 118: The embodiment of any of embodiments 97-117, further comprising determining, based on the label, a source of the query.

Embodiment 119: The embodiment as in the embodiment 118, further comprising, validating output of a mass spectrometer system based on the source of the query.

Embodiment 120: An apparatus one or more processors and a memory storing processor-executable instructions that, when executed by the one or more processors, cause the apparatus to perform any of the Embodiments 91-119.

Embodiment 121: One or more non-transitory computer-readable media storing processor-executable instructions thereon that, when executed by a processor, cause the processor to perform any of the Embodiments 91-119. Embodiment 122: A system comprising a computing device and a plurality of sources configured to perform any of the Embodiments 91-119.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

Those skilled in the art will recognize or be able to ascertain using no more than routine experimentation, many equivalents to the specific embodiments of the method and compositions described herein. Such equivalents are intended to be encompassed by the following claims.

TABLE 1

| | | SEQ |
|---|---|---|
| | | ID |
| Source | Peptide | NO |
| uniform | VFSFIDDV | 6 |
| uniform | YPMFGVTA | 7 |
| uniform | KPQWTFVL | 8 |
| uniform | HLGRCMLK | 9 |
| uniform | KVNLQQGQ | 10 |
| uniform | FTMWHGCQ | 11 |
| uniform | GLCWSMEE | 12 |
| uniform | GCYPVLML | 13 |
| uniform | CGGYISTH | 14 |
| uniform | AVTQQSKL | 15 |
| uniform | SYDVMFWK | 16 |
| uniform | NRSKGTYG | 17 |
| uniform | KMWMFVRT | 18 |
| uniform | SQSKKEFR | 19 |
| uniform | WQFMHGMM | 20 |
| uniform | YCVESQHQ | 21 |
| uniform | FCQMTSCP | 22 |
| uniform | WDPANSWE | 23 |
| uniform | PTCWAKQG | 24 |
| uniform | TWIKKDMA | 25 |
| uniform | LHQTGQTC | 26 |
| uniform | HWQTGWLQ | 27 |
| uniform | EDRPYMPA | 28 |
| uniform | DIVIWHFL | 29 |
| uniform | WRFHSVDH | 30 |
| uniform | HAWWYGFL | 31 |
| uniform | YRIVAEHM | 32 |
| uniform | QWMWSRYP | 33 |
| uniform | EPLFCLLA | 34 |
| uniform | CFMVSGME | 35 |
| uniform | LFYMREWS | 36 |
| uniform | WEQCEHVQ | 37 |
| uniform | DHVGNSER | 38 |
| uniform | GERHGTKY | 39 |
| uniform | RPIDHRSN | 40 |
| uniform | LAYWYDQH | 41 |
| uniform | EFGQEKVC | 42 |
| uniform | RKNGNSVF | 43 |
| uniform | MRYWQFEY | 44 |
| uniform | HPHSGNND | 45 |
| uniform | WYRDPDPR | 46 |
| uniform | PNKSQPGM | 47 |
| uniform | GWGQASAI | 48 |
| uniform | EDKNYMTI | 49 |
| uniform | AWENHKGK | 50 |
| uniform | QIYHNLCA | 51 |
| uniform | MEPKLYRH | 52 |
| uniform | QRNEMEPR | 53 |
| uniform | CLVVFRYW | 54 |
| uniform | TYTHGCES | 55 |
| uniform | NTLIFENY | 56 |
| uniform | CPNFPNFY | 57 |
| uniform | DHMETWDA | 58 |
| uniform | FWMGENTE | 59 |
| uniform | PWGIGKVI | 60 |
| uniform | KKLNCCTI | 61 |
| uniform | PQYICWLM | 62 |
| uniform | KDYNSQFK | 63 |
| uniform | RLWIHHYI | 64 |
| uniform | DGGYPMHY | 65 |
| uniform | LKLPYVQV | 66 |
| uniform | QQTNQSMI | 67 |
| uniform | RAWMDGWY | 68 |
| uniform | PTMINNYI | 69 |
| uniform | FNNKFDKG | 70 |
| uniform | ASADTYNV | 71 |
| uniform | LWHFNMSL | 72 |
| uniform | RGNMPFQS | 73 |
| uniform | EYVGVMPD | 74 |
| uniform | SDETPAIE | 75 |
| uniform | EDKLFQYW | 76 |
| uniform | VTRMDRFI | 77 |
| uniform | PPPWEEMP | 78 |
| uniform | MWNRIYGM | 79 |
| uniform | CARNVYHA | 80 |
| uniform | GCAQLGWY | 81 |
| uniform | DKPHDVCW | 82 |
| uniform | LWAYTLTD | 83 |
| uniform | RLGDQPDS | 84 |
| uniform | DEDLVAVG | 85 |
| uniform | GYCYHFES | 86 |
| uniform | ICSQIQPF | 87 |
| uniform | HPSVVDSC | 88 |
| uniform | KCDPKTVH | 89 |
| uniform | GMPLKPYC | 90 |
| uniform | QKMSLRMQ | 91 |
| uniform | TLITVFVV | 92 |
| uniform | WIQWCACC | 93 |
| uniform | IDPPIKEY | 94 |
| uniform | IWVTYKFL | 95 |
| uniform | ATNKQGID | 96 |
| uniform | AQLFMIPF | 97 |
| uniform | IRINGGMT | 98 |
| uniform | LRKFYRPQ | 99 |
| uniform | IYWQESPA | 100 |
| uniform | MIYQRIMT | 101 |
| uniform | CRMRTLRG | 102 |
| uniform | VWTIWKDK | 103 |
| uniform | LNEISEPE | 104 |
| uniform | WMWPSRNW | 105 |
| uniform | DRQVCYSQ | 106 |
| uniform | TYISCTED | 107 |
| uniform | YCMSRNFF | 108 |
| uniform | SNKSDTAA | 109 |
| uniform | PTDSHHMR | 110 |
| uniform | HGLHSIHG | 111 |
| uniform | PPLPIDQC | 112 |
| uniform | FTELCYIC | 113 |
| uniform | GWEYPDTA | 114 |
| uniform | TWYNDSMQ | 115 |
| uniform | HNTNSAAQ | 116 |
| uniform | EQQMTEWT | 117 |
| uniform | SRDCAIWP | 118 |
| uniform | RVAVAVLQ | 119 |
| uniform | RDVASGAP | 120 |
| uniform | CWYCEDAN | 121 |
| uniform | AIWSQGVA | 122 |
| uniform | AMRFPRAA | 123 |
| uniform | DNKLKNRL | 124 |
| uniform | KFPTDPCD | 125 |
| uniform | ACAGQVKV | 126 |
| uniform | ITKFETNK | 127 |
| uniform | LTCFMHLI | 128 |
| uniform | HLTDCHEW | 129 |
| uniform | IMMLILME | 130 |
| uniform | PNKCERQM | 131 |
| uniform | EMWFMPAW | 132 |
| uniform | CSDDLKSR | 133 |
| uniform | QQIQYGMT | 134 |
| uniform | LLQMHLDK | 135 |
| uniform | DETCSKIR | 136 |
| uniform | PYRHEWGF | 137 |
| uniform | MCCWATVF | 138 |
| uniform | PCDHMDRN | 139 |
| uniform | NYEEGYNF | 140 |
| uniform | ACEVAHRE | 141 |
| uniform | HKHKNVVI | 142 |
| uniform | YQEHEEQW | 143 |
| uniform | GRRSSDCD | 144 |
| uniform | YDNHNLRV | 145 |
| uniform | RCYMMDPR | 146 |
| uniform | IHTPEMNQ | 147 |
| uniform | GFFGRYWT | 148 |
| uniform | GWNEYACP | 149 |
| uniform | RFHQWHDA | 150 |
| uniform | NVSYAFPV | 151 |
| uniform | GFWEYPAN | 152 |
| uniform | DTNKYFWE | 153 |
| uniform | EQGRCEFC | 154 |
| uniform | AFHSHDLT | 155 |
| uniform | QETILNKH | 156 |
| uniform | FMFHCRNN | 157 |
| uniform | HRFDVPAA | 158 |
| uniform | ASDAGGPF | 159 |
| uniform | VWLFLNTN | 160 |
| uniform | WPHDSLCC | 161 |
| uniform | VMNVRIHW | 162 |
| uniform | DTRDITVT | 163 |
| uniform | RHDNDDMD | 164 |
| uniform | WRWTSIEA | 165 |
| uniform | YIAHNMDG | 166 |
| uniform | NMHCKEEM | 167 |
| uniform | EWMYGCVM | 168 |
| uniform | LPLKDWYY | 169 |
| uniform | TVNWLTLR | 170 |
| uniform | IMKRGRPD | 171 |
| uniform | YFYEKDCW | 172 |
| uniform | LFVNEGWD | 173 |
| uniform | DFVFQQNI | 174 |
| uniform | KYEMQPVM | 175 |
| uniform | LRNQMNCS | 176 |
| uniform | LATPCVAL | 177 |
| uniform | VLFRACPN | 178 |
| uniform | GTIHAGEG | 179 |
| uniform | MTQYYPII | 180 |
| uniform | RPNQGPDH | 181 |
| uniform | ELNDDVRP | 182 |
| uniform | WTLMHGWA | 183 |
| uniform | NKHGCEPY | 184 |
| uniform | YPCHTWIM | 185 |
| uniform | YTNKRMPC | 186 |
| uniform | YLYYWTVS | 187 |
| uniform | VLRRLMGE | 188 |
| uniform | GKPKYMFH | 189 |
| uniform | QGVTTIMA | 190 |
| uniform | ISLWPHMH | 191 |
| uniform | QHQLSQEY | 192 |
| uniform | GIGTHREN | 193 |
| uniform | QAARPDNN | 194 |
| uniform | DCETTGWG | 195 |
| uniform | DIQPNNII | 196 |
| uniform | PGAYSYFM | 197 |
| uniform | TDPATGNW | 198 |
| uniform | VYSKNTCS | 199 |
| uniform | KDSCMWQT | 200 |
| uniform | WLICDQQV | 201 |
| uniform | NSEGDYSV | 202 |
| uniform | NCHQWCWQ | 203 |
| uniform | YPMNTWRA | 204 |
| uniform | AGECNYRA | 205 |
| uniform | HQKYFTPC | 206 |
| uniform | RLELSNLQ | 207 |
| uniform | VKGYCKWH | 208 |
| uniform | RYRFQYMI | 209 |
| uniform | NILHLEVT | 210 |
| uniform | CQVYMNNG | 211 |
| uniform | WKQNRAVR | 212 |
| uniform | MFMWWCSE | 213 |
| uniform | QTSAAGWF | 214 |
| uniform | YIWALRDW | 215 |
| uniform | QDRFWCWA | 216 |
| uniform | KIKEWRSM | 217 |
| uniform | CMFSADTG | 218 |
| uniform | HQMLYMKY | 219 |
| uniform | QRMNKVMN | 220 |
| uniform | VWHKCGRN | 221 |
| uniform | PARIRWYE | 222 |
| uniform | CMCYSLIR | 223 |
| uniform | VNWYTMIW | 224 |
| uniform | VHLSQHTI | 225 |
| uniform | KESLKSYG | 226 |
| uniform | PKQREICW | 227 |
| uniform | GEVWEYGM | 228 |
| uniform | LEHVDNDA | 229 |
| uniform | QSKLIDGH | 230 |
| uniform | MYHREDAQ | 231 |
| uniform | PMLAPVHC | 232 |
| uniform | RRMIHFLV | 233 |
| uniform | YFCWVMTD | 234 |
| uniform | YDPAACVD | 235 |
| uniform | GYMWYWYA | 236 |
| uniform | DYWRKKSC | 237 |
| uniform | DSWCLRME | 238 |
| uniform | RDFCLLKW | 239 |
| uniform | KYHHPCAS | 240 |
| uniform | NHECFCIT | 241 |
| uniform | HKRPEYHQ | 242 |
| uniform | FYWKMHIP | 243 |
| uniform | IYAIISEM | 244 |
| uniform | WEWHRYIM | 245 |
| uniform | AGHFNLAF | 246 |
| uniform | CGKIDATK | 247 |
| uniform | HCVNAEHL | 248 |
| uniform | IMRQLGSS | 249 |
| uniform | EVPHMNTC | 250 |
| uniform | LCVFPGRC | 251 |
| uniform | TDWEKNDY | 252 |
| uniform | PVGPPIRG | 253 |
| uniform | CVCHCRND | 254 |
| uniform | DNADWMMQ | 255 |
| uniform | CHVQWIMP | 256 |
| uniform | GEEEYDPV | 257 |
| uniform | IDAHNARF | 258 |
| uniform | WQTHVTPL | 259 |
| uniform | YDGQADLT | 260 |
| uniform | VYYLFNDK | 261 |
| uniform | IERKIDGW | 262 |
| uniform | FSYGSPVK | 263 |
| uniform | YWEHYQEF | 264 |
| uniform | CKVAHSST | 265 |
| uniform | QCHEMQLA | 266 |
| uniform | WDVWPSNS | 267 |
| uniform | ACTCSYCW | 268 |
| uniform | KVKQCLKL | 269 |
| uniform | AWWMFNIF | 270 |
| uniform | PGPQQARA | 271 |
| uniform | ACHYKMLT | 272 |
| uniform | MTNLGHLI | 273 |
| uniform | TGFYVGGR | 274 |
| uniform | ENYCFYLQ | 275 |
| uniform | ECYVPGHC | 276 |
| uniform | RSVDDGIH | 277 |
| uniform | CDCVNLCW | 278 |
| uniform | PHAQFPLH | 279 |
| uniform | QDNTHKLM | 28 |
| uniform | GIQTWEGG | 281 |
| uniform | QNRFEPTR | 282 |
| uniform | CAGQGRHE | 283 |
| uniform | PTMICTWV | 284 |
| uniform | ATMLDCKL | 285 |
| uniform | NCYVKADV | 286 |
| uniform | SGRDIDWQ | 287 |
| uniform | NQRVTNME | 288 |
| uniform | FNMTSDQY | 289 |
| uniform | NKLKMDPW | 290 |
| uniform | GWYFVGVY | 291 |
| uniform | VQENLEWH | 292 |
| uniform | LEKHENRS | 293 |
| uniform | HHITGNMW | 294 |
| uniform | QPNVTHFC | 295 |
| uniform | CLRTNNTM | 296 |
| uniform | RFDCIVDE | 297 |
| uniform | TEEKIFRM | 298 |
| uniform | FQDDRFIW | 299 |
| uniform | ASNCEQEG | 300 |
| uniform | TMQMQPSW | 301 |
| uniform | GTFGEAEH | 302 |
| uniform | KQVAMTPP | 303 |
| uniform | GGNPSDDY | 304 |
| uniform | NNPDHMKT | 305 |
| uniform | ERPDWQCE | 306 |
| uniform | RPENKAEV | 307 |
| uniform | WQKNLACE | 308 |
| uniform | RCGSITRM | 309 |
| uniform | WWTSLVTW | 310 |
| uniform | CFVILAQG | 311 |
| uniform | PGYFCYPG | 312 |
| uniform | GCSCNEMC | 313 |
| uniform | FVGVRPDW | 314 |
| uniform | FQVRKQCN | 315 |
| uniform | CYLGGVFQ | 316 |
| uniform | QLQKPISA | 317 |
| uniform | SYCMIRLT | 318 |
| uniform | GQWCSHSS | 319 |
| uniform | KSEHLLYS | 320 |
| uniform | MQACMYGI | 321 |
| uniform | RKSDSQPI | 322 |
| uniform | CQALFKQF | 323 |
| uniform | SLYGEMTQ | 324 |
| uniform | LQYPKNNG | 325 |
| uniform | CWQGQIYE | 326 |
| uniform | NIFIKGHA | 327 |
| uniform | DSCQFWPS | 328 |
| uniform | NKKIEVGE | 329 |
| uniform | TMPHQNDN | 330 |
| uniform | MNIVTFWY | 331 |
| uniform | NQLVMDTA | 332 |
| uniform | LSYLGGEH | 333 |
| uniform | ICRMHQYS | 334 |
| uniform | STYPVREQ | 335 |
| uniform | QVIQKKRD | 336 |
| uniform | PAGIQSFK | 337 |
| uniform | VQSGQIYY | 338 |
| uniform | PERWTRHI | 339 |
| uniform | TCKEPKYK | 340 |
| uniform | FMQTFCVI | 341 |
| uniform | HCNFCWSE | 342 |
| uniform | SNPVRPVK | 343 |
| uniform | YLWMLPND | 344 |
| uniform | HEYYNKRP | 345 |
| uniform | ARKDDQID | 346 |
| uniform | GQYEMGSY | 347 |
| uniform | IKDKHCDN | 348 |
| uniform | AQRDKSRP | 349 |
| uniform | QVMDLSIR | 350 |
| uniform | DAIQEIGG | 351 |
| uniform | QKPYAKVY | 352 |
| uniform | TVDSEYRT | 353 |
| uniform | YQGMAYTT | 354 |
| uniform | CDAPHEGC | 355 |
| uniform | GYDVDCKI | 356 |
| uniform | ARYGSLYQ | 357 |
| uniform | YVDRRMIA | 358 |
| uniform | QGWRDCCH | 359 |
| uniform | SSPVLFGP | 360 |
| uniform | LTLVTTPF | 361 |
| uniform | SEGKSFRG | 362 |
| uniform | MAVQKHIV | 363 |
| uniform | YNCWKKYG | 364 |
| uniform | EAREIPFT | 365 |
| uniform | ELWMSEHH | 366 |
| uniform | SILQCVCW | 367 |
| uniform | WKTPGYTL | 368 |
| uniform | PTSLAAQM | 369 |
| uniform | GFLTRSDW | 370 |
| uniform | GCLISGFM | 371 |
| uniform | SRDFLVLA | 372 |
| uniform | RVIFITRE | 373 |
| uniform | HNFIYNRH | 374 |
| uniform | INCFDMMF | 375 |
| uniform | HEHFHEQN | 376 |
| uniform | RYMCCMHD | 377 |
| uniform | SNANPWEN | 378 |
| uniform | DDGVEIPQ | 379 |
| uniform | APKMRSWE | 380 |
| uniform | QVETQGDY | 381 |
| uniform | MALDSVRK | 382 |
| uniform | QKSHKDPN | 383 |
| uniform | TLSNFINL | 384 |
| uniform | IRQMNKPK | 385 |
| uniform | CCWEESVC | 386 |
| uniform | MIQRSIIE | 387 |
| uniform | YSGSKNWC | 388 |
| uniform | KKIENEYI | 389 |
| uniform | RGRGCFKW | 390 |
| uniform | LNAGRAFT | 391 |
| uniform | ICSDPWMV | 392 |
| uniform | KCEYWQIF | 393 |
| uniform | CHVYKVLE | 394 |
| uniform | LVSITYIH | 395 |
| uniform | FICLEHYG | 396 |
| uniform | KWRYCCNH | 397 |
| uniform | RGRFPVCG | 398 |
| uniform | CWQVPELN | 399 |
| uniform | NLGKKLDR | 400 |
| uniform | SFANDFCH | 401 |
| uniform | RWISAQWV | 402 |
| uniform | YHTGHMLW | 403 |
| uniform | DKPFEWLK | 404 |
| uniform | YWFGNVVP | 405 |
| uniform | WRSGNNLE | 406 |
| uniform | VWAWANER | 407 |
| uniform | EQCAQCDI | 408 |
| uniform | GPKVWHKF | 409 |
| uniform | VYWLKVGS | 410 |
| uniform | QFWSWAGK | 411 |
| uniform | LEWPSHPD | 412 |
| uniform | VETWKTSL | 413 |
| uniform | SYHIFIES | 414 |
| uniform | PMPCYNLP | 415 |
| uniform | FNLWVNFT | 416 |
| uniform | NLGYTWQT | 417 |
| uniform | LMSIIQID | 418 |
| uniform | IFLTDTYP | 419 |
| uniform | WWAIEIIC | 420 |
| uniform | PLMPRWFQ | 421 |
| uniform | EQAPTQYI | 422 |
| uniform | LGVDAKGY | 423 |
| uniform | FCFGKTDT | 424 |
| uniform | KPLSTFNC | 425 |
| uniform | VIDERVVN | 426 |
| uniform | LYHLNWYE | 427 |
| uniform | HKVICLEV | 428 |
| uniform | VGDQSEKM | 429 |
| uniform | LVRQCQRC | 430 |
| uniform | WFRHPDHK | 431 |
| uniform | KPAMTVLR | 432 |
| uniform | QMADSWQN | 433 |
| uniform | GIKPFCHH | 434 |
| uniform | RPHQRFRI | 435 |
| uniform | MSREAREQ | 436 |
| uniform | HGTEGREL | 437 |
| uniform | MQERGIWE | 438 |
| uniform | IYANDVDF | 439 |
| uniform | PSIVAMAE | 440 |
| uniform | EFMCKNAG | 441 |
| uniform | AYGATRKE | 442 |
| uniform | DEPYAQFN | 443 |
| uniform | NMSVNFHI | 444 |
| uniform | EKNKQAES | 445 |
| uniform | VLAMLFPF | 446 |
| uniform | VWDPFFLS | 447 |
| uniform | RSINFNWE | 448 |
| uniform | DQCDYNEK | 449 |
| uniform | CWAALQEM | 450 |
| uniform | YDQESLLE | 451 |
| uniform | LPFYAMGS | 452 |
| uniform | NCHWSNRR | 453 |
| uniform | GMYEMRTR | 454 |
| uniform | ELWVHCSI | 455 |
| uniform | FPSCRHRM | 456 |
| uniform | KGICYWRV | 457 |
| uniform | NEGNGPWD | 458 |
| uniform | TQTDEEFT | 459 |
| uniform | CPVYHRVR | 460 |
| uniform | FYSHRSMA | 461 |
| uniform | GCQNGQQH | 462 |
| uniform | TGLHANMA | 463 |
| uniform | FEPHSVTA | 464 |
| uniform | HCKLNMQW | 465 |
| uniform | AMPDVHRF | 466 |
| uniform | MWGTIIFE | 467 |
| uniform | LLITVCYC | 468 |
| uniform | KMKSRELN | 469 |
| uniform | LNFQYYSQ | 470 |
| uniform | LNSACYDR | 471 |
| uniform | VSIKERIE | 472 |
| uniform | VWSVAKCF | 473 |
| uniform | WTKFLLLC | 474 |
| uniform | PARQFYIM | 475 |
| uniform | EAPRPDVC | 476 |
| uniform | QVNLLVTR | 477 |
| uniform | VMTTGRMC | 478 |
| uniform | WGEPDCLC | 479 |
| uniform | HDHPLQHV | 480 |
| uniform | TNPPRHKC | 481 |
| uniform | AAEGGQYK | 482 |
| uniform | MHYELDNP | 483 |
| uniform | EFQMYIMI | 484 |
| uniform | VERCSTYN | 485 |
| uniform | RMEMIDWE | 486 |
| uniform | TREPMQLP | 487 |
| uniform | VKMSWHSW | 488 |
| uniform | CQMKSLMI | 489 |
| uniform | NLFQSYEV | 490 |
| uniform | DQPDETQN | 491 |
| uniform | DECRDVGQ | 492 |
| uniform | DKHVNMRH | 493 |
| uniform | QLTIFWWI | 494 |
| uniform | KEGWPVSH | 495 |
| uniform | HEIRYPWF | 496 |
| uniform | CGGWMYEM | 497 |
| uniform | VCMQILGM | 498 |
| uniform | YYMVTTAG | 499 |
| uniform | MQQWQVPQ | 500 |
| uniform | TCNQVQQT | 501 |
| uniform | HIDEDGPQ | 502 |
| uniform | ISEYWPAD | 503 |
| uniform | QDGNCCGK | 504 |
| uniform | DNEPIQIY | 505 |
| uniform | CAAMMENN | 506 |
| uniform | MSEVFWWE | 507 |
| uniform | KMHPKPYI | 508 |
| uniform | GNSCKVAD | 509 |
| uniform | NYYSCIPM | 510 |
| uniform | YSWHIFFP | 511 |
| uniform | DSILSHTY | 512 |
| uniform | SEKWIFCP | 513 |
| uniform | KRQIKALT | 514 |
| uniform | CGHKMTTY | 515 |
| uniform | QDTVGRTH | 516 |
| uniform | RGEFTNIH | 517 |
| uniform | NQKQTQRY | 518 |
| uniform | PPFDSNRL | 519 |
| uniform | HAVGRSTD | 520 |
| uniform | DRLGSMEF | 521 |
| uniform | FWSDHGYQ | 522 |
| uniform | TFHRFTYC | 523 |
| uniform | PKRNGWIF | 524 |
| uniform | GLNIPRFW | 525 |
| uniform | MHKSLHAD | 526 |
| uniform | YWAPSHGE | 527 |
| uniform | VWIVFVSS | 528 |
| uniform | SVTSATNE | 529 |
| uniform | IDYLLCCW | 530 |
| uniform | FMHCISFI | 531 |
| uniform | AFWSELDW | 532 |
| uniform | VRLGRMDD | 533 |
| uniform | WAMLKDYG | 534 |
| uniform | PYRAGVWI | 535 |
| uniform | YAKERQKV | 536 |
| uniform | FWQLNFNE | 537 |
| uniform | MSDQYPSM | 538 |
| uniform | SWEAGNLK | 539 |
| uniform | LLSFELMD | 540 |
| uniform | SESVKFQI | 541 |
| uniform | LCQHDYEW | 542 |
| uniform | TYWSQVEA | 543 |
| uniform | THCCQTWA | 544 |
| uniform | AETQDSMC | 545 |
| uniform | WVTLPAFY | 546 |
| uniform | NSNYKYEG | 547 |
| uniform | TPSPQWPV | 548 |
| uniform | QKLWCFCS | 549 |
| uniform | TMEWAYSK | 550 |
| uniform | RTIFEVFS | 551 |
| uniform | ESTKQPMH | 552 |
| uniform | CFQTMISF | 553 |
| uniform | YIAYWWKD | 554 |
| uniform | WAVATYME | 555 |
| uniform | TYHNIDRP | 556 |
| uniform | SVAKNTNW | 557 |
| uniform | KGCCIWFG | 558 |
| uniform | EDDNEHSN | 559 |
| uniform | NGDTRDFQ | 560 |
| uniform | TDIERLIW | 561 |
| uniform | NRQQKYVK | 562 |
| uniform | MIDYWQVP | 563 |
| uniform | VHIILVLV | 564 |
| uniform | LEISHMYQ | 565 |
| uniform | CMYACMDF | 566 |
| uniform | QDKPIYRA | 567 |
| uniform | CHWSTWCR | 568 |
| uniform | NMHDSPDK | 569 |
| uniform | CDYDGKVQ | 570 |
| uniform | HWNRDGVN | 571 |
| uniform | AQKHLHVA | 572 |
| uniform | DWYVWKEQ | 573 |
| uniform | WEFQESAS | 574 |
| uniform | DDSSGGHG | 575 |
| uniform | TANLLLPQ | 576 |
| uniform | KEWVHSQM | 577 |
| uniform | SVHLDRLH | 578 |
| uniform | ELTDTQLM | 579 |
| uniform | PDQVGGQT | 580 |
| uniform | SYRRMNDS | 581 |
| uniform | WYYGYPPS | 582 |
| uniform | LWDLSERN | 583 |
| uniform | IHTQFHVE | 584 |
| uniform | RHNQPCHP | 585 |
| uniform | SPYQDRMQ | 586 |
| uniform | PSSLPANF | 587 |
| uniform | HFYFYIHS | 588 |
| uniform | FYTTVAQG | 589 |
| uniform | SHYHAAIE | 590 |
| uniform | LLMSMATQ | 591 |
| uniform | KPKFAPNC | 592 |
| uniform | SLVKYGFV | 593 |
| uniform | RYALIPTV | 594 |
| uniform | WYKHTPEE | 595 |
| uniform | LHKIYDMM | 596 |
| uniform | WHMEYCLL | 597 |
| uniform | PKQIWMHS | 598 |
| uniform | SPFFGGPA | 599 |
| uniform | WNINLGSV | 600 |
| uniform | TLPAHFKT | 601 |
| uniform | LAEGAALH | 602 |
| uniform | FKNRDKFM | 603 |
| uniform | FYPVLSWR | 604 |
| uniform | WQRIKAAV | 605 |
| uniform | KMFKEGEL | 606 |
| uniform | WSEGDTPD | 60 |
| uniform | LRLGHLPI | 608 |
| uniform | RHDNEVYE | 609 |
| uniform | AITANSMS | 610 |
| uniform | RSHDIDLR | 611 |
| uniform | NAEDDTQE | 612 |
| uniform | ILFQNEKE | 613 |
| uniform | GKPRIKCT | 614 |
| uniform | TSCNVVYN | 615 |
| uniform | VTYCYKKK | 616 |
| uniform | TLWMITPF | 617 |
| uniform | WPTFREKR | 618 |
| uniform | TYPTFVGD | 619 |
| uniform | ECRDRGNS | 620 |
| uniform | AQGWYGVP | 621 |
| uniform | MYPILAMD | 622 |
| uniform | QVAVYIFH | 623 |
| uniform | DECADTMW | 624 |
| uniform | NTLLQIGP | 625 |
| uniform | IVHRCNSI | 626 |
| uniform | TQVEASIG | 627 |
| uniform | PYNNFLFH | 628 |
| uniform | RKFGGWYD | 629 |
| uniform | PEYISHTQ | 630 |
| uniform | IHRAKRHF | 631 |
| uniform | HEAMEFPW | 632 |
| uniform | IFLGHDRD | 633 |
| uniform | DMCVDDST | 634 |
| uniform | HENWCMLF | 635 |
| uniform | WELIYTCC | 636 |
| uniform | IDIGTEYA | 637 |
| uniform | MWPDKMIY | 638 |
| uniform | EWMVVKNR | 639 |
| uniform | MKFIPYCW | 640 |
| uniform | AVPNSCVE | 641 |
| uniform | KCAREHLM | 642 |
| uniform | IPVVNDCN | 643 |
| uniform | NNRADLLF | 644 |
| uniform | WGGFSFKV | 645 |
| uniform | SNDQRIKT | 646 |
| uniform | WRQGARDH | 647 |
| uniform | WMQTRGNI | 648 |
| uniform | CAWPFLNE | 649 |
| uniform | THEMPAWI | 650 |
| uniform | YSASFRPD | 651 |
| uniform | DSIFHKCG | 652 |
| uniform | SMQFPSSY | 653 |
| uniform | AAINRGHP | 654 |
| uniform | WGDSMSFT | 655 |
| uniform | KQWWMMCH | 656 |
| uniform | MCDEEFYN | 657 |
| uniform | LSGVFNSD | 658 |
| uniform | FQGWMNHP | 659 |
| uniform | ECPKDPYY | 660 |
| uniform | EEQNRYCG | 661 |
| uniform | HRKKLGHH | 662 |
| uniform | RYMPFTVT | 663 |
| uniform | IINWRHYC | 664 |
| uniform | KLRHRWRG | 665 |
| uniform | GKMNVIEH | 666 |
| uniform | HWIQDGPP | 667 |
| uniform | CRMARIQS | 668 |
| uniform | MVQQVHAW | 669 |
| uniform | PQIRHGFI | 670 |
| uniform | QNMSKFHA | 671 |
| uniform | DFLIGWPT | 672 |
| uniform | WPWYFHNA | 673 |
| uniform | YFLNVSSA | 674 |
| uniform | GPFSYTQG | 675 |
| uniform | VIMYMKFA | 676 |
| uniform | FFDQWYIH | 677 |
| uniform | SWDGSMDK | 678 |
| uniform | PQYGQRKR | 679 |
| uniform | WQWVSRYW | 680 |
| uniform | SDQWVAWQ | 681 |
| uniform | EYNRSACM | 682 |
| uniform | SCVYYWSQ | 683 |
| uniform | AATNWWNS | 684 |
| uniform | YPPMAFAD | 685 |
| uniform | CYQTQMCT | 686 |
| uniform | TKERLRKH | 687 |
| uniform | LPIWRLAI | 688 |
| uniform | WSFEPCCC | 689 |
| uniform | VQLNLCLT | 690 |
| uniform | YHNGQPFF | 691 |
| uniform | SFKGAWII | 692 |
| uniform | MWGVREWQ | 693 |
| uniform | YLADLQCH | 694 |
| uniform | WSDHPDGN | 695 |
| uniform | QLNGRMRS | 696 |
| uniform | WCGYNVVC | 697 |
| uniform | HSYGATGP | 698 |
| uniform | MTISKPWD | 699 |
| uniform | DIPNWEHN | 700 |
| uniform | VNPVTRPC | 701 |
| uniform | EKMRAARD | 702 |
| uniform | FHVEGFNM | 703 |
| uniform | DEQMLSHR | 704 |
| uniform | HHQHNVLI | 705 |
| uniform | IAKFPTTA | 706 |
| uniform | MGQSMCSI | 707 |
| uniform | GNNEYRNV | 708 |
| uniform | LHCNKPAM | 709 |
| uniform | ACTMKIIP | 710 |
| uniform | FDQALLVY | 711 |
| uniform | HTLNANIQ | 712 |
| uniform | QLYEREVY | 713 |
| uniform | LEESMLWP | 714 |
| uniform | NSDSNTHL | 715 |
| uniform | TYKWMPEE | 716 |
| uniform | NFTSWMEG | 717 |
| uniform | SQEVSYPY | 718 |
| uniform | SNKPVGDV | 719 |
| uniform | YDDNIIYE | 720 |
| uniform | RYEFVGPD | 721 |
| uniform | FWIGVELD | 722 |
| uniform | ADLFQHAA | 723 |
| uniform | NDFSPDPR | 724 |
| uniform | DQCDPLCP | 725 |
| uniform | WEKWSTCE | 726 |
| uniform | VKSKRHVA | 727 |
| uniform | FQSRVNDC | 728 |
| uniform | VFHHYQWE | 729 |
| uniform | MHRMGLIF | 730 |
| uniform | HCLWLAGQ | 731 |
| uniform | PIMAWHAH | 732 |
| uniform | ECNWDDPC | 733 |
| uniform | VVNNSQIF | 734 |
| uniform | CSDLMGTG | 735 |
| uniform | CNHTLNDY | 736 |
| uniform | PPQPHWQV | 737 |
| uniform | PYPIFLFW | 738 |
| uniform | VPVNLSTL | 739 |
| uniform | FYLLEFYL | 740 |
| uniform | MINFEQSW | 741 |
| uniform | TETHQRRV | 742 |
| uniform | HYNKPYKA | 743 |
| uniform | KWVRVCHI | 744 |
| uniform | GYDHMSWW | 745 |
| uniform | GNPVNNFT | 746 |
| uniform | HPMQVGPI | 747 |
| uniform | VCWRYAVE | 748 |
| uniform | CLKRVKAM | 749 |
| uniform | PGHFHMRC | 750 |
| uniform | GWERKWHT | 751 |
| uniform | KVSGRYQL | 752 |
| uniform | GFSARGTY | 753 |
| uniform | DYSRSVCP | 754 |
| uniform | SWMEHLLA | 755 |
| uniform | QYHMLLCT | 756 |
| uniform | PSKSYYIH | 757 |
| uniform | DCFFKWTL | 758 |
| uniform | QDMNQKML | 759 |
| uniform | EFFCCSTE | 760 |
| uniform | SRKVSWMY | 761 |
| uniform | ISHRADKD | 762 |
| uniform | ALVLPAFN | 763 |
| uniform | FSIWGDTC | 764 |
| uniform | SRKNSTAE | 765 |
| uniform | NPWAIEHK | 766 |
| uniform | LHVDWEIR | 767 |
| uniform | EIGDQMAQ | 768 |
| uniform | IPPMMYHD | 769 |
| uniform | AHWINVDT | 770 |
| uniform | FMEKFDSP | 771 |
| uniform | DASHKRNG | 772 |
| uniform | PEKHECQR | 773 |
| uniform | QGREIDKG | 774 |
| uniform | PSEECTVE | 775 |
| uniform | CVPMCYPN | 776 |
| uniform | YDIVIKHG | 777 |
| uniform | PMVTHSCA | 778 |
| uniform | AEACSEGF | 779 |
| uniform | SGVRLCGN | 780 |
| uniform | WNKTAYLN | 781 |
| uniform | GMIFKYEP | 782 |
| uniform | MFPMPGAA | 783 |
| uniform | QWPVIGST | 784 |
| uniform | SDDVYCHC | 785 |
| uniform | YKTDVAVL | 786 |
| uniform | HKPEEYRK | 787 |
| uniform | DEDTYQGC | 788 |
| uniform | CILVNNTR | 789 |
| uniform | ELGRARGT | 790 |
| uniform | HLRYKKIN | 791 |
| uniform | ITTGIFQI | 792 |
| uniform | AMAMYLIP | 793 |
| uniform | GFQIYMLL | 794 |
| uniform | ILKEQIVH | 795 |
| uniform | YLAAKQYE | 796 |
| uniform | GMPQVCHN | 797 |
| uniform | FLNWLSLE | 798 |
| uniform | HYGAQLFC | 799 |
| uniform | RPSWWCQG | 800 |
| uniform | HAKVENHA | 801 |
| uniform | EFCPYPSI | 802 |
| uniform | WITLEPDS | 803 |
| uniform | YSVNNDMP | 804 |
| uniform | CRGQWCNE | 805 |
| uniform | SHCIRAMK | 806 |
| uniform | NRYIMHQT | 807 |
| uniform | PLSNTVAE | 808 |
| uniform | KQQTYQGC | 809 |
| uniform | LGSWQCKN | 810 |
| uniform | CFYCEHKC | 811 |
| uniform | KIRPVMPV | 812 |
| uniform | CDKCLCIC | 813 |
| uniform | STYTSREI | 814 |
| uniform | EAFYWIVM | 815 |
| uniform | MGDWPPTD | 816 |
| uniform | MYRAMFSA | 817 |
| uniform | SWCQAMPK | 818 |
| uniform | AMKDRGRT | 819 |
| uniform | IFRIHIQF | 820 |
| uniform | AVILREEF | 821 |
| uniform | QSVFFVHH | 822 |
| uniform | DWCVEGIL | 823 |
| uniform | HWDIFLNS | 824 |
| uniform | VDLHVVHH | 825 |
| uniform | HVMKVDAT | 826 |
| uniform | PKWRLHCV | 827 |
| uniform | AYSPMRKG | 828 |
| uniform | WEKFLYAG | 829 |
| uniform | VSCHGCAI | 830 |
| uniform | GIHWFNFE | 831 |
| uniform | GKSAKGTH | 832 |
| uniform | PWEVNYDW | 833 |
| uniform | FLWINHQH | 834 |
| uniform | LGKEGGLA | 835 |
| uniform | TKWKDRQE | 836 |
| uniform | VLRVHMLH | 837 |
| uniform | SGPSWSTP | 838 |
| uniform | DCPPATHQ | 839 |
| uniform | QSESKMRF | 840 |
| uniform | IMQTYLFF | 841 |
| uniform | MTFPIWHL | 842 |
| uniform | FYVNCDAD | 843 |
| uniform | VCHVPSFS | 844 |
| uniform | KVDCQATG | 845 |
| uniform | NCIASCMR | 846 |
| uniform | INTYDDFAD | 847 |
| uniform | TFAGHDDK | 848 |
| uniform | CDCLQPYI | 849 |
| uniform | NFGMSRCQ | 850 |
| uniform | NQHHKKRS | 851 |
| uniform | TTADIYED | 852 |
| uniform | IPMNQFCL | 853 |
| uniform | DCHWTPPL | 854 |
| uniform | NQADDDNP | 855 |
| uniform | LQCWSTCS | 856 |
| uniform | IVIQPCRR | 857 |
| uniform | MMVASAKG | 858 |
| uniform | MVSCHDTN | 859 |
| uniform | YGPNSEYP | 860 |
| uniform | ESWPDVTS | 861 |
| uniform | YECVFTTM | 862 |
| uniform | AGNYAKAI | 863 |
| uniform | HRADVQNK | 864 |
| uniform | STDQSNHK | 865 |
| uniform | CKPGFAEG | 866 |
| uniform | QMCMDQPR | 867 |
| uniform | CLCLFYFL | 868 |
| uniform | RIDCSRPK | 869 |
| uniform | LMFRCFSC | 870 |
| uniform | PPGVSVSE | 871 |
| uniform | RSHYLGWV | 872 |
| uniform | PMNNDGTR | 873 |
| uniform | SCHQRCNH | 874 |
| uniform | PRQHINKQ | 875 |
| uniform | KYKCAMKQ | 876 |
| uniform | HMWQEMIF | 877 |
| uniform | AYDAWQME | 878 |
| uniform | CDMMNIDC | 879 |
| uniform | SWQDTRSN | 880 |
| uniform | NQAKCPQS | 881 |
| uniform | KWFCIYNL | 882 |
| uniform | EDIDEDTS | 883 |
| uniform | VKEFRAEG | 884 |
| uniform | DTRNDSNI | 885 |
| uniform | AYIDSDYH | 886 |
| uniform | PWEEHRRR | 887 |
| uniform | VHRSGQHD | 888 |
| uniform | MHGEHHNR | 889 |
| uniform | LSIMGCQW | 890 |
| uniform | GAACTNCM | 891 |
| uniform | IGMIYLIH | 892 |
| uniform | IISPWIWM | 893 |
| uniform | CSQVCWHY | 894 |
| uniform | NQCLQWCH | 895 |
| uniform | KQSYMSII | 896 |
| uniform | WLHNGVLW | 897 |
| uniform | SPTFRGIK | 898 |
| uniform | KVAVFWWD | 899 |
| uniform | YETESEAH | 900 |
| uniform | ENIMCKVT | 901 |
| uniform | MSFRQFCP | 902 |
| uniform | YTTFCRAR | 903 |
| uniform | VWRDWKAA | 904 |
| uniform | HNWRYNFQ | 905 |
| uniform | YEKYSPGE | 906 |
| uniform | TDVCDTVL | 907 |
| uniform | DELSFGYL | 908 |
| uniform | GTCMARAI | 909 |
| uniform | MVVDMRII | 910 |
| uniform | ETFMQDVT | 911 |
| uniform | PYENYSLG | 912 |
| uniform | PRMRWIDK | 913 |
| uniform | KMTEFDYC | 914 |
| uniform | CLHIILNH | 915 |
| uniform | YDELLIIK | 916 |
| uniform | SILCDVID | 917 |
| uniform | RIVMGVGW | 918 |
| uniform | QVAEQRLG | 919 |
| uniform | LQEPGCFI | 920 |
| uniform | KMSAPIAT | 921 |
| uniform | QSQTVQSW | 922 |
| uniform | DTLAHDFV | 923 |
| uniform | QEILDPIQ | 92 |
| uniform | IESRDQFT | 925 |
| uniform | HNHAGCKN | 926 |
| uniform | LIMWTHMG | 927 |
| uniform | DPGNSWGS | 928 |
| uniform | STNNVYKK | 929 |
| uniform | VSRSQQQM | 930 |
| uniform | AWWRLGGQ | 931 |
| uniform | AEWAYPPH | 932 |
| uniform | CDSAFEHQ | 933 |
| uniform | YRMTYRFI | 934 |
| uniform | PMHMEQCV | 935 |
| uniform | VQPWLTWR | 936 |
| uniform | WMIHMELH | 937 |
| uniform | PFPWVIMT | 938 |
| uniform | DGDDHPKR | 939 |
| uniform | KVENHEQL | 940 |
| uniform | PVYMGLLA | 941 |
| uniform | KQGDHLDE | 942 |
| uniform | HGSFFTTS | 943 |
| uniform | HLLVWWKG | 944 |
| uniform | HIGPWIVK | 945 |
| uniform | AHGFNIRW | 946 |
| uniform | HKGPTYME | 947 |
| uniform | MAVSRCNP | 948 |
| uniform | DRHFRNEA | 949 |
| uniform | RCMINAWD | 950 |
| uniform | RTNQYHLP | 951 |
| uniform | SKFFHAIM | 952 |
| uniform | YAYPIACS | 953 |
| uniform | LWDSSETK | 954 |
| uniform | IYEEEWHV | 955 |
| uniform | YNIIWCAL | 956 |
| uniform | KLERYIPF | 957 |
| uniform | AWHANLDP | 958 |
| uniform | QEPHDKKE | 959 |
| uniform | EIKDATQY | 960 |
| uniform | TEEMMSNQ | 961 |
| uniform | GLHQNHTD | 962 |
| uniform | ADRCKFKS | 963 |
| uniform | QNWRHIKW | 964 |
| uniform | TIVINDWH | 965 |
| uniform | GYRGKWEP | 966 |
| uniform | YRQIRTCD | 967 |
| uniform | ELQEQFRY | 968 |
| uniform | METVMLVF | 969 |
| uniform | DDLQISGM | 970 |
| uniform | SAPFKDTL | 971 |
| uniform | PIDFDTPK | 972 |
| uniform | LVRWKRLW | 973 |
| uniform | ITPASCQE | 974 |
| uniform | RAFHYHVI | 975 |
| uniform | MWRSFWRS | 976 |
| uniform | SNKWRLFY | 977 |
| uniform | KALGMFDY | 978 |
| uniform | TFSRAAIY | 979 |
| uniform | DMIYVKQA | 980 |
| uniform | LQDIGMYW | 981 |
| uniform | VAQWLQVG | 982 |
| uniform | DHVHTQCF | 983 |
| uniform | YKLFREEG | 984 |
| uniform | QIIWKDNA | 985 |
| uniform | GGGDMKYA | 986 |
| uniform | YAEGLRED | 987 |
| uniform | PYTFPRHA | 988 |
| uniform | AYGWGYIK | 989 |
| uniform | AFPDPFVD | 990 |
| uniform | IEVLVGFS | 991 |
| uniform | DMWMYNFA | 992 |
| uniform | HGSVKSPN | 993 |
| uniform | RTQAKVNC | 994 |
| uniform | NGKWGPCC | 995 |
| uniform | WCICLRPE | 996 |
| uniform | TIFTVRWT | 997 |
| uniform | CSGPWHMD | 998 |
| uniform | EGCAITKI | 999 |
| uniform | VCVNHRAP | 1000 |
| uniform | SNLDHKCL | 1001 |
| uniform | HDMDRYTI | 1002 |
| uniform | HPEFKSTV | 1003 |
| uniform | FSRFGWIF | 1004 |
| uniform | LGVDFGIQ | 1005 |
| uniform | WLAWCDCRL | 1006 |
| uniform | LTDKEHQFT | 1007 |
| uniform | NDVLWEPVT | 1008 |
| uniform | DVRFLCPLG | 1009 |
| uniform | PDEDRYLDI | 1010 |
| uniform | VFACRPKFF | 1011 |
| uniform | KVCKKRKLF | 1012 |
| uniform | ISMLRRDMM | 1013 |
| uniform | ISNITHNHC | 1014 |
| uniform | MNFYESLFF | 1015 |
| uniform | YDFMQLILT | 1016 |
| uniform | WWSKDDEDN | 1017 |
| uniform | GTRETFCAL | 1018 |
| uniform | PEASTLKSE | 1019 |
| uniform | QHLYVESCR | 1020 |
| uniform | MSCYALGGG | 1021 |
| uniform | WENYIMYHE | 1022 |
| uniform | HPNEVKRCP | 1023 |
| uniform | GPSRLNAAE | 1024 |
| uniform | SYQIRDYEI | 1025 |
| uniform | RLCNGNIVL | 1026 |
| uniform | PHQLAHTYY | 1027 |
| uniform | KIVNCCAPL | 1028 |
| uniform | LWTNEFHIV | 1029 |
| uniform | ICNYCWMPR | 1030 |
| uniform | AYWTWENSA | 1031 |
| uniform | EVWDPWIVL | 1032 |
| uniform | DMKHFYAHG | 1033 |
| uniform | IGYPSRWTL | 1034 |
| uniform | NHTDIYFKP | 1035 |
| uniform | WRESVTITM | 1036 |
| uniform | FEEGQMWRH | 1037 |
| uniform | CYGLELKVH | 1038 |
| uniform | YGESMKAEF | 1039 |
| uniform | NMIWHVTYK | 1040 |
| uniform | TIVCDKWTV | 1041 |
| uniform | HYLMREHWE | 1042 |
| uniform | PHVVFSYHR | 1043 |
| uniform | PDTVCLPKT | 1044 |
| uniform | ILESPGRDT | 1045 |
| uniform | PERILDFHW | 1046 |
| uniform | FESWRESPY | 1047 |
| uniform | SEYCADNQI | 1048 |
| uniform | VVVSKQKGT | 1049 |
| uniform | CDEWFDHIM | 1050 |
| uniform | GMPFSGGVP | 1051 |
| uniform | DKELSCLAF | 1052 |
| uniform | KLESCDHAY | 1053 |
| uniform | NCRISGHIY | 1054 |
| uniform | PDACDLPDV | 1055 |
| uniform | RLYLHSVNG | 1056 |
| uniform | HGQRFRVKD | 1057 |
| uniform | FWVIAYFVP | 1058 |
| uniform | SWELCHLGA | 1059 |
| uniform | SCYWNRHCD | 1060 |
| uniform | NYGAECYFS | 1061 |
| uniform | GPYFRHLTI | 1062 |
| uniform | SYPQVQHMV | 1063 |
| uniform | TAAQYRAKG | 1064 |
| uniform | QHAQKTLHS | 1065 |
| uniform | RWSCMLHMN | 1066 |
| uniform | APWTIENPM | 1067 |
| uniform | MRWLGDSWH | 1068 |
| uniform | RTHVRCYIN | 1069 |
| uniform | GQALLDTQV | 1070 |
| uniform | DDFSCDRVA | 1071 |
| uniform | SVACLIVQC | 1072 |
| uniform | LKTLREIPS | 1073 |
| uniform | RTTWDSCDE | 1074 |
| uniform | WYDKKRQGW | 1075 |
| uniform | IESENHRMT | 1076 |
| uniform | LSNYLMAKD | 1077 |
| uniform | FWCWVPSMF | 1078 |
| uniform | EQWTYSCFY | 1079 |
| uniform | HICPHDIWK | 1080 |
| uniform | KMFVEWLHY | 1081 |
| uniform | FVGNCCAPK | 1082 |
| uniform | RMEYRVGQP | 1083 |
| uniform | MFFQWFVLQ | 1084 |
| uniform | QQAHDTVMK | 1085 |
| uniform | CRHKCNPMY | 1086 |
| uniform | RYYQGAVKR | 1087 |
| uniform | QKSFTGGWC | 1088 |
| uniform | HRHAMKDGG | 1089 |
| uniform | PKDVPADRH | 1090 |
| uniform | QYSTQRFKR | 1091 |
| uniform | GSHALGEAW | 1092 |
| uniform | VNFTDGMMV | 1093 |
| uniform | RCRFCYVKE | 1094 |
| uniform | PENRIHILV | 1095 |
| uniform | IWWTFNMIE | 1096 |
| uniform | DVHCLNHND | 1097 |
| uniform | DSIAGAINY | 1098 |
| uniform | CSNDNKLYS | 1099 |
| uniform | VYTQSCMIP | 1100 |
| uniform | FDMRCIKSW | 1101 |
| uniform | TVFSKHHSP | 1102 |
| uniform | WDFWSSWLY | 1103 |
| uniform | YNSMNIHDP | 1104 |
| uniform | TFCNYKGSC | 1105 |
| uniform | PCLCMKIWR | 1106 |
| uniform | DCGDHMDDA | 1107 |
| uniform | SERWNQMAP | 1108 |
| uniform | YTTCETPCW | 1109 |
| uniform | CVCEHECMQ | 1110 |
| uniform | CHAFEACQT | 1111 |
| uniform | WNTLDTDYN | 1112 |
| uniform | RRARTWQWW | 1113 |
| uniform | PPNNKWIAK | 1114 |
| uniform | ITIGLFNDK | 1115 |
| uniform | ITLKSTGAQ | 1116 |
| uniform | HWFWLGWLI | 1117 |
| uniform | YGLFEARDL | 1118 |
| uniform | HVPREDNQM | 1119 |
| uniform | GFCGSYQQE | 1120 |
| uniform | NFKEYGDKH | 1121 |
| uniform | WNFWGIDDQ | 1122 |
| uniform | YAYLTVDGV | 1123 |
| uniform | LAPVGMRCQ | 1124 |
| uniform | WAEWRLAYY | 1125 |
| uniform | LNFTRIEVK | 1126 |
| uniform | TMINTMALV | 1127 |
| uniform | WDKNPQKAV | 1128 |
| uniform | YAYMCYFWL | 1129 |
| uniform | YKSGIWHGY | 1130 |
| uniform | FRQRYHLTP | 1131 |
| uniform | IHRCAAFSW | 1132 |
| uniform | YRCHTLMKM | 1133 |
| uniform | AWWMYVPSL | 1134 |
| uniform | QQGSLDCHD | 1135 |
| uniform | LHRCYVDNS | 1136 |
| uniform | HTSGKAQHP | 1137 |
| uniform | HAYAHLRNV | 1138 |
| uniform | TTLNNATDT | 1139 |
| uniform | QRKEVVQYG | 1140 |
| uniform | HNCMAAGQS | 1141 |
| uniform | NADNQRCSW | 1142 |
| uniform | CVCPQVIRS | 1143 |
| uniform | IRKMCKGSD | 1144 |
| uniform | VNRVLLGIN | 1145 |
| uniform | CQDCQVFAH | 1146 |
| uniform | SFYFQNIHQ | 1147 |
| uniform | WKYFNHVVN | 1148 |
| uniform | ILGHTRFME | 1149 |
| uniform | PKSFVYKWD | 1150 |
| uniform | SAHHSPEMW | 1151 |
| uniform | NMTWKESCG | 1152 |
| uniform | LEFQGCYMK | 1153 |
| uniform | TKDSIIKFG | 1154 |
| uniform | LVHWNATYW | 1155 |
| uniform | QLGEYGDIT | 1156 |
| uniform | CLWNIMIWK | 1157 |
| uniform | EWQHCHSYQ | 1158 |
| uniform | FCTVHRDTE | 1159 |
| uniform | PRLRHIAIC | 1160 |
| uniform | QATGVKCHM | 1161 |
| uniform | RAAENKDFW | 1162 |
| uniform | QEMQWQDFH | 1163 |
| uniform | YIARIECWQ | 1164 |
| uniform | PSWSWPTIH | 1165 |
| uniform | RYWIPGTDP | 1166 |
| uniform | QEPLWQNNF | 1167 |
| uniform | SRPWPANTI | 1168 |
| uniform | CDWVNKANG | 1169 |
| uniform | YWVVVLGFS | 1170 |
| uniform | PKCHSVRNH | 1171 |
| uniform | ELMHEHEID | 1172 |
| uniform | WTWMSTNGQ | 1173 |
| uniform | CDKSRVCFM | 1174 |
| uniform | THCDRQRDK | 1175 |
| uniform | GTYNPKAMQ | 1176 |
| uniform | NTPMSCMER | 1177 |
| uniform | NIRHGKACW | 1178 |
| uniform | HVCNKAKRS | 1179 |
| uniform | TEFRIVSET | 1180 |
| uniform | DAGIVYCFC | 1181 |
| uniform | NWDWVCSDH | 1182 |
| uniform | IYSVFCEAH | 1183 |
| uniform | EGYRHCHKH | 1184 |
| uniform | YMAGCRTLQ | 1185 |
| uniform | IYVKIEDVR | 1186 |
| uniform | KASYIREYN | 1187 |
| uniform | IQDLYKKFG | 1188 |
| uniform | WPTWVQRNT | 1189 |
| uniform | DKLKTCRIF | 1190 |
| uniform | EYDGCYKIY | 1191 |
| uniform | GFYVMRIGD | 1192 |
| uniform | KRWDLCTLS | 1193 |
| uniform | TVLAYVQKR | 1194 |
| uniform | VGKSFKNWL | 1195 |
| uniform | DDYFCVKID | 1196 |
| uniform | YWIRTWDSI | 1197 |
| uniform | NLWYRSYVF | 1198 |
| uniform | TEYVSCMWK | 1199 |
| uniform | IIALIPFVT | 1200 |
| uniform | DMKQCWAFM | 1201 |
| uniform | VALAWHVWF | 1202 |
| uniform | RNQCHQSMP | 1203 |
| uniform | KYIWDGYTR | 1204 |
| uniform | DLLVYMMNI | 1205 |
| uniform | FNRDDASDW | 1206 |
| uniform | TPSCTWIGD | 1207 |
| uniform | WNFLRLAEV | 1208 |
| uniform | LHDANHFGQ | 1209 |
| uniform | THSAMHLNN | 1210 |
| uniform | PIVIHTSTG | 1211 |
| uniform | NGPDLSQNR | 1212 |
| uniform | RKTWWQVVI | 1213 |
| uniform | RNIVCITHL | 1214 |
| uniform | YAICEQHPT | 1215 |
| uniform | LWINMTGLP | 1216 |
| uniform | YAGNLKWVC | 1217 |
| uniform | CSSVLMTEL | 1218 |
| uniform | TIRDCMDCI | 1219 |
| uniform | THMDYGMSK | 1220 |
| uniform | HRGCDMYDH | 1221 |
| uniform | EEHMICHVF | 1222 |
| uniform | CWMFFQGKV | 1223 |
| uniform | WRKHEQVDP | 1224 |
| uniform | MWVCECSPP | 1225 |
| uniform | HSSETWGDQ | 1226 |
| uniform | EHLISDRTE | 1227 |
| uniform | CLWEAIHGV | 1228 |
| uniform | REHRRGFYP | 1229 |
| uniform | DPLCAIDKQ | 1230 |
| uniform | LWWVVRCPF | 1231 |
| uniform | TTKVDIVFK | 1232 |
| uniform | PDQFSVCPH | 1233 |
| uniform | VCWFPYFKP | 1234 |
| uniform | GLFNRMHGA | 1235 |
| uniform | GELFFTMSD | 1236 |
| uniform | LPAHAMPGF | 1237 |
| uniform | NQDEDADQT | 1238 |
| uniform | IEEYEPVCM | 1239 |
| uniform | AAVRKIRSM | 1240 |
| uniform | SFHRYSCWV | 1241 |
| uniform | WFLYTKYRC | 1242 |
| uniform | ITRASLRQY | 1243 |
| uniform | PTISEIDRG | 1244 |
| uniform | FKSTWHTMA | 1245 |
| uniform | GLKGCLVPK | 1246 |
| uniform | MCSAVYIIQ | 1247 |
| uniform | KYDDEACYN | 1248 |
| uniform | NTFMLDNER | 1249 |
| uniform | SGKWGTSNP | 1250 |
| uniform | WWTFRCPWP | 1251 |
| uniform | PVPLETFNW | 1252 |
| uniform | FDRKPKCVW | 1253 |
| uniform | SKRYVVYKK | 1254 |
| uniform | CHLRNSEIA | 1255 |
| uniform | NLRSLQYWC | 1256 |
| uniform | WVECRFQLY | 1257 |
| uniform | MGEDIAKLG | 1258 |
| uniform | MRMYLKWLF | 1259 |
| uniform | KNGDKWMFH | 1260 |
| uniform | KYQAWYSGI | 1261 |
| uniform | YIQFFNSIK | 1262 |
| uniform | GFVNREVAR | 1263 |
| uniform | RFKSLGCWQ | 1264 |
| uniform | LGNERITRR | 1265 |
| uniform | HEDVQDGSQ | 1266 |
| uniform | GTQEVKGTM | 1267 |
| uniform | KVMRTDNFG | 1268 |
| uniform | NTWNQCHET | 1269 |
| uniform | VKMIDFFIA | 1270 |
| uniform | QGRYTKVCG | 1271 |
| uniform | WHWGWVKGE | 1272 |
| uniform | IMVVQEAWH | 1273 |
| uniform | FMVKKQDIY | 1274 |
| uniform | PKQRNYTHT | 1275 |
| uniform | VLFQQLHPI | 1276 |
| uniform | KYSTQYKWE | 1277 |
| uniform | GFDCYWMIQ | 1278 |
| uniform | RQKVELNYQ | 1279 |
| uniform | MDFFWPPPM | 1280 |
| uniform | TWGYWNCDP | 1281 |
| uniform | RICQYWYQI | 1282 |
| uniform | REYVGFPAK | 1283 |
| uniform | SVNPHSGMY | 1284 |
| uniform | VAACYELMC | 1285 |
| uniform | PTYCKPFTN | 1286 |
| uniform | ETERFMKML | 1287 |
| uniform | FCMMMFDCE | 1288 |
| uniform | AMRHDLSEQ | 1289 |
| uniform | AGQKECMLQ | 1290 |
| uniform | YDTSWHFSC | 1291 |
| uniform | HGAHWLSLY | 1292 |
| uniform | WKDGSIECM | 1293 |
| uniform | HTAEKPGDP | 1294 |
| uniform | YKWVIYFQY | 1295 |
| uniform | QAAHCLIGF | 1296 |
| uniform | VFRWCIPWQ | 1297 |
| uniform | DGIEFIEWT | 1298 |
| uniform | QGSAEGEKP | 1299 |
| uniform | MVHVAQLAK | 1300 |
| uniform | AGLYCCQYA | 1301 |
| uniform | RIRGFPINV | 1302 |
| uniform | VLCGTNQKC | 1303 |
| uniform | DMFTCEASE | 1304 |
| uniform | YINGEMWQT | 1305 |
| uniform | MSSKLCISY | 1306 |
| uniform | DINHFDNWL | 1307 |
| uniform | ECCYHQRDS | 1308 |
| uniform | FTMSWDYKC | 1309 |
| uniform | VEHGCLYAR | 1310 |
| uniform | ERDDGNPSR | 1311 |
| uniform | QFVGFFAFD | 1312 |
| uniform | DMHIWFAIV | 1313 |
| uniform | MAIHGKMLF | 1314 |
| uniform | SAGHRCHQS | 1315 |
| uniform | MWECYYYQD | 1316 |
| uniform | GCACLYWHN | 1317 |
| uniform | ESIMIQYQM | 1318 |
| uniform | GCGWHDHQN | 1319 |
| uniform | SISQLKEGI | 1320 |
| uniform | ENTWEDETD | 1321 |
| uniform | AQTCLNGRA | 1322 |
| uniform | GEYDQMLRM | 1323 |
| uniform | GSYWQAVWG | 1324 |
| uniform | GNFTTFQMN | 1325 |
| uniform | IFPYTVYHQ | 1326 |
| uniform | RAQWNGKNR | 1327 |
| uniform | RNPVKPCNC | 1328 |
| uniform | CPCKVYWLM | 1329 |
| uniform | PPQIGFRDG | 1330 |
| uniform | KWAQAQAHD | 1331 |
| uniform | VNVCINVYT | 1332 |
| uniform | FDLYIQRWV | 1333 |
| uniform | PGWMRNALW | 1334 |
| uniform | SDYPRPLQM | 1335 |
| uniform | CKKKVDQWF | 1336 |
| uniform | LARSHKPEC | 1337 |
| uniform | HGVDLTGHM | 1338 |
| uniform | VVSRLLTHY | 1339 |
| uniform | YTTSDRAVP | 1340 |
| uniform | EAYLAKSRP | 1341 |
| uniform | LACFPDRVC | 1342 |
| uniform | KNRLEEDFQ | 1343 |
| uniform | NCWACKWMF | 1344 |
| uniform | NWSCQSNTM | 1345 |
| uniform | CHSLCGPPE | 1346 |
| uniform | LIDAFLKMG | 1347 |
| uniform | HYMCAKWGI | 1348 |
| uniform | CPNHVMMWQ | 1349 |
| uniform | CPEGFAHDR | 1350 |
| uniform | SVAYTDTAS | 1351 |
| uniform | LCLYYRFEY | 1352 |
| uniform | MRMWTACCN | 1353 |
| uniform | QQSAVEYDM | 1354 |
| uniform | SDQNDSYMN | 1355 |
| uniform | HAYYCFIPQ | 1356 |
| uniform | NVPQSKSMM | 1357 |
| uniform | MLPRDYSFY | 1358 |
| uniform | KPIYHDRAT | 1359 |
| uniform | MDYRCYYRY | 1360 |
| uniform | TSKKNRQKY | 1361 |
| uniform | VKERWYYLL | 1362 |
| uniform | PSMICGIVE | 1363 |
| uniform | MQKMLMTTL | 1364 |
| uniform | INLPPAWWD | 1365 |
| uniform | LSNPFVVTE | 1366 |
| uniform | GWFSVSNIN | 1367 |
| uniform | KSPNMNPKD | 1368 |
| uniform | TDLIIRYWF | 1369 |
| uniform | TGWNPGWFN | 1370 |
| uniform | DFAIFVFLD | 1371 |
| uniform | NALDYNNMH | 1372 |
| uniform | EKYHFVLCL | 1373 |
| uniform | GSGSLWITF | 1374 |
| uniform | ANCLLLMDQ | 1375 |
| uniform | YVNEGFPGP | 1376 |
| uniform | QMQMCEKTS | 1377 |
| uniform | VGQQLWETL | 1378 |
| uniform | SIRKCRVDE | 1379 |
| uniform | GPWSHDTCW | 1380 |
| uniform | QQMWIPLMK | 1381 |
| uniform | SLAVMIQVY | 1382 |
| uniform | NELKSKRCL | 1383 |
| uniform | YKKMYFGYF | 1384 |
| uniform | DCARMELSI | 1385 |
| uniform | GCRQHYAEH | 1386 |
| uniform | KFDMDKHAH | 1387 |
| uniform | LTHPFINLT | 1388 |
| uniform | HFINCMTWN | 1389 |
| uniform | LEEEAFKSH | 1390 |
| uniform | LCNVDHSMT | 1391 |
| uniform | DNRCLWKCD | 1392 |
| uniform | LSNRGWIHW | 1393 |
| uniform | EAYVYRWHV | 1394 |
| uniform | PCHTSSQVK | 1395 |
| uniform | VFQKNCYGD | 1396 |
| uniform | FKNDHWSQQ | 1397 |
| uniform | NMDAYKNND | 1398 |
| uniform | YDSTWVWIP | 1399 |
| uniform | GRFALEWAI | 1400 |
| uniform | VMWFKMMRI | 1401 |
| uniform | MTAAKSEQG | 1402 |
| uniform | LCSDWMRVW | 1403 |
| uniform | RAGPKLNQL | 1404 |
| uniform | IWLRGHCLC | 1405 |
| uniform | WKDCVTDKS | 1406 |
| uniform | GYNGCATGL | 1407 |
| uniform | CGYQKMTRG | 1408 |
| uniform | EWAPQFPVH | 1409 |
| uniform | YKPTLGMSN | 1410 |
| uniform | EMEYECNEC | 1411 |
| uniform | EHMSSIMQG | 1412 |
| uniform | TMITRCNKF | 1413 |
| uniform | LIFCRHRSR | 1414 |
| uniform | PPKAGRITD | 1415 |
| uniform | KHHAHGEGT | 1416 |
| uniform | QEANQIKPE | 1417 |
| uniform | MNPSYWFWF | 1418 |
| uniform | GKAKHVKQS | 1419 |
| uniform | FLMSNIPVM | 1420 |
| uniform | WNGASNGWH | 1421 |
| uniform | HSSVLQHWK | 1422 |
| uniform | FKAYYYWHT | 1423 |
| uniform | QCQWSHYRG | 1424 |
| uniform | TAWDIVQCW | 1425 |
| uniform | EQQFYMYAS | 1426 |
| uniform | YPIHEGLTH | 1427 |
| uniform | CELGDSPRE | 1428 |
| uniform | DFLWCHWLM | 1429 |
| uniform | GCNYMVSSN | 1430 |
| uniform | AGNGSQHFC | 1431 |
| uniform | RRWRYSCGH | 1432 |
| uniform | DKDHMNSWQ | 1433 |
| uniform | MVNMDKGKQ | 1434 |
| uniform | SSQCGAYHW | 1435 |
| uniform | VFMRYPRHM | 1436 |
| uniform | PPDFMRNRN | 1437 |
| uniform | PSAWTEYTQ | 1438 |
| uniform | TRQEPIHRF | 1439 |
| uniform | QHGGDNQWC | 1440 |
| uniform | PVVSFCSNI | 1441 |
| uniform | QCAYGGAFR | 1442 |
| uniform | WFDMGLSME | 1443 |
| uniform | TFCTRPFQH | 1444 |
| uniform | GRKKWYFKN | 1445 |
| uniform | AIYPDMIFM | 1446 |
| uniform | ERDYKCSPQ | 1447 |
| uniform | IITAADKFF | 1448 |
| uniform | ASTYHSYVQ | 1449 |
| uniform | DNNLFSHKR | 1450 |
| uniform | VKKYVHHWV | 1451 |
| uniform | DIQLKNIGC | 1452 |
| uniform | ACFIKHKLG | 1453 |
| uniform | VEHVGHVIN | 1454 |
| uniform | AKWYDEGSN | 1455 |
| uniform | CWDYTMISH | 1456 |
| uniform | TCCGVCGPV | 1457 |
| uniform | SEHRHSGQF | 1458 |
| uniform | FYNAYKMHR | 1459 |
| uniform | KNWTCAFLP | 1460 |
| uniform | GHFYNVCMQ | 1461 |
| uniform | KQEKDYNWM | 1462 |
| uniform | CTHQFLTQT | 1463 |
| uniform | QMDKQLGMM | 1464 |
| uniform | KDTMYFTWA | 1465 |
| uniform | AIWRKPIPG | 1466 |
| uniform | FYWGSFGGA | 1467 |
| uniform | KTVSQEKWY | 1468 |
| uniform | HIMQLENRS | 1469 |
| uniform | CCHVSCYPT | 1470 |
| uniform | KDRGVEHIA | 1471 |
| uniform | DRDLILSCQ | 1472 |
| uniform | HAAGKFFYW | 1473 |
| uniform | CQMVRKHNF | 1474 |
| uniform | ERHRAHVHQ | 1475 |
| uniform | SNSYIYPVY | 1476 |
| uniform | HPGPEQAYR | 1477 |
| uniform | LYAKTARHE | 1478 |
| uniform | SPLPCCLHA | 1479 |
| uniform | LKSCDGKPA | 1480 |
| uniform | PELEWWPPD | 1481 |
| uniform | MCKIGHEVR | 1482 |
| uniform | LFTEQPHSD | 1483 |
| uniform | MDYHTWLKS | 1484 |
| uniform | TETHSPPAN | 1485 |
| uniform | LTMPAVVFL | 1486 |
| uniform | YAVLSVCPM | 1487 |
| uniform | DWGAVMIWP | 1488 |
| uniform | MGAGEVNCE | 1489 |
| uniform | KLAEKLCDK | 1490 |
| uniform | DTFKYAQYM | 1491 |
| uniform | LHQQRGVPF | 1492 |
| uniform | IIRETLYFA | 1493 |
| uniform | YHAEIMKCW | 1494 |
| uniform | RKEQDKRRL | 1495 |
| uniform | EWPNIYIWP | 1496 |
| uniform | YPEGYNHPQ | 1497 |
| uniform | WSRKGLIKN | 1498 |
| uniform | ASRQVFREN | 1499 |
| uniform | WKPGITALV | 1500 |
| uniform | RTAQFAGKK | 1501 |
| uniform | TSCNHKTSG | 1502 |
| uniform | PWLNKMPRD | 1503 |
| uniform | NGWNCPMPY | 1504 |
| uniform | LWYNTEEFQ | 1505 |
| uniform | TQKCVWMFT | 1506 |
| uniform | AGEMMMEGF | 1507 |
| uniform | NTVCNDISP | 1508 |
| uniform | SVDRKVNRN | 1509 |
| uniform | DNKKQPINE | 1510 |
| uniform | TEWEDFKTS | 1511 |
| uniform | VTSVRIYYG | 1512 |
| uniform | PSFDNVTEY | 1513 |
| uniform | VLFVWEASM | 1514 |
| uniform | WLEGFKTLM | 1515 |
| uniform | FGTNNKANE | 1516 |
| uniform | LFWPSFTGF | 1517 |
| uniform | VSNIKVTNC | 1518 |
| uniform | CRRAWVRIK | 1519 |
| uniform | QFLRPTLLP | 1520 |
| uniform | ANQCEEPED | 1521 |
| uniform | ARAVYIITA | 1522 |
| uniform | QPVDEDWLA | 1523 |
| uniform | NQWCVKVFF | 1524 |
| uniform | VGFMWIENQ | 1525 |
| uniform | SELEHNTNF | 1526 |
| uniform | WQQDSYNGD | 1527 |
| uniform | NWPQNIVTC | 1528 |
| uniform | VQFSYDTMS | 1529 |
| uniform | QGNFRCESV | 1530 |
| uniform | QRRHSMMTS | 1531 |
| uniform | HCSQRVIAN | 1532 |
| uniform | HRWWCNNGF | 1533 |
| uniform | TNENNRMWY | 1534 |
| uniform | IPLRKCPKW | 1535 |
| uniform | ANELLRPVK | 1536 |
| uniform | MYRAMDMCE | 1537 |
| uniform | GADTWMWKI | 1538 |
| uniform | RPPQPVAMP | 1539 |
| uniform | PFINSSNYM | 1540 |
| uniform | SHCFPLLEG | 1541 |
| uniform | LHRFSIPLN | 1542 |
| uniform | NDACQVNSI | 1543 |
| uniform | HTKPINEFT | 1544 |
| uniform | GFHDQLQVR | 1545 |
| uniform | ACHDQSYSN | 1546 |
| uniform | QCQVCVELM | 1547 |
| uniform | DDQWKLWSP | 1548 |
| uniform | KCHYMAMKE | 1549 |
| uniform | PWHCRTTLF | 1550 |
| uniform | KQQPSHKQP | 1551 |
| uniform | YTNIDAVSV | 1552 |
| uniform | QAKMEEGKW | 1553 |
| uniform | ACTYECFMH | 1554 |
| uniform | GTNWFAFIH | 1555 |
| uniform | AFMDFEREE | 1556 |
| uniform | FALPHRQNA | 1557 |
| uniform | FDNMRVQGF | 1558 |
| uniform | TILIKWSPN | 1559 |
| uniform | FDVADYGRG | 1560 |
| uniform | QKQEFLAGP | 1561 |
| uniform | VNQWSQMMY | 1562 |
| uniform | EKRNDTFVF | 1563 |
| uniform | MAGAYAWMA | 1564 |
| uniform | QQPDREAHY | 1565 |
| uniform | CTPETLPRW | 1566 |
| uniform | NASLHEESK | 1567 |
| uniform | WFDWNHGPH | 1568 |
| uniform | DKYHSTVFM | 1569 |
| uniform | RVIYAHMET | 1570 |
| uniform | LLFVMAQGR | 1571 |
| uniform | GRSMCFKSS | 1572 |
| uniform | VLARGRYTC | 1573 |
| uniform | LNWPNVGGM | 1574 |
| uniform | QDNKEHTLM | 1575 |
| uniform | MRGHVHSIV | 1576 |
| uniform | LITILGEKI | 1577 |
| uniform | HGQTWNSRY | 1578 |
| uniform | EMNYQDLHR | 1579 |
| uniform | AYNHMHDKF | 1580 |
| uniform | WGVVMGRGN | 1581 |
| uniform | HEKKPHFIN | 1582 |
| uniform | EGYGMRKPV | 1583 |
| uniform | QVDDHDWWG | 1584 |
| uniform | FWPTTTVVG | 1585 |
| uniform | TWATQTERN | 1586 |
| uniform | YMILNKPAE | 1587 |
| uniform | DSYCVIKKK | 1588 |
| uniform | KCSVAIPKL | 1589 |
| uniform | KRYHTMTWG | 1590 |
| uniform | VAKMVHCLH | 1591 |
| uniform | ICFYFVTHA | 1592 |
| uniform | FAIQDYREA | 1593 |
| uniform | WQCLPLTLQ | 1594 |
| uniform | HIFCYIQHG | 1595 |
| uniform | CHYRQCVCT | 1596 |
| uniform | VHMSNFMRF | 1597 |
| uniform | SETAADDIE | 1598 |
| uniform | IVRMAEWSD | 1599 |
| uniform | QGLCPQKTS | 1600 |
| uniform | RQGMASVMY | 1601 |
| uniform | YIRECNGTP | 1602 |
| uniform | AWDISYEED | 1603 |
| uniform | MWKMFACHK | 1604 |
| uniform | HQGFPPCIR | 1605 |
| uniform | RQSPRSEHY | 1606 |
| uniform | MVFWTKNQE | 1607 |
| uniform | VFWRQDHCR | 1608 |
| uniform | HMMVFMNLI | 1609 |
| uniform | VEWDYQWTF | 1610 |
| uniform | QLIFMDEIG | 1611 |
| uniform | PTCFPTAFQ | 1612 |
| uniform | NEKDEYNAN | 1613 |
| uniform | QFTYPYTVI | 1614 |
| uniform | IRSKAFFPP | 1615 |
| uniform | WVPYHRKEN | 1616 |
| uniform | PVSFEFWDV | 1617 |
| uniform | TVIPDNFTV | 1618 |
| uniform | HMQEMNPPQ | 1619 |
| uniform | DSPTPWRDV | 1620 |
| uniform | ESLMIQTSN | 1621 |
| uniform | DQCDWDPLT | 1622 |
| uniform | LNHASISSH | 1623 |
| uniform | GKVQVPCTG | 1624 |
| uniform | CCLFWFSGV | 1625 |
| uniform | VFYVTRAPT | 1626 |
| uniform | SKPQVAWHI | 1627 |
| uniform | YLSEHRHNL | 1628 |
| uniform | NQISWVNQC | 1629 |
| uniform | KPIAQDPGP | 1630 |
| uniform | PSEGRPLFE | 1631 |
| uniform | INTDVFCEY | 1632 |
| uniform | HPWGAIILY | 1633 |
| uniform | VPHGGRNDV | 1634 |
| uniform | DSYRTMWLF | 1635 |
| uniform | SIEGRYPDQ | 1636 |
| uniform | TLPHEHMQL | 1637 |
| uniform | GIFGFFWIR | 1638 |
| uniform | HHLGFHWDP | 1639 |
| uniform | HCDPTLEFW | 1640 |
| uniform | CMEDDSSGN | 1641 |
| uniform | MPNYNDIAF | 1642 |
| uniform | TKQEEKQLI | 1643 |
| uniform | PSTTYQHVF | 1644 |
| uniform | EGESCGPHT | 1645 |
| uniform | INTYMEPNTR | 1646 |
| uniform | GWMVLMIRM | 1647 |
| uniform | HSNNCGNGP | 1648 |
| uniform | CDQWRAQYN | 1649 |
| uniform | ENSIHVGEM | 1650 |
| uniform | FNDEAFYGY | 1651 |
| uniform | CLIEIHIWI | 1652 |
| uniform | TWGSKEAIF | 1653 |
| uniform | YAISWNSCC | 1654 |
| uniform | ALITRKMFT | 1655 |
| uniform | CTWLKFPYF | 1656 |
| uniform | GLPLVFNRW | 1657 |
| uniform | EARSHACTP | 1658 |
| uniform | CWCTSIEAV | 1659 |
| uniform | HPEPPAACD | 1660 |
| uniform | CHLPHTVHY | 1661 |
| uniform | RMANMPSWK | 1662 |
| uniform | MGPFRPSTW | 1663 |
| uniform | TWSAYRCKW | 1664 |
| uniform | YQHLEFFLY | 1665 |
| uniform | EWTTNLYYG | 1666 |
| uniform | GNVMCHDKP | 1667 |
| uniform | EASTKGLVI | 1668 |
| uniform | ANETPLRWN | 1669 |
| uniform | NVKTTTYQW | 1670 |
| uniform | RHVPVENNP | 1671 |
| uniform | YPKFTCMMW | 1672 |
| uniform | RHDGMGAWA | 1673 |
| uniform | FCPMAFHMK | 1674 |
| uniform | RSSHVDCFF | 1675 |
| uniform | HEVFFKWVK | 1676 |
| uniform | DWFWEIPFA | 1677 |
| uniform | DETAMLFDE | 1678 |
| uniform | AGPRFHRRH | 1679 |
| uniform | KPDHNHMLN | 1680 |
| uniform | INAQWQHHQ | 1681 |
| uniform | FQPRDQENQ | 1682 |
| uniform | NMMKSYQTI | 1683 |
| uniform | GFFYIQSGS | 1684 |
| uniform | QKHWPPLGR | 1685 |
| uniform | IWFHTHHNL | 1686 |
| uniform | YSKEVAICL | 1687 |
| uniform | KRANRLCLN | 1688 |
| uniform | VFTACKQPN | 1689 |
| uniform | DWKVNDRTN | 1690 |
| uniform | FNALVIGIM | 1691 |
| uniform | INQLQNLLM | 1692 |
| uniform | WMENYWHLQ | 1693 |
| uniform | GWLIQMCCW | 1694 |
| uniform | ECDPATGQW | 1695 |
| uniform | GNLEGECPL | 1696 |
| uniform | QWQQGGDKY | 1697 |
| uniform | MNELNQNRN | 1698 |
| uniform | EGFFGDLPG | 1699 |
| uniform | RPGRFKLPK | 1700 |
| uniform | KVGKNYFVK | 1701 |
| uniform | VIKKRYCVA | 1702 |
| uniform | MHWKQTCWC | 1703 |
| uniform | EYAEGQQQM | 1704 |
| uniform | PQTIGSFTR | 1705 |
| uniform | NDREEPVDT | 1706 |
| uniform | GLCWYLMKQ | 1707 |
| uniform | IAHQESRVF | 1708 |
| uniform | GDQQPFPWF | 1709 |
| uniform | TINNGWRMA | 1710 |
| uniform | NWIDTPYGH | 1711 |
| uniform | VIFRKQWLQ | 1712 |
| uniform | MFVGQDCVW | 1713 |
| uniform | SRTLIITKC | 1714 |
| uniform | ERGYINGMM | 1715 |
| uniform | RMPPMGNFK | 1716 |
| uniform | WKQMMTETT | 1717 |
| uniform | GQPSWLSMF | 1718 |
| uniform | FTAMPVTMA | 1719 |
| uniform | LAIRCKALQ | 1720 |
| uniform | QLSCPQECW | 1721 |
| uniform | HAPAVVTER | 1722 |
| uniform | EEKMNKSEA | 1723 |
| uniform | SDGAVLAFT | 1724 |
| uniform | FVQSVHILD | 1725 |
| uniform | NNPVADLKL | 1726 |
| uniform | RPRLVTDQV | 1727 |
| uniform | PRGTYDRQV | 1728 |
| uniform | GGRKELTYM | 1729 |
| uniform | PEAFATHRI | 1730 |
| uniform | KLFANNETT | 1731 |
| uniform | IMNGTRWSC | 1732 |
| uniform | NVLCFIFCA | 1733 |
| uniform | KILIKRDQN | 1734 |
| uniform | HQNSAIVCL | 1735 |
| uniform | RANIHWQIT | 1736 |
| uniform | TGANNWEMM | 1737 |
| uniform | DMFNVCKDA | 1738 |
| uniform | GTQHFRPSA | 1739 |
| uniform | NVMSTEEYS | 1740 |
| uniform | KGKYASYPN | 1741 |
| uniform | LMYCWYFFV | 1742 |
| uniform | GMVVHHRFG | 1743 |
| uniform | CHDFKKQWM | 1744 |
| uniform | HVFGDLSDS | 1745 |
| uniform | RYVESDMRK | 1746 |
| uniform | TYSIHYFTS | 1747 |
| uniform | LQYGSPVMM | 1748 |
| uniform | FDAVWPTDR | 1749 |
| uniform | LWHDTWWLM | 1750 |
| uniform | LVEPFHEAK | 1751 |
| uniform | RSAELDFRP | 1752 |
| uniform | ESERVIVNC | 1753 |
| uniform | SNCYQKTFN | 1754 |
| uniform | GWRFHGVLK | 1755 |
| uniform | SIAYFSSCR | 1756 |
| uniform | IQTRMPGMW | 1757 |
| uniform | CKITAETPS | 1758 |
| uniform | DGVPFGQLL | 1759 |
| uniform | DEYAQAACA | 1760 |
| uniform | VMQPRFRSC | 1761 |
| uniform | HNFYMIGCT | 1762 |
| uniform | DQVPGPRGQ | 1763 |
| uniform | SIHICLSCC | 1764 |
| uniform | CACNAMSVK | 1765 |
| uniform | CYWFPWTWN | 1766 |
| uniform | AAMMTVYHW | 1767 |
| uniform | PRTVARVFF | 1768 |
| uniform | RLHSHDIYH | 1769 |
| uniform | WHTRGAVHY | 1770 |
| uniform | GHRNLSIWF | 1771 |
| uniform | QQWCDVWMV | 1772 |
| uniform | PHCPLCHAR | 1773 |
| uniform | LTDSFACYA | 1774 |
| uniform | QWEKGDRFL | 1775 |
| uniform | MKQQPMVDC | 1776 |
| uniform | HNPLYQGFI | 1777 |
| uniform | FAHCFQLMF | 1778 |
| uniform | FERAKHVGG | 1779 |
| uniform | IQREDIVFM | 1780 |
| uniform | ERCSTGPVP | 1781 |
| uniform | SLVCRSKVG | 1782 |
| uniform | QDWMTMNMT | 1783 |
| uniform | ETWNCLGQI | 1784 |
| uniform | DAYFTLWIN | 1785 |
| uniform | WVHNGCRWS | 1786 |
| uniform | CLATECHYP | 1787 |
| uniform | NWNRLVPCA | 1788 |
| uniform | VDLFLCKEY | 1789 |
| uniform | MWRWWSHNP | 1790 |
| uniform | ERPLSGTEK | 1791 |
| uniform | KEDRGRGAE | 1792 |
| uniform | DFGAARTYG | 1793 |
| uniform | FCCNTQCFW | 1794 |
| uniform | HFVVCVFAW | 1795 |
| uniform | CGQGVFDVA | 1796 |
| uniform | LENNFPQMM | 1797 |
| uniform | MFEFALTPM | 1798 |
| uniform | CQSAALIAC | 1799 |
| uniform | CFWVYHFTM | 1800 |
| uniform | WFAHSNEEP | 1801 |
| uniform | NLNDKFWKG | 1802 |
| uniform | WVTYHRRCK | 1803 |
| uniform | PHYFMLTMV | 1804 |
| uniform | FMITNYGDM | 1805 |
| uniform | MNRIAIHNW | 1806 |
| uniform | FSWMIYNML | 1807 |
| uniform | EWGIFDDTN | 1808 |
| uniform | TIREGHFCL | 1809 |
| uniform | KHRSFDEST | 1810 |
| uniform | QGWKEKHAP | 1811 |
| uniform | CNVDHCCSC | 1812 |
| uniform | GFSKNIVLH | 1813 |
| uniform | ICNDCCECM | 1814 |
| uniform | MDTWEQVIM | 1815 |
| uniform | GSWGNLQNG | 1816 |
| uniform | NDALHCALY | 1817 |
| uniform | KHTDWCKCG | 1818 |
| uniform | MFQVNEREM | 1819 |
| uniform | HGAGPKSIT | 1820 |
| uniform | LRFVDMCPK | 1821 |
| uniform | NQHKMYNLI | 1822 |
| uniform | EIDVGGCIE | 1823 |
| uniform | AISFRCMAS | 1824 |
| uniform | MCMDDKEAP | 1825 |
| uniform | HNGFGHYTR | 1826 |
| uniform | YYINQQRFG | 1827 |
| uniform | FPFQNQFWR | 1828 |
| uniform | MMNFGLTLY | 1829 |
| uniform | MAPPNIDLW | 1830 |
| uniform | CNEFNNVFC | 1831 |
| uniform | YDAKCKVED | 1832 |
| uniform | RSPHMLWEF | 1833 |
| uniform | KYSGYWPDE | 1834 |
| uniform | NVDMGIMFD | 1835 |
| uniform | KLSWNMWYN | 1836 |
| uniform | ACEVTAPAA | 1837 |
| uniform | HHSIWEWPQ | 1838 |
| uniform | LQPVRYIKE | 1839 |
| uniform | MFRTKQWSD | 1840 |
| uniform | HGMGKTASF | 1841 |
| uniform | FYWDRTPTV | 1842 |
| uniform | VNGLCSNVR | 1843 |
| uniform | MMFMDQDGP | 1844 |
| uniform | NTQDMSFKY | 1845 |
| uniform | KPSWMVCKF | 1846 |
| uniform | EWLIHEHDL | 1847 |
| uniform | SGCLECGED | 1848 |
| uniform | QNMYDRQW | 1849 |
| uniform | WMTLYGAHI | 1850 |
| uniform | VRAMMNDTR | 1851 |
| uniform | MYNFEEYGL | 1852 |
| uniform | LFGWLFGKM | 1853 |
| uniform | LNHGKTING | 1854 |
| uniform | KWLQYGPFY | 1855 |
| uniform | YRVGSIPEA | 1856 |
| uniform | RWCVLFGGA | 1857 |
| uniform | ISNSDICSL | 1858 |
| uniform | QFGTCFTTK | 1859 |
| uniform | RDHYFIDGP | 1860 |
| uniform | SCIKDGTFE | 1861 |
| uniform | LDCVVLRQR | 1862 |
| uniform | HINIQLNFH | 1863 |
| uniform | QPVWPGFYD | 1864 |
| uniform | AYDISNVWN | 1865 |
| uniform | IKFADCPIL | 1866 |
| uniform | PGFTSFFYP | 1867 |
| uniform | GHKDNTTGC | 1868 |
| uniform | EPDTLQKVR | 1869 |
| uniform | PCDTRSNEH | 1870 |
| uniform | ERPRHEYYA | 1871 |
| uniform | CLAQRSEVQ | 1872 |
| uniform | CLCLGKASR | 1873 |
| uniform | TLRYHEGMI | 1874 |
| uniform | HCKILLDKD | 1875 |
| uniform | DDCDIQDRM | 1876 |
| uniform | YDYHSILGK | 1877 |
| uniform | QWMQAFHKV | 1878 |
| uniform | SRYDWIITC | 1879 |
| uniform | WGSISSTQQ | 1880 |
| uniform | LKFMYYEYY | 1881 |
| uniform | YEKENHAIH | 1882 |
| uniform | ILVGLVANS | 1883 |
| uniform | KPEERWDRA | 1884 |
| uniform | PCDMMRWYI | 1885 |
| uniform | YYKYWEGWG | 1886 |
| uniform | NQKQSMRPT | 1887 |
| uniform | IDYEAVNID | 1888 |
| uniform | CKATGVFQA | 1889 |
| uniform | GFRRDRGLN | 1890 |
| uniform | QTENFHKAY | 1891 |
| uniform | ELQDRFNGH | 1892 |
| uniform | QRVPAALVR | 1893 |
| uniform | KVVWFKILV | 1894 |
| uniform | HNDSEKGFN | 1895 |
| uniform | DARLVHEAD | 1896 |
| uniform | KLVLQTAIE | 1897 |
| uniform | ALGTIFNRK | 1898 |
| uniform | VAFPLKCFN | 1899 |
| uniform | GSGRDGWAQ | 1900 |
| uniform | WQNTEYPED | 1901 |
| uniform | HPDSYVFWE | 1902 |
| uniform | GSLLFYVEI | 1903 |
| uniform | CIGACCFWY | 1904 |
| uniform | NCKFCLRLY | 1905 |
| uniform | NYSNIMNMF | 1906 |
| uniform | GMYAYHEEQ | 1907 |
| uniform | QIRTLLAVD | 1908 |
| uniform | ERDIKKLSW | 1909 |
| uniform | EQAFPGLSV | 1910 |
| uniform | HFRPYDTLE | 1911 |
| uniform | IWNMYHCWG | 1912 |
| uniform | DIWRSPLYL | 1913 |
| uniform | VANNIGVTT | 1914 |
| uniform | NSIEGMGIH | 1915 |
| uniform | DDASWCYNV | 1916 |
| uniform | CYLFYPKTF | 1917 |
| uniform | VHYWLMTEM | 1918 |
| uniform | KDCEMPVFR | 1919 |
| uniform | KPEHPFCNT | 1920 |
| uniform | ERHQPWPDS | 1921 |
| uniform | EDNVTHVPQ | 1922 |
| uniform | GGLCHRCCS | 1923 |
| uniform | CKAAGVAQF | 1924 |
| uniform | QKQFELPGT | 1925 |
| uniform | YWGTCWAHE | 1926 |
| uniform | PEFRHYDQN | 1927 |
| uniform | GRATLAEWQ | 1928 |
| uniform | FWNLKAKWS | 1929 |
| uniform | VVVLGSCDP | 1930 |
| uniform | MVWVMWWCE | 1931 |
| uniform | YMHNQKAYM | 1932 |
| uniform | NQRRVAKGF | 1933 |
| uniform | GDLGPGDTN | 1934 |
| uniform | DVAWHNCSG | 1935 |
| uniform | CEWRHWFAN | 1936 |
| uniform | GYITYVRRC | 1937 |
| uniform | WPAFKPSQT | 1938 |
| uniform | RQNVGEVFF | 1939 |
| uniform | QMSHAGGIA | 1940 |
| uniform | LPVYCNHPP | 1941 |
| uniform | SRDIRTRHQ | 1942 |
| uniform | VWHMGEIFY | 1943 |
| uniform | SKTVGDPEY | 1944 |
| uniform | ADVMPFFDP | 1945 |
| uniform | MCKREWFWM | 1946 |
| uniform | PVYNCHHQV | 1947 |
| uniform | AFDGSLKYG | 1948 |
| uniform | FTTNGLCVQ | 1949 |
| uniform | DDWFCTNIM | 1950 |
| uniform | QNRYMFGNR | 1951 |
| uniform | CPKKTHWMH | 1952 |
| uniform | VVVPMHHKQ | 1953 |
| uniform | TITFHHANV | 1954 |
| uniform | KMSWGPNCN | 1955 |
| uniform | QCIWSKKAG | 1956 |
| uniform | PTTIHHIIF | 1957 |
| uniform | YEQWFQPDT | 1958 |
| uniform | SNQNVAEVL | 1959 |
| uniform | RPGQAYESF | 1960 |
| uniform | TIGLANPYP | 1961 |
| uniform | GLYLDIYFN | 1962 |
| uniform | PEGKQNFHA | 1963 |
| uniform | YAYMCPNAC | 1964 |
| uniform | YAGTNEFQH | 1965 |
| uniform | CTTKVTCFT | 1966 |
| uniform | KMNHAWHSA | 1967 |
| uniform | SNDFTGEYW | 1968 |
| uniform | RHFIITCAN | 1969 |
| uniform | RCDAMVGEY | 1970 |
| uniform | MVVNSKAGC | 1971 |
| uniform | WKFAMNTQW | 1972 |
| uniform | YEYERPPTG | 1973 |
| uniform | ATKIYVNMF | 1974 |
| uniform | NCDEKLLML | 1975 |
| uniform | KEFCIVSAK | 1976 |
| uniform | PDREYHRGT | 1977 |
| uniform | SRDDNYKSQ | 1978 |
| uniform | NVTGEPSSV | 1979 |
| uniform | TAPAAWHSN | 1980 |
| uniform | ASGNADTAI | 1981 |
| uniform | TVSIYMDVF | 1982 |
| uniform | NKWMVPKLC | 1983 |
| uniform | THIDSEPPR | 1984 |
| uniform | GEQEQTIVG | 1985 |
| uniform | TTSKHKWQF | 1986 |
| uniform | NLKTEEFDD | 1987 |
| uniform | SRHFFRCAW | 1988 |
| uniform | KFMNRVGET | 1989 |
| uniform | IPHYFQNRC | 1990 |
| uniform | RTQWLKDLG | 1991 |
| uniform | MWKYNEMWH | 1992 |
| uniform | HMMHLMCFD | 1993 |
| uniform | LGFTPRNCY | 1994 |
| uniform | LEGMVRMWA | 1995 |
| uniform | GAKPHAHAW | 1996 |
| uniform | DSELKLDRK | 1997 |
| uniform | HLGVNCCEC | 1998 |
| uniform | IFHTTITNR | 1999 |
| uniform | NVPHYRLWP | 2000 |
| uniform | YPFMSSYHF | 2001 |
| uniform | QDQGCRIEC | 2002 |
| uniform | IIRVFYLHS | 2003 |
| uniform | NCKCLCYLA | 2004 |
| uniform | VMHGSDVFQ | 2005 |
| uniform | KMSCPWSYWT | 2006 |
| uniform | YGPKDVYNNS | 2007 |
| uniform | SFKWNFFAKT | 2008 |
| uniform | NHSQTRQNED | 2009 |
| uniform | SGFLVLCSDY | 2010 |
| uniform | VYRKARTVTY | 2011 |
| uniform | YVCKKQAPND | 2012 |
| uniform | GPQKKNTMIY | 2013 |
| uniform | QQCQISWCPL | 2014 |
| uniform | AVYYSINQML | 2015 |
| uniform | VPERSHRDFW | 2016 |
| uniform | EGENCHLIIA | 2017 |
| uniform | DDSIRVDCTH | 2018 |
| uniform | YCGTEKLFLK | 2019 |
| uniform | GTRILEWMEI | 2020 |
| uniform | ERFKGWLIAY | 2021 |
| uniform | HHERRTHFEK | 2022 |
| uniform | QSGRSMYNSP | 2023 |
| uniform | RVFVVNPPRG | 2024 |
| uniform | TIVQSDAPRH | 2025 |
| uniform | HEWDQSVGWS | 2026 |
| uniform | VTQIYMFGYC | 2027 |
| uniform | PVVHMNFSWS | 2028 |
| uniform | SICADHQKYW | 2029 |
| uniform | VFIHPQCSYR | 2030 |
| uniform | IRRQNNEGAY | 2031 |
| uniform | WWMHHTTPCR | 2032 |
| uniform | SLKATQVCFP | 2033 |
| uniform | PIQQDPHSWW | 2034 |
| uniform | QGERHAIMLN | 2035 |
| uniform | VIAPIWGADH | 2036 |
| uniform | TEPNLTANMQ | 2037 |
| uniform | IRMGLGTYMQ | 2038 |
| uniform | VGTWTTFCKG | 2039 |
| uniform | SICSIPNWCM | 2040 |
| uniform | PKSAMQHFVQ | 2041 |
| uniform | KCCWQYYAKC | 2042 |
| uniform | IMPDHPDKFQ | 2043 |
| uniform | TMCKWVAWMD | 2044 |
| uniform | CTADCLMVLF | 2045 |
| uniform | HCLWKIQRQS | 2046 |
| uniform | NDTWKVHKIA | 2047 |
| uniform | FMEWHVCGHD | 2048 |
| uniform | SMLHHDVCPE | 2049 |
| uniform | KRLVWSTPQS | 2050 |
| uniform | VLIFCAIEAI | 2051 |
| uniform | WIDLHKIGCR | 2052 |
| uniform | VQWNCNLMDG | 2053 |
| uniform | CQTLGKMDGL | 2054 |
| uniform | LWSNLCIPRE | 2055 |
| uniform | HAIYYFPEDY | 2056 |
| uniform | ARASDRYAWV | 2057 |
| uniform | HKHYHYSMAH | 2058 |
| uniform | VAPPEKPMWT | 2059 |
| uniform | PHNRASNHLA | 2060 |
| uniform | RIEVLWFSHN | 2061 |
| uniform | NGTPVGMEWM | 2062 |
| uniform | YCQVHCRTPG | 2063 |
| uniform | DVNDPGYHDN | 2064 |
| uniform | HDYENIFLPF | 2065 |
| uniform | KLMIACHSPK | 2066 |
| uniform | SGEIMEWTRP | 2067 |
| uniform | QFWTEKIVWM | 2068 |
| uniform | RLQQRWWSCV | 2069 |
| uniform | HWHPKEWQRA | 2070 |
| uniform | TKADPTAAFR | 2071 |
| uniform | AQTFIVLHNQ | 2072 |
| uniform | REPWNHEGKA | 2073 |
| uniform | FNEYMYRAHV | 2074 |
| uniform | HGRIHAMFVV | 2075 |
| uniform | CSTIHNVWWL | 2076 |
| uniform | SDAIFFYRTN | 2077 |
| uniform | DGFFHEKPYT | 2078 |
| uniform | PQCEVIDAMP | 2079 |
| uniform | KKSEVFRAED | 2080 |
| uniform | HCIKDKVYCY | 2081 |
| uniform | ISMLPGMNIN | 2082 |
| uniform | CGQEFMHIFP | 2083 |
| uniform | IQCGAAHGSQ | 2084 |
| uniform | NYFLNMFANE | 2085 |
| uniform | PLLSHRDLPG | 2086 |
| uniform | YPIVHAALYW | 2087 |
| uniform | NKNPVVAHEC | 2088 |
| uniform | AVFGESDFLM | 2089 |
| uniform | NFPPQRLKSW | 2090 |
| uniform | GTRIEYIDEW | 2091 |
| uniform | AAQHHDSECH | 2092 |
| uniform | MSARYWHHYC | 2093 |
| uniform | ENDMVDKIPT | 2094 |
| uniform | QNILAKRDMH | 2095 |
| uniform | KELVKLCAFD | 2096 |
| uniform | LTQAMANTKV | 2097 |
| uniform | HTWPYHGCEE | 2098 |
| uniform | RIAQEEVWSW | 2099 |
| uniform | AYFIHISWHR | 2100 |
| uniform | PRPLGNIHEN | 2101 |
| uniform | WYMNVTNKNT | 2102 |
| uniform | INTIMAQAPQ | 2103 |
| uniform | QINDGCKQVR | 2104 |
| uniform | CGICWRQWPG | 2105 |
| uniform | FIIASDPPYY | 2106 |
| uniform | AVIQEWNRFM | 2107 |
| uniform | CKVHYWYSWW | 2108 |
| uniform | IEIYCMFAQY | 2109 |
| uniform | KEMVAAGTCI | 2110 |
| uniform | MVPINKRHKH | 2111 |
| uniform | SCHHEMGPCP | 2112 |
| uniform | AFVEYWGLPT | 2113 |
| uniform | IWRASEWPFN | 2114 |
| uniform | WLDYCFVMMM | 2115 |
| uniform | TARCVWMGYC | 2116 |
| uniform | ILWPGKQTRCQ | 2117 |
| uniform | MYMPNMNRYT | 2118 |
| uniform | WSMFFSTEWV | 2119 |
| uniform | CEIEKPVPCV | 2120 |
| uniform | ADTCGIIEDD | 2121 |
| uniform | KYRQERLDHC | 2122 |
| uniform | PFIWRRTGCI | 2123 |
| uniform | WHLATCMPFH | 2124 |
| uniform | IIHNPEWRVT | 2125 |
| uniform | NWFPWIHAME | 2126 |
| uniform | QYQFAVWSKR | 2127 |
| uniform | GTSGHVKGCH | 2128 |
| uniform | EGAMMQQSVY | 2129 |
| uniform | TQKGGGFGCG | 2130 |
| uniform | LWTSFHAEII | 2131 |
| uniform | DIGCVIIIAM | 2132 |
| uniform | HKDTPVTMEG | 2133 |
| uniform | FWPAFCLCFF | 2134 |
| uniform | TDIRTSIDNR | 2135 |
| uniform | FQSNKNFMGI | 2136 |
| uniform | LAQHTTRHYT | 2137 |
| uniform | MIMMLVHPLN | 2138 |
| uniform | NQVMMKDMTH | 2139 |
| uniform | TICQRCSYLQ | 2140 |
| uniform | NAGNFWRHAM | 2141 |
| uniform | RIYPPRQQYK | 2142 |
| uniform | IYGCSKAWSD | 2143 |
| uniform | QGAYMLFVLF | 2144 |
| uniform | RFFWSHRRWY | 2145 |
| uniform | RWGWSVWIGM | 2146 |
| uniform | WPDIETGLHR | 2147 |
| uniform | HKLHRGSYQQ | 2148 |
| uniform | GEGNIQSCYI | 2149 |
| uniform | LKAHFAIQLH | 2150 |
| uniform | LYIFVQKFPK | 2151 |
| uniform | PQSKDWAHWF | 2152 |
| uniform | TAVVWPFSRF | 2153 |
| uniform | TKDQANTPHR | 2154 |
| uniform | FWAIQCGWIP | 2155 |
| uniform | VHVKTYDVIN | 2156 |
| uniform | LGVSKPLEIC | 2157 |
| uniform | AQTKYWYANT | 2158 |
| uniform | FGMNDVEQVH | 2159 |
| uniform | WQFKPARQTE | 2160 |
| uniform | MPQVKISTYA | 2161 |
| uniform | IWNNKKNQHN | 2162 |
| uniform | QLHDIAGQNV | 2163 |
| uniform | AVKYTWMGYI | 2164 |
| uniform | QGHVSILPNL | 2165 |
| uniform | EYHKPNHKHH | 2166 |
| uniform | LCISMAVCMH | 2167 |
| uniform | EAFIPWMCQL | 2168 |
| uniform | EMAVMATMRN | 2169 |
| uniform | YGHHDSQMLF | 2170 |
| uniform | LHPNWKNGYC | 2171 |
| uniform | HAKKPMYNRV | 2172 |
| uniform | VEKVNFLCKP | 2173 |
| uniform | LVSDEPTINN | 2174 |
| uniform | CSNWNNRIEL | 2175 |
| uniform | HRMNVMPFIN | 2176 |
| uniform | NKSQQSMIRD | 2177 |
| uniform | VFCGILQLPV | 2178 |
| uniform | SNQRQMCNWG | 2179 |
| uniform | DVRPHAEWYK | 2180 |
| uniform | PGRIKWKFQM | 2181 |
| uniform | AYHELENWET | 2182 |
| uniform | FECKNLVDKW | 2183 |
| uniform | HASSSWVTGC | 2184 |
| uniform | RYVECIRDGM | 2185 |
| uniform | TVFTYSDWHV | 2186 |
| uniform | EQSRVLSHCE | 2187 |
| uniform | GKDCPVKPRI | 2188 |
| uniform | RRCNAFKCDR | 2189 |
| uniform | KFDCKWKMAT | 2190 |
| uniform | HYASIMMDLC | 2191 |
| uniform | TDEPLNRVEP | 2192 |
| uniform | FEAGWMYVHA | 2193 |
| uniform | ETLYKCGWNR | 2194 |
| uniform | FMERMTEYTL | 2195 |
| uniform | NTAICFDHHS | 2196 |
| uniform | QLEPQNYLDY | 2197 |
| uniform | NATDPDPCKK | 2198 |
| uniform | GIIAFNFVVP | 2199 |
| uniform | HRRWFHIHPA | 2200 |
| uniform | YNCTKSFSQW | 2201 |
| uniform | EENAQMEWDM | 2202 |
| uniform | PWSRPDFEKS | 2203 |
| uniform | SHQHKVSCHR | 2204 |
| uniform | IYRRYQWWRL | 2205 |
| uniform | VVPGMSSFPP | 2206 |
| uniform | CWKGSGQEYT | 2207 |
| uniform | VLFAWWEFVN | 2208 |
| uniform | VCNHSEVQTP | 2209 |
| uniform | NHTNRYGKTV | 2210 |
| uniform | TQVQIQWDYL | 2211 |
| uniform | GRVPMEDNVG | 2212 |
| uniform | EHPYPMLFLR | 2213 |
| uniform | ELGIHLPNLI | 2214 |
| uniform | GQNPAYYRVV | 2215 |
| uniform | GSSREWRTEG | 2216 |
| uniform | CAMFQWCEFA | 2217 |
| uniform | PFDPSWQWDI | 2218 |
| uniform | KVRTNWEDQQ | 2219 |
| uniform | QSFKQQYFIP | 2220 |
| uniform | YRPFWHRKIF | 2221 |
| uniform | NYDERIGDIH | 2222 |
| uniform | CCIYESHQPV | 2223 |
| uniform | YCSEENCQGP | 2224 |
| uniform | TCYEKPMRQL | 2225 |
| uniform | TQVVYFDSKD | 2226 |
| uniform | KQPMACDEDI | 2227 |
| uniform | IWAHVWRTWM | 2228 |
| uniform | RTCRKLLRDI | 2229 |
| uniform | CYKSSWDPS | 2230 |
| uniform | HHCDVAHKEM | 2231 |
| uniform | VCPTGYGLYW | 2232 |
| uniform | IKCTPPTGCE | 2233 |
| uniform | WEDGFPHWWN | 2234 |
| uniform | YKPQGLWIMS | 2235 |
| uniform | AIFQEEDSDA | 2236 |
| uniform | LKGGQAILNS | 2237 |
| uniform | CMWLHVCYEP | 2238 |
| uniform | RGCLISHGQA | 2239 |
| uniform | AQMCMQHPIL | 2240 |
| uniform | DFVLSYWCSN | 2241 |
| uniform | GESQKATDDE | 2242 |
| uniform | CLRIDCFMFN | 2243 |
| uniform | WFTAHWKTPD | 2244 |
| uniform | MTYTDGEKFE | 2245 |
| uniform | YCRWYLSRML | 2246 |
| uniform | YWKAYCWQQK | 2247 |
| uniform | QWFSTYYLSM | 2248 |
| uniform | TTFGCVHWGM | 2249 |
| uniform | ISAICELGWE | 2250 |
| uniform | EHTTHYPAQN | 2251 |
| uniform | NGKWLNYAPD | 2252 |
| uniform | TMFHHSWLPI | 2253 |
| uniform | HEADLCYKIL | 2254 |
| uniform | NWYVFQTESA | 2255 |
| uniform | LDWWHLGDFV | 2256 |
| uniform | DVTPVECKRD | 2257 |
| uniform | SMWPFYSCWS | 2258 |
| uniform | HSRKWYNNDG | 2259 |
| uniform | RLSPTSRDSS | 2260 |
| uniform | YGENNFYCKT | 2261 |
| uniform | MVGFWTFPHW | 2262 |
| uniform | ASFHLYEPPA | 2263 |
| uniform | WWINIRDDIN | 2264 |
| uniform | CKWMKRACHN | 2265 |
| uniform | FGSYNFIVRR | 2266 |
| uniform | LEVIVEIPFH | 2267 |
| uniform | AFRSKWQMAL | 2268 |
| uniform | RDGRECKVLN | 2269 |
| uniform | HGKPYPEPDE | 2270 |
| uniform | VKYYRTDQTD | 2271 |
| uniform | YYTKTIYPCG | 2272 |
| uniform | HKYKDWLISR | 2273 |
| uniform | HYTGAKPPIP | 2274 |
| uniform | AWKKPPKQVL | 2275 |
| uniform | HACVPCYVDQ | 2276 |
| uniform | RVMCWAVFLK | 2277 |
| uniform | TWDNHRLSEH | 2278 |
| uniform | FHHGVDVYWV | 2279 |
| uniform | GAEGSCCWWF | 2280 |
| uniform | SRFEPRLSTT | 2281 |
| uniform | CCWQNGKTCV | 2282 |
| uniform | QTEPQKDSII | 2283 |
| uniform | NTDIDQVSSI | 2284 |
| uniform | SNSEEWYDEV | 2285 |
| uniform | DGVDDVTADK | 2286 |
| uniform | TNECIRVLKK | 2287 |
| uniform | CGSNNKHEIV | 2288 |
| uniform | TIATCQKKTV | 2289 |
| uniform | HVIGRCFCLS | 2290 |
| uniform | GLDFFKQDMD | 2291 |
| uniform | NPERQTHHGV | 2292 |
| uniform | VSPFCNPSYN | 2293 |
| uniform | WLNHTNSNVF | 2294 |
| uniform | RNRKFFWPFM | 2295 |
| uniform | RHHAHQHRNG | 2296 |
| uniform | RSDYQQFWPT | 2297 |
| uniform | DHSLMISSLA | 2298 |
| uniform | QLRGSRPKYA | 2299 |
| uniform | AGWVDARQYE | 2300 |
| uniform | KPDNYMFRCL | 2301 |
| uniform | DAYRYLLAGN | 2302 |
| uniform | INFGTMNWYL | 2303 |
| uniform | NNDHLAQSAR | 2304 |
| uniform | RVPQNHHHRD | 2305 |
| uniform | GAMTVQIKGY | 2306 |
| uniform | VDFTNFAIFD | 2307 |
| uniform | PFWALTQDHL | 2308 |
| uniform | GRGPEQPLMF | 2309 |
| uniform | KEVWRTCRDI | 2310 |
| uniform | ARLKDRFVNN | 2311 |
| uniform | MFAMLQCIWL | 2312 |
| uniform | FVCEGDCDRI | 2313 |
| uniform | LMVNWNLYAV | 2314 |
| uniform | QDCHNMHAPD | 2315 |
| uniform | QHSFTDCACC | 2316 |
| uniform | IEGIGMDINQ | 2317 |
| uniform | PGAAETPFIR | 2318 |
| uniform | MGKGVGMFSL | 2319 |
| uniform | RMTLPGWLMK | 2320 |
| uniform | SKLHPWNVYD | 2321 |
| uniform | QLKQIWYYNS | 2322 |
| uniform | ESNGAHITVE | 2323 |
| uniform | MLLDSCFQSI | 2324 |
| uniform | VVFQMGDCYN | 2325 |
| uniform | CTQYNTCTYK | 2326 |
| uniform | LDPNPREYGY | 2327 |
| uniform | LYQTSIYHNK | 2328 |
| uniform | GSYGCAVDGE | 2329 |
| uniform | MFYAVGSSST | 2330 |
| uniform | DIYWTHNPMY | 2331 |
| uniform | MYGAVPTLAN | 2332 |
| uniform | KLAEPYQQKP | 2333 |
| uniform | EALQSQDHEC | 2334 |
| uniform | WARENMCYGN | 2335 |
| uniform | NSRPWCLRWE | 2336 |
| uniform | TDIYLMYISR | 2337 |
| uniform | TQQTIGLEQN | 2338 |
| uniform | FYTTCMEMDW | 2339 |
| uniform | HNYTEHHEQE | 2340 |
| uniform | HGSAKTQVVS | 2341 |
| uniform | EIGKGVVDHK | 2342 |
| uniform | VKMKCHVYSW | 2343 |
| uniform | CPCRPVVMLM | 2344 |
| uniform | KLTFSVEGQN | 2345 |
| uniform | FLCIGAMMFQ | 2346 |
| uniform | YMIAKCFKCE | 2347 |
| uniform | GTPFAMFDVH | 2348 |
| uniform | YKFVAAVMTF | 2349 |
| uniform | MKDCWSVAVA | 2350 |
| uniform | IKWNEPVAWV | 2351 |
| uniform | LFTRNYMFYR | 2352 |
| uniform | IKVFDARSHI | 2353 |
| uniform | AMPDPCYCPL | 2354 |
| uniform | YCPYHKTGNH | 2355 |
| uniform | FKYFMQINDK | 2356 |
| uniform | MQDFMPCAGD | 2357 |
| uniform | NYSMTKMGEI | 2358 |
| uniform | LLDRKYRITY | 2359 |
| uniform | HVHAVWVLYW | 2360 |
| uniform | LNDQNDTTHD | 2361 |
| uniform | QQNIFSKALH | 2362 |
| uniform | NGFKPAGEWI | 2363 |
| uniform | YLHFPSFQFM | 2364 |
| uniform | PEGNDYSTQV | 2365 |
| uniform | FWLCWWKALL | 2366 |
| uniform | FDRCSDPGGN | 2367 |
| uniform | IQVVGQRYHC | 2368 |
| uniform | HPMFPMMLEC | 2369 |
| uniform | GTMWLQFDLK | 2370 |
| uniform | PWAHYYYSHG | 2371 |
| uniform | KYGCFMERVR | 2372 |
| uniform | KDRCAKEIVP | 2373 |
| uniform | ENVFIMDEHV | 2374 |
| uniform | RLSGYVAERM | 2375 |
| uniform | LQASIWWFGF | 2376 |
| uniform | DPRPIVVGHW | 2377 |
| uniform | FHETDKKLPL | 2378 |
| uniform | TKECNVGSYA | 2379 |
| uniform | WIVNMGAMFQ | 2380 |
| uniform | YEALRLQYIR | 2381 |
| uniform | KPHWIRLEKT | 2382 |
| uniform | TMQENMNQRG | 2383 |
| uniform | SDECWSIFCT | 2384 |
| uniform | FNWRVHFLCR | 2385 |
| uniform | KDRMNKYQFH | 2386 |
| uniform | PTFAMSFNGM | 2387 |
| uniform | MMKSHPHYHF | 2388 |
| uniform | LVQVHSEWSW | 2389 |
| uniform | CILPVYIDFV | 2390 |
| uniform | ITYRWRARND | 2391 |
| uniform | WQEKNGSLHF | 2392 |
| uniform | KDYCTTTVFL | 2393 |
| uniform | IIAKQRYTKE | 2394 |
| uniform | PWNMIKWSSW | 2395 |
| uniform | ADTWCAADAP | 2396 |
| uniform | MKSWWEWAWL | 2397 |
| uniform | RGAAHDWYTQ | 2398 |
| uniform | YMCCAWWFIT | 2399 |
| uniform | FWGTIMDKWW | 2400 |
| uniform | TPWAVMNGGK | 2401 |
| uniform | AICFVEIIPF | 2402 |
| uniform | MCHMQKVMYE | 2403 |
| uniform | MPNIKGKRKH | 2404 |
| uniform | GEYMVLDCLS | 2405 |
| uniform | TLYIINVVQE | 2406 |
| uniform | TNRMCTHVKS | 2407 |
| uniform | CVAVNDAGNP | 2408 |
| uniform | AFGPKVEDTD | 2409 |
| uniform | IDMVEMLDFQ | 2410 |
| uniform | VNGDSEAYEH | 2411 |
| uniform | CYFFQWSLCA | 2412 |
| uniform | SETMYTYYYL | 2413 |
| uniform | DPIGRMFRHR | 2414 |
| uniform | KHALRWEANC | 2415 |
| uniform | RPNYSRKCNP | 2416 |
| uniform | IYWHYGGKHH | 2417 |
| uniform | RCRYQYDMVI | 2418 |
| uniform | CAMHVFWYHD | 2419 |
| uniform | FGEIAGMNKF | 2420 |
| uniform | YSEESSGLYC | 2421 |
| uniform | SWQDIMVQAW | 2422 |
| uniform | SKEEQNFDWV | 2423 |
| uniform | TAYMHREWYY | 2424 |
| uniform | EHVMMLAVGC | 2425 |
| uniform | CKTWDDNVMP | 2426 |
| uniform | VNAIVQFIQK | 2427 |
| uniform | AGIRQQKPGL | 2428 |
| uniform | CYMWVSNRPD | 2429 |
| uniform | DYPHGTACCM | 2430 |
| uniform | HTPCDDAYFS | 2431 |
| uniform | CFGTDAGSEW | 2432 |
| uniform | YCACGLIAHV | 2433 |
| uniform | WKMWPIDFCH | 2434 |
| uniform | LHMFQHFTQI | 2435 |
| uniform | GRRYGCNLFF | 2436 |
| uniform | KCDYAMYVMM | 2437 |
| uniform | EDWWGVWQCI | 2438 |
| uniform | WMMIAHEGSD | 2439 |
| uniform | CVQNVINEIH | 2440 |
| uniform | PDYPEMQSID | 2441 |
| uniform | HQHAREQWHS | 2442 |
| uniform | KQQEELIYTR | 2443 |
| uniform | WEMRDWHDWV | 2444 |
| uniform | FMGSGRQCMS | 2445 |
| uniform | EEHAHSTPHH | 2446 |
| uniform | RWGRYTIDFN | 2447 |
| uniform | LDSMVNIHWG | 2448 |
| uniform | YQLATCWEST | 2449 |
| uniform | YRWECLEGVR | 2450 |
| uniform | GDHKSQQCGK | 2451 |
| uniform | HDSLFEERSL | 2452 |
| uniform | WKHHRECTSP | 2453 |
| uniform | KWCFPWMMHH | 2454 |
| uniform | ILHCMPRHYM | 2455 |
| uniform | VEDGDNDQMH | 2456 |
| uniform | ERGSMFFYKI | 2457 |
| uniform | TAIKFVWDLK | 2458 |
| uniform | INCCTPPTYT | 2459 |
| uniform | VTKCNLQALA | 2460 |
| uniform | PAPNANNQLC | 2461 |
| uniform | IWNIWRRQQE | 2462 |
| uniform | FCTCDVWRKG | 2463 |
| uniform | FNKFCGVDYL | 2464 |
| uniform | GFCPWAMGSK | 2465 |
| uniform | WKACWGVKEL | 2466 |
| uniform | NWAQAHGKLT | 2467 |
| uniform | RDQYCRVADD | 2468 |
| uniform | RTSHYMMHTT | 2469 |
| uniform | MNQTGVRPDL | 2470 |
| uniform | TFWYQLAHGY | 2471 |
| uniform | GNTECCIRKA | 2472 |
| uniform | NLQMHFNKID | 2473 |
| uniform | FHNDFYSTIV | 2474 |
| uniform | KNAMGMWLPK | 2475 |
| uniform | LTYHSFWACH | 2476 |
| uniform | CHLDQWTANH | 2477 |
| uniform | GFCRATGYHS | 2478 |
| uniform | YDGKDKTMSQ | 2479 |
| uniform | DHMGNHPAGL | 2480 |
| uniform | EESDSITKFH | 2481 |
| uniform | PDIEKPHAEQ | 2482 |
| uniform | VWCKCNQNYQ | 2483 |
| uniform | QIKHSVTLSI | 2484 |
| uniform | TPVYFDWPFP | 2485 |
| uniform | YLAHNTCIFT | 2486 |
| uniform | TRWNWEAVAH | 2487 |
| uniform | PCPKEFFMFR | 2488 |
| uniform | HFIKRENVDI | 2489 |
| uniform | QFDQYERAHN | 2490 |
| uniform | VVPWIQQSTS | 2491 |
| uniform | KYMPCQSHAK | 2492 |
| uniform | YLEVSRAGKD | 2493 |
| uniform | MWKTRCLAPD | 2494 |
| uniform | YYLVGVMKHC | 2495 |
| uniform | ANKATAEGSL | 2496 |
| uniform | EDNCKSDVYI | 2497 |
| uniform | SWSWYEEITW | 2498 |
| uniform | AQHQDMSCHM | 2499 |
| uniform | PPSKVHRRVG | 2500 |
| uniform | WTIDVQYGDA | 2501 |
| uniform | HIDDYNKRCY | 2502 |
| uniform | HIGQYVDLYW | 2503 |
| uniform | DTNTLNSTKH | 2504 |
| uniform | SEIAVLIMIR | 2505 |
| uniform | ELAAPDENLR | 2506 |
| uniform | WMMLWKTLPE | 2507 |
| uniform | DQWVRWHNCI | 2508 |
| uniform | EIMTKYVFWK | 2509 |
| uniform | NKECLINYEM | 2510 |
| uniform | YGNLFNHAER | 2511 |
| uniform | NERNWLCLHN | 2512 |
| uniform | QHYAMEQGNR | 2513 |
| uniform | IVCCNADCVV | 2514 |
| uniform | NKDCHSFSMT | 2515 |
| uniform | LKHYMIEQVS | 2516 |
| uniform | HWDLVQHFMM | 2517 |
| uniform | YLAIRHVAFW | 2518 |
| uniform | ILYNPNYIMN | 2519 |
| uniform | NHGIFKQDSA | 2520 |
| uniform | ITEAPYQRYS | 2521 |
| uniform | LDGKIPRNKA | 2522 |
| uniform | CSSMGYLPCP | 2523 |
| uniform | VCDMSGQYCD | 2524 |
| uniform | NFDKRDRLDQ | 2525 |
| uniform | YHEYSLPEWN | 2526 |
| uniform | YERPSNKGNH | 2527 |
| uniform | RTNGEIFSYS | 2528 |
| uniform | HWALCFMLKG | 2529 |
| uniform | CFCSANTEDA | 2530 |
| uniform | LVCYVWGWSA | 2531 |
| uniform | CTQQGPNRKF | 2532 |
| uniform | KIEEEDHQRF | 2533 |
| uniform | CKSHLYEVKT | 2534 |
| uniform | NPCPSHKYDV | 2535 |
| uniform | FLGPAINPHT | 2536 |
| uniform | EMPYFVHWNQ | 2537 |
| uniform | QYRDFVAVNR | 2538 |
| uniform | MMDNWNFKQR | 2539 |
| uniform | TKPFPNWAYW | 2540 |
| uniform | MIWGAPKLNP | 2541 |
| uniform | HLGMKHGNFN | 2542 |
| uniform | QIVAQHKPVF | 2543 |
| uniform | QPDMSTLSRP | 2544 |
| uniform | SVATAVSQAD | 2545 |
| uniform | CCVMERYNKK | 2546 |
| uniform | MGCFPVCIHI | 2547 |
| uniform | MMMKIAIPVK | 2548 |
| uniform | CQWIGDARLR | 2549 |
| uniform | DTSVDFMMQL | 2550 |
| uniform | VQYSPNPYIE | 2551 |
| uniform | LDKEGATKKC | 2552 |
| uniform | RFMRMRLWAQ | 2553 |
| uniform | YSGYTHHSVE | 2554 |
| uniform | IIPSVIQVQI | 2555 |
| uniform | DSADTDTAER | 2556 |
| uniform | CCSFYPDQCL | 2557 |
| uniform | CRDRVGTYFD | 2558 |
| uniform | KTFCNDRNFQ | 2559 |
| uniform | KYRWKDKSWT | 2560 |
| uniform | DMTYPYKQLN | 2561 |
| uniform | REISCVAWYL | 2562 |
| uniform | QQRDFLSDCW | 2563 |
| uniform | QHRFPLRGRK | 2564 |
| uniform | IQQPVPVTYT | 2565 |
| uniform | TMMLEPLLVN | 2566 |
| uniform | HWCANIDDYY | 2567 |
| uniform | SDQCERIAFH | 2568 |
| uniform | YNPHLKSVSY | 2569 |
| uniform | ECIWHEMWWC | 2570 |
| uniform | CTCTRCSRDP | 2571 |
| uniform | SKGHDIGATY | 2572 |
| uniform | YDIMNKLSGH | 2573 |
| uniform | FFKSDVPVVW | 2574 |
| uniform | HPARWWISHM | 2575 |
| uniform | IMAEYRMMNP | 2576 |
| uniform | RPKYRAIWGE | 2577 |
| uniform | SSQRVLPQYE | 2578 |
| uniform | CFYAVSDYAN | 2579 |
| uniform | MASLNTIHGA | 2580 |
| uniform | TYCATMHYME | 2581 |
| uniform | QLKTQDFQIA | 2582 |
| uniform | SMFYGFPIYT | 2583 |
| uniform | CTWGHGKCPN | 2584 |
| uniform | ACACLQAFTH | 2585 |
| uniform | NKQRNNNEYA | 2586 |
| uniform | LQRGSHEKEL | 2587 |
| uniform | VCMRDTFWPC | 2588 |
| uniform | RSGEDSPGLN | 2589 |
| uniform | MQWWKSFPTY | 2590 |
| uniform | HPRVTIYILV | 2591 |
| uniform | ANYISQFHGT | 2592 |
| uniform | HFLCIYEHGY | 2593 |
| uniform | CWKGDLRTMF | 2594 |
| uniform | SRRIFWVFAW | 2595 |
| uniform | NITMALHKFF | 2596 |
| uniform | KPTMRTDEHT | 2597 |
| uniform | TCNHTVKAVL | 2598 |
| uniform | LIRRTFFNNP | 2599 |
| uniform | LAHMMRPKQQ | 2600 |
| uniform | LNNDWAIAEL | 2601 |
| uniform | GIWNRHDDGC | 2602 |
| uniform | NGQFSQWHMK | 2603 |
| uniform | AGVIHNAEQD | 2604 |
| uniform | SFDFKQPFHM | 2605 |
| uniform | TDDKNWLRTI | 2606 |
| uniform | LFQYLLFCNM | 2607 |
| uniform | QAEHPTGQIV | 2608 |
| uniform | VSPTNMAQGQ | 2609 |
| uniform | PEDVTNGGLR | 2610 |
| uniform | ILFRDVQFFN | 2611 |
| uniform | MEYVFRFDKV | 2612 |
| uniform | DDGNVIFSCV | 2613 |
| uniform | FMRMSANVTA | 2614 |
| uniform | IVCGAEYAFY | 2615 |
| uniform | LVYSGTHNFM | 2616 |
| uniform | QMARLTYQKD | 2617 |
| uniform | RLMERKYTGL | 2618 |
| uniform | HTYAWYFRWK | 2619 |
| uniform | GPVSIFEPVV | 2620 |
| uniform | IMHLWYHEME | 2621 |
| uniform | EELKALPHIE | 2622 |
| uniform | DSGVCTQWYP | 2623 |
| uniform | MDRTFEWLVY | 2624 |
| uniform | CMVTWVKSYC | 2625 |
| uniform | WNEADKDFVG | 2626 |
| uniform | KWANRRGILM | 2627 |
| uniform | YFCIQEYWQQ | 2628 |
| uniform | VMVKKPYIDV | 2629 |
| uniform | ESMWFNCVGL | 2630 |
| uniform | AWKYHFFCTF | 2631 |
| uniform | TPNKTSSWEC | 2632 |
| uniform | ITTIDYTALF | 2633 |
| uniform | FHGPCDVNIE | 2634 |
| uniform | CKKQGWSTWD | 2635 |
| uniform | DDYKVGFECN | 2636 |
| uniform | RSLWILAQRG | 2637 |
| uniform | ASHYDDWHIH | 2638 |
| uniform | QHPPPHSDNH | 2639 |
| uniform | AECIWWEKDI | 2640 |
| uniform | RTHGTMAVGT | 2641 |
| uniform | YLIGTGANTG | 2642 |
| uniform | QVCLQNKNAG | 2643 |
| uniform | ICSWHGMLNP | 2644 |
| uniform | AESIRVCKTL | 2645 |
| uniform | FYGHHHSTIL | 2646 |
| uniform | RWHERVDGLA | 2647 |
| uniform | GGIGRGWKQE | 2648 |
| uniform | GNRLPSYNRK | 2649 |
| uniform | LHFSRWLMQT | 2650 |
| uniform | HTDHTLLTCF | 2651 |
| uniform | KMFEYWTQIE | 2652 |
| uniform | VYYWYSDIDR | 2653 |
| uniform | LIRYIYIGVD | 2654 |
| uniform | ECMSISEPMC | 2655 |
| uniform | ITNSATFGFN | 2656 |
| uniform | IAGREKCRAR | 2657 |
| uniform | MHWRDYEFST | 2658 |
| uniform | WWFSQETIYM | 2659 |
| uniform | ASGGIDAVNM | 2660 |
| uniform | MPSKNEKDIC | 2661 |
| uniform | SKKLTNTQHG | 2662 |
| uniform | ILNRCRMIER | 2663 |
| uniform | YWKYSMEMAS | 2664 |
| uniform | GFNLPTRGTS | 2665 |
| uniform | EHNVAIMWIL | 2666 |
| uniform | ICDRPLAGFV | 2667 |
| uniform | ILRSEDMENE | 2668 |
| uniform | SRRLEMSIEE | 2669 |
| uniform | GTQICPLVGC | 2670 |
| uniform | GSPQKMNNGT | 2671 |
| uniform | FMDYFQKTTH | 2672 |
| uniform | TFYGFVPKYT | 2673 |
| uniform | QLCWHSLVCK | 2674 |
| uniform | EQPSQMECQT | 2675 |
| uniform | CMRQKEPYRP | 2676 |
| uniform | WYCNITSRVE | 2677 |
| uniform | PEWRDDICML | 2678 |
| uniform | HWSSAYQAEC | 2679 |
| uniform | PTDQRWRYET | 2680 |
| uniform | IQRNYMRDMK | 2681 |
| uniform | QACYSNAPEA | 2682 |
| uniform | NQIAVHKNMV | 2683 |
| uniform | DNIAQYCHFE | 2684 |
| uniform | EAAIHQHINF | 2685 |
| uniform | HETLMYCTHG | 2686 |
| uniform | ECEKYCCMVL | 2687 |
| uniform | KHNMLFKCSK | 2688 |
| uniform | HRYMSEKVVR | 2689 |
| uniform | QGCAEKYHNF | 2690 |
| uniform | FMWFSHCNNE | 2691 |
| uniform | PQHKYWEDLF | 2692 |
| uniform | HKNMKLWPNF | 2693 |
| uniform | GIGAYDSRDD | 2694 |
| uniform | PFYFQPSHNN | 2695 |
| uniform | IKNMKKCLWK | 2696 |
| uniform | REMSGMNFNN | 2697 |
| uniform | ESTGIWSPSF | 2698 |
| uniform | QTETCLYHME | 2699 |
| uniform | SAGQMMYLME | 2700 |
| uniform | AVHQYFVMQN | 2701 |
| uniform | SMNRRCYQFM | 2702 |
| uniform | HEQTKLTATF | 2703 |
| uniform | GHLLYFQFTE | 2704 |
| uniform | SCRNEHMANQ | 2705 |
| uniform | RVNYAEALNN | 2706 |
| uniform | WHACMTFKTD | 2707 |
| uniform | EFWLQGEVTM | 2708 |
| uniform | QRQWYPHHIT | 2709 |
| uniform | RTDAYGYHML | 2710 |
| uniform | GAALRPWPYR | 2711 |
| uniform | CRTDNPWDEF | 2712 |
| uniform | ERAYWVMVCT | 2713 |
| uniform | QLHAVNNYRC | 2714 |
| uniform | RWMSMAEWCS | 2715 |
| uniform | MHPGIMTNCN | 2716 |
| uniform | YFRIWCHDAK | 2717 |
| uniform | CKMSSQHTDK | 2718 |
| uniform | FQSLMVMYSL | 2719 |
| uniform | DGFCAFPSPP | 2720 |
| uniform | ASFNHDMVNV | 2721 |
| uniform | PYKRWTPMTG | 2722 |
| uniform | FVFDQEKTST | 2723 |
| uniform | TQHDRVMKQN | 2724 |
| uniform | TTVRVTFIVQ | 2725 |
| uniform | ATLDCCQQVW | 2726 |
| uniform | ASDQGHTQQA | 2727 |
| uniform | HCVGKNSSQT | 2728 |
| uniform | NCYLPRMQQT | 2729 |
| uniform | AICGIFQWPM | 2730 |
| uniform | LLCNAIGCAL | 2731 |
| uniform | TTDVFENNEQ | 2732 |
| uniform | MEMINFKELN | 2733 |
| uniform | ITSNKAELCC | 2734 |
| uniform | KWPEFQNIHA | 2735 |
| uniform | PAYHCWETCM | 2736 |
| uniform | WMMARDMCVN | 2737 |
| uniform | HSNNHTQYTY | 2738 |
| uniform | WPETWYGETR | 2739 |
| uniform | FTNTYHFCMF | 2740 |
| uniform | SCTSDSAYWL | 2741 |
| uniform | TGEYSNTDEE | 2742 |
| uniform | LLKCETCGTI | 2743 |
| uniform | TPIPASKQRQ | 2744 |
| uniform | IYHIVGYWLH | 2745 |
| uniform | HGKCTAHMET | 2746 |
| uniform | DFDLLFDFLV | 2747 |
| uniform | VPYVRTICAQ | 2748 |
| uniform | GRLYYYKRDN | 2749 |
| uniform | FLWYNTSLHS | 2750 |
| uniform | ISIRLFNKPI | 2751 |
| uniform | EDYSTWLFQW | 2752 |
| uniform | LNAYTPMCFS | 2753 |
| uniform | VWSLRNFTLK | 2754 |
| uniform | RENNSNCTDC | 2755 |
| uniform | RMYTDFVYGL | 2756 |
| uniform | MSLGQPASDA | 2757 |
| uniform | HATIIKCRTF | 2758 |
| uniform | GLMEMLSLPG | 2759 |
| uniform | DVINLGFIIY | 2760 |
| uniform | IARKRACPAG | 2761 |
| uniform | LECNSYSWVA | 2762 |
| uniform | CAGCHLQMDW | 2763 |
| uniform | EGGSRQQNHQ | 2764 |
| uniform | IHHCACEHAA | 2765 |
| uniform | KTAFVMQPKS | 2766 |
| uniform | RTGNKGLWSN | 2767 |
| uniform | SFQYQGRNWD | 2768 |
| uniform | WEKKASDTSH | 2769 |
| uniform | QNAWCLVTIT | 2770 |
| uniform | RHLIEKQAAI | 2771 |
| uniform | RYYDPYTKNE | 2772 |
| uniform | AHIVWPDGTV | 2773 |
| uniform | AGRSETNLQK | 2774 |
| uniform | HNGSYVDHRR | 2775 |
| uniform | AREMLARLQT | 2776 |
| uniform | YKPDKVCTYC | 2777 |
| uniform | PTKDVNIQWE | 2778 |
| uniform | TYQKATVIFN | 2779 |
| uniform | NAFYTYYSQD | 2780 |
| uniform | QPWMPPEENE | 2781 |
| uniform | DALHSNMGED | 2782 |
| uniform | LDGFKEQFTC | 2783 |
| uniform | FLPYLTGGLF | 2784 |
| uniform | DEGVGCDHHK | 2785 |
| uniform | ECCFPMPLAQ | 2786 |
| uniform | WMVMNPYGFC | 2787 |
| uniform | RADDTIKFPY | 2788 |
| uniform | PQKVHGQFDQ | 2789 |
| uniform | VYMWGASSHH | 2790 |
| uniform | FYCQGMNYSF | 2791 |
| uniform | IHQSDMYKSR | 2792 |
| uniform | NIDHGPLPIP | 2793 |
| uniform | WIIPSDPNAY | 2794 |
| uniform | TNCKGLCVPM | 2795 |
| uniform | IQPAANGVMF | 2796 |
| uniform | ARYQRHEWPM | 2797 |
| uniform | VDCKMKQELW | 2798 |
| uniform | RAPQGMEAVE | 2799 |
| uniform | LWVAARTFQT | 2800 |
| uniform | PKDLDFNHHG | 2801 |
| uniform | KTFRSLGWEQ | 2802 |
| uniform | CWSNYESCHH | 2803 |
| uniform | HGHKFNSRGL | 2804 |
| uniform | WKEDKGWRAN | 2805 |
| uniform | RSDDAIKQYW | 2806 |
| uniform | LVGFHKNVHD | 2807 |
| uniform | SPFYMYKFKF | 2808 |
| uniform | FSVLHEYANH | 2809 |
| uniform | KEEPMQDHDQ | 2810 |
| uniform | GDRIKVITIR | 2811 |
| uniform | DISNEWTDYL | 2812 |
| uniform | THGMNKLTHV | 2813 |
| uniform | AMCSDRHHWC | 2814 |
| uniform | DQQMETKIPY | 2815 |
| uniform | WCWWGKVKMW | 2816 |
| uniform | AMGSDWWEER | 2817 |
| uniform | KRLVQRGMHP | 2818 |
| uniform | AIYVKAPRIK | 2819 |
| uniform | KLEVPMHMAW | 2820 |
| uniform | KSSAFPVCFW | 2821 |
| uniform | LWDNICERFV | 2822 |
| uniform | NPPSPWMTYA | 2823 |
| uniform | LWMKKNELPR | 2824 |
| uniform | IPDVDPQRQL | 2825 |
| uniform | QTIVWGTWNY | 2826 |
| uniform | TYCEGRWWQA | 2827 |
| uniform | FCSMDYSLRW | 2828 |
| uniform | FGLAPWWPGQ | 2829 |
| uniform | HCAMELRRPW | 2830 |
| uniform | VTFLNYRPLH | 2831 |
| uniform | RPLLLIDAQT | 2832 |
| uniform | FYENEVYWIE | 2833 |
| uniform | FFMQFCQQPG | 2834 |
| uniform | HNERVISFNI | 2835 |
| uniform | ITYARITNYS | 2836 |
| uniform | YYTVQRCMEY | 2837 |
| uniform | AMMHDEINAF | 2838 |
| uniform | GNCRFLNPGI | 2839 |
| uniform | SLAWNCWLEV | 2840 |
| uniform | WLEHQEHFIQ | 2841 |
| uniform | NQKLNKKDYN | 2842 |
| uniform | MSNTRRSDHM | 2843 |
| uniform | VNIIWIEEAK | 2844 |
| uniform | RFVVEQAVRE | 2845 |
| uniform | CGLEFDILSD | 2846 |
| uniform | ESKFPWKMRG | 2847 |
| uniform | KYIFHDFWSS | 2848 |
| uniform | TYFKVNMAQR | 2849 |
| uniform | YRHMKELDWC | 2850 |
| uniform | CYVAESYAFM | 2851 |
| uniform | VDDPWRNYEP | 2852 |
| uniform | GPKFLEHEWF | 2853 |
| uniform | LCKRGYHIEF | 2854 |
| uniform | SPLYQNKELQ | 2855 |
| uniform | ALNIKKIYSE | 2856 |
| uniform | YVRPRMEFIR | 2857 |
| uniform | EYSQVPLIVL | 2858 |
| uniform | IGKVKGPKLD | 2859 |
| uniform | CNQVYVFSHP | 2860 |
| uniform | PQDTSQFADM | 2861 |
| uniform | WIMESATPHV | 2862 |
| uniform | AINWSMVETV | 2863 |
| uniform | RPPSSCLIQR | 2864 |
| uniform | CSEPQWYGGW | 2865 |
| uniform | HKYSCCPADW | 2866 |
| uniform | FVEMAATAVF | 2867 |
| uniform | IICVSIGTFI | 2868 |
| uniform | QVASACTFSK | 2869 |
| uniform | DWSIQKSWYP | 2870 |
| uniform | KALGLNDSNY | 2871 |
| uniform | FPCAVCMGLC | 2872 |
| uniform | RMDYWPSVMI | 2873 |
| uniform | QDLNWDWHGC | 2874 |
| uniform | CQRWHEFSFY | 2875 |
| uniform | TAQYDCRTQT | 2876 |
| uniform | KYDFHYDDKT | 2877 |
| uniform | NTRLCMGEAW | 2878 |
| uniform | RNNYKAQKIW | 2879 |
| uniform | EMVWWPEFGP | 2880 |
| uniform | FVIKEERMFE | 2881 |
| uniform | HAQPMEMIVA | 2882 |
| uniform | MLLHRTGGCW | 2883 |
| uniform | CVGLRNFMVQ | 2884 |
| uniform | YESRHFFCEI | 2885 |
| uniform | QEAAQYAWVC | 2886 |
| uniform | ACFYWMPFDD | 2887 |
| uniform | ARPNTCFFYI | 2888 |
| uniform | PYTEAHPMAY | 2889 |
| uniform | YLISTMYEHD | 2890 |
| uniform | YWSFQATHPW | 2891 |
| uniform | WNLTPNIWIV | 2892 |
| uniform | YSVHEKMEFA | 2893 |
| uniform | QNHDNWKDFI | 2894 |
| uniform | INANDKSDVY | 2895 |
| uniform | NEQQFRFDFE | 2896 |
| uniform | PWNVVAKMPK | 2897 |
| uniform | FLYVRINTMH | 2898 |
| uniform | RMTHLDAMIT | 2899 |
| uniform | LMLTYIYWFT | 2900 |
| uniform | FVLYHHMPFP | 2901 |
| uniform | QMHRSEKSDD | 2902 |
| uniform | FQFHHQYDAK | 2903 |
| uniform | FQQGHCYFNV | 2904 |
| uniform | LWGMQDHCGG | 2905 |
| uniform | APDQRGITGQ | 2906 |
| uniform | DIKEWMPMDH | 2907 |
| uniform | DWYAKELKVC | 2908 |
| uniform | DYMIGGDFEC | 2909 |
| uniform | ATHVWLLRNY | 2910 |
| uniform | YAAHQGICKW | 2911 |
| uniform | EKRPVGHAPI | 2912 |
| uniform | MWIYLRYFIF | 2913 |
| uniform | ITAWCMNKWK | 2914 |
| uniform | LPMHHVYHST | 2915 |
| uniform | VWCYGEAYAD | 2916 |
| uniform | CTDNCNTCEF | 2917 |
| uniform | NCCCGAMRRP | 2918 |
| uniform | GEYCRYMCRF | 2919 |
| uniform | PWDMHCHPCE | 2920 |
| uniform | YNAHMAKTWH | 2921 |
| uniform | KDRDEPKSPQ | 2922 |
| uniform | GKVGASGQGW | 2923 |
| uniform | LGKKMKEPSY | 2924 |
| uniform | MFFSLRNKYD | 2925 |
| uniform | SHIPDGTCRE | 2926 |
| uniform | MQCFAGPIVC | 2927 |
| uniform | EDTLSQPHRM | 2928 |
| uniform | IHAQYWQKRG | 2929 |
| uniform | TENGDCQHHI | 2930 |
| uniform | RDVSAWDING | 2931 |
| uniform | DENMEMAIEI | 2932 |
| uniform | NELGKWEIII | 2933 |
| uniform | LDQRIDIECH | 2934 |
| uniform | GMWAETTTVW | 2935 |
| uniform | PYPDTCWRWS | 2936 |
| uniform | JEYRQQSSEPD | 2937 |
| uniform | QRIPDCMFQS | 2938 |
| uniform | DPWLQKMAMH | 2939 |
| uniform | MTRMQMTLNN | 2940 |
| uniform | SLISWVFPTN | 2941 |
| uniform | IHMNFDVNIR | 2942 |
| uniform | KHVWNLRCPQ | 2943 |
| uniform | DPPWLWPNVA | 2944 |
| uniform | DRPPDCHSVR | 2945 |
| uniform | TKKVDCGGCI | 2946 |
| uniform | WANFDRLIAN | 2947 |
| uniform | REAVMRQLKQ | 2948 |
| uniform | KPLSEAMKCP | 2949 |
| uniform | GVGIKNTTTS | 2950 |
| uniform | GQTFMNSHKD | 2951 |
| uniform | PPGESKYGWN | 2952 |
| uniform | FIREYHTLGC | 2953 |
| uniform | RYIHVYGNPN | 2954 |
| uniform | CMLRSCICLN | 2955 |
| uniform | YYREAEKFGF | 2956 |
| uniform | EHQYVPNTPD | 2957 |
| uniform | GPCPFLILQN | 2958 |
| uniform | LRIPVQAPWT | 2959 |
| uniform | PFKSFFHEAW | 2960 |
| uniform | MWRTINTWWA | 2961 |
| uniform | DIPYGWFMGL | 2962 |
| uniform | FEDIQMGILR | 2963 |
| uniform | HDQQRQLCQP | 2964 |
| uniform | LRYHMICGCP | 2965 |
| uniform | QMMPAAGAHE | 2966 |
| uniform | RYSEEAELAS | 2967 |
| uniform | KMWKYTAMDA | 2968 |
| uniform | EMCHTVPNCR | 2969 |
| uniform | IFNDSVETDR | 2970 |
| uniform | GDHTWDPNIH | 2971 |
| uniform | GANKNVMRYE | 2972 |
| uniform | ERAMFLPKRR | 2973 |
| uniform | SVLRADGKGY | 2974 |
| uniform | CDLAVGGNGR | 2975 |
| uniform | YYLLMQSKEF | 2976 |
| uniform | LFEGDTMFKC | 2977 |
| uniform | MSDAQTVEGH | 2978 |
| uniform | AKTLCNEW | 2979 |
| uniform | YLVCNPTDNK | 2980 |
| uniform | YRWRIFNWDE | 2981 |
| uniform | SAWWYPDRMT | 2982 |
| uniform | PGYTDWALAV | 2983 |
| uniform | GQGFEPKKIG | 2984 |
| uniform | FHLMKRLWST | 2985 |
| uniform | VAQCNRKNAF | 2986 |
| uniform | VHWGGILWRG | 2987 |
| uniform | QTKNTWKECY | 2988 |
| uniform | IHIFMQFCAP | 2989 |
| uniform | DHKDEEKDYF | 2990 |
| uniform | TFLPLTNTIV | 2991 |
| uniform | RKKFPVMYKL | 2992 |
| uniform | LITNDRWPSD | 2993 |
| uniform | LDFQGSKMKM | 2994 |
| uniform | VCNWVMCAMN | 2995 |
| uniform | AVQFQCMRVI | 2996 |
| uniform | YHQFCIHHWH | 2997 |
| uniform | NSFIKWPTIQ | 2998 |
| uniform | TTFWVYGQND | 2999 |
| uniform | RDMAHNSQHF | 3000 |
| uniform | DWKYTSGFDW | 3001 |
| uniform | RKNRTCKGAR | 3002 |
| uniform | LFPIHSKHFF | 3003 |
| uniform | FTNVTSFMIL | 3004 |
| uniform | FDLGSSTKYC | 3005 |
| uniform | QHMENTVVVVC | 3006 |
| uniform | NASWHRYRVIQ | 3007 |
| uniform | HEGFSQFADFV | 3008 |
| uniform | LYAVCEPFEQM | 3009 |
| uniform | SVYLTFAFYGL | 3010 |
| uniform | RDFMQLGKMRD | 3011 |
| uniform | KAKDGRKCSFV | 3012 |
| uniform | FNLPQISLNST | 3013 |
| uniform | GGISIRAEVSH | 3014 |
| uniform | RFETINKGRPG | 3015 |
| uniform | QMRIAIWGPAF | 3016 |
| uniform | ACAFRYVQQYM | 3017 |
| uniform | KSEIMVYYLAC | 3018 |
| uniform | PKQKQCGDDNK | 3019 |
| uniform | VRCMSAIFNKP | 3020 |
| uniform | PEHPSREVTKS | 3021 |
| uniform | WLCEQFSWEKG | 3022 |
| uniform | VTELYGVNWTM | 3023 |
| uniform | NHAWWRSWTKH | 3024 |
| uniform | PCYQSKELLFE | 3025 |
| uniform | VPFSIPYLKDM | 3026 |
| uniform | YRYHWQNINLC | 3027 |
| uniform | PDCYSVTRKNQ | 3028 |
| uniform | EHCRAFCSHKI | 3029 |
| uniform | LFHSHSMCDHE | 3030 |
| uniform | PVNYFGQDQLF | 3031 |
| uniform | EMYAIPKTRTF | 3032 |
| uniform | GHHNAGFVTPV | 3033 |
| uniform | RKGYYSYHYDS | 3034 |
| uniform | IMKPGRFSTLQ | 3035 |
| uniform | WRSITLCPCRS | 3036 |
| uniform | WQEEFFQWEAF | 3037 |
| uniform | LELSTQIMCDQ | 3038 |
| uniform | ISQYWVNEPAH | 3039 |
| uniform | KDQEAMPWIWR | 3040 |
| uniform | MYQDEYWGMSM | 3041 |
| uniform | KCVCGRDTKTS | 3042 |
| uniform | VKNEIIDWCQF | 3043 |
| uniform | PCRFPGLMLIA | 3044 |
| uniform | NKRHYGNFGCS | 3045 |
| uniform | LDRVHSSLVFR | 3046 |
| uniform | IQGTTLVKGVK | 3047 |
| uniform | HYVQWMVAYCC | 3048 |
| uniform | YAHDFGVVCNQ | 3049 |
| uniform | MSMNSMPSMDK | 3050 |
| uniform | FPCCIIVNPCL | 3051 |
| uniform | NTFSFTFYIWT | 3052 |
| uniform | PLMVQKGFFWQ | 3053 |
| uniform | KVRPKMCFFAM | 3054 |
| uniform | MYIFQCTTMPY | 3055 |
| uniform | MNNIQGKCFEP | 3056 |
| uniform | WADTHCTRAPQ | 3057 |
| uniform | APTNVEPHKTP | 3058 |
| uniform | SCLMMYSSADN | 3059 |
| uniform | GFPEYNLCWRN | 3060 |
| uniform | RHNNNVMFQKQ | 3061 |
| uniform | EQEHMQVSPYI | 3062 |
| uniform | LGDKERHTVKV | 3063 |
| uniform | YACDVQCIHTG | 3064 |
| uniform | AFCALWQQMKA | 3065 |
| uniform | LNSSEFPDYET | 3066 |
| uniform | RHLQMNLHVSY | 3067 |
| uniform | GERWKSLTLEA | 3068 |
| uniform | PQACPPPQMNS | 3069 |
| uniform | LSHNPMVYRCG | 3070 |
| uniform | SQAPAEMVFQP | 3071 |
| uniform | HFGPNRINPYW | 3072 |
| uniform | AHIMGKMFFRN | 3073 |
| uniform | EVVIPGTHGSS | 3074 |
| uniform | FTSHKQRGEMR | 3075 |
| uniform | MYAMSLIDFKH | 3076 |
| uniform | CYTRVLLEYHQ | 3077 |
| uniform | TDHDFNQKEVV | 3078 |
| uniform | MIVYLGCATPY | 3079 |
| uniform | NKWWNVVPKFG | 3080 |
| uniform | NEKVFPVAPTP | 3081 |
| uniform | CKVVEGVNRFG | 3082 |
| uniform | LKCKLLFQDSG | 3083 |
| uniform | MCCCQFEILYD | 3084 |
| uniform | SLLWCNSYLCI | 3085 |
| uniform | QMFRVCWDANG | 3086 |
| uniform | SQSIYNHFQKK | 3087 |
| uniform | GTNLYVHTMYH | 3088 |
| uniform | TGQSPVHSCAR | 3089 |
| uniform | HNHADVFFFQA | 3090 |
| uniform | DLSNYTDGRFI | 3091 |
| uniform | QKFEMGEVATD | 3092 |
| uniform | GGLQPCPSNRG | 3093 |
| uniform | SGEMHWKMLFR | 3094 |
| uniform | MMCSISVSFPH | 3095 |
| uniform | QAVIMAVVFSI | 3096 |
| uniform | VAGIEENWLID | 3097 |
| uniform | YDSNQKSKVTH | 3098 |
| uniform | CTWTHVTHINQ | 3099 |
| uniform | LRGEFNILPHY | 3100 |
| uniform | HCNGAIRIEVD | 3101 |
| uniform | VPKEDCSRHES | 3102 |
| uniform | EAAHIKYYHLD | 3103 |
| uniform | NQKCLKSGSTN | 3104 |
| uniform | LGNDFGFWCIR | 3105 |
| uniform | IMAYMVCAHDY | 3106 |
| uniform | KHWGWHNMQEK | 3107 |
| uniform | DNMTEERFWLD | 3108 |
| uniform | FWHWPATNMNY | 3109 |
| uniform | FHDTTPWVVQC | 3110 |
| uniform | MNPNECTHFYN | 3111 |
| uniform | IFITMKTAPRN | 3112 |
| uniform | CWSQVPMKRYL | 3113 |
| uniform | ADLRRFLEFNG | 3114 |
| uniform | LHFVCSTHSNP | 3115 |
| uniform | GKIQWCNCYRH | 3116 |
| uniform | IENEGYMYYYK | 3117 |
| uniform | AADMKQGSGMA | 3118 |
| uniform | YRLMVEAAHSK | 3119 |
| uniform | MHVYKHVLLPT | 3120 |
| uniform | MCFKDQKWMKT | 3121 |
| uniform | FQMSYSGGFSW | 3122 |
| uniform | CQVHYYPHFTN | 3123 |
| uniform | WQWIRFNPIRC | 3124 |
| uniform | SCKFDHAMDQN | 3125 |
| uniform | NDNFKHGGSGK | 3126 |
| uniform | WPDIHFSAYNG | 3127 |
| uniform | ENTKWAQCDVK | 3128 |
| uniform | QFDPERVLFWM | 3129 |
| uniform | VKKATKQKTHQ | 3130 |
| uniform | VINKKKDGCRC | 3131 |
| uniform | ACGYECIHKML | 3132 |
| uniform | PDFVFDSELQS | 3133 |
| uniform | IHVCYSMKWPT | 3134 |
| uniform | SQSVPDCTEES | 3135 |
| uniform | LVMQHTFWDFD | 3136 |
| uniform | VYQRRTCEFQT | 3137 |
| uniform | EMLVVPCYADW | 3138 |
| uniform | DLQVWAHEMQL | 3139 |
| uniform | LGWSPYSEYIS | 3140 |
| uniform | YDMEAAWMYTW | 3141 |
| uniform | WSVATRSYKSN | 3142 |
| uniform | TSCDMQSAREY | 3143 |
| uniform | VFTHMRWLFAK | 3144 |
| uniform | QSKHCIVYCRN | 3145 |
| uniform | VIHFYDHHNVE | 3146 |
| uniform | CQTPEKIKYSL | 3147 |
| uniform | IRAQIYLPWPD | 3148 |
| uniform | DNYTLSQINVM | 3149 |
| uniform | QQEKALIDLYY | 3150 |
| uniform | CIMQGKRTDGA | 3151 |
| uniform | NGAFFRKQFTN | 3152 |
| uniform | YVVAQTPSYWI | 3153 |
| uniform | EKHLVQTWYIL | 3154 |
| uniform | FNAEYPVESPA | 3155 |
| uniform | PFCITTAFHVF | 3156 |
| uniform | WFFLEVGWHYC | 3157 |
| uniform | DKLDRGDMVFT | 3158 |
| uniform | AVLSWNTLKTT | 3159 |
| uniform | GDHNNEIFNVQ | 3160 |
| uniform | EMMWFPSLDFR | 3161 |
| uniform | KEVVFYCARCC | 3162 |
| uniform | FNHWLEVVEYI | 3163 |
| uniform | FEEAHCQHTTK | 3164 |
| uniform | RTDRPCSIIQH | 3165 |
| uniform | AYTTYWHGKRF | 3166 |
| uniform | SMKNHQGIGTN | 3167 |
| uniform | IYMTKMASCTE | 3168 |
| uniform | TFKLQPRWNRC | 3169 |
| uniform | FREAGQGCQWP | 3170 |
| uniform | MYFPYWWPMFK | 3171 |
| uniform | KCASSEDFSWN | 3172 |
| uniform | GFWWWNHAWTH | 3173 |
| uniform | PLPLQDATMKA | 3174 |
| uniform | MRQTTPPVGTL | 3175 |
| uniform | FEENDHQTMPG | 3176 |
| uniform | ESYPQGRCCPR | 3177 |
| uniform | AMSFYNFELHL | 3178 |
| uniform | IQGTFEVETYL | 3179 |
| uniform | EQLKQAVWRCV | 3180 |
| uniform | SENDKGSWPID | 3181 |
| uniform | DHASKFLRDEE | 3182 |
| uniform | YSILISLEPGL | 3183 |
| uniform | NNCRRVVKSKW | 3184 |
| uniform | HERSHNLPQNS | 3185 |
| uniform | WVFHQDCDRNV | 3186 |
| uniform | LGCEMAIDQER | 3187 |
| uniform | YIYNIYRLKLE | 3188 |
| uniform | QIIGWEAEESQ | 3189 |
| uniform | DGYFWCMCCKD | 3190 |
| uniform | IPFSQDHHFQL | 3191 |
| uniform | KYADKRKERCK | 3192 |
| uniform | NMELECSQGGY | 3193 |
| uniform | PHTMSYNKWRV | 3194 |
| uniform | MASQFSKSNHR | 3195 |
| uniform | QSCRVTNYTVG | 3196 |
| uniform | MKKKDHQIHLK | 3197 |
| uniform | CMHVQWDTYWY | 3198 |
| uniform | CHSHLRCHWIG | 3199 |
| uniform | CMGMLSQSKNG | 3200 |
| uniform | ALFEIGATAVS | 3201 |
| uniform | NMPHQGVCCYT | 3202 |
| uniform | CMEKSALHPCC | 3203 |
| uniform | RTLIRYWMWYM | 3204 |
| uniform | MFKQHTFSCHR | 3205 |
| uniform | MDENCDDYNIW | 3206 |
| uniform | NEHAVKAPRLP | 3207 |
| uniform | CAWSDYAQFQF | 3208 |
| uniform | HRSWSDFEANE | 3209 |
| uniform | MDDEAWKAPNS | 3210 |
| uniform | WSTFPTIPSRD | 3211 |
| uniform | PLMNYYRYQAR | 3212 |
| uniform | PSNPCALCLVG | 3213 |
| uniform | YCGVGDKEQVE | 3214 |
| uniform | QQLGNSSTGCD | 3215 |
| uniform | TEGAKDVQVYR | 3216 |
| uniform | FECSGNIFDHN | 3217 |
| uniform | HCQQVYGSVRD | 3218 |
| uniform | AIWAIWLAAAE | 3219 |
| uniform | HHLCAVAEGYI | 3220 |
| uniform | DAFCGDQFGFH | 3221 |
| uniform | EPMINHNMMYA | 3222 |
| uniform | ILLKVVRVRCC | 3223 |
| uniform | VDMRNTCHVKR | 3224 |
| uniform | GLKYGNFFWEM | 3225 |
| uniform | PGEFIDQKWDH | 3226 |
| uniform | ACYLCHQENGE | 3227 |
| uniform | NKVIMYGKWNI | 3228 |
| uniform | VCKDCYWQLQQ | 3229 |
| uniform | TTPLKGDVPDR | 3230 |
| uniform | FHCAPTCEGEV | 3231 |
| uniform | PLNKFGVKQAE | 3232 |
| uniform | QQMHFFQSWCH | 3233 |
| uniform | MLLMFIHKPNL | 3234 |
| uniform | TYMNPRMIYWP | 3235 |
| uniform | CCATMGDHTNS | 3236 |
| uniform | FMVTSYWAKAM | 3237 |
| uniform | RHGAFDVWWLG | 3238 |
| uniform | KFWLSKCTNKR | 3239 |
| uniform | AQMTRPEYYLC | 3240 |
| uniform | IGDTFVEDASY | 3241 |
| uniform | WGMWMKVNMMC | 3242 |
| uniform | WIGFGIERFLL | 3243 |
| uniform | NLGKKYTHTLM | 3244 |
| uniform | GIPLPPYYTWF | 3245 |
| uniform | IWYESLGERYK | 3246 |
| uniform | WEDCCYIRTYR | 3247 |
| uniform | STGDPFILHQG | 3248 |
| uniform | FPQPLLNEDET | 3249 |
| uniform | KMTTSCGETGY | 3250 |
| uniform | YPRAMQLDHVM | 3251 |
| uniform | LNFILGQLCEE | 3252 |
| uniform | LRNVCFHFFYL | 3253 |
| uniform | IVMCGMLPEEA | 3254 |
| uniform | IHMIPVVKWFY | 3255 |
| uniform | YDHCRSLLNRE | 3256 |
| uniform | YSWGGGTNWND | 3257 |
| uniform | VIMFCHISKYL | 3258 |
| uniform | PMCYFYAFRTC | 3259 |
| uniform | NRNHARPHPQM | 3260 |
| uniform | FTMNCQCQEPI | 3261 |
| uniform | VMPTTLYPKHD | 3262 |
| uniform | CLCRICGKHML | 3263 |
| uniform | EDNICQGDRWI | 3264 |
| uniform | SFWDITCVHAI | 3265 |
| uniform | PLIPFVAFVLY | 3266 |
| uniform | ENNCLASWVTC | 3267 |
| uniform | YGMLWHSIRYY | 3268 |
| uniform | CLRMSSRKSPK | 3269 |
| uniform | GPLMGFHCVVN | 3270 |
| uniform | GNTGIGHCTQF | 3271 |
| uniform | GLNGNKLRYRM | 3272 |
| uniform | VLVKCGIRWNH | 3273 |
| uniform | CQFWVEREDKW | 3274 |
| uniform | GGWFNFCHCCH | 3275 |
| uniform | HSGRPWPRIQV | 3276 |
| uniform | TQPEHNLGKWM | 3277 |
| uniform | CIKTVINYVGV | 3278 |
| uniform | QCVYTQLHKSF | 3279 |
| uniform | GIKMCSGPMQG | 3280 |
| uniform | CAFMFYKCLLS | 3281 |
| uniform | KGIMGMWMGWY | 3282 |
| uniform | NRKYEQVNSAI | 3283 |
| uniform | GFKLNLYQSWA | 3284 |
| uniform | HDMGNAVRNNT | 3285 |
| uniform | HSTPIQLDGWS | 3286 |
| uniform | NDPAHKHACLY | 3287 |
| uniform | AQNCNSMEQMQ | 3288 |
| uniform | AKQAPHANGSK | 3289 |
| uniform | VIKIFDLPMEY | 3290 |
| uniform | FMWSYASHKKM | 3291 |
| uniform | WCGPFPHMTPN | 3292 |
| uniform | HLDKMWRLMSD | 3293 |
| uniform | LMITRPRSENN | 3294 |
| uniform | PCTDTAAVPGD | 3295 |
| uniform | AHCPFETMFGI | 3296 |
| uniform | TNCCYKTGTIS | 3297 |
| uniform | QEKSNYSNNNF | 3298 |
| uniform | APLLFVVVIGH | 3299 |
| uniform | IMFECFMDCYP | 3300 |
| uniform | QLYETESPYGL | 3301 |
| uniform | SRQLYHMCANW | 3302 |
| uniform | DGRACQHKGDW | 3303 |
| uniform | HRGNNWMPKWE | 3304 |
| uniform | NQHYMYQFTKM | 3305 |
| uniform | VNEGYECIIPA | 3306 |
| uniform | RDSPSKPKNIN | 3307 |
| uniform | LTADENPTIFD | 3308 |
| uniform | CTHAMRNIHDE | 3309 |
| uniform | VMLNWQIRNCP | 3310 |
| uniform | HFTHPAWEDYW | 3311 |
| uniform | IIRIHNNCDEG | 3312 |
| uniform | EGLARTQHTRQ | 3313 |
| uniform | CRYPIAGEFDR | 3314 |
| uniform | CFFYEFAHLPL | 3315 |
| uniform | WEPPPHLWMWK | 3316 |
| uniform | RGTLWGNIEIF | 3317 |
| uniform | MRRCVRWVRIW | 3318 |
| uniform | AKPIRSFQCLT | 3319 |
| uniform | DNPSYSGYFGM | 3320 |
| uniform | CNCQDIAMHLC | 3321 |
| uniform | KYPQPKMVTCF | 3322 |
| uniform | TERVHGAKRPL | 3323 |
| uniform | KQKHDYCVMSK | 3324 |
| uniform | YNQYPAIKNQW | 3325 |
| uniform | TMWRTADSNPY | 3326 |
| uniform | SYEMVAGQQDQ | 3327 |
| uniform | KSASKSDPFDL | 3328 |
| uniform | GWQYPNTLMQW | 3329 |
| uniform | PSWWTHMSKKR | 3330 |
| uniform | IIMDPKGDIIK | 3331 |
| uniform | WFLGKLPWTHH | 3332 |
| uniform | IFDPSGMLAAL | 3333 |
| uniform | KGCIRAAMHFM | 3334 |
| uniform | QQVVNEPYKAS | 3335 |
| uniform | STANMWFRIKP | 3336 |
| uniform | RDPHELMCASE | 3337 |
| uniform | YFIWRGRIPMI | 3338 |
| uniform | FLPFNSYLVMG | 3339 |
| uniform | SFKDYMAPLYE | 3340 |
| uniform | CHDWVRDFMNS | 3341 |
| uniform | WGEMRRWLPST | 3342 |
| uniform | RMFASCNCLPR | 3343 |
| uniform | FMDCKRDDVCT | 3344 |
| uniform | LAPDMNKCCLE | 3345 |
| uniform | YTWVHMTCLPQ | 3346 |
| uniform | IEGTVCYPDAA | 3347 |
| uniform | PLNGHGLWCRI | 3348 |
| uniform | YGPLQHCNNQQ | 3349 |
| uniform | WSNWGSCTWLR | 3350 |
| uniform | WCNYTMGPCQA | 3351 |
| uniform | HGGVYEQTAPQ | 3352 |
| uniform | TFMAAFCDISF | 3353 |
| uniform | EQQVHSTNTSY | 3354 |
| uniform | DDLADIWNPSK | 3355 |
| uniform | TVIKIVWVHMQ | 3356 |
| uniform | LLWVMFYWKSR | 3357 |
| uniform | YKGQFMMTMTC | 3358 |
| uniform | HFALEQIHPYS | 3359 |
| uniform | FKITPKYDIPE | 3360 |
| uniform | SARMCQSTTIK | 3361 |
| uniform | VITFGCSSMGI | 3362 |
| uniform | MNQMFSQICTR | 3363 |
| uniform | PPWHDHHPGPM | 3364 |
| uniform | WRRHVPDHPNE | 3365 |
| uniform | CPTFNFEMANL | 3366 |
| uniform | FKMFMAKLTFL | 3367 |
| uniform | DYMEPCFCEAN | 3368 |
| uniform | YQSISTHLAQK | 3369 |
| uniform | ECRHCPKRYQQ | 3370 |
| uniform | KFERDVIVPNL | 3371 |
| uniform | ELYLPGWSCIL | 3372 |
| uniform | LLEIYIYLFPC | 3373 |
| uniform | LAHRFYYMKHN | 3374 |
| uniform | WVWRECSVFNC | 3375 |
| uniform | TAYAPSNQMWY | 3376 |
| uniform | LQECMFDGPQS | 3377 |
| uniform | QMHYWHWPEST | 3378 |
| uniform | IWPREYFVSLH | 3379 |
| uniform | TRVEEWPRQMD | 3380 |
| uniform | DTCGAHCIRNY | 3381 |
| uniform | PFTMLDLQHEK | 3382 |
| uniform | YIKESMIMKKT | 3383 |
| uniform | PVEIADDHLYC | 3384 |
| uniform | ACWCPGYPHTL | 3385 |
| uniform | SKTWHFLCHND | 3386 |
| uniform | ETTMNWNSNNG | 3387 |
| uniform | ILDKHQRVRKS | 3388 |
| uniform | IYMHPKMLMQN | 3389 |
| uniform | IIVGEYYRIAP | 3390 |
| uniform | FVWGDQNWSSE | 3391 |
| uniform | DHKPNGEESRL | 3392 |
| uniform | KKSDQFQPKTD | 3393 |
| uniform | LSFGGFNAFKH | 3394 |
| uniform | FYDNSYIPMFR | 3395 |
| uniform | VRNLLHMFQFD | 3396 |
| uniform | TMPNECSDQYQ | 3397 |
| uniform | SIAPIAFIEGV | 3398 |
| uniform | FWDTDLDNLVF | 3399 |
| uniform | DWLQHAKFVTV | 3400 |
| uniform | FKQLIMNLWMK | 3401 |
| uniform | MKYKSFYVNCH | 3402 |
| uniform | QHDNNIEVCYW | 3403 |
| uniform | LFSKPWAVEPN | 3404 |
| uniform | LIPVSFHENVH | 3405 |
| uniform | YNGTITFWPWH | 3406 |
| uniform | WPGVWFGMYIS | 3407 |
| uniform | LIRINVKYGMQ | 3408 |
| uniform | ICAWELQHICY | 3409 |
| uniform | NFPHFQRTVFQ | 3410 |
| uniform | FDRVCGREMWT | 3411 |
| uniform | TSRFAANSKIL | 3412 |
| uniform | EFMKEAVNTRR | 3413 |
| uniform | WFTDSFFTSQS | 3414 |
| uniform | YPDFMLGSNSG | 3415 |
| uniform | WPSNSSQVDHK | 3416 |
| uniform | DKPYSLMHETE | 3417 |
| uniform | IPPMYCILQTI | 3418 |
| uniform | EAQWRFRKKSF | 3419 |
| uniform | RDSVCNHCKCP | 3420 |
| uniform | NYILRHCSGSC | 3421 |
| uniform | GCRKGIWGPNI | 3422 |
| uniform | DIYRKEGKYMK | 3423 |
| uniform | RSAKRDNSWYQ | 3424 |
| uniform | MTYMVIQWHRR | 3425 |
| uniform | APVYQEGDDIN | 3426 |
| uniform | ICRYGPTFDQE | 3427 |
| uniform | ITMGIWAAHVS | 3428 |
| uniform | QKLLDDFSMWR | 3429 |
| uniform | LQYLDGAVSLQ | 3430 |
| uniform | FHRLQHTRQAV | 3431 |
| uniform | LWNCCGMRMNE | 3432 |
| uniform | CILDLIPAIMW | 3433 |
| uniform | YREYHMKPITL | 3434 |
| uniform | HDGTFSNKKRE | 3435 |
| uniform | NFLCNGGPGAP | 3436 |
| uniform | LGTAKDGRNHT | 3437 |
| uniform | EVIGVYYSESE | 3438 |
| uniform | SQKPGNQTWYY | 3439 |
| uniform | QCPLWPKYTPM | 3440 |
| uniform | VRIWSGTGNEK | 3441 |
| uniform | YSEPPMIVRQN | 3442 |
| uniform | RRCMWCMFIWF | 3443 |
| uniform | DGPKKLCFISL | 3444 |
| uniform | EAGNADNHECV | 3445 |
| uniform | VCKFPFCCHWF | 3446 |
| uniform | CGAAFPEPCIF | 3447 |
| uniform | LYEGMVDWRTS | 3448 |
| uniform | SPQIDLSGNED | 3449 |
| uniform | GADHSKVTVYT | 3450 |
| uniform | MWYHYRNECVM | 3451 |
| uniform | EMFHCYYVMVL | 3452 |
| uniform | HAMKYASVLNR | 3453 |
| uniform | RWCIWERLWLD | 3454 |
| uniform | GDTVEVSNRHG | 3455 |
| uniform | YIIIIEQWYFK | 3456 |
| uniform | SAAETTVSLRY | 3457 |
| uniform | YTYYKGKMTKM | 3458 |
| uniform | AYPTNFEGLAD | 3459 |
| uniform | AKINDMMTDGK | 3460 |
| uniform | HYMRGPRPSDP | 3461 |
| uniform | KNFQCEPVTCH | 3462 |
| uniform | CNMHCCGGHAP | 3463 |
| uniform | SSKMFYKHNHV | 3464 |
| uniform | ADMGVPWEMAL | 3465 |
| uniform | QFKKHWNTGKV | 3466 |
| uniform | WWILEWQIMAQ | 3467 |
| uniform | ACRYCTTQPDK | 3468 |
| uniform | HINTGTEGSQF | 3469 |
| uniform | SRYTWMWLATA | 3470 |
| uniform | QCQWWTFNLVY | 3471 |
| uniform | GRWMPSVYSCR | 3472 |
| uniform | YVCQSWQHWCS | 3473 |
| uniform | HQWSDWQSGWP | 3474 |
| uniform | HLYQESRQTGC | 3475 |
| uniform | DTCWVPYYDDA | 3476 |
| uniform | KGADAPGFHMT | 3477 |
| uniform | PCVEDPVCGHQ | 3478 |
| uniform | GWKFREYSTNK | 3479 |
| uniform | KDMPQICPTNV | 3480 |
| uniform | TMSLLFAKIAK | 3481 |
| uniform | KYHPTSTGGRR | 3482 |
| uniform | IHPRTSCVMVM | 3483 |
| uniform | PESWENYIQWQ | 3484 |
| uniform | PAYHGIWKQVT | 3485 |
| uniform | KEACHRECKSM | 3486 |
| uniform | TWKGRHCYREF | 3487 |
| uniform | MDCTDMNERCA | 3488 |
| uniform | ANWMYKLHKRG | 3489 |
| uniform | NHDIMSYNTMQ | 3490 |
| uniform | KHFDKIMIQDR | 3491 |
| uniform | HKQDDNFMWLT | 3492 |
| uniform | MCADWFDDIVS | 3493 |
| uniform | CTLPRNMGVDL | 3494 |
| uniform | VQTAYQLFENH | 3495 |
| uniform | KTIIQMLKMIR | 3496 |
| uniform | IGDTQAQYYGA | 3497 |
| uniform | VMKKWNPYHSA | 3498 |
| uniform | FTVCEERMHAM | 3499 |
| uniform | CQADLWESGGA | 3500 |
| uniform | ALGPVVWRVAV | 3501 |
| uniform | AQKKCRKVARN | 3502 |
| uniform | RWCPYGFWSRL | 3503 |
| uniform | WFIMIVVLCKN | 3504 |
| uniform | KIVSRMEAENQ | 3505 |
| uniform | HKRTPWCKICM | 3506 |
| uniform | NYHAFKGEYQT | 3507 |
| uniform | KFIRQLDCYGM | 3508 |
| uniform | WMEMSMWGGNL | 3509 |
| uniform | ASMQNVEIWKM | 3510 |
| uniform | KSFPWCCCGCY | 3511 |
| uniform | YFVDHSLYSDE | 3512 |
| uniform | MSHPRSQSRSL | 3513 |
| uniform | LATNCMWIDGW | 3514 |
| uniform | FTRDAIKYMVP | 3515 |
| uniform | WNRNYAKDVEI | 3516 |
| uniform | CYHELWAHKLC | 3517 |
| uniform | PYWLFLMNTCI | 3518 |
| uniform | CIYECMFRQAA | 3519 |
| uniform | DWRMDCQVHAC | 3520 |
| uniform | ENNHRKCRPWQ | 3521 |
| uniform | NFYVLNYSLHP | 3522 |
| uniform | MITVRHKLHQM | 3523 |
| uniform | KKAPTCHGPTY | 3524 |
| uniform | QYYQSMFGCIM | 3525 |
| uniform | PLQLVAREPFM | 3526 |
| uniform | WGSLSPPMYMK | 3527 |
| uniform | TTLVTQNASLA | 3528 |
| uniform | IMAPGQMTILR | 3529 |
| uniform | TRWKFDAWEEM | 3530 |
| uniform | CDCVNARFTDI | 3531 |
| uniform | HKSWAKRHKQI | 3532 |
| uniform | QTVIPLPVEFC | 3533 |
| uniform | YMWPVIPHAEP | 3534 |
| uniform | GWAQEHMAEAR | 3535 |
| uniform | PSGLRTLVLIM | 3536 |
| uniform | FAPVEQHTFCD | 3537 |
| uniform | QVPNQWVNCMW | 3538 |
| uniform | WNELPHDFGEE | 3539 |
| uniform | VWMLVLWQECW | 3540 |
| uniform | HIYCCPSTKYC | 3541 |
| uniform | AKLRPDRFTCN | 3542 |
| uniform | STISCCQECIR | 3543 |
| uniform | KNCAWTERFIP | 3544 |
| uniform | VCIQTEIFMIN | 3545 |
| uniform | LVVLGDDLDAC | 3546 |
| uniform | MIHFWKQVKTI | 3547 |
| uniform | GYVLAYWSIVH | 3548 |
| uniform | YKTAAAFRHRL | 3549 |
| uniform | SHKENPHRNCQ | 3550 |
| uniform | HPRKWFKNPVK | 3551 |
| uniform | LWYPILDFQND | 3552 |
| uniform | QEEERQVFSEE | 3553 |
| uniform | EYIKAEVCTEQ | 3554 |
| uniform | KVNHGLKKVWQ | 3555 |
| uniform | HMTYMFIDVHD | 3556 |
| uniform | QRCDEEAQMKS | 3557 |
| uniform | TYHMPPREWFW | 3558 |
| uniform | DPHYQVHLNNF | 3559 |
| uniform | VDASMAYLWLY | 3560 |
| uniform | RTQSQGIWMVM | 3561 |
| uniform | APWNHGIYIHD | 3562 |
| uniform | PIMTNDTYPED | 3563 |
| uniform | LAMETAWVGYY | 3564 |
| uniform | KAWWTIQDYWA | 3565 |
| uniform | IHRAGPQYAFC | 3566 |
| uniform | TIGEQSDKVFV | 3567 |
| uniform | KMTMMQMETGN | 3568 |
| uniform | AWNTPYNSPEY | 3569 |
| uniform | AYIRCYPESAK | 3570 |
| uniform | CIEHRQFMALR | 3571 |
| uniform | IRYAELAPSGH | 3572 |
| uniform | EYVIALPKFQR | 3573 |
| uniform | IWGFAVMTYVF | 3574 |
| uniform | WWNQDHVPLYE | 3575 |
| uniform | FMTRSQHHEVF | 3576 |
| uniform | IGQQKARFAFI | 3577 |
| uniform | AWLLSIFPNVN | 3578 |
| uniform | KHQYQDPQQML | 3579 |
| uniform | AKFAYEYFHYI | 3580 |
| uniform | QSEVVRTRNIC | 3581 |
| uniform | GCNMKMKFCNI | 3582 |
| uniform | GYDGYNLSYHH | 3583 |
| uniform | DVEQTGYSWAF | 3584 |
| uniform | GFQINGWREWE | 3585 |
| uniform | CLYDGSSGSCG | 3586 |
| uniform | PKVQMFCIVDD | 3587 |
| uniform | RYSAKLESYSS | 3588 |
| uniform | YYPDDLYQNDP | 3589 |
| uniform | REKNFVNCRCW | 3590 |
| uniform | KNNWKINNFDA | 3591 |
| uniform | RPIMWGLHDKH | 3592 |
| uniform | MSFQIVTGLHN | 3593 |
| uniform | PARYTHRESYM | 3594 |
| uniform | LTNYVNVFCRH | 3595 |
| uniform | YRHFSQDWTTG | 3596 |
| uniform | IHHVDHLPTTE | 3597 |
| uniform | SWFKTCCGQAQ | 3598 |
| uniform | NKLATTFIDEE | 3599 |
| uniform | FVSCKMLQPPK | 3600 |
| uniform | AYYEPGSGMTA | 3601 |
| uniform | AANLYTEEICL | 3602 |
| uniform | RWFREFASPFS | 3603 |
| uniform | NWCIVWNALQG | 3604 |
| uniform | QCIMMNIATHN | 3605 |
| uniform | MHDTKKNMDMT | 3606 |
| uniform | CQHIQGDFPIN | 3607 |
| uniform | HLDYKGPDSNY | 3608 |
| uniform | GEFNVNVGRVW | 3609 |
| uniform | TVSRWNSVKTL | 3610 |
| uniform | HKMILKHWAHY | 3611 |
| uniform | NSHKWYKAHYT | 3612 |
| uniform | DPVQYDFEFQW | 3613 |
| uniform | HETRICGLGAN | 3614 |
| uniform | TMYRVLKIKEQ | 3615 |
| uniform | LAPPVWWTRDW | 3616 |
| uniform | PEIHWEKIVTH | 3617 |
| uniform | MERKCFEATHL | 3618 |
| uniform | QLPVNPYMFVN | 3619 |
| uniform | NGAWCNKMDYF | 3620 |
| uniform | CGMDWNFKIYD | 3621 |
| uniform | SCTYWCKEITN | 3622 |
| uniform | NDERDRQTKSK | 3623 |
| uniform | GNGMPYEIAPA | 3624 |
| uniform | LRQMSTYPIVG | 3625 |
| uniform | GNWTVTQNWRD | 3626 |
| uniform | NKRNMVRQEFV | 3627 |
| uniform | YHFRLLIIHEA | 3628 |
| uniform | TAEWMEVFQHD | 3629 |
| uniform | KNLHGDDERWI | 3630 |
| uniform | WWSHWLNDMTI | 3631 |
| uniform | PSDCKAMKHLL | 3632 |
| uniform | RYMIPIPRWGQ | 3633 |
| uniform | LLKEPGKWMTN | 3634 |
| uniform | CLMAHINMFNC | 3635 |
| uniform | HCQAFVPDMDY | 3636 |
| uniform | DGKGHCPPRFD | 3637 |
| uniform | RRSCFTREWNP | 3638 |
| uniform | MWIQEMFYHGW | 3639 |
| uniform | YHNFWEEFKNF | 3640 |
| uniform | PIYMQDFERHF | 3641 |
| uniform | ENPDLWKNTDS | 3642 |
| uniform | GQADNFYYDRS | 3643 |
| uniform | PHQLGWFLPAV | 3644 |
| uniform | RRRVGKICYAV | 3645 |
| uniform | ECHEKCPQVTP | 3646 |
| uniform | APCAQVTPTAA | 3647 |
| uniform | TRKVMGPHQTY | 3648 |
| uniform | NVFVCDQSMFT | 3649 |
| uniform | ITWEPKRVCPI | 3650 |
| uniform | QGFEHVEVIWQ | 3651 |
| uniform | TIICEQSMKME | 3652 |
| uniform | IYPWIEPKNLN | 3653 |
| uniform | FMVVMVQKHDS | 3654 |
| uniform | KYLPLKREHIW | 3655 |
| uniform | CCMLITNSGKA | 3656 |
| uniform | SHLVSCHNQNM | 3657 |
| uniform | SVANGDYPQED | 3658 |
| uniform | MGIQWKQMPRG | 3659 |
| uniform | YQMRKFDWTDM | 3660 |
| uniform | WCPTQMALWEP | 3661 |
| uniform | MRQGIYINPYM | 3662 |
| uniform | WFHYMDVVQLE | 3663 |
| uniform | KNYKNRESKCW | 3664 |
| uniform | GKMNYMWAHRC | 3665 |
| uniform | QIVWPALPCLN | 3666 |
| uniform | DSANQVHHRIV | 3667 |
| uniform | GMYYMHYHNRN | 3668 |
| uniform | DGNCTDAYFYL | 3669 |
| uniform | DAKDSESVMLG | 3670 |
| uniform | GSISSWEAPGN | 3671 |
| uniform | EYNAPEPRMET | 3672 |
| uniform | KAQYRNIGTQL | 3673 |
| uniform | YRHCRWELGSS | 3674 |
| uniform | CTRWDDEKPWE | 3675 |
| uniform | PENAICWSEMG | 3676 |
| uniform | LGVIVDMTEDY | 3677 |
| uniform | PTMARSWYTCA | 3678 |
| uniform | CHYFPAQRGLV | 3679 |
| uniform | CCDMCVNFSIN | 3680 |
| uniform | DMVCICGTVAW | 3681 |
| uniform | RCDNQIKRIGS | 3682 |
| uniform | WKWDHQAPLWC | 3683 |
| uniform | HWDPNCWLLAD | 3684 |
| uniform | KGVHMFVAWWR | 3685 |
| uniform | IEDVIFRHRWM | 3686 |
| uniform | WVYCHIQLIAT | 3687 |
| uniform | LMLEVQPDNMS | 3688 |
| uniform | SGSCVWLNNTE | 3689 |
| uniform | HKPEGESVFVK | 3690 |
| uniform | IMTSIMMNPCF | 3691 |
| uniform | HFDQRYFEIQH | 3692 |
| uniform | DPEATAYQVSD | 3693 |
| uniform | GFKMLYSRLMA | 3694 |
| uniform | EWTTHTLYPNE | 3695 |
| uniform | DFIWHEEQNNK | 3696 |
| uniform | CMTESYHPNDP | 3697 |
| uniform | KKRAFALQRAH | 3698 |
| uniform | VLHLWRPWNGL | 3699 |
| uniform | ATDAPERSKEH | 3700 |
| uniform | EGLRSSAFTEW | 3701 |
| uniform | WALSWRIGSLT | 3702 |
| uniform | WTQTIHCMQIN | 3703 |
| uniform | CNNLMANTWIY | 3704 |
| uniform | ACYNGPTEAYS | 3705 |
| uniform | TSYSMVEPDAI | 3706 |
| uniform | RSTRFLKLWWM | 3707 |
| uniform | YIQSQCGITAV | 3708 |
| uniform | RNAKKCYGGTW | 3709 |
| uniform | QHLWMMGADMD | 3710 |
| uniform | GHFYSPYWPIP | 3711 |
| uniform | KHNIVMIDNKA | 3712 |
| uniform | FQIFDVSENVV | 3713 |
| uniform | IAQADWLKSKP | 3714 |
| uniform | HKCWQPPFLWN | 3715 |
| uniform | RDHPGHVYDNM | 3716 |
| uniform | TFTETPNLQPA | 3717 |
| uniform | NKAEENIKEQW | 3718 |
| uniform | AWMMWETFYYE | 3719 |
| uniform | GFSCFASRVME | 3720 |
| uniform | GRIIIKYMYPL | 3721 |
| uniform | DMVVYYWIPMN | 3722 |
| uniform | QQYHKGRYFSK | 3723 |
| uniform | THWMAGFDSFV | 3724 |
| uniform | CQHEDWPVYKA | 3725 |
| uniform | IVVFKGVANQM | 3726 |
| uniform | VLYQSCFIQND | 3727 |
| uniform | FKHGSTMSHNR | 3728 |
| uniform | GNCRDRLFIAN | 3729 |
| uniform | LTMQIEDSENM | 3730 |
| uniform | GLGVTKYYNQA | 3731 |
| uniform | ATEFHRRCCGT | 3732 |
| uniform | CAPEHNQHRMC | 3733 |
| uniform | CIYMSATALES | 3734 |
| uniform | RNMAAKANFGP | 3735 |
| uniform | VMMMQKVTFLK | 3736 |
| uniform | CNYDDNKNWSC | 3737 |
| uniform | NVTVFLGHETG | 3738 |
| uniform | YMDMVYQTAYR | 3739 |
| uniform | HCGMPYNWQRC | 3740 |
| uniform | PTACMYAMSNF | 3741 |
| uniform | RDHMAYLGSWD | 3742 |
| uniform | DAQRPWKRVIK | 3743 |
| uniform | MWSRTFYMDFT | 3744 |
| uniform | HVEFRMTYTQD | 3745 |
| uniform | YDQQRDQNPSF | 3746 |
| uniform | VGMGFVVGKDW | 3747 |
| uniform | QARVYSASNFN | 3748 |
| uniform | GMQMKLNMHYS | 3749 |
| uniform | GDWNKWHRKDR | 3750 |
| uniform | DPDGLLFCTNG | 3751 |
| uniform | SQIDDNWQMPN | 3752 |
| uniform | MSALPKAMYIA | 3753 |
| uniform | DHGYAWADADM | 3754 |
| uniform | EGNHIYYNCND | 3755 |
| uniform | MVPNVHDPNWR | 3756 |
| uniform | PWETRTSYIGF | 3757 |
| uniform | IRIVGPGMDEE | 3758 |
| uniform | KQWSYVPYFVM | 3759 |
| uniform | LHTSTWWIWWK | 3760 |
| uniform | YWYIFACTHPS | 3761 |
| uniform | AEDDCLPQHPK | 3762 |
| uniform | DICSNHEEQMN | 3763 |
| uniform | STMDISCTQLH | 3764 |
| uniform | TSPRELEVPPC | 3765 |
| uniform | MQHYMNDHSGF | 3766 |
| uniform | RRTYVIYVMKR | 3767 |
| uniform | DSLTYLAPDRG | 3768 |
| uniform | TWDHSHQWPHY | 3769 |
| uniform | QKHFNRLLRSQ | 3770 |
| uniform | WHAQTKNKQKK | 3771 |
| uniform | ICPHEDYESVL | 3772 |
| uniform | MSEMQEPMLYG | 3773 |
| uniform | MGDNWNLAVLA | 3774 |
| uniform | AVFVGDHNWAT | 3775 |
| uniform | YICSVAVVITC | 3776 |
| uniform | SYAATKTTGQH | 3777 |
| uniform | GREFGNHIFFH | 3778 |
| uniform | KIPSYKQFTCQ | 3779 |
| uniform | KMDSPSGGGKF | 3780 |
| uniform | AELKKMRDEQC | 3781 |
| uniform | MPRMWVHDKID | 3782 |
| uniform | QIPFRREFVWD | 3783 |
| uniform | VMPDNQYFSDV | 3784 |
| uniform | CHLYATNRDFE | 3785 |
| uniform | VYRDTCSEPWE | 3786 |
| uniform | ETGNVMSIDAC | 3787 |
| uniform | CGMPKTFIAVG | 3788 |
| uniform | KFMHRFSFIFH | 3789 |
| uniform | ITNTFLHCPWE | 3790 |
| uniform | REVEFSGKPAT | 3791 |
| uniform | YEMVQFNKFLT | 3792 |
| uniform | VTIWITPYDYH | 3793 |
| uniform | KWLEPHFCNKM | 3794 |
| uniform | LMMLYSREGYI | 3795 |
| uniform | KAFGENTIQPA | 3796 |
| uniform | IQCSQHHPWKS | 3797 |
| uniform | TPSKKTNFEES | 3798 |
| uniform | HNHCWHCWELG | 3799 |
| uniform | CARRYKIQFVK | 3800 |
| uniform | PRMNQTLTYPQ | 3801 |
| uniform | TGGWTKHQGTA | 3802 |
| uniform | QGQYINVPTFM | 3803 |
| uniform | TGWKPNCSLAC | 3804 |
| uniform | AIRFKCYYEPQ | 3805 |
| uniform | AGFMWYVEWYP | 3806 |
| uniform | YANIQMNDSDN | 3807 |
| uniform | NYNIWFDNIYP | 3808 |
| uniform | WVQFEFDCRPL | 3809 |
| uniform | ECIWHFSEFLF | 3810 |
| uniform | TRHYRNGAGHN | 3811 |
| uniform | DMLLCYGIREK | 3812 |
| uniform | SPRADHHYWHQ | 3813 |
| uniform | DWHSCHDDNKE | 3814 |
| uniform | SFGAVVDTTWQ | 3815 |
| uniform | EWNMRCGVPWS | 3816 |
| uniform | YGMHVNMDMSD | 3817 |
| uniform | TCKWFTNWKKH | 3818 |
| uniform | HYGNLPVSYNT | 3819 |
| uniform | RCKALSYHHMS | 3820 |
| uniform | FHHKFPRIMPY | 3821 |
| uniform | RKNNITHRHPN | 3822 |
| uniform | EDYSFHCDWHI | 3823 |
| uniform | RQAWGMNFFWV | 3824 |
| uniform | DKSFRDKLKYF | 3825 |
| uniform | MLALYLKIRYP | 3826 |
| uniform | KTNCISVLDDN | 3827 |
| uniform | NFYVFHAEDGY | 3828 |
| uniform | SFLDSCNRTQG | 3829 |
| uniform | MVNVCFRGEAP | 3830 |
| uniform | CSNCDKRSEGR | 3831 |
| uniform | RSFHMHTIAWM | 3832 |
| uniform | FLIEISILGKP | 3833 |
| uniform | TDWIDPMWKPW | 3834 |
| uniform | ARLHCGDCIVE | 3835 |
| uniform | TYKDMPGNETG | 3836 |
| uniform | RYAWCQLTEEN | 3837 |
| uniform | FKGDPIKCFWH | 3838 |
| uniform | PIAIAIKLMLP | 3839 |
| uniform | DLTPTPLITVS | 3840 |
| uniform | TNRIAYKAQLP | 3841 |
| uniform | MWMAINKHGWY | 3842 |
| uniform | HQHHVSATEQF | 3843 |
| uniform | IKPPSRFKPVM | 3844 |
| uniform | FTSIHIASPLT | 3845 |
| uniform | GGHYRQYKNIS | 3846 |
| uniform | QTGLRYTLWAE | 3847 |
| uniform | KLPHCNNWLWD | 3848 |
| uniform | SGMRKSDMLTQ | 3849 |
| uniform | FGWTECRAMRK | 3850 |
| uniform | DRWVQKEWRPF | 3851 |
| uniform | CMEIQCMGCVD | 3852 |
| uniform | QENWMVCYNDR | 3853 |
| uniform | GMSFWGFEVCL | 3854 |
| uniform | VKCMCWQEENY | 3855 |
| uniform | ESGQYDEAMEW | 3856 |
| uniform | IQQKICEKVEC | 3857 |
| uniform | LFFYTFVCFLV | 3858 |
| uniform | HGPQEIGECNP | 3859 |
| uniform | KEVFSCCFWMM | 3860 |
| uniform | RGELENNDGYG | 3861 |
| uniform | SIATWWNVTSF | 3862 |
| uniform | ANHMIVSLIMM | 3863 |
| uniform | FVTEVEQPSLV | 3864 |
| uniform | TPGVDCFKAQV | 3865 |
| uniform | TLPEKWCKGFN | 3866 |
| uniform | CCRWNQFWYTY | 3867 |
| uniform | KNFKSSKAHRF | 3868 |
| uniform | SKIFTYLIHMM | 3869 |
| uniform | DKLRISRGGYM | 3870 |
| uniform | CINEACDIWAL | 3871 |
| uniform | MPAYTQQRMLY | 3872 |
| uniform | KVNRWQMNYKP | 3873 |
| uniform | IDWMLMCYCRG | 3874 |
| uniform | LYMGDACYYPM | 3875 |
| uniform | SKYCPRIYQFM | 3876 |
| uniform | CHEWNFNRDAH | 3877 |
| uniform | GACTDGGAHGC | 3878 |
| uniform | MNGYDHECCTF | 3879 |
| uniform | TEHVAIRSPGY | 3880 |
| uniform | IIPDAMTAMMC | 3881 |
| uniform | WCLYNDLALWS | 3882 |
| uniform | WWNGIGLFDDG | 3883 |
| uniform | CLWREHYAPDL | 3884 |
| uniform | NMTTWNGLPMG | 3885 |
| uniform | HGLPMPPMAEV | 3886 |
| uniform | PNIQGCIAADE | 3887 |
| uniform | SMEAYSDCYPE | 3888 |
| uniform | RNIMLGLCTMC | 3889 |
| uniform | CDDDREDWGVT | 3890 |
| uniform | ELEWISMIFIY | 3891 |
| uniform | SFYAAYVYCQR | 3892 |
| uniform | YCVMLHIHPHT | 3893 |
| uniform | GVTMMYECVTI | 3894 |
| uniform | LHGIAINMGFM | 3895 |
| uniform | ESQFANSCGEV | 3896 |
| uniform | AQMSTVITVPM | 3897 |
| uniform | VICMGTWLSHM | 3898 |
| uniform | IWDEDQRKQHK | 3899 |
| uniform | AGQEQAEMVSK | 3900 |
| uniform | GQIANLKFSRQ | 3901 |
| uniform | IMPPGKFSSGG | 3902 |
| uniform | WIVMSLESMGV | 3903 |
| uniform | FMLWATTMIVW | 3904 |
| uniform | DTSPIKLHHKQ | 3905 |
| uniform | NCICLITLYQR | 3906 |
| uniform | YPDHNKCHESW | 3907 |
| uniform | HFRIKKPPVWD | 3908 |
| uniform | QAFTYKIDCRE | 3909 |
| uniform | WCHHYTCYFNM | 3910 |
| uniform | DLPTQKTQFRD | 3911 |
| uniform | TWDKFISMMSP | 3912 |
| uniform | QHDEDMNREKD | 3913 |
| uniform | GTWFKPKVMAS | 3914 |
| uniform | KFHGTDNARNC | 3915 |
| uniform | CGHQAFCSNFD | 3916 |
| uniform | RWTVPDQMYVP | 3917 |
| uniform | PWPCCMPCTIF | 3918 |
| uniform | ASNEWDMGFTG | 3919 |
| uniform | HWDAKYNVKRY | 3920 |
| uniform | PTESCSHLLVH | 3921 |
| uniform | NTAMLIIKTMD | 3922 |
| uniform | GGGPLIEEAAA | 3923 |
| uniform | AQVFEFCELKD | 3924 |
| uniform | KFMMLYHEMWG | 3925 |
| uniform | YHHQEHYWSQP | 3926 |
| uniform | TLFCVKGIVGF | 3927 |
| uniform | FGGGCTEYNEE | 3928 |
| uniform | QYMEDYWRIAC | 3929 |
| uniform | PGIQYQYMWWM | 3930 |
| uniform | WRINEFAIPYP | 3931 |
| uniform | TCQVWAHCMSH | 3932 |
| uniform | TNYAISECHKN | 3933 |
| uniform | ARILTLDTVWD | 3934 |
| uniform | NFGMKIYQASQ | 3935 |
| uniform | CCQTVPHLHIP | 3936 |
| uniform | FWRWACEIWES | 3937 |
| uniform | YTKFGCMKWRP | 3938 |
| uniform | HCIQQGGNVQC | 3939 |
| uniform | RYYMETRGGRC | 3940 |
| uniform | GHYCFQYPESF | 3941 |
| uniform | YVFSIRVRPVI | 3942 |
| uniform | EDLNCHGPFRV | 3943 |
| uniform | NMKVSAQNINP | 3944 |
| uniform | INAWPRHTYVF | 3945 |
| uniform | INVHGDNAPNK | 3946 |
| uniform | THEDWFRGWFV | 3947 |
| uniform | PKMYYMYHANG | 3948 |
| uniform | SNRIPHGWHLQ | 3949 |
| uniform | NRVRFLDYWRI | 3950 |
| uniform | TYPVNKGVIRC | 3951 |
| uniform | YDKLTRNIEGG | 3952 |
| uniform | HRCKNTSSNFA | 3953 |
| uniform | QWTPEAAIYCV | 3954 |
| uniform | LSNPLHDDSWF | 3955 |
| uniform | RDCHAEQLHFP | 3956 |
| uniform | HGIKTDYVRCN | 3957 |
| uniform | QGDIGQACCYT | 3958 |
| uniform | SRHYTCCHSAP | 3959 |
| uniform | MCMEIYDCHQR | 3960 |
| uniform | NMCPCEMVRMW | 3961 |
| uniform | CDCAFRIVVEA | 3962 |
| uniform | IVCETQWTPKF | 3963 |
| uniform | KPNFTVLSVDC | 3964 |
| uniform | HKHYGTPVFGN | 3965 |
| uniform | WLCDSCGNSCI | 3966 |
| uniform | RVIHFVCKVGA | 3967 |
| uniform | CLONEKFHHEI | 3968 |
| uniform | HIMASCHRKSK | 3969 |
| uniform | YLMYQWACSSI | 3970 |
| uniform | VGIPHKIFSSG | 3971 |
| uniform | LHWGGSIIYVW | 3972 |
| uniform | VATNNNERFDD | 3973 |
| uniform | MRYKPYTERIW | 3974 |
| uniform | CKWMKCSLIYA | 3975 |
| uniform | SFSIDSPQISH | 3976 |
| uniform | VYTGDWGMSGV | 3977 |
| uniform | TWIMPITPSYL | 3978 |
| uniform | MKLPDRDRTDV | 3979 |
| uniform | SWEWFELQRKQ | 3980 |
| uniform | CQPGVNEMSEF | 3981 |
| uniform | RRSCPINPIET | 3982 |
| uniform | QLPTSSCKITP | 3983 |
| uniform | FCEICILPKET | 3984 |
| uniform | ACCQFKGSQQL | 3985 |
| uniform | SAHTKLVREPG | 3986 |
| uniform | ATLAVRSYPRY | 3987 |
| uniform | ETMAGATDACI | 3988 |
| uniform | EKGEGVETRNQ | 3989 |
| uniform | HHETSIVHYQQ | 3990 |
| uniform | DKFWATYQFAE | 3991 |
| uniform | WTHVWWMQFSF | 3992 |
| uniform | GQRKRMTRAVQ | 3993 |
| uniform | SFTDECWPMQG | 3994 |
| uniform | SEKEKISNSSQ | 3995 |
| uniform | HGVKSVMFDFP | 3996 |
| uniform | CCVRMNQKCKI | 3997 |
| uniform | IGTLLINHRPM | 3998 |
| uniform | CEANRYHLONW | 3999 |
| uniform | SHQNHMFLYQG | 4000 |
| uniform | CPKPKHCAPCP | 4001 |
| uniform | WISPWQSWKPE | 4002 |
| uniform | MRQAMPCEAWM | 4003 |
| uniform | IRCCPPHWQST | 4004 |
| uniform | MTPKYKFVRFI | 4005 |
| uniform | CWDYVVKINCEE | 4006 |
| uniform | AKHTGLSFHFFW | 4007 |
| uniform | LDIKHQRLNRRD | 4008 |
| uniform | RFRMRGFHLFEY | 4009 |
| uniform | GDYPVRRQERKI | 4010 |
| uniform | NHMRYSLWMKLD | 4011 |
| uniform | EMKEMQSQYTFT | 4012 |
| uniform | AKCGQENIGYQY | 4013 |
| uniform | QRYALQLVTDAC | 4014 |
| uniform | KRDDKSPHSMWW | 4015 |
| uniform | TIDPRQFKTHQF | 4016 |
| uniform | QKOGAEMTAIKP | 4017 |
| uniform | MSRRASROCIHY | 4018 |
| uniform | YVQILWRRLEKI | 4019 |
| uniform | SDIDPAGLPANQ | 4020 |
| uniform | WWATKCEFLRQC | 4021 |
| uniform | CNMAREMHPLFW | 4022 |
| uniform | VCLSDRTTNHLA | 4023 |
| uniform | GIPNNDFIVESR | 4024 |
| uniform | EFWWHRIPWLVH | 4025 |
| uniform | YKEDPFLSIMYN | 4026 |
| uniform | GLLIKDLRPFDQ | 4027 |
| uniform | MPIRECLWHFTT | 4028 |
| uniform | AKVFLQGYPAGC | 4029 |
| uniform | HQHHVPPQKNYG | 4030 |
| uniform | MDYCIIKQNCWC | 4031 |
| uniform | AQPHLQWQPMMY | 4032 |
| uniform | FKGMDRPYPCAC | 4033 |
| uniform | VKEQYVCHEKVS | 4034 |
| uniform | EIGLPMLWVPFL | 4035 |
| uniform | GGSQKVWSDPFY | 4036 |
| uniform | SDRFIDGWYDLT | 4037 |
| uniform | QLYRVTFFVWEM | 4038 |
| uniform | CWQYYPNQDIVM | 4039 |
| uniform | CARRVKQCGRYM | 4040 |
| uniform | MPARDCDSNMLD | 4041 |
| uniform | TFMFFEHMEPAA | 4042 |
| uniform | QSATRVFWLVQG | 4043 |
| uniform | ALMMMESAQMRD | 4044 |
| uniform | RGHHNTWTVMEQ | 4045 |
| uniform | IYEFETPNVTQA | 4046 |
| uniform | WCNEPQGTWMGR | 4047 |
| uniform | KREWHAMIVEKR | 4048 |
| uniform | NDADRRGGAIHH | 4049 |
| uniform | APETTTYLRIVT | 4050 |
| uniform | FYPCYFWYVTAF | 4051 |
| uniform | RCKLWSCNFTGY | 4052 |
| uniform | FECNQDEQLYVA | 4053 |
| uniform | IMAQDCTELYVE | 4054 |
| uniform | FTEGHCMPMIKH | 4055 |
| uniform | NSMNLGALNLYN | 4056 |
| uniform | CEILFNTHSNWI | 4057 |
| uniform | RMCRMPNTTQER | 4058 |
| uniform | NAEEHWLQRKRP | 4059 |
| uniform | ESHLTNMNIIGQ | 4060 |
| uniform | EIPDREGIKGYP | 4061 |
| uniform | HYRECPCKYWAQ | 4062 |
| uniform | PEEPAKCNFTDL | 4063 |
| uniform | MFREFQSDRIGD | 4064 |
| uniform | VDMHCFQTNMAH | 4065 |
| uniform | TFPSDRDFEILN | 4066 |
| uniform | HDAVEVHNPGLT | 4067 |
| uniform | VWTDCAIAYRTS | 4068 |
| uniform | ACTIYDKIAFER | 4069 |
| uniform | CAILWVWIGPTG | 4070 |
| uniform | GRFTMPGCKEFW | 4071 |
| uniform | VGPFTDRAFSCA | 4072 |
| uniform | RAIDNPKHGIIH | 4073 |
| uniform | DQYMILWHRMFW | 4074 |
| uniform | QWNYKLDHHFAA | 4075 |
| uniform | KEEDNWWRAYER | 4076 |
| uniform | RADGFSVQGYKV | 4077 |
| uniform | FSSAFQAQWTPK | 4078 |
| uniform | VNGYVAARAMRE | 4079 |
| uniform | HRTHCVTQPSCH | 4080 |
| uniform | VFPNPCMTTKQI | 4081 |
| uniform | MCWQFHRDHEMV | 4082 |
| uniform | SDMRSVFFNMPY | 4083 |
| uniform | MMQAKQHPGHCN | 4084 |
| uniform | GWASAQTRPISI | 4085 |
| uniform | TQRTLWAISPGE | 4086 |
| uniform | MKKKRYDRDLYP | 4087 |
| uniform | FTWHEWHHDEGR | 4088 |
| uniform | PYHQHVYTCVKD | 4089 |
| uniform | GCTHFILYHRVR | 4090 |
| uniform | KHESNCWHGNEM | 4091 |
| uniform | CDDNPMNHQRDC | 4092 |
| uniform | QYMHWFFIQMFF | 4093 |
| uniform | IEGQNINAQDRG | 4094 |
| uniform | WDPGMYDSLYMG | 4095 |
| uniform | WGGLKFMVNCME | 4096 |
| uniform | MFRATMTARQHF | 4097 |
| uniform | KYLNGNVSIKFN | 4098 |
| uniform | QSFALHPCGKTW | 4099 |
| uniform | KQNEVRGPYMGK | 4100 |
| uniform | WMVQNDTPAWET | 4101 |
| uniform | NGCVVENKAEFH | 4102 |
| uniform | KWCPCEVCLRYK | 4103 |
| uniform | KGVKDWYFCAQS | 4104 |
| uniform | PDDGVRHRYFWP | 4105 |
| uniform | TREADFAYVTNK | 4106 |
| uniform | LWLIFILKLCLM | 4107 |
| uniform | ITLWNEMFKYIG | 4108 |
| uniform | EHQWSFATQDDL | 4109 |
| uniform | LHSMVGKAFPAV | 4110 |
| uniform | LRMWAVSHPMLG | 4111 |
| uniform | CDSGWASRTIIC | 4112 |
| uniform | MFQEGYSQPPMM | 4113 |
| uniform | NYMANSHQGTTG | 4114 |
| uniform | NHEIMIIVAYPE | 4115 |
| uniform | ETQDLPIEWGML | 4116 |
| uniform | LKWMPMEEGPRM | 4117 |
| uniform | QCVTAVHVPHIF | 4118 |
| uniform | FEDGHKATSICC | 4119 |
| uniform | DKPVPTSVGESE | 4120 |
| uniform | PQYAYRAGFNVE | 4121 |
| uniform | LSNVGYESRYDT | 4122 |
| uniform | WGKDINCYWDRV | 4123 |
| uniform | PACIVWFRASDF | 4124 |
| uniform | IQLEKHKSSPYG | 4125 |
| uniform | GMNGTFQSGYPM | 4126 |
| uniform | AVFRWIDITADA | 4127 |
| uniform | VKYHQCDPLAHF | 4128 |
| uniform | IGKPELNTLLRK | 4129 |
| uniform | ESHGYLYEIDHN | 4130 |
| uniform | DYCHLPWNNTYM | 4131 |
| uniform | IHNHLLETPKVL | 4132 |
| uniform | QHRDRIYYEDHQ | 4133 |
| uniform | GDRDNAIRCFPR | 4134 |
| uniform | RCDFLNRCRTDW | 4135 |
| uniform | HMWCKSLTWFPP | 4136 |
| uniform | VMWDANWHNEPW | 4137 |
| uniform | YMFIELIIPLQL | 4138 |
| uniform | PPEEIYDHKEIV | 4139 |
| uniform | QFILSRPAIVSS | 4140 |
| uniform | HSKVVPLITEAQ | 4141 |
| uniform | ATRWYVKTEKGF | 4142 |
| uniform | DSWVPDQCADYR | 4143 |
| uniform | TIMCYIFVCHQG | 4144 |
| uniform | DMDTLKLGFTLE | 4145 |
| uniform | VRCGCMIFIGAW | 4146 |
| uniform | QEGSLVSMMFTS | 4147 |
| uniform | RGPYCQTYYCEC | 4148 |
| uniform | MECAMNVTRRVV | 4149 |
| uniform | YYCGMQKMMHTK | 4150 |
| uniform | YESPCDDMMGIW | 4151 |
| uniform | FSHAPCVCHDEG | 4152 |
| uniform | SIWQKMSPLVDL | 4153 |
| uniform | YPAQTLVMIDYS | 4154 |
| uniform | TYSWGRPGESVL | 4155 |
| uniform | DIGMVASKSGWA | 4156 |
| uniform | FVWTCNKPHHVD | 4157 |
| uniform | IIRFCEVKQVYG | 4158 |
| uniform | QEHIMVAKWVET | 4159 |
| uniform | VLECPNDAQQSA | 4160 |
| uniform | WPCKADTVEGFH | 4161 |
| uniform | WKQVGAITMKGN | 4162 |
| uniform | VHVCEQDYMQGK | 4163 |
| uniform | KVPICYKLVLTK | 4164 |
| uniform | TYPFLLHHQIIY | 4165 |
| uniform | EQRELYKKARAI | 4166 |
| uniform | YACYFFSCDVVS | 4167 |
| uniform | ACFFNSPSGLWM | 4168 |
| uniform | TNWDVRRRNCET | 4169 |
| uniform | ENRIFWNNIGTN | 4170 |
| uniform | IQLPALQEIQGS | 4171 |
| uniform | LKQSWQDTDPPE | 4172 |
| uniform | DTILWIETSGRW | 4173 |
| uniform | YYRTFNQHPRTA | 4174 |
| uniform | VACGQSCCRSTF | 4175 |
| uniform | VYQPVDQPPWCG | 4176 |
| uniform | QDVCFRVWTFMA | 4177 |
| uniform | MEVKYKANRQLT | 4178 |
| uniform | HLAVVQKIGGLW | 4179 |
| uniform | IAVIKECGHSGG | 4180 |
| uniform | LASRDKFPELMF | 4181 |
| uniform | QNNRWKMRQCMA | 4182 |
| uniform | GGRPICIAMVFP | 4183 |
| uniform | MFEDVMHYHQDP | 4184 |
| uniform | MPGSWKPSPATG | 4185 |
| uniform | CSNRWAFYYYMP | 4186 |
| uniform | CSMSNWLQMRHT | 4187 |
| uniform | VRSIAWFNTPSG | 4188 |
| uniform | YRGCSWYPYHQC | 4189 |
| uniform | YYHMKMLNNSIV | 4190 |
| uniform | RGDCNQSTRWEY | 4191 |
| uniform | TWQWNVSRWCYP | 4192 |
| uniform | RGHNTAPLFRKF | 4193 |
| uniform | RHLDMVRQIADA | 4194 |
| uniform | SEGACLMKAGFG | 4195 |
| uniform | YQWAAFRWFCPW | 4196 |
| uniform | MWTYFWQWWDQY | 4197 |
| uniform | HASFHQCQKNHF | 4198 |
| uniform | TLAKNKRWGPHY | 4199 |
| uniform | GDGLMSQLGFDC | 4200 |
| uniform | LIPRQNVGGWMY | 4201 |
| uniform | GIGTPWCMCPNM | 4202 |
| uniform | LGYFVIPCQNYM | 4203 |
| uniform | GLKIALIPNIKF | 4204 |
| uniform | DWALEWWTVVMG | 4205 |
| uniform | TPPNPGLEHQGC | 4206 |
| uniform | LWGSFHIVNQQI | 4207 |
| uniform | AQMAANVNGLDM | 4208 |
| uniform | VSFWAHQPEYYY | 4209 |
| uniform | IHKQCQWGTNGW | 4210 |
| uniform | QFWSTFSHMCII | 4211 |
| uniform | YEGQGDGNCGLG | 4212 |
| uniform | YGCVVHHHGHSL | 4213 |
| uniform | NQHWSDKQDSLQ | 4214 |
| uniform | WTCNGKCPIPDL | 4215 |
| uniform | ISYVVGGAVGRT | 4216 |
| uniform | VDARWGDIVPIS | 4217 |
| uniform | SLNTIFLEAKSH | 4218 |
| uniform | PEPDPSCACNEW | 4219 |
| uniform | KHLKRVNDSDGD | 4220 |
| uniform | AAGCEARQNDPC | 4221 |
| uniform | VTSQPHTGVAES | 4222 |
| uniform | VHRNPEVWRIQQ | 4223 |
| uniform | FAQNFHDKSWVK | 4224 |
| uniform | LAREDTLYKGHP | 4225 |
| uniform | WYCEYPEFDWIE | 4226 |
| uniform | WHQDGLHKTEML | 4227 |
| uniform | IGFFTDRLSWRK | 4228 |
| uniform | VVPGHPTQTTLM | 4229 |
| uniform | CVYITMDLIVGR | 4230 |
| uniform | KSANIFTKNRNI | 4231 |
| uniform | THSKYSKTSAGL | 4232 |
| uniform | HLAFDAHTKKLY | 4233 |
| uniform | RWISTRHCIRLP | 4234 |
| uniform | VEYSCFMNKSEK | 4235 |
| uniform | DIYPHQGAALHC | 4236 |
| uniform | RLFNQKAMLPSC | 4237 |
| uniform | KAVECLSTLWYW | 4238 |
| uniform | IIMITLNHSTPI | 4239 |
| uniform | KPQSHCHPCCCD | 4240 |
| uniform | IVEPRPGRCLNL | 4241 |
| uniform | MKDKCKPDWKVL | 4242 |
| uniform | DQITDLGAICIP | 4243 |
| uniform | SSMIADQQFFKN | 4244 |
| uniform | WNLFIANHRVQQ | 4245 |
| uniform | HLCFKRLCRIFR | 4246 |
| uniform | CLHPSNARWYRS | 4247 |
| uniform | KKALDAWSCIFF | 4248 |
| uniform | NGSCHGVLSRPV | 4249 |
| uniform | RQLHCGSCSVSC | 4250 |
| uniform | VSSKYGSVPCLP | 4251 |
| uniform | CWSSFTDFIQYI | 4252 |
| uniform | LFWVIQTCAFAE | 4253 |
| uniform | VTCVDLTMFQLA | 4254 |
| uniform | RHDCFHQMGIQL | 4255 |
| uniform | NHTOCARKIIYM | 4256 |
| uniform | PKKAWMRNEVGA | 4257 |
| uniform | ILVGMALQGRLN | 4258 |
| uniform | CWYWDDYYADPS | 4259 |
| uniform | MKGGKHTISSVP | 4260 |
| uniform | GKPWRYTWEDRP | 4261 |
| uniform | ICGGLPDGDESS | 4262 |
| uniform | CHCMTFYAYCVG | 4263 |
| uniform | GYAYRVVWVEDV | 4264 |
| uniform | INMGAFWHWLFE | 4265 |
| uniform | EGAFILPGKSSS | 4266 |
| uniform | EYPYGTYLIDRL | 4267 |
| uniform | VWEHKCCKTRRE | 4268 |
| uniform | MPGVETFWKSQK | 4269 |
| uniform | AYQQVMEWLWMY | 4270 |
| uniform | LMGHQDVYIFAH | 4271 |
| uniform | LTKCKNSWMSEF | 4272 |
| uniform | KCLKQDDKYTAP | 4273 |
| uniform | HWDVYEIAWINDI | 4274 |
| uniform | NILKDSHEEQPR | 4275 |
| uniform | SWTVIVYYLENL | 4276 |
| uniform | ESLSLVLSFHDC | 4277 |
| uniform | MHCWFMQVWPLF | 4278 |
| uniform | MKKITLWVIFHL | 4279 |
| uniform | DNEDAGERIQRM | 4280 |
| uniform | STHHHHEWSCVE | 4281 |
| uniform | YVCHDITRHHPP | 4282 |
| uniform | CLVSPKTLCWGH | 4283 |
| uniform | PKQIMHPDKRHQ | 4284 |
| uniform | SEIKDEPLGVME | 4285 |
| uniform | EYSYSEIIVEIA | 4286 |
| uniform | EIRSFSMDGCRV | 4287 |
| uniform | QKMWGRKDGYTY | 4288 |
| uniform | KWCGTHKKHQFD | 4289 |
| uniform | HQTNEQGYFALY | 4290 |
| uniform | AHCAWRALISVC | 4291 |
| uniform | MGFAEVNWPIYN | 4292 |
| uniform | LFFVNNKWVQVI | 4293 |
| uniform | CTNVAYSLLHEE | 4294 |
| uniform | QNLPWWRQNHFE | 4295 |
| uniform | HTFTAGVLYFCY | 4296 |
| uniform | YCVHIHDIMRPR | 4297 |
| uniform | KNPLNYMSGQQS | 4298 |
| uniform | YHYHPRMWLHYH | 4299 |
| uniform | IQLPWAKWMLGC | 4300 |
| uniform | GTEWNLRSDYTE | 4301 |
| uniform | VQYEKLKDVMQI | 4302 |
| uniform | SCIKTWDEKFCV | 4303 |
| uniform | TVWMFTVEDEEN | 4304 |
| uniform | MQAVTWAIKCFY | 4305 |
| uniform | KLIWFCHWHCMF | 4306 |
| uniform | IKDWQYNFLIKF | 4307 |
| uniform | LHEELMTNIIES | 4308 |
| uniform | WIIEGSCRFSHF | 4309 |
| uniform | HEGPALLEFLYF | 4310 |
| uniform | CQSPPKWYYYMD | 4311 |
| uniform | YWMTEAATCKDF | 4312 |
| uniform | EVFFGPMKVDVL | 4313 |
| uniform | KDGFLIYQADCR | 4314 |
| uniform | HTKNKIGYAYGM | 4315 |
| uniform | HFYEQLCSVFNK | 4316 |
| uniform | KDMLFAMIIQEV | 4317 |
| uniform | CVAQCHMAYCII | 4318 |
| uniform | PVDQKSTARSGG | 4319 |
| uniform | KLCPQNWQNEWR | 4320 |
| uniform | PWSAWNNYPIWQ | 4321 |
| uniform | STHQFWWQLPEV | 4322 |
| uniform | ENCSPDCQMHGV | 4323 |
| uniform | WTADDQDQWMLT | 4324 |
| uniform | SMPSERQAKWKY | 4325 |
| uniform | WDDLTFFLHNVI | 4326 |
| uniform | LQMDTGDDLWEL | 4327 |
| uniform | SCAVFWKPTYEV | 4328 |
| uniform | SMWCHQAEGHDF | 4329 |
| uniform | NFKLFGETVKMW | 4330 |
| uniform | FECDVNDEMHKN | 4331 |
| uniform | MICYGQFVELRM | 4332 |
| uniform | WPNRRHAYAFYR | 4333 |
| uniform | KKKRWLRGTNGK | 4334 |
| uniform | HSSYCCAVGHVT | 4335 |
| uniform | DHPQYHKTIYIQ | 4336 |
| uniform | LPATCKSPPWRS | 4337 |
| uniform | CDYECWDDHEPI | 4338 |
| uniform | LFFANQQGYTTW | 4339 |
| uniform | HCEWHVMQLVMS | 4340 |
| uniform | EWPQETYCSLWP | 4341 |
| uniform | MWSCSDWICCYL | 4342 |
| uniform | PFTESNAYPITA | 4343 |
| uniform | VRLVSNNKWEDY | 4344 |
| uniform | PVYWDPNHCEHG | 4345 |
| uniform | PDIYLRLFVIFD | 4346 |
| uniform | VCHKWESIRLRN | 4347 |
| uniform | GQLYLMVYEMDE | 4348 |
| uniform | EMRNRAKFYRPL | 4349 |
| uniform | DWSDSWNIQQCR | 4350 |
| uniform | WMFTVCCAPKRH | 4351 |
| uniform | TLWLCHRVYCIS | 4352 |
| uniform | QPCMPCKVSETQ | 4353 |
| uniform | MDWEKEDTWWDN | 4354 |
| uniform | PTCRYMRGPMND | 4355 |
| uniform | QMLPRHILGAPP | 4356 |
| uniform | SRLSVTACRYHK | 4357 |
| uniform | SIGMDFLEADWY | 4358 |
| uniform | RINIRRFDLAFS | 4359 |
| uniform | DYHRPIGPCRLS | 4360 |
| uniform | ATDHSFGYIDQT | 4361 |
| uniform | IPVLLSNWRVTG | 4362 |
| uniform | GIKSGLPRWMDY | 4363 |
| uniform | EMMIDLNNTVEE | 4364 |
| uniform | YKTCDETMLSGA | 4365 |
| uniform | MLPDVISYTMYS | 4366 |
| uniform | FADQTQGTCIRE | 4367 |
| uniform | RLWTVEQWRKAE | 4368 |
| uniform | DMDWIGGMWIHS | 4369 |
| uniform | AISHFAPRLQMQ | 4370 |
| uniform | YGYGWFQYPLIG | 4371 |
| uniform | ADYHMRGYEEGQ | 4372 |
| uniform | ALWVTEQLCQGQ | 4373 |
| uniform | AENCLEPHAHTQ | 4374 |
| uniform | APKAISLGIWGM | 4375 |
| uniform | VYKGETQNDSEP | 4376 |
| uniform | GKRYWDRWQHGR | 4377 |
| uniform | FDCTENKMKCIS | 4378 |
| uniform | AQIGDPKEPASQ | 4379 |
| uniform | AGDVRCDQYCRS | 4380 |
| uniform | QMAVYQMQRIMC | 4381 |
| uniform | SIVWAIYKHYWY | 4382 |
| uniform | GEAVEDTGMRNG | 4383 |
| uniform | DYVTCPSWCNLI | 4384 |
| uniform | DFHPINGCVDMF | 4385 |
| uniform | SSKMMMIAESSR | 4386 |
| uniform | FMQGMRLEMNKY | 4387 |
| uniform | FSAIVEYLWEFV | 4388 |
| uniform | DTWVYGLRDEVK | 4389 |
| uniform | MLYDTKFRAYLP | 4390 |
| uniform | SITNSNDCRCVP | 4391 |
| uniform | GCARWRRDQQHV | 4392 |
| uniform | TLNRRAPPREVN | 4393 |
| uniform | MDYKVAEGIYCC | 4394 |
| uniform | CQCEHQNRDCAP | 4395 |
| uniform | VLSEIIPVKFKP | 4396 |
| uniform | DDEHGKWDLRGI | 4397 |
| uniform | IDPPMEVLYHKH | 4398 |
| uniform | KPACTPQKGKKN | 4399 |
| uniform | KKKARMHWVGCH | 4400 |
| uniform | DIYDKNKDCNRC | 4401 |
| uniform | SRMNCHDNMLEP | 4402 |
| uniform | TKLPLKVHIGDK | 4403 |
| uniform | DILWSNIYFPAV | 4404 |
| uniform | HMEPGVFEYNEY | 4405 |
| uniform | WSRWVITTHVRW | 4406 |
| uniform | QFFRDSLLVAPQ | 4407 |
| uniform | NEESVQVKMRDT | 4408 |
| uniform | NEASDVSAFDRE | 4409 |
| uniform | NTDHSWIPVYWG | 4410 |
| uniform | YFYHHKYPYDQM | 4411 |
| uniform | GHKGRVPKEDSG | 4412 |
| uniform | DSSDQGFMGLTD | 4413 |
| uniform | VPGFKRMYMWCC | 4414 |
| uniform | DFSEWNKVVRPY | 4415 |
| uniform | WYATLPNPMQPL | 4416 |
| uniform | KMMEDDWMSLLN | 4417 |
| uniform | PPFFAALPHSFR | 4418 |
| uniform | KWNSFNFSSGHK | 4419 |
| uniform | QSFWHARFMAEL | 4420 |
| uniform | GYQSPQHVCNVT | 4421 |
| uniform | IIIGTDMWEMNI | 4422 |
| uniform | DEKGWLEPFSHQ | 4423 |
| uniform | IDLTAKMHVHNM | 4424 |
| uniform | ADYIQDYFSTHE | 4425 |
| uniform | QNKIEMAWDGWR | 4426 |
| uniform | HKCTGNDWRSVI | 4427 |
| uniform | CESPTLLCQLGV | 4428 |
| uniform | AICRAFMHAYHI | 4429 |
| uniform | HNSGGALDTASY | 4430 |
| uniform | LDEVRLMGKTEF | 4431 |
| uniform | IPEKNMQSNIDC | 4432 |
| uniform | TVINGWRHPKER | 4433 |
| uniform | AASSVPCSKWLI | 4434 |
| uniform | PMMGSRCFWHVL | 4435 |
| uniform | TQEMNKLYLSWM | 4436 |
| uniform | FHTIKKHNLKTS | 4437 |
| uniform | KFTYKHITPFYD | 4438 |
| uniform | HHVTSSFCGYQP | 4439 |
| uniform | CKGMEIQDMTMP | 4440 |
| uniform | MIKMFQWVYRNP | 4441 |
| uniform | LTDGWRDRSAMH | 4442 |
| uniform | FGKTWPQCNRIS | 4443 |
| uniform | LESWDPAANAIM | 4444 |
| uniform | FTFEITVNLSTT | 4445 |
| uniform | GVTIWLFQHFHS | 4446 |
| uniform | CAGRQFCMWTTR | 4447 |
| uniform | RTLPPHTSEYNL | 4448 |
| uniform | SFVINSMPNYNV | 4449 |
| uniform | GSREGPCDDHCF | 4450 |
| uniform | PHFWRCNDCKLI | 4451 |
| uniform | TMIISWSCGILC | 4452 |
| uniform | VDGCEQATPPHD | 4453 |
| uniform | WQKAVWSYWRYN | 4454 |
| uniform | MPIAQSDYFSRP | 4455 |
| uniform | QWCCDPMWKLQF | 4456 |
| uniform | SALMRPFYQMPI | 4457 |
| uniform | KFRNYHIQIQCW | 4458 |
| uniform | QKPQHRCPQTDP | 4459 |
| uniform | YWWVIQTPCSKE | 4460 |
| uniform | EEMAMAKRFHWK | 4461 |
| uniform | PGEEMYADLVHC | 4462 |
| uniform | NCMPNEYFHSPD | 4463 |
| uniform | HNRKNLWDSYWI | 4464 |
| uniform | YYIFGELCAQME | 4465 |
| uniform | PNKYDICGWMAM | 4466 |
| uniform | WQLMFWVQPLHR | 4467 |
| uniform | ENREMPKVRKHD | 4468 |
| uniform | VCQWQCKEFNKD | 4469 |
| uniform | MHHVIQETDCHA | 4470 |
| uniform | KNDFECRLIAPQ | 4471 |
| uniform | VVHRAFAHTMQA | 4472 |
| uniform | WTQVKGCARQGL | 4473 |
| uniform | ANMFNPGHLKPS | 4474 |
| uniform | WCLEIFQGWQSS | 4475 |
| uniform | GSMGTRQTMYYG | 4476 |
| uniform | GKQTTDALIYYR | 4477 |
| uniform | RTVCHSMYTQEH | 4478 |
| uniform | DFQHYPLRAWFA | 4479 |
| uniform | FNWKNCHQFIFN | 4480 |
| uniform | QVRVELFSEPWS | 4481 |
| uniform | CMLSGTQRGNYW | 4482 |
| uniform | IRCPKPCYEQWW | 4483 |
| uniform | WQDIPDWYECAN | 4484 |
| uniform | PECRWDCLNRNQ | 4485 |
| uniform | TLDHPRPSIMAG | 4486 |
| uniform | RWDRKYLSTEHS | 4487 |
| uniform | GKYQMWSPTCPW | 4488 |
| uniform | SIFQPMCCMRFY | 4489 |
| uniform | QKMNSPFTDADI | 4490 |
| uniform | TSRIYHFRAVWQ | 4491 |
| uniform | VNNFKKRRAFNS | 4492 |
| uniform | KFGVGCMHYQFH | 4493 |
| uniform | IDFINCFCPHTN | 4494 |
| uniform | ICNNPPHNSNRN | 4495 |
| uniform | HTLMTWDDDGHQ | 4496 |
| uniform | LVSMADMVYYFN | 4497 |
| uniform | NMICHMLQCTVQ | 4498 |
| uniform | PPYNWCRKAPWW | 4499 |
| uniform | TPREDKWRLECL | 4500 |
| uniform | PQLAKCPQWQPF | 4501 |
| uniform | LNGYVTGYVGYA | 4502 |
| uniform | MNFPKYGEVISY | 4503 |
| uniform | LCLNNFYQKADY | 4504 |
| uniform | MDVREYRGKMAH | 4505 |
| uniform | AGPLNAYIGVHG | 4506 |
| uniform | QQKGCDVHCCDE | 4507 |
| uniform | FMNIQRQEALYK | 4508 |
| uniform | LLRQGCIEPNMR | 4509 |
| uniform | WWKKDLPCINTQ | 4510 |
| uniform | HHQLRILSKLAW | 4511 |
| uniform | EPESHTWVVNYE | 4512 |
| uniform | QKAPVSISPLKH | 4513 |
| uniform | MEEDKCIVPYMI | 4514 |
| uniform | RTSSGMTSDRRP | 4515 |
| uniform | TWVLRYSFSDHM | 4516 |
| uniform | APGHTTNFIAHI | 4517 |
| uniform | HLQQPWKWAELH | 4518 |
| uniform | KQSWQIAWFDCR | 4519 |
| uniform | STNLAANYHLVP | 4520 |
| uniform | QASKGIAHAVEC | 4521 |
| uniform | NGQKECSRQTFE | 4522 |
| uniform | EKNNHAHNLRWI | 4523 |
| uniform | NKISVSNWGKGL | 4524 |
| uniform | KEARIEWYKCPV | 4525 |
| uniform | EHKDLFQNTPKY | 4526 |
| uniform | EMTCYQCYWGNT | 4527 |
| uniform | LFFYSTGDWHYI | 4528 |
| uniform | MTPIHEPQPWMV | 4529 |
| uniform | HQTNPTCCCWLC | 4530 |
| uniform | TIQNDMATVRNM | 4531 |
| uniform | MYGAFNPEGHVF | 4532 |
| uniform | DQDVEAKYDYWF | 4533 |
| uniform | LLDLGNLKEGDR | 4534 |
| uniform | SFKKVATTNGDS | 4535 |
| uniform | SVPSTIISWEPK | 4536 |
| uniform | CACNYCDMTRLR | 4537 |
| uniform | PLFPAYVKKQGY | 4538 |
| uniform | CRGWERMYCFCR | 4539 |
| uniform | ITFSGPHWEAWA | 4540 |
| uniform | IVVHGFGIRKCI | 4541 |
| uniform | IEIQGRSWEDSP | 4542 |
| uniform | VKCNQWKHSWCP | 4543 |
| uniform | LGTTPFLHDTTM | 4544 |
| uniform | LHWPQHDICAFM | 4545 |
| uniform | KNISEQGLWPQG | 4546 |
| uniform | ICHAHKIMWNWC | 4547 |
| uniform | QCTSYWTHMDYR | 4548 |
| uniform | KAYIINTHKGTV | 4549 |
| uniform | SCDAVTYCAYPY | 4550 |
| uniform | GALCQCFHTPHN | 4551 |
| uniform | NTPHIDWFLDMA | 4552 |
| uniform | SPNRPFTSHIIV | 4553 |
| uniform | LESKQTVTMTGI | 4554 |
| uniform | GLRVAIEMTFHD | 4555 |
| uniform | RETKHKTCYLVW | 4556 |
| uniform | ANHGTGGVCADM | 4557 |
| uniform | QLDQGIVLGLLV | 4558 |
| uniform | INDIVIKEYTFVD | 4559 |
| uniform | FCWDNDWTFMNG | 4560 |
| uniform | YRSFNIFTSTHL | 4561 |
| uniform | CLQVLENPHQPI | 4562 |
| uniform | ILREKTWLIRSV | 4563 |
| uniform | MRIHKFSWPLAT | 4564 |
| uniform | DWPMVQQRAKLE | 4565 |
| uniform | HEDKQPYCLPLS | 4566 |
| uniform | HPTYHQSKVGIN | 4567 |
| uniform | MHQTKWNCYCGV | 4568 |
| uniform | TNTIPWPAYCDR | 4569 |
| uniform | KYTNGQYWYRLR | 4570 |
| uniform | RSRVMFERQGTE | 4571 |
| uniform | LISAHIGQVKGG | 4572 |
| uniform | PIDCECVDKQGV | 4573 |
| uniform | SEPNMMDGQRPT | 4574 |
| uniform | AFKTVWKMEYIF | 4575 |
| uniform | DPTTLIFPTPDP | 4576 |
| uniform | DQQEMALWKIAC | 4577 |
| uniform | AIHKCDWNKNQP | 4578 |
| uniform | GTHTNDQMRSET | 4579 |
| uniform | YNFATWGCWYWV | 4580 |
| uniform | VEGSTKEAMCTH | 4581 |
| uniform | FWYFPWNTAGYP | 4582 |
| uniform | QWVEVYYVWFFQ | 4583 |
| uniform | LRQKMMWPYCNH | 4584 |
| uniform | LVLTRCYSEVMH | 4585 |
| uniform | CCDADENPALQM | 4586 |
| uniform | NHNFEIATNKVK | 4587 |
| uniform | TVIFMKKWLTYC | 4588 |
| uniform | VMAGTFNFGRTG | 4589 |
| uniform | FVWPCAPIHEEY | 4590 |
| uniform | CVWYVMCIYPPQ | 4591 |
| uniform | CRIMALTEQVPD | 4592 |
| uniform | RPDWVRCMYLLS | 4593 |
| uniform | AVVQPCIQTVDA | 4594 |
| uniform | APWDPGRSKIEM | 4595 |
| uniform | MKSQTSQRRKMY | 4596 |
| uniform | QAEDNNDCLKWH | 4597 |
| uniform | SVLNYPIGSCSS | 4598 |
| uniform | KYHGIYNTNQYP | 4599 |
| uniform | VRGPNYRYVTFM | 4600 |
| uniform | HKKWLWWCFVLD | 4601 |
| uniform | LYCQRKLVDDDL | 4602 |
| uniform | KYKQAISSKGAD | 4603 |
| uniform | EHEGQEMVCWYN | 4604 |
| uniform | NWVLTCKSREEA | 4605 |
| uniform | KEASTEMQFDDY | 4606 |
| uniform | RGTDHTNYTVHY | 4607 |
| uniform | VFRGCAMVTTDI | 4608 |
| uniform | EGEAVPCYTRPH | 4609 |
| uniform | MVGIFTLDMCIA | 4610 |
| uniform | QPNRLIMAQNAE | 4611 |
| uniform | HEKNLTRVWNNT | 4612 |
| uniform | LADPMRCRALND | 4613 |
| uniform | YINKCSFKDINE | 4614 |
| uniform | KIKSSGHEHVDS | 4615 |
| uniform | VPHKPIVIMHNF | 4616 |
| uniform | LEISQGFAWMGS | 4617 |
| uniform | VVAFIVQRYPGL | 4618 |
| uniform | LTCCFIDENQPY | 4619 |
| uniform | SANCPEGPAWVA | 4620 |
| uniform | PKKSCRHEECLI | 4621 |
| uniform | CPSNMHSNGFKW | 4622 |
| uniform | FCGTRLLFLVYE | 4623 |
| uniform | CKEMPRPARTMN | 4624 |
| uniform | LVQMNMYKIYMA | 4625 |
| uniform | ELDDDYCENEIK | 4626 |
| uniform | GTQAWKCPRAGC | 4627 |
| uniform | GWDRPNFYHPPF | 4628 |
| uniform | YYMRSQIYHEKA | 4629 |
| uniform | GWVGMTYEPVRP | 4630 |
| uniform | YFERQGLPWMPV | 4631 |
| uniform | AIWSHKSNNMTV | 4632 |
| uniform | TRNWDFRHVVDC | 4633 |
| uniform | CHPFDSVGVSKI | 4634 |
| uniform | DTMHEISEFPQQ | 4635 |
| uniform | PVCAPSQWTRYF | 4636 |
| uniform | MVTRRHKGYQVS | 4637 |
| uniform | QWWCPCYRPMCL | 4638 |
| uniform | LNFVKETGHAGK | 4639 |
| uniform | WPHVMYQRHVLC | 4640 |
| uniform | GVSNSVMCQTSN | 4641 |
| uniform | KIWYWKHMIEFM | 4642 |
| uniform | YFIQFAWDFPGE | 4643 |
| uniform | ETDPYLYENKMD | 4644 |
| uniform | WDCMHTLDQVMN | 4645 |
| uniform | MMGAVIEDERRW | 4646 |
| uniform | LALFVQKYKMFW | 4647 |
| uniform | RWGVHVPLVIMI | 4648 |
| uniform | FAQEKHFRGWGV | 4649 |
| uniform | KIMFVSITLLQM | 4650 |
| uniform | VFNYDFQSPPCY | 4651 |
| uniform | YGAENEWGSQDI | 4652 |
| uniform | RTMYFVHPPTNN | 4653 |
| uniform | ILPLPILWTGGH | 4654 |
| uniform | ECTTGTSPAMPC | 4655 |
| uniform | QAGHPGNKYVVS | 4656 |
| uniform | LILWGLQKRIEY | 4657 |
| uniform | VQQLVMQRWQDN | 4658 |
| uniform | PMWWHFRREDGG | 4659 |
| uniform | EVHHPITRRWNH | 4660 |
| uniform | VRMAFHCRDMSC | 4661 |
| uniform | LEMMYENRCEFQ | 4662 |
| uniform | QNVHWESAMRDC | 4663 |
| uniform | MHEYDKPLLMTF | 4664 |
| uniform | APSRCLYSIRQS | 4665 |
| uniform | IEQRCSQHNGCL | 4666 |
| uniform | GMNRKPIDETGW | 4667 |
| uniform | YKVGIGFEGVML | 4668 |
| uniform | DRQLGFIQQNCR | 4669 |
| uniform | FPYQDFGSKRVF | 4670 |
| uniform | LKTTYACHFDRD | 4671 |
| uniform | NWGPAEQYPTLP | 4672 |
| uniform | HWYASAWTEGLK | 4673 |
| uniform | VGYRLFHQPGIK | 4674 |
| uniform | RLLFCPFGWTEN | 4675 |
| uniform | GVTCLHVRNHAG | 4676 |
| uniform | YSYMFQGGSMTT | 4677 |
| uniform | IKFKEQLACMHM | 4678 |
| uniform | VKFMQTKPKKMS | 4679 |
| uniform | YYMCFFDTDFDG | 4680 |
| uniform | RIWDLIRHEVCD | 4681 |
| uniform | QAKKSIGELHWC | 4682 |
| uniform | FDQLDRNPYYFM | 4683 |
| uniform | YEPGKNTFVTDH | 4684 |
| uniform | SRLRITAWATVS | 4685 |
| uniform | AFWECPVFASTW | 4686 |
| uniform | LRMGGSDDQLHW | 4687 |
| uniform | NTVNNYFVKDCI | 4688 |
| uniform | CRYMDYALQLWS | 4689 |
| uniform | CDMVVRGDQLHA | 4690 |
| uniform | DRKYCYHSGKVD | 4691 |
| uniform | KGCAVPPFAYET | 4692 |
| uniform | IGYINSYWAHTW | 4693 |
| uniform | YVVIITIWRTEK | 4694 |
| uniform | GAWQQKAMRHLW | 4695 |
| uniform | EFTIEDIAPDHN | 4696 |
| uniform | HPLEREHLLNME | 4697 |
| uniform | RDTFMTWRGHSK | 4698 |
| uniform | LAELSLGFCNGY | 4699 |
| uniform | IIERNWALNECL | 4700 |
| uniform | KGDTCFLDNAFH | 4701 |
| uniform | TPRIVTMNDYWK | 4702 |
| uniform | FHCDDATFIHHV | 4703 |
| uniform | AEGHCHLRIYQS | 4704 |
| uniform | PLQHTRASTFLS | 4705 |
| uniform | HWWESHCHYLYN | 4706 |
| uniform | TVFSSMGMCCPD | 4707 |
| uniform | FFQIVELITWNV | 4708 |
| uniform | CDHPEEQRKLKS | 4709 |
| uniform | FSMQFDGVRPAP | 4710 |
| uniform | FYFFFIDCRFAG | 4711 |
| uniform | KDLGNPVKVVLM | 4712 |
| uniform | AKHVGLCTHPWH | 4713 |
| uniform | GKKGIRKIQVEE | 4714 |
| uniform | EEESWHCGIPMT | 4715 |
| uniform | WKRMQFDGTRYV | 4716 |
| uniform | KMMISENSCNPE | 4717 |
| uniform | IKYEIRRTENQW | 4718 |
| uniform | DDAEQSFENPNN | 4719 |
| uniform | RMVEWTHTHAME | 4720 |
| uniform | GPNLAEVYAKQT | 4721 |
| uniform | DEIIDVDCMTAC | 4722 |
| uniform | KQVRHIEEEFTV | 4723 |
| uniform | STKMGAAGMVCP | 4724 |
| uniform | KMILHALTFDVK | 4725 |
| uniform | NVCHELADMPGS | 4726 |
| uniform | IAPSIEYKPWVK | 4727 |
| uniform | IFMSAWCDHQKD | 4728 |
| uniform | PHAQCFLRWATI | 4729 |
| uniform | GSISHCVKQNQV | 4730 |
| uniform | HCQRVEQHVTGE | 4731 |
| uniform | IVTQNTESLKEC | 4732 |
| uniform | YCHMVCGSRMEI | 4733 |
| uniform | GAHMEVDQNSWI | 4734 |
| uniform | DKTIQRYHERNP | 4735 |
| uniform | NQCKNKQRCRLI | 4736 |
| uniform | VHQNFSVSPCQN | 4737 |
| uniform | QKWARKLIPMHE | 4738 |
| uniform | DNQHLCFHGDIW | 4739 |
| uniform | PHIHWRGHEQVR | 4740 |
| uniform | HEYQTDGIFYEK | 4741 |
| uniform | YRYEWYAQVNAY | 4742 |
| uniform | PSIAHYGPQKTH | 4743 |
| uniform | RPAIILLGVIYR | 4744 |
| uniform | MGSFILYVAWMW | 4745 |
| uniform | QYELQVDDLTVQ | 4746 |
| uniform | WCLCVSMNTNEC | 4747 |
| uniform | CMLQVPTVTVAD | 4748 |
| uniform | IHEMSFVWIKHM | 4749 |
| uniform | TKCQWSMIMYCW | 4750 |
| uniform | TSPSIEKEITKH | 4751 |
| uniform | ADSQFQCRNQYW | 4752 |
| uniform | NGTLVHRMTCYC | 4753 |
| uniform | NNPREDQHMLCN | 4754 |
| uniform | CGFKPVMYKFMQ | 4755 |
| uniform | VKRHFKIVGITC | 4756 |
| uniform | YYEGGMHVDNRI | 4757 |
| uniform | RGYHQATMLFFL | 4758 |
| uniform | SYMEYCDAYRLG | 4759 |
| uniform | SAGYQHYIYCSG | 4760 |
| uniform | TLEMASFPPFYS | 4761 |
| uniform | WCSLPDWQKWYS | 4762 |
| uniform | TNNYLNQCWVNT | 4763 |
| uniform | EMQGAEAEVHIR | 4764 |
| uniform | EYGQIPKACGSW | 4765 |
| uniform | CKRCWHYFRHCG | 4766 |
| uniform | MVMRQNQEYEDD | 4767 |
| uniform | WGTREFGYGCKM | 4768 |
| uniform | FERTGWYAWEGE | 4769 |
| uniform | LVEIAVAFHGVG | 4770 |
| uniform | PWGLWRCAFFES | 4771 |
| uniform | SPRQAKVDMNGG | 4772 |
| uniform | PAKRTNRVVCLL | 4773 |
| uniform | WARYPWLPMDER | 4774 |
| uniform | VATPCFTARAQH | 4775 |
| uniform | ELEAWSYGNRST | 4776 |
| uniform | KKETCESACNPF | 4777 |
| uniform | CSFLHVDYYHEW | 4778 |
| uniform | MLIHPTYALWTV | 4779 |
| uniform | HTMVLCDVFDFN | 4780 |
| uniform | TETRECCHGTNA | 4781 |
| uniform | QACISSHSTDGI | 4782 |
| uniform | RLKQFLNYSMHQ | 4783 |
| uniform | RYPHPKMGTCDH | 4784 |
| uniform | RIVLCELMSSGI | 4785 |
| uniform | HRSFVLSSQAFL | 4786 |
| uniform | MNLYHLLCDFQP | 4787 |
| uniform | KNIIREFRIHAC | 4788 |
| uniform | FLYTKATWRGTS | 4789 |
| uniform | VGIKNMSIEQFR | 4790 |
| uniform | RLIFHNFDQWVV | 4791 |
| uniform | PQDADQQEAHGA | 4792 |
| uniform | DVLNHLAVHDFE | 4793 |
| uniform | CKEDKRILQNQN | 4794 |
| uniform | SELKVYNPICAI | 4795 |
| uniform | HKIGEYPYCGQM | 4796 |
| uniform | YCLWKLLIKQCP | 4797 |
| uniform | IICQVFVFAWHY | 4798 |
| uniform | GVGHISQSAKFC | 4799 |
| uniform | IVINHERVTVQM | 4800 |
| uniform | SRGEPQTAYPTN | 4801 |
| uniform | HMQHSSTQLKWN | 4802 |
| uniform | QLHDKSWCPKMR | 4803 |
| uniform | QYNGRENGWDMQ | 4804 |
| uniform | RMTFRPESCHAI | 4805 |
| uniform | MACRTATACEIL | 4806 |
| uniform | TPINGVYIKSMF | 4807 |
| uniform | IYKTISHWEKWC | 4808 |
| uniform | GPGQENQCGVAI | 4809 |
| uniform | WVSHWRTNIYKE | 4810 |
| uniform | RFCKHYMWPVWK | 4811 |
| uniform | CMSALAVQPWLF | 4812 |
| uniform | SDLLTGMDVQYA | 4813 |
| uniform | FQWPNPENVHFP | 4814 |
| uniform | QCHWSMATCIMD | 4815 |
| uniform | GKMWATYYRTSD | 4816 |
| uniform | CDSNNIFCNKKI | 4817 |
| uniform | NSGVRRTPFVPQ | 4818 |
| uniform | DYTFLLTSQYER | 4819 |
| uniform | NCVMPGRHFIMG | 4820 |
| uniform | STOGAQPGRCGQ | 4821 |
| uniform | DYWLTKELAICH | 4822 |
| uniform | HFCHYTPHRYAG | 4823 |
| uniform | KDAYSGPTNEGV | 4824 |
| uniform | KAETKRFCLRNC | 4825 |
| uniform | GQKCPPTTPEQG | 4826 |
| uniform | DRQQMDMDMPIK | 4827 |
| uniform | GWIHTMWAMDSQ | 4828 |
| uniform | GGDVQNMCYPHK | 4829 |
| uniform | NDKCWVLIAKMV | 4830 |
| uniform | YANHIPRRPQFT | 4831 |
| uniform | LDDYIDTAHPVE | 4832 |
| uniform | LKNKSRQMITHE | 4833 |
| uniform | DLYTINFCQCKP | 4834 |
| uniform | IGNSCSTTYHDN | 4835 |
| uniform | DQTKVTLIAARQ | 4836 |
| uniform | HIMKYKTPNIPT | 4837 |
| uniform | CTNCICNCLPMC | 4838 |
| uniform | TIDYLNYSKYMT | 4839 |
| uniform | YECDLAKARKKF | 4840 |
| uniform | IHQKGWYLHSWE | 4841 |
| uniform | KKILWAQASLIP | 4842 |
| uniform | GDWKIRQEGFHD | 4843 |
| uniform | YTEDKETHRCMD | 4844 |
| uniform | MWNRCVPPPIES | 4845 |
| uniform | AEKKIYDFMATA | 4846 |
| uniform | RAPCNSHRRAVE | 4847 |
| uniform | PPVKRDNYDPSK | 4848 |
| uniform | QWRFGFTVMINF | 4849 |
| uniform | CCFMDISIIGNK | 4850 |
| uniform | CTCDLVLTENHC | 4851 |
| uniform | CWSNWMNTDSML | 4852 |
| uniform | MHFYWLQEYPVW | 4853 |
| uniform | GYCAVACWTVVG | 4854 |
| uniform | LKDWEWAFGAGQ | 4855 |
| uniform | PKDNSQLGSGNQ | 4856 |
| uniform | NFWIKTNFMIMD | 4857 |
| uniform | HSHMWVLAGMDK | 4858 |
| uniform | GKSEDMWRIGCH | 4859 |
| uniform | THWMQGRMQHAH | 4860 |
| uniform | TTNCMKRTKPWN | 4861 |
| uniform | ELYLSMRLEFAW | 4862 |
| uniform | CIKCLRGPIVCA | 4863 |
| uniform | IQRHSSQNRWAV | 4864 |
| uniform | RSSICCLYDWTY | 4865 |
| uniform | VKFDWGKMQWSP | 4866 |
| uniform | FGMQTAKETHFC | 4867 |
| uniform | PKRFWYAEEPNL | 4868 |
| uniform | FESFNINCAWFK | 4869 |
| uniform | FDTHIEAQMQNT | 4870 |
| uniform | CTQLWNNDNDLH | 4871 |
| uniform | VLQPYGPCELPI | 4872 |
| uniform | YFAMAACEGTHM | 4873 |
| uniform | FEGWDLWEEHFF | 4874 |
| uniform | NCESKNHYYNEA | 4875 |
| uniform | VCCQIVIAVRLS | 4876 |
| uniform | IDWAWFCSLSRM | 4877 |
| uniform | EHAWEWQTWMVY | 4878 |
| uniform | SNTKAGISGEMK | 4879 |
| uniform | HYDLFIYILKYQ | 4880 |
| uniform | NAKRRYSMECPM | 4881 |
| uniform | DKDCIYDAIYGH | 4882 |
| uniform | GKRMCTDAWANQ | 4883 |
| uniform | QAKNNIQQYRMN | 4884 |
| uniform | EVEVQTYMETNS | 4885 |
| uniform | VHTCYAYINWAM | 4886 |
| uniform | RGDENFTKQNKM | 4887 |
| uniform | HIHVEMACMGTF | 4888 |
| uniform | RLEQFYPVHPPP | 4889 |
| uniform | YSSKYNKPFEVH | 4890 |
| uniform | RQCSLVTLVYPE | 4891 |
| uniform | SLGAHCSKILDI | 4892 |
| uniform | GLWTNPEPKYDD | 4893 |
| uniform | CMPCIQTHRVTV | 4894 |
| uniform | YLLQRQMEEFTY | 4895 |
| uniform | VQRIYQICTGMT | 4896 |
| uniform | ITCDRVVSIHQW | 4897 |
| uniform | NSVDVKIDLGFS | 4898 |
| uniform | SHLTDIRKICCW | 4899 |
| uniform | KVDLCVSHCRRT | 4900 |
| uniform | RTHHLEWLPTYH | 4901 |
| uniform | TFALIYALQDFP | 4902 |
| uniform | MPSWVVEPNAVG | 4903 |
| uniform | SHRWTPMTYTVQ | 4904 |
| uniform | RISDLRRVCWFN | 4905 |
| uniform | EHPFIWMVERMW | 4906 |
| uniform | ANLYGWMAIGIS | 4907 |
| uniform | GKRKYVVNSRNC | 4908 |
| uniform | DLHPGEAHVDDS | 4909 |
| uniform | IEDDCNKWKCYW | 4910 |
| uniform | LYWQRLYHCKTW | 4911 |
| uniform | LFHDPTITERSD | 4912 |
| uniform | LTVLTVFFPQFP | 4913 |
| uniform | NFLFVKHKERSD | 4914 |
| uniform | FTSAQEDDMEKF | 4915 |
| uniform | EMVFQKTGVSWI | 4916 |
| uniform | PAMYIHYQYWLH | 4917 |
| uniform | HLVWDKQNDQIW | 4918 |
| uniform | PLLGMAMTGGTP | 4919 |
| uniform | ILFEQTYQMMPF | 4920 |
| uniform | MMTYMIHYQIPG | 4921 |
| uniform | KSWHVYPACPCT | 4922 |
| uniform | MRHAVFYYSNTR | 4923 |
| uniform | YMAKSRDHIGQP | 4924 |
| uniform | GLIEWNKWEGDN | 4925 |
| uniform | RCKHQCEHVQFP | 4926 |
| uniform | VRSLILTAMTKV | 4927 |
| uniform | LAIGLVFKDVWD | 4928 |
| uniform | WNEQENGVGLGC | 4929 |
| uniform | VFLMLYKCRGNK | 4930 |
| uniform | WGEGGFIKIQKM | 4931 |
| uniform | SNGSMEDLYCKA | 4932 |
| uniform | AFWYPFMCKEIA | 4933 |
| uniform | VLCQNWTRFPYI | 4934 |
| uniform | VDEREENPSCVP | 4935 |
| uniform | LMSQYWIDTRVR | 4936 |
| uniform | FFCYPIYADNTM | 4937 |
| uniform | RILVFEKNHRAK | 4938 |
| uniform | WHRYCVNFNPHY | 4939 |
| uniform | MFLLWHDEKKLQ | 4940 |
| uniform | AIPVKKKWASAF | 4941 |
| uniform | EDFIQDPHDQCS | 4942 |
| uniform | YAMHDDSPNIDW | 4943 |
| uniform | CICHEITELFVY | 4944 |
| uniform | CSCNDNCGPELR | 4945 |
| uniform | RTNQDCDAYLVM | 4946 |
| uniform | NQYFAQTEEDGP | 4947 |
| uniform | CCLEFAEVKHVL | 4948 |
| uniform | RCPPIGPHILRP | 4949 |
| uniform | KFPSREAMMWND | 4950 |
| uniform | PFCQGDLYGAQC | 4951 |
| uniform | ITNWDDVCVWSK | 4952 |
| uniform | ILNFRIRFIDAQ | 4953 |
| uniform | QARLSGQEFIGG | 4954 |
| uniform | AWSRVMAQGNRD | 4955 |
| uniform | DQQTEFTKNYYA | 4956 |
| uniform | DGYRDYYDHQVS | 4957 |
| uniform | IIDTWMWNILWG | 4958 |
| uniform | PADLGMVSDDQW | 4959 |
| uniform | DKKERQNWCVCC | 4960 |
| uniform | FATRCGDPGAIN | 4961 |
| uniform | TLMEHSHICDLR | 4962 |
| uniform | PCWPKQGEQQSG | 4963 |
| uniform | CLMQNYNADMLR | 4964 |
| uniform | KTDHPTNWAYGW | 4965 |
| uniform | VWRATVCLEGIY | 4966 |
| uniform | DGDNNMGILRGN | 4967 |
| uniform | ASRMMTTHTYQE | 4968 |
| uniform | EWGQNERGAKRY | 4969 |
| uniform | DCCCHYYDVISI | 4970 |
| uniform | ALLPHFRKITSP | 4971 |
| uniform | WTAVCIPKMCHM | 4972 |
| uniform | EEHHYQNYRDWP | 4973 |
| uniform | SVYVDQAHHDND | 4974 |
| uniform | CMIVWQNEWAYK | 4975 |
| uniform | CWEINFVLCRRT | 4976 |
| uniform | VCAEDLDPPTLA | 4977 |
| uniform | KSHDPYSGKNYL | 4978 |
| uniform | DFIRTREHCGKG | 4979 |
| uniform | DTKPMWDGQMPL | 4980 |
| uniform | FPFFGMMPCQGQ | 4981 |
| uniform | FIIYFVFVFREM | 4982 |
| uniform | WGYAKPAKRTSE | 4983 |
| uniform | SRGHDHLSCSMR | 4984 |
| uniform | HNNYMWVLCMQE | 4985 |
| uniform | HCCVDRHKVIPQ | 4986 |
| uniform | GEIIQLCGKRQE | 4987 |
| uniform | QCVYMKEEPFDD | 4988 |
| uniform | FENTAIIVVKLS | 4989 |
| uniform | LVCNYQVTQNIE | 4990 |
| uniform | AEWWMTWFTDIR | 4991 |
| uniform | FEQPSIHKFWFT | 4992 |
| uniform | AWHMINCLLQKS | 4993 |
| uniform | WLCNQCHQLVFD | 4994 |
| uniform | RNEHVWINHNDY | 4995 |
| uniform | VWRQQTTFQRGD | 4996 |
| uniform | TWNNVIGLPADC | 4997 |
| uniform | DPHACHACENYW | 4998 |
| uniform | VTKRETQVEGPA | 4999 |
| uniform | NCACSKTTMSHM | 5000 |
| uniform | NVAQHNSPWYYC | 5001 |
| uniform | IPFWKHHEQLQP | 5002 |
| uniform | HYFNLKTHWGPT | 5003 |
| uniform | VNVCLNHWAFRE | 5004 |
| uniform | WARVKFGPQPNQ | 5005 |
| weighted | GDKFPCST | 5006 |
| weighted | KIAFNHLL | 5007 |
| weighted | ESITTMFS | 5008 |
| weighted | QDEFSWAR | 5009 |
| weighted | TPARGGNV | 5010 |
| weighted | RERCAANR | 5011 |
| weighted | QQCHHVSE | 5012 |
| weighted | VIHEVGEG | 5013 |
| weighted | KNHNLVFR | 5014 |
| weighted | MSRKGLDQ | 5015 |
| weighted | LYNQPPSV | 5016 |
| weighted | VSQFFYMR | 5017 |
| weighted | IRVKPGQR | 5018 |
| weighted | VDRSESCA | 5019 |
| weighted | WIVLELMM | 5020 |
| weighted | YLQETDMF | 5021 |
| weighted | SMGYSSIS | 5022 |
| weighted | PNALDAGA | 5023 |
| weighted | VVFRRSAK | 5024 |
| weighted | EIARLAPE | 5025 |
| weighted | WDLTRAGL | 5026 |
| weighted | VWASYKST | 5027 |
| weighted | EYELAELE | 5028 |
| weighted | TRTVFFGN | 5029 |
| weighted | QMFIASDH | 5030 |
| weighted | AVSLEEHS | 5031 |
| weighted | SLDHLICR | 5032 |
| weighted | MALIAKVR | 5033 |
| weighted | DTIAVFSV | 5034 |
| weighted | VSRREDIE | 5035 |
| weighted | VLGEDQTP | 5036 |
| weighted | EESEFTQR | 5037 |
| weighted | QKDKVIDS | 5038 |
| weighted | IPELGTTG | 5039 |
| weighted | ALPMRQIG | 5040 |
| weighted | KMYQGRPT | 5041 |
| weighted | KRRTERIQ | 5042 |
| weighted | NEETIGLK | 5043 |
| weighted | PMSIQMLD | 5044 |
| weighted | PFNMSNEY | 5045 |
| weighted | TDLLGLEF | 5046 |
| weighted | AVNTASGI | 5047 |
| weighted | PERVWMSY | 5048 |
| weighted | ATTDTTFQ | 5049 |
| weighted | VREPAVGS | 5050 |
| weighted | FRVSHHIP | 5051 |
| weighted | KEENIMKI | 5052 |
| weighted | LCDEEEIR | 5053 |
| weighted | VHPAQEWE | 5054 |
| weighted | RGPYKRLS | 5055 |
| weighted | FPKLPVLW | 5056 |
| weighted | HQINLLGP | 5057 |
| weighted | RVLCGRKM | 5058 |
| weighted | PSPFRIVH | 5059 |
| weighted | AMEELFEL | 5060 |
| weighted | QFNNAVIK | 5061 |
| weighted | VELLQTAS | 5062 |
| weighted | PVILDGQN | 5063 |
| weighted | VEHADIDS | 5064 |
| weighted | GWEYLNRE | 5065 |
| weighted | FTSTGAIG | 5066 |
| weighted | ATPAFDVS | 5067 |
| weighted | NVETEQAE | 5068 |
| weighted | QQRLEPNE | 5069 |
| weighted | AIITGTMD | 5070 |
| weighted | LVGTTHVQ | 5071 |
| weighted | EHGPSLEP | 5072 |
| weighted | AAHQPQPT | 5073 |
| weighted | PELTELLI | 5074 |
| weighted | KSPEQVLP | 5075 |
| weighted | LVSSFVHK | 5076 |
| weighted | VLHENRRP | 5077 |
| weighted | IRRLKNGM | 5078 |
| weighted | YQRWRSKA | 5079 |
| weighted | KHTGGDRV | 5080 |
| weighted | SPQGLSVE | 5081 |
| weighted | VDILREIP | 5082 |
| weighted | EPVKVECG | 5083 |
| weighted | RPSNTCLQ | 5084 |
| weighted | PLQEQEVV | 5085 |
| weighted | NAKETWEQ | 5086 |
| weighted | LGLAMIAE | 5087 |
| weighted | GAGGRLPG | 5088 |
| weighted | SVTQHKEG | 5089 |
| weighted | VTEFNKSS | 5090 |
| weighted | FSLRRQRD | 5091 |
| weighted | GGLGKSGL | 5092 |
| weighted | WTGAETSD | 5093 |
| weighted | GLKQGAYN | 5094 |
| weighted | MPENEECV | 5095 |
| weighted | VLLGDKKG | 5096 |
| weighted | RSMVLCNP | 5097 |
| weighted | VKPTLIHI | 5098 |
| weighted | KSMMSLRE | 5099 |
| weighted | PDVLKAYK | 5100 |
| weighted | IPVICGLN | 5101 |
| weighted | PPEHCPST | 5102 |
| weighted | IYGRKQQP | 5103 |
| weighted | SIISYSLV | 5104 |
| weighted | LQESTALV | 5105 |
| weighted | PVKGVGFG | 5106 |
| weighted | ESKDVTIF | 5107 |
| weighted | AFQFSESA | 5108 |
| weighted | ISKLPMSD | 5109 |
| weighted | RVWLLVLS | 5110 |
| weighted | EHVIITAM | 5111 |
| weighted | IEALRFMQ | 5112 |
| weighted | SPQAKASS | 5113 |
| weighted | VASPALPP | 5114 |
| weighted | ERGFERGP | 5115 |
| weighted | IAAVPEPP | 5116 |
| weighted | LLAYQPHN | 5117 |
| weighted | LQDDLLAK | 5118 |
| weighted | ITTYLRLD | 5119 |
| weighted | LKGSVDET | 5120 |
| weighted | NGQDPNSQ | 5121 |
| weighted | VQLQGHAV | 5122 |
| weighted | LGPDKADR | 5123 |
| weighted | EALQKSRG | 5124 |
| weighted | RPCETLON | 5125 |
| weighted | NHPTFESC | 5126 |
| weighted | ERPKPSRV | 5127 |
| weighted | SNSMVKFQ | 5128 |
| weighted | MPGSNESE | 5129 |
| weighted | FPQQEADM | 5130 |
| weighted | QETEKCIK | 5131 |
| weighted | LNGLSQSL | 5132 |
| weighted | FTSYRMDQ | 5133 |
| weighted | EQHAVEAG | 5134 |
| weighted | AIAENCFQ | 5135 |
| weighted | IGPVTTCR | 5136 |
| weighted | ARAWQMVT | 5137 |
| weighted | PIGQLDGN | 5138 |
| weighted | YGISFATP | 5139 |
| weighted | GALSDGPM | 5140 |
| weighted | LLPADQVP | 5141 |
| weighted | AVTSNRLT | 5142 |
| weighted | LALVVSKL | 5143 |
| weighted | VEAMMGKL | 5144 |
| weighted | LLVKESPS | 5145 |
| weighted | KPTSLDGK | 5146 |
| weighted | MVSTHREV | 5147 |
| weighted | TLVYVKSL | 5148 |
| weighted | PPEVDALH | 5149 |
| weighted | SSKELTYD | 5150 |
| weighted | GTLYAIQL | 5151 |
| weighted | LGPASTSK | 5152 |
| weighted | GNVIYFGS | 5153 |
| weighted | SSQFDSKS | 5154 |
| weighted | VSLDTFSK | 5155 |
| weighted | MERHCHDA | 5156 |
| weighted | KHCSTKDA | 5157 |
| weighted | LVRPQSFA | 5158 |
| weighted | ISNPMQSG | 5159 |
| weighted | SGGQLVLA | 5160 |
| weighted | AKDIYLDL | 5161 |
| weighted | TEDSKSAQ | 5162 |
| weighted | QMVPTKLG | 5163 |
| weighted | RPNRSNPC | 5164 |
| weighted | GLVWSKSL | 5165 |
| weighted | VPVRQGGP | 5166 |
| weighted | PGASAPGQ | 5167 |
| weighted | VIKPAVFS | 5168 |
| weighted | ETWSMEEP | 5169 |
| weighted | QVAATVRM | 5170 |
| weighted | FADIRQLL | 5171 |
| weighted | KLQYNDLS | 5172 |
| weighted | AQLAIGAW | 5173 |
| weighted | LISFSLAI | 5174 |
| weighted | WVPPDWAY | 5175 |
| weighted | WDNQLILL | 5176 |
| weighted | QVLEKSQP | 5177 |
| weighted | PCFPQPLI | 5178 |
| weighted | SYFTNGQV | 5179 |
| weighted | LFMTESEL | 5180 |
| weighted | QSFCDLNP | 5181 |
| weighted | SSEAKTAT | 5182 |
| weighted | WASHCKLL | 5183 |
| weighted | QIQCWMSR | 5184 |
| weighted | PEFIHVMQ | 5185 |
| weighted | SSYKADLC | 5186 |
| weighted | AVFEAHMS | 5187 |
| weighted | LKFAPPTR | 5188 |
| weighted | GDPGSSER | 5189 |
| weighted | ESLFLCYL | 5190 |
| weighted | TSLDNEGW | 5191 |
| weighted | QKTAAGSC | 5192 |
| weighted | LGLASLSG | 5193 |
| weighted | SINSIECL | 5194 |
| weighted | ELEQYSRS | 5195 |
| weighted | SNLDLGDE | 5196 |
| weighted | ADAIERDP | 5197 |
| weighted | SPTAVKYP | 5198 |
| weighted | PDKSTTQF | 5199 |
| weighted | GEIIRTCD | 5200 |
| weighted | TDSPFQYR | 5201 |
| weighted | QDLLLVPL | 5202 |
| weighted | AEPAEPYP | 5203 |
| weighted | QQKCSCRK | 5204 |
| weighted | MHIRESNA | 5205 |
| weighted | QKIVQTGA | 5206 |
| weighted | ILKLRKAKC | 5207 |
| weighted | GLHDQLSA | 5208 |
| weighted | YIRNWRER | 5209 |
| weighted | KNVPNEHM | 5210 |
| weighted | FQLNPMVE | 5211 |
| weighted | KKVHTENL | 5212 |
| weighted | LKLLMGEY | 5213 |
| weighted | EPQEQSSQ | 5214 |
| weighted | SPNVHLPV | 5215 |
| weighted | EISIPPEL | 5216 |
| weighted | DGNPRGSS | 5217 |
| weighted | LFAIKMNL | 5218 |
| weighted | WKKNMAET | 5219 |
| weighted | DSQLDHDF | 5220 |
| weighted | ILQLIDAR | 5221 |
| weighted | NKPGSSTG | 5222 |
| weighted | GDQRLLET | 5223 |
| weighted | HVETSISK | 5224 |
| weighted | NKAYLEFR | 5225 |
| weighted | MAARAVQR | 5226 |
| weighted | PKEENKWD | 5227 |
| weighted | PNVTDDGL | 5228 |
| weighted | SVLPLNRS | 5229 |
| weighted | HDGPSKQT | 5230 |
| weighted | GRLINVAG | 5231 |
| weighted | CSGRSCSN | 5232 |
| weighted | WILGELGV | 5233 |
| weighted | RLVLPNLE | 5234 |
| weighted | KEQLRVMI | 5235 |
| weighted | SDFLPYKC | 5236 |
| weighted | KSQHSVNE | 5237 |
| weighted | SYLTMLRK | 5238 |
| weighted | DVRELAYP | 5239 |
| weighted | IADAGFNE | 5240 |
| weighted | GKGVVGKR | 5241 |
| weighted | TQFTPRRI | 5242 |
| weighted | QPRCEKGA | 5243 |
| weighted | YGYSALSV | 5244 |
| weighted | ARTITVEL | 5245 |
| weighted | GEVTFELS | 5246 |
| weighted | PVSPQEEL | 5247 |
| weighted | GYNSEKVI | 5248 |
| weighted | GQHSCESP | 5249 |
| weighted | PQVDGRVF | 5250 |
| weighted | ISIQERWT | 5251 |
| weighted | SVSCALRA | 5252 |
| weighted | DDFKKRYS | 5253 |
| weighted | SEKNPFLI | 5254 |
| weighted | QVRLVCQG | 5255 |
| weighted | NQLGAQAL | 5256 |
| weighted | RLSPRSRS | 5257 |
| weighted | VIACAFQS | 5258 |
| weighted | LGPNRFDV | 5259 |
| weighted | PNRSHENP | 5260 |
| weighted | VVPAFSPG | 5261 |
| weighted | FESVRAQE | 5262 |
| weighted | SGDQANYL | 5263 |
| weighted | TTAKWESV | 5264 |
| weighted | VKAQGLLP | 5265 |
| weighted | FHSLLPIS | 5266 |
| weighted | AFRRAFLT | 5267 |
| weighted | TYTGNVLQ | 5268 |
| weighted | EFELMMCN | 5269 |
| weighted | STVNGQYS | 5270 |
| weighted | GVNEKDCT | 5271 |
| weighted | QAPKACTM | 5272 |
| weighted | LLTAFALL | 5273 |
| weighted | HSMSINEA | 5274 |
| weighted | HRLQALEP | 5275 |
| weighted | ERLLVNVV | 5276 |
| weighted | PLVQERRL | 5277 |
| weighted | VLKPLNET | 5278 |
| weighted | PYTEDQHN | 5279 |
| weighted | LLTGGQGL | 5280 |
| weighted | KHATPVIK | 5281 |
| weighted | FEKTHQGR | 5282 |
| weighted | VLVVSKRA | 5283 |
| weighted | SSSVVPLD | 5284 |
| weighted | GCFEGRTI | 5285 |
| weighted | EETSIWAS | 5286 |
| weighted | DVQKQVIS | 5287 |
| weighted | AALIGLPA | 5288 |
| weighted | RHMWFAKV | 5289 |
| weighted | LSGEDDNA | 5290 |
| weighted | QVGEKVRG | 5291 |
| weighted | FKGLDWGQ | 5292 |
| weighted | GVEPDRAQ | 5293 |
| weighted | TALESEVL | 5294 |
| weighted | RTRMCVSQ | 5295 |
| weighted | GCTFTGPD | 5296 |
| weighted | MWREHTFQ | 5297 |
| weighted | LQSSEMQQ | 5298 |
| weighted | GAVPSTQE | 5299 |
| weighted | LESVKGHV | 5300 |
| weighted | SSMTDTGA | 5301 |
| weighted | AQLWTAPK | 5302 |
| weighted | INFVGTEK | 5303 |
| weighted | RTDDDLSS | 5304 |
| weighted | LLIAGIGA | 5305 |
| weighted | KSCHPVDE | 5306 |
| weighted | TKSQFEGG | 5307 |
| weighted | VSILEVFV | 5308 |
| weighted | FSECVVNM | 5309 |
| weighted | PRVANAIM | 5310 |
| weighted | SDVSRPIF | 5311 |
| weighted | LQSEECAE | 5312 |
| weighted | VNIYPSNL | 5313 |
| weighted | IPKKYSRH | 5314 |
| weighted | RGRDPNHL | 5315 |
| weighted | IQLSEINI | 5316 |
| weighted | LCLPTDMQ | 5317 |
| weighted | SVNRNSLP | 5318 |
| weighted | AQESLNEA | 5319 |
| weighted | TDTPDAAQ | 5320 |
| weighted | PSVHWTPM | 5321 |
| weighted | DRSTDKGH | 5322 |
| weighted | EVPEQSDV | 5323 |
| weighted | FLRLPKVI | 5324 |
| weighted | EPNHKPLE | 5325 |
| weighted | RACEVSDL | 5326 |
| weighted | QELSSGLI | 5327 |
| weighted | LGAMVSST | 5328 |
| weighted | AGNSWEII | 5329 |
| weighted | ERLWVLNT | 5330 |
| weighted | LGTKLQRP | 5331 |
| weighted | NPMAKASG | 5332 |
| weighted | GSYEAGEL | 5333 |
| weighted | AFVPKGPH | 5334 |
| weighted | AQFFYFLP | 5335 |
| weighted | RKWRYIYK | 5336 |
| weighted | LTDGHGAV | 5337 |
| weighted | LAGLQPGI | 5338 |
| weighted | SSQQKVHA | 5339 |
| weighted | LLTHKLEG | 5340 |
| weighted | FYLPDIHV | 5341 |
| weighted | QGQEWFVE | 5342 |
| weighted | GPARLSAR | 5343 |
| weighted | QVSRLVTS | 5344 |
| weighted | GASVQLAS | 5345 |
| weighted | EYSEDYSF | 5346 |
| weighted | GTVQSRSK | 5347 |
| weighted | FLARPLKQ | 5348 |
| weighted | PYTTLSAS | 5349 |
| weighted | RPVITGNN | 5350 |
| weighted | PKSTTHLK | 5351 |
| weighted | AIDPDEFA | 5352 |
| weighted | PFTEQEKP | 5353 |
| weighted | LDDVILVK | 5354 |
| weighted | QLVGHSGT | 5355 |
| weighted | IVEQSLGY | 5356 |
| weighted | AGTTHILL | 5357 |
| weighted | IVNTFLVY | 5358 |
| weighted | PKPSLDFS | 5359 |
| weighted | ANPQKIAP | 5360 |
| weighted | HSLGAAPF | 5361 |
| weighted | YYCLKTKE | 5362 |
| weighted | SGDVASPL | 5363 |
| weighted | RAKPQAPF | 5364 |
| weighted | VMSIRLSA | 5365 |
| weighted | SVDEELTA | 5366 |
| weighted | KPENREVK | 5367 |
| weighted | DHGYGSES | 5368 |
| weighted | NILPHAMV | 5369 |
| weighted | QPIAVNEF | 5370 |
| weighted | WDAGICNP | 5371 |
| weighted | TGDYDQNS | 5372 |
| weighted | LEVVESQW | 5373 |
| weighted | TDVQEEVR | 5374 |
| weighted | EVSMGMHP | 5375 |
| weighted | KPEPGQIR | 5376 |
| weighted | IEMSVKSC | 5377 |
| weighted | ANAEEEFT | 5378 |
| weighted | MAYNWETC | 5379 |
| weighted | PALDVGQE | 5380 |
| weighted | LRMHEYRV | 5381 |
| weighted | IRKEPIQW | 5382 |
| weighted | RQRTPKPA | 5383 |
| weighted | DFIGLYTE | 5384 |
| weighted | DGPGIVAP | 5385 |
| weighted | DVASKKSR | 5386 |
| weighted | QMSEKAWT | 5387 |
| weighted | RLLEPLSL | 5388 |
| weighted | LQEFVIGY | 5389 |
| weighted | PHADTRPL | 5390 |
| weighted | TSNLKSDC | 5391 |
| weighted | QSPFALCP | 5392 |
| weighted | LDLTKGFC | 5393 |
| weighted | LHAPHTSG | 5394 |
| weighted | TETIVKSA | 5395 |
| weighted | YYRSRLPE | 5396 |
| weighted | LNRYAGKS | 5397 |
| weighted | DLAKLLSV | 5398 |
| weighted | PDDFLYLQ | 5399 |
| weighted | IRNVFLYV | 5400 |
| weighted | PIVDFSTA | 5401 |
| weighted | VPPCATKL | 5402 |
| weighted | EEPSVPVK | 5403 |
| weighted | IIGQAIAP | 5404 |
| weighted | CIFQKTSV | 5405 |
| weighted | LFDKAVGR | 5406 |
| weighted | LKKGKSVL | 5407 |
| weighted | QAKRSKEA | 5408 |
| weighted | MDQPHVLS | 5409 |
| weighted | THALSDYH | 5410 |
| weighted | KPSKSVLA | 5411 |
| weighted | GGGHDNLE | 5412 |
| weighted | DDVEFTAP | 5413 |
| weighted | GNLKVPVE | 5414 |
| weighted | SPLLGGGN | 5415 |
| weighted | VNLEDFRP | 5416 |
| weighted | CLRVVGVI | 5417 |
| weighted | RLACGTEH | 5418 |
| weighted | SVLEGHPL | 5419 |
| weighted | AWYDIRQT | 5420 |
| weighted | AIPEFLNA | 5421 |
| weighted | SNSPMSCQ | 5422 |
| weighted | VDRYRTQQ | 5423 |
| weighted | YILESGTS | 5424 |
| weighted | PQGVAHEI | 5425 |
| weighted | LKESILVY | 5426 |
| weighted | TLEEIARE | 5427 |
| weighted | GLRHVSLD | 5428 |
| weighted | HGIANRLS | 5429 |
| weighted | MDELANQE | 5430 |
| weighted | EESGKVCE | 5431 |
| weighted | IMLSIRIG | 5432 |
| weighted | NLSRHPSI | 5433 |
| weighted | RVIPKMSR | 5434 |
| weighted | GKSLSFGL | 5435 |
| weighted | CTAEPSRG | 5436 |
| weighted | PPRESQHV | 5437 |
| weighted | TGKQYKPA | 5438 |
| weighted | IGGSFLAT | 5439 |
| weighted | TPLWVELK | 5440 |
| weighted | NEVTPGHD | 5441 |
| weighted | DKPSFKQV | 5442 |
| weighted | LMIFDLVL | 5443 |
| weighted | KFSLNLRK | 5444 |
| weighted | WDKSIKSR | 5445 |
| weighted | ERLGWMGF | 5446 |
| weighted | THALMQKG | 5447 |
| weighted | ALNLSSYP | 5448 |
| weighted | EVYEKYLD | 5449 |
| weighted | RAHERAPT | 5450 |
| weighted | LPQFRTPV | 5451 |
| weighted | ETKDKKLE | 5452 |
| weighted | QGEFCCAP | 5453 |
| weighted | FQFDPYAT | 5454 |
| weighted | KLPQLREY | 5455 |
| weighted | LKTEVSLR | 5456 |
| weighted | CATVVEIV | 5457 |
| weighted | TGVVFKQK | 5458 |
| weighted | QFRELKEY | 5459 |
| weighted | REESDNDM | 5460 |
| weighted | APFGYSSA | 5461 |
| weighted | LKPQWRDS | 5462 |
| weighted | YSGTNGDT | 5463 |
| weighted | LKMGGLKS | 5464 |
| weighted | LQESYAKV | 5465 |
| weighted | AKKACQDC | 5466 |
| weighted | KVQIMRFY | 5467 |
| weighted | VQIQATIM | 5468 |
| weighted | HIDLLSFH | 5469 |
| weighted | LASLEQSD | 5470 |
| weighted | SGLGGRSD | 5471 |
| weighted | LKDTCYVG | 5472 |
| weighted | NVTLQRDK | 5473 |
| weighted | RIKGVGAA | 5474 |
| weighted | SGLVMKLK | 5475 |
| weighted | NVPKDNLI | 5476 |
| weighted | VKKSFYLD | 5477 |
| weighted | GLGPFIGA | 5478 |
| weighted | SEIPNGLA | 5479 |
| weighted | LCCTDGPV | 5480 |
| weighted | FGWKEIDL | 5481 |
| weighted | ASSQATCN | 5482 |
| weighted | SGKVALPT | 5483 |
| weighted | EFLHMVRT | 5484 |
| weighted | TVLIKTKW | 5485 |
| weighted | RGQEENKE | 5486 |
| weighted | DQRGARSA | 5487 |
| weighted | RDFTEVDR | 5488 |
| weighted | QGLKLYTN | 5489 |
| weighted | HFKLAVFS | 5490 |
| weighted | IEICSQRP | 5491 |
| weighted | RSLSKASL | 5492 |
| weighted | QELKYIMD | 5493 |
| weighted | NERSVINS | 5494 |
| weighted | NAEIHMLG | 5495 |
| weighted | INTSHPRI | 5496 |
| weighted | EQEAADLT | 5497 |
| weighted | QFTSMKRH | 5498 |
| weighted | TSESKKPF | 5499 |
| weighted | TGVGEVLF | 5500 |
| weighted | EVTTEMRA | 5501 |
| weighted | LIIVARIF | 5502 |
| weighted | ARLSGYFG | 5503 |
| weighted | ALEISIGQ | 5504 |
| weighted | ERFCDPLP | 5505 |
| weighted | FFTELSNT | 5506 |
| weighted | IGELLGGY | 5507 |
| weighted | LWSAEEGA | 5508 |
| weighted | VGHVIDAE | 5509 |
| weighted | VKGAHPGQ | 5510 |
| weighted | GSLASPAH | 5511 |
| weighted | EQPLNASP | 5512 |
| weighted | IGLREASE | 5513 |
| weighted | PLALDLLE | 5514 |
| weighted | KFPIVCET | 5515 |
| weighted | CFYVVAVP | 5516 |
| weighted | EQVVALEI | 5517 |
| weighted | VQADTIPG | 5518 |
| weighted | KITENPSN | 5519 |
| weighted | LPKDSSKF | 5520 |
| weighted | LPGIIEFA | 5521 |
| weighted | TNCHYNTQ | 5522 |
| weighted | RWFRVKHN | 5523 |
| weighted | KPPATYFS | 5524 |
| weighted | GVLRLNDV | 5525 |
| weighted | PEVIELCH | 5526 |
| weighted | IVALAARL | 5527 |
| weighted | VSSTGWTP | 5528 |
| weighted | ECDFLCQP | 5529 |
| weighted | TSNQPDVK | 5530 |
| weighted | EAGPWAFC | 5531 |
| weighted | CMAGCSDS | 5532 |
| weighted | QEIGPDDC | 5533 |
| weighted | TSPVDCNP | 5534 |
| weighted | HKVMLMRV | 5535 |
| weighted | GQIGANFK | 5536 |
| weighted | ADEDESER | 5537 |
| weighted | LCNHRLEE | 5538 |
| weighted | SAGGAYES | 5539 |
| weighted | SFLRFDVK | 5540 |
| weighted | FSPSRLIY | 5541 |
| weighted | LTLLEDEF | 5542 |
| weighted | NPLDISLS | 5543 |
| weighted | SMNVRDVI | 5544 |
| weighted | ALMLIQEK | 5545 |
| weighted | AFKVLLFH | 5546 |
| weighted | TMPQTPYD | 5547 |
| weighted | PRAHVPKQ | 5548 |
| weighted | AFVFACIV | 5549 |
| weighted | PPYPSRFP | 5550 |
| weighted | TKQALRVE | 5551 |
| weighted | PGYCKLSK | 5552 |
| weighted | EKMHKFSH | 5553 |
| weighted | LLEGTMIG | 5554 |
| weighted | AMSNYQSS | 5555 |
| weighted | NAALPHNS | 5556 |
| weighted | GRPPEIVS | 5557 |
| weighted | GTSVRFLE | 5558 |
| weighted | EVTLAWRS | 5559 |
| weighted | HAASAQRT | 5560 |
| weighted | FFPWLEGK | 5561 |
| weighted | KDLPSNAN | 5562 |
| weighted | QEKVVGSG | 5563 |
| weighted | GRSPTIVV | 5564 |
| weighted | VGPAVVMD | 5565 |
| weighted | LRPLTLEK | 5566 |
| weighted | SALMLTIY | 5567 |
| weighted | CWRSEKDR | 5568 |
| weighted | LSHPRLSL | 5569 |
| weighted | KLKRRKCN | 5570 |
| weighted | EGLPTNWN | 5571 |
| weighted | MSKSLSVI | 5572 |
| weighted | FTQKRSKS | 5573 |
| weighted | PNLNAIRI | 5574 |
| weighted | YSDLQPES | 5575 |
| weighted | RQLDSFLG | 5576 |
| weighted | DAPQEISS | 5577 |
| weighted | GQFELNDE | 5578 |
| weighted | EDGTYQED | 5579 |
| weighted | HLLQYNAI | 5580 |
| weighted | PIQSLVIP | 5581 |
| weighted | GRGPKLFA | 5582 |
| weighted | SLNEHKPG | 5583 |
| weighted | ATNQTNAV | 5584 |
| weighted | LRIKLPNE | 5585 |
| weighted | WDALFPMD | 5586 |
| weighted | PFESNEGT | 5587 |
| weighted | LNTGKCVL | 5588 |
| weighted | PLIDPWDP | 5589 |
| weighted | SVGEAGWI | 5590 |
| weighted | VAGKDKQP | 5591 |
| weighted | PYFSQHRP | 5592 |
| weighted | WEKLNKKR | 5593 |
| weighted | IYQELHTD | 5594 |
| weighted | GLESGKEI | 5595 |
| weighted | EPKIELQL | 5596 |
| weighted | PPEEQNEV | 5597 |
| weighted | GSFGWDDK | 5598 |
| weighted | LHIMANGA | 5599 |
| weighted | QARHEILI | 5600 |
| weighted | QFPTPLVM | 5601 |
| weighted | VGSFHLEF | 5602 |
| weighted | SRLVAEQK | 5603 |
| weighted | MTAFLCRA | 5604 |
| weighted | IKVPTTIK | 5605 |
| weighted | IAIDDGAN | 5606 |
| weighted | QCTVIQPG | 5607 |
| weighted | INYENKQV | 5608 |
| weighted | PGQLPQGQ | 5609 |
| weighted | MLCLLPDL | 5610 |
| weighted | MYQTCAWR | 5611 |
| weighted | GCANHPIL | 5612 |
| weighted | VYIAPRVA | 5613 |
| weighted | ALHGNLML | 5614 |
| weighted | HDLIDELC | 5615 |
| weighted | DGFGTKAS | 5616 |
| weighted | LLGRESLV | 5617 |
| weighted | RRKSIPWN | 5618 |
| weighted | QINSDQFD | 5619 |
| weighted | QDLVATPF | 5620 |
| weighted | FKRVANLE | 5621 |
| weighted | LIKNDTFT | 5622 |
| weighted | ALPPALPE | 5623 |
| weighted | KYMNEQGV | 5624 |
| weighted | TSLGIVRG | 5625 |
| weighted | EMILFIMI | 5626 |
| weighted | FVTSGQHD | 5627 |
| weighted | AAPLGVEV | 5628 |
| weighted | LGGFAGGI | 5629 |
| weighted | VEFYTVYL | 5630 |
| weighted | TSKHHTLW | 5631 |
| weighted | LIITIKKY | 5632 |
| weighted | FNMHEKEC | 5633 |
| weighted | PLPDFFEA | 5634 |
| weighted | HLQATQYI | 5635 |
| weighted | VREPLSFK | 5636 |
| weighted | YLARHSIT | 5637 |
| weighted | IQLGFVFL | 5638 |
| weighted | KSDWDDVS | 5639 |
| weighted | RVKAELGL | 5640 |
| weighted | LSTRSHLE | 5641 |
| weighted | DKQLIEEQ | 5642 |
| weighted | TSAQAPEP | 5643 |
| weighted | AEGINPAS | 5644 |
| weighted | LPAEKLGP | 5645 |
| weighted | VALENDCE | 5646 |
| weighted | STLLOMPY | 5647 |
| weighted | KEHRQHCA | 5648 |
| weighted | ATLSGDDD | 5649 |
| weighted | ANPGGDRC | 5650 |
| weighted | VVSVHVKT | 5651 |
| weighted | LLFWNVSM | 5652 |
| weighted | TTDADPLL | 5653 |
| weighted | AEVNLPEK | 5654 |
| weighted | HWVEPYNG | 5655 |
| weighted | GIQWLGVP | 5656 |
| weighted | VPAARSLF | 5657 |
| weighted | LQLRNFCV | 5658 |
| weighted | DARSSQTT | 5659 |
| weighted | KSHEGGAA | 5660 |
| weighted | QYKANRRG | 5661 |
| weighted | LSFKENIH | 5662 |
| weighted | STYMSPTI | 5663 |
| weighted | LVHLIQAC | 5664 |
| weighted | RVIFARTM | 5665 |
| weighted | AAEGFVYI | 5666 |
| weighted | NCAECIEK | 5667 |
| weighted | KTLKKAEF | 5668 |
| weighted | QWNEEEHA | 5669 |
| weighted | PGLKKAAL | 5670 |
| weighted | TSQKAMLK | 5671 |
| weighted | ETQSLFQS | 5672 |
| weighted | GACGPSVS | 5673 |
| weighted | KLGGGPHD | 5674 |
| weighted | ALPGKSTS | 5675 |
| weighted | FPVNHARG | 5676 |
| weighted | ECDRLDKS | 5677 |
| weighted | ESDLKTHR | 5678 |
| weighted | PDPLAMIC | 5679 |
| weighted | FMVLVLSL | 5680 |
| weighted | WDTPDAEV | 5681 |
| weighted | YNKGATDR | 5682 |
| weighted | GKRRPLLV | 5683 |
| weighted | AENIDTSG | 5684 |
| weighted | SRDQVVLT | 5685 |
| weighted | EFRGTRIF | 5686 |
| weighted | HFEVPLPQ | 5687 |
| weighted | YKARLAFH | 5688 |
| weighted | LIEVEVTS | 5689 |
| weighted | IAGKPINS | 5690 |
| weighted | HKIEEDKY | 5691 |
| weighted | VVKIFRLP | 5692 |
| weighted | KLLAGSRW | 5693 |
| weighted | QAPNTDTI | 5694 |
| weighted | GYAGFRYL | 5695 |
| weighted | LKSLEVYV | 5696 |
| weighted | ELKEVIPL | 5697 |
| weighted | DVDTIKTS | 5698 |
| weighted | AVSVILCD | 5699 |
| weighted | LKDIGEVS | 5700 |
| weighted | LGNGPLRG | 5701 |
| weighted | GLERLRID | 5702 |
| weighted | EPQPMLDM | 5703 |
| weighted | YHPEDIKF | 5704 |
| weighted | YLSEWDGW | 5705 |
| weighted | DHGGMTAS | 5706 |
| weighted | DIGGATAQ | 5707 |
| weighted | DAAILLSS | 5708 |
| weighted | MWLCFIKV | 5709 |
| weighted | DLFNDTQG | 5710 |
| weighted | LMLGSFKL | 5711 |
| weighted | EINLQVAK | 5712 |
| weighted | MVAGFEFN | 5713 |
| weighted | GTRLFLTA | 5714 |
| weighted | KGEPAPAA | 5715 |
| weighted | KYDVDREE | 5716 |
| weighted | PQRLTKLI | 5717 |
| weighted | QRNGSIQS | 5718 |
| weighted | SSEPARKA | 5719 |
| weighted | PMGQTATV | 5720 |
| weighted | AADQPWAS | 5721 |
| weighted | LDLRPIEI | 5722 |
| weighted | LGDHIVNH | 5723 |
| weighted | STFKVQRV | 5724 |
| weighted | SLAQAFMD | 5725 |
| weighted | VPNVTGIN | 5726 |
| weighted | VVSSIQDT | 5727 |
| weighted | TRTHIGET | 5728 |
| weighted | NIEDSYLV | 5729 |
| weighted | QISSELWL | 5730 |
| weighted | QQKITSIR | 5731 |
| weighted | WRGLLRKL | 5732 |
| weighted | KKAADPAF | 5733 |
| weighted | SMNGQADL | 5734 |
| weighted | GSGDPEFQ | 5735 |
| weighted | TGQCGRQA | 5736 |
| weighted | EAPQFSSI | 5737 |
| weighted | EPVDKYHQ | 5738 |
| weighted | SALTVLFS | 5739 |
| weighted | YICHQAEP | 5740 |
| weighted | DNNESKLS | 5741 |
| weighted | KLLEPGVG | 5742 |
| weighted | ATLSDICC | 5743 |
| weighted | TKWYGIPL | 5744 |
| weighted | SRVIPVVV | 5745 |
| weighted | EKSFNSSL | 5746 |
| weighted | FLEKDKLT | 5747 |
| weighted | RGSPDATL | 5748 |
| weighted | GFKPMAYD | 5749 |
| weighted | YADDMMLE | 5750 |
| weighted | GLCAEQLP | 5751 |
| weighted | FYKQSAPR | 5752 |
| weighted | SQLVGKPG | 5753 |
| weighted | PDSVEKRL | 5754 |
| weighted | QKTKLGCA | 5755 |
| weighted | RPLVDNNT | 5756 |
| weighted | GVSTGMSR | 5757 |
| weighted | LLLPPSPE | 5758 |
| weighted | QATALKEW | 5759 |
| weighted | PINTFVCG | 5760 |
| weighted | ETEQEAVE | 5761 |
| weighted | YDNFNRRP | 5762 |
| weighted | HALNPGRE | 5763 |
| weighted | VQSSRHTL | 5764 |
| weighted | RQGANKLI | 5765 |
| weighted | AIILYFMG | 5766 |
| weighted | LGVGWLSR | 5767 |
| weighted | VQTLEKVD | 5768 |
| weighted | VNEALPKC | 5769 |
| weighted | HPFQYSDA | 5770 |
| weighted | LMGAKSAH | 5771 |
| weighted | NYIRLCQF | 5772 |
| weighted | ISEFKPSV | 5773 |
| weighted | GELNMQRE | 5774 |
| weighted | RIYTWETC | 5775 |
| weighted | MSKSSSHE | 5776 |
| weighted | APMDSMDL | 5777 |
| weighted | DGYQTYHW | 5778 |
| weighted | ELVFHHCK | 5779 |
| weighted | PPQGRTKG | 5780 |
| weighted | RLAQRSLG | 5781 |
| weighted | TCVGSVHI | 5782 |
| weighted | TPRDLFKK | 5783 |
| weighted | KKKATGFA | 5784 |
| weighted | VLKGGVHS | 5785 |
| weighted | KLVKDESA | 5786 |
| weighted | MAYIACTF | 5787 |
| weighted | SPILMLPV | 5788 |
| weighted | LGQRILPP | 5789 |
| weighted | LNDCESQG | 5790 |
| weighted | ATERADES | 5791 |
| weighted | LAIRHSFA | 5792 |
| weighted | SCQDAINS | 5793 |
| weighted | AKDEQKVY | 5794 |
| weighted | SDQCIGQS | 5795 |
| weighted | PETGCLKV | 5796 |
| weighted | PLLRFGLY | 5797 |
| weighted | KPGVMAPG | 5798 |
| weighted | GGISSTSQ | 5799 |
| weighted | PSEKLCGF | 5800 |
| weighted | IGEVYIET | 5801 |
| weighted | AHPNDIAA | 5802 |
| weighted | EQKWNFRE | 5803 |
| weighted | LENLMTRP | 5804 |
| weighted | PVAATGAE | 5805 |
| weighted | DILSFVPL | 5806 |
| weighted | HEWTSIAT | 5807 |
| weighted | GKSSCQDV | 5808 |
| weighted | AQEGLIGH | 5809 |
| weighted | EDKRVLTK | 5810 |
| weighted | WLGTEATL | 5811 |
| weighted | VRALKFTD | 5812 |
| weighted | VEATKLGQ | 5813 |
| weighted | AHKRFVYP | 5814 |
| weighted | HVYENGIP | 5815 |
| weighted | KFFSNFYE | 5816 |
| weighted | LCDHVEAT | 5817 |
| weighted | QKIIIRHQ | 5818 |
| weighted | RSQFQKDW | 5819 |
| weighted | SLEGRTLE | 5820 |
| weighted | IEMTAAPT | 5821 |
| weighted | QLFQRLTL | 5822 |
| weighted | KIASMSPV | 5823 |
| weighted | STRAPRAL | 5824 |
| weighted | LLWKMSLV | 5825 |
| weighted | LEEGPAIG | 5826 |
| weighted | ALGMILLH | 5827 |
| weighted | NMLGHQDL | 5828 |
| weighted | RRGTSLRD | 5829 |
| weighted | KLFKPTNG | 5830 |
| weighted | WSLNLRGN | 5831 |
| weighted | VFGGLKEC | 5832 |
| weighted | DLSTRAEN | 5833 |
| weighted | RLITDKQQ | 5834 |
| weighted | PRYTTRRL | 5835 |
| weighted | HSNFTCCE | 5836 |
| weighted | RGLLPSVV | 5837 |
| weighted | WKTDRLEG | 5838 |
| weighted | VRIYVHVL | 5839 |
| weighted | DFAAKTKA | 5840 |
| weighted | LDWALDVS | 5841 |
| weighted | ELFENCRL | 5842 |
| weighted | SPALAHPS | 5843 |
| weighted | SAFSDYSI | 5844 |
| weighted | HMTRILAN | 5845 |
| weighted | NTTYITGG | 5846 |
| weighted | GALRTCLI | 5847 |
| weighted | NSRDRIIE | 5848 |
| weighted | SVSTQALR | 5849 |
| weighted | ALRVEEKK | 5850 |
| weighted | RAVTELQK | 5851 |
| weighted | TSRLTYRK | 5852 |
| weighted | ELDPSDLG | 5853 |
| weighted | VEVEGQLR | 5854 |
| weighted | SRKMKLDC | 5855 |
| weighted | HDLLPLRI | 5856 |
| weighted | TRADQAEG | 5857 |
| weighted | TIKCLLCI | 5858 |
| weighted | VPSGPYYM | 5859 |
| weighted | VGWPGFSL | 5860 |
| weighted | DTFIPKRV | 5861 |
| weighted | FHEETLVK | 5862 |
| weighted | SGTPTWEK | 5863 |
| weighted | IVGLAILT | 5864 |
| weighted | TSLSCASA | 5865 |
| weighted | RSTRPLTD | 5866 |
| weighted | VIIDESVT | 5867 |
| weighted | KGPFRFTR | 5868 |
| weighted | EDTSRFAC | 5869 |
| weighted | KRVQPEAL | 5870 |
| weighted | KSSTKAMY | 5871 |
| weighted | LGYAHNOS | 5872 |
| weighted | DVNKTMTP | 5873 |
| weighted | KGVSTEHC | 5874 |
| weighted | VERAVAPE | 5875 |
| weighted | IARTIPVS | 5876 |
| weighted | PPELPSS | 5877 |
| weighted | TTLRRALS | 5878 |
| weighted | YYRADPDS | 5879 |
| weighted | KLGSGGPA | 5880 |
| weighted | TAEFEALK | 5881 |
| weighted | LPFLFSEG | 5882 |
| weighted | NECPPQGL | 5883 |
| weighted | RADVGVLK | 5884 |
| weighted | AQLRGCMG | 5885 |
| weighted | LCSKSSTP | 5886 |
| weighted | GKHAELLS | 5887 |
| weighted | AGNPANEV | 5888 |
| weighted | VFIPVRFK | 5889 |
| weighted | YPSVLGGS | 5890 |
| weighted | RQHLLNAS | 5891 |
| weighted | LQGLYPES | 5892 |
| weighted | TTVAMYGH | 5893 |
| weighted | APVGQEIR | 5894 |
| weighted | QMFSVRMK | 5895 |
| weighted | TRHADADS | 5896 |
| weighted | STFTSTAL | 5897 |
| weighted | KLVPAEEV | 5898 |
| weighted | LSTRFTLP | 5899 |
| weighted | IKRTQRLG | 5900 |
| weighted | AFLNLLSD | 5901 |
| weighted | AVENEIPF | 5902 |
| weighted | FTTPTKEM | 5903 |
| weighted | LYVGEKTS | 5904 |
| weighted | HLSRGPRF | 5905 |
| weighted | QADGDRQL | 5906 |
| weighted | LTLNLIGP | 5907 |
| weighted | SLGRTAHD | 5908 |
| weighted | NNNSRNAL | 5909 |
| weighted | KYILPEIS | 5910 |
| weighted | QQRNASEG | 5911 |
| weighted | LQEWPPVK | 5912 |
| weighted | SLRPVQLG | 5913 |
| weighted | KPSIACSG | 5914 |
| weighted | RDLWDAES | 5915 |
| weighted | EGKVYTLK | 5916 |
| weighted | LPEKNNLP | 5917 |
| weighted | HLSTLTTQ | 5918 |
| weighted | QTCHPSML | 5919 |
| weighted | GSGQVVRQ | 5920 |
| weighted | DPVCEAAE | 5921 |
| weighted | RLAGVDLL | 5922 |
| weighted | LLAVDPSF | 5923 |
| weighted | IFHSHHLV | 5924 |
| weighted | QEEALIRA | 5925 |
| weighted | APKAASIN | 5926 |
| weighted | APSYPRPI | 5927 |
| weighted | TFQLRSDF | 5928 |
| weighted | HTIFCIYR | 5929 |
| weighted | HGLQARSK | 5930 |
| weighted | YLVYDVYF | 5931 |
| weighted | PEDPLSDD | 5932 |
| weighted | SEGQQLAW | 5933 |
| weighted | CTAVDLIV | 5934 |
| weighted | SFLQEFWL | 5935 |
| weighted | VEPICRPK | 5936 |
| weighted | KLLKHPMY | 5937 |
| weighted | GEVLTGLV | 5938 |
| weighted | TKLKGPAC | 5939 |
| weighted | PSSSSKIL | 5940 |
| weighted | LAVADLQY | 5941 |
| weighted | TMFNAPDP | 5942 |
| weighted | LNLALHLA | 5943 |
| weighted | IVTGRRVG | 5944 |
| weighted | TSRKISEG | 5945 |
| weighted | TGYCQSKP | 5946 |
| weighted | PYNERKLA | 5947 |
| weighted | HIIESVRI | 5948 |
| weighted | SRQTGIVA | 5949 |
| weighted | RLITAAVR | 5950 |
| weighted | FVGMYIHG | 5951 |
| weighted | DLTANFQY | 5952 |
| weighted | REWAERLK | 5953 |
| weighted | EYELQSLP | 5954 |
| weighted | SFEPKSDS | 5955 |
| weighted | SEIQRGGI | 5956 |
| weighted | TTPPVVAV | 5957 |
| weighted | PGINDPTY | 5958 |
| weighted | IIAKLSSV | 5959 |
| weighted | INEHSGRR | 5960 |
| weighted | FMKNRLTY | 5961 |
| weighted | IKQANKLL | 5962 |
| weighted | ATISGESS | 5963 |
| weighted | ELQALFHL | 5964 |
| weighted | GKESESTL | 5965 |
| weighted | PARELPKH | 5966 |
| weighted | QKAVPWDS | 5967 |
| weighted | LHPMPPEL | 5968 |
| weighted | ARQCSQIN | 5969 |
| weighted | CCRLLAET | 5970 |
| weighted | LLDFGAGI | 5971 |
| weighted | RQTGPARG | 5972 |
| weighted | ELAPSPHS | 5973 |
| weighted | SLSQSGGE | 5974 |
| weighted | RINGTLMA | 5975 |
| weighted | KAFPERCL | 5976 |
| weighted | ALFPQEKY | 5977 |
| weighted | KKLKSGKH | 5978 |
| weighted | TANLTVPY | 5979 |
| weighted | KQDKEPLD | 5980 |
| weighted | QQSQIEEY | 5981 |
| weighted | FDHKLEID | 5982 |
| weighted | PGWKTGSQ | 5983 |
| weighted | ERPDALSV | 5984 |
| weighted | FLWPNRAY | 5985 |
| weighted | CMDRLLET | 5986 |
| weighted | PVLMLNPA | 5987 |
| weighted | DEVGKMVS | 5988 |
| weighted | PQDHEKLS | 5989 |
| weighted | RIDGYLPI | 5990 |
| weighted | SCHYKLVG | 5991 |
| weighted | DANIPLAL | 5992 |
| weighted | RVKISPIS | 5993 |
| weighted | VTLEGCRL | 5994 |
| weighted | SKYQEFTL | 5995 |
| weighted | QWTGAQRP | 5996 |
| weighted | GVDCHDHF | 5997 |
| weighted | DEPALGDF | 5998 |
| weighted | PVKNITKA | 5999 |
| weighted | YWEKAPGD | 6000 |
| weighted | SKIQSGEG | 6001 |
| weighted | RSGHREKA | 6002 |
| weighted | PRETTAHL | 6003 |
| weighted | GISHFLYD | 6004 |
| weighted | QDSAVKVL | 6005 |
| weighted | AESNESSTM | 6006 |
| weighted | KACVQDRLR | 6007 |
| weighted | SVSGLQSAK | 6008 |
| weighted | SQCVIGMLQ | 6009 |
| weighted | KICVAQING | 6010 |
| weighted | HQGNSESWP | 6011 |
| weighted | DAFGCNPQV | 6012 |
| weighted | LQHRWVDHQ | 6013 |
| weighted | KPPTTPFYT | 6014 |
| weighted | LTVTAEYWV | 6015 |
| weighted | KTGOLFVRS | 6016 |
| weighted | FNKGRAEPV | 6017 |
| weighted | SFIVLNELG | 6018 |
| weighted | INSGVAQEL | 6019 |
| weighted | KDPSHGLVS | 6020 |
| weighted | PSQLMTTTG | 6021 |
| weighted | VLRYFDAQA | 6022 |
| weighted | TFEMTMALM | 6023 |
| weighted | SVQTRATGC | 6024 |
| weighted | LVTYTPKIA | 6025 |
| weighted | ITSESKEAL | 6026 |
| weighted | NMGSFLDDT | 6027 |
| weighted | RVSCMAYKI | 6028 |
| weighted | IKLDESKWK | 6029 |
| weighted | DLQEQPSAV | 6030 |
| weighted | LHIIKPFRE | 6031 |
| weighted | KSNGLMGGD | 6032 |
| weighted | GQSHVKLSS | 6033 |
| weighted | SQGVCVEAI | 6034 |
| weighted | LHGDTLPNS | 6035 |
| weighted | QVNSYRKPH | 6036 |
| weighted | FVCIVLPAE | 6037 |
| weighted | NADQQRAVE | 6038 |
| weighted | ESDHSHLGF | 6039 |
| weighted | PVVFAPVRD | 6040 |
| weighted | DHGVASMPR | 6041 |
| weighted | GSHDGMFVS | 6042 |
| weighted | GAPYEYDHA | 6043 |
| weighted | GPCILMLRR | 6044 |
| weighted | RFFMSLRPL | 6045 |
| weighted | GRRSHHKDL | 6046 |
| weighted | CRNAYLLTG | 6047 |
| weighted | HIFSTLILL | 6048 |
| weighted | FDLPRERKE | 6049 |
| weighted | VSSGLEGRD | 6050 |
| weighted | LRGSVAKYE | 6051 |
| weighted | GMFQTKPMV | 6052 |
| weighted | NSYNGAQTV | 6053 |
| weighted | RTRRGTEEW | 6054 |
| weighted | QWLKALFDK | 6055 |
| weighted | QQERAISGP | 6056 |
| weighted | LCRGLGKGS | 6057 |
| weighted | DYFAALAAL | 6058 |
| weighted | NRVFARYIL | 6059 |
| weighted | HLSNPLFGI | 6060 |
| weighted | VMSKEKEMS | 6061 |
| weighted | PVCLSLVGD | 6062 |
| weighted | IAHEAGPGI | 6063 |
| weighted | KYETAEYFK | 6064 |
| weighted | ILRYQLGLI | 6065 |
| weighted | AGVDCRPAT | 6066 |
| weighted | SPNLANPHD | 6067 |
| weighted | EPLLGNTDL | 6068 |
| weighted | PLLIIKATK | 6069 |
| weighted | CLGCEESEP | 6070 |
| weighted | GAESVRVAL | 6071 |
| weighted | SRRQENEVT | 6072 |
| weighted | ADIHISQDS | 6073 |
| weighted | VSRTSTKAG | 6074 |
| weighted | ESSCVLTLV | 6075 |
| weighted | HTDTSVSRT | 6076 |
| weighted | GLVDRLDMD | 6077 |
| weighted | SNLQIVWIM | 6078 |
| weighted | SNKENQAIL | 6079 |
| weighted | TGRLSKFKL | 6080 |
| weighted | PEIGQRGIL | 6081 |
| weighted | KRYGYHVPA | 6082 |
| weighted | AKIFLTTAL | 6083 |
| weighted | FVQRTYPTF | 6084 |
| weighted | GRSYRPVEI | 6085 |
| weighted | YSIISAASR | 6086 |
| weighted | SAPLSLDSD | 6087 |
| weighted | GGFGRILAG | 6088 |
| weighted | SLGGDEEVD | 6089 |
| weighted | CKFNIAGKN | 6090 |
| weighted | IKGAACRVA | 6091 |
| weighted | SDAVEGGLY | 6092 |
| weighted | ARGKLIQHE | 6093 |
| weighted | IPSFEATPL | 6094 |
| weighted | TPCEYYSAS | 6095 |
| weighted | LSKLFQIPA | 6096 |
| weighted | DSPYPGQIK | 6097 |
| weighted | NERLEQICL | 6098 |
| weighted | FLKLGTRQC | 6099 |
| weighted | VGHDAAKRH | 6100 |
| weighted | ETGENKAHN | 6101 |
| weighted | KDILLVLEH | 6102 |
| weighted | TVKNTNQLN | 6103 |
| weighted | AHQVRARPK | 6104 |
| weighted | HELRPPPLP | 6105 |
| weighted | QEFGKELGA | 6106 |
| weighted | SVHAIEVGT | 6107 |
| weighted | AYTLILTLL | 6108 |
| weighted | REPYESQET | 6109 |
| weighted | ERMLSVSAA | 6110 |
| weighted | GGKASGVVG | 6111 |
| weighted | WEELGKKIH | 6112 |
| weighted | DPVLGVKSA | 6113 |
| weighted | KLSITLSSK | 6114 |
| weighted | VVLLAGPLT | 6115 |
| weighted | TRSKYIDHG | 6116 |
| weighted | TFTHNKELR | 6117 |
| weighted | GLILQLFSM | 6118 |
| weighted | LAPDLIRES | 6119 |
| weighted | QAKVEARMP | 6120 |
| weighted | AGSGQEPKP | 6121 |
| weighted | DYRRLWSSK | 6122 |
| weighted | PTNPGLRLT | 6123 |
| weighted | DYSVFTLMG | 6124 |
| weighted | GYGFQHHVV | 6125 |
| weighted | TAESVAVDV | 6126 |
| weighted | PISVLEALS | 6127 |
| weighted | SETFQTYVV | 6128 |
| weighted | MQGPAGQPQ | 6129 |
| weighted | LAVILRLDH | 6130 |
| weighted | MKIAMAQAK | 6131 |
| weighted | EAKQLSLSD | 6132 |
| weighted | IIEGGASTK | 6133 |
| weighted | MSAKAPMCE | 6134 |
| weighted | SMMLKPRTV | 6135 |
| weighted | KFYDLFSNT | 6136 |
| weighted | TQQGALYDS | 6137 |
| weighted | LRKCLGIQS | 6138 |
| weighted | LSHPHSTFD | 6139 |
| weighted | AGVLGVEQA | 6140 |
| weighted | SGLRRASLE | 6141 |
| weighted | RTKSDDFNS | 6142 |
| weighted | GGVNSHNPK | 6143 |
| weighted | DVLGILCAF | 6144 |
| weighted | DGALPGSQL | 6145 |
| weighted | DHVPCQHET | 6146 |
| weighted | KTELGLAGQ | 6147 |
| weighted | CLWGGSVGA | 6148 |
| weighted | VPKTGMDRG | 6149 |
| weighted | TALNQTSQI | 6150 |
| weighted | NPVKEPKEP | 6151 |
| weighted | VRAPEGLAD | 6152 |
| weighted | TQPSEDYSR | 6153 |
| weighted | IENGDEGQL | 6154 |
| weighted | YTNQPINQE | 6155 |
| weighted | TDGTSDRSC | 6156 |
| weighted | VVPSSPKPC | 6157 |
| weighted | QLRASIKLA | 6158 |
| weighted | SDFKVRVFA | 6159 |
| weighted | ENEEPAHVS | 6160 |
| weighted | QQAESRGNV | 6161 |
| weighted | LDLSEKTMH | 6162 |
| weighted | IRVPNGTEL | 6163 |
| weighted | PLTDMTLHV | 6164 |
| weighted | VSVCQEPSP | 6165 |
| weighted | ELFLLLSQN | 6166 |
| weighted | NECRSLLIE | 6167 |
| weighted | SFMTTHVSR | 6168 |
| weighted | LTASSRSLP | 6169 |
| weighted | HEGQLKRQE | 6170 |
| weighted | VSLTEAEVK | 6171 |
| weighted | SPIAYPGNT | 6172 |
| weighted | RMDTQDFSL | 6173 |
| weighted | GEPAQGGLR | 6174 |
| weighted | DVSDFRGEQ | 6175 |
| weighted | PLGAVKLNV | 6176 |
| weighted | NDYGSSEAL | 6177 |
| weighted | DKLTLDLLK | 6178 |
| weighted | KNRGKMISA | 6179 |
| weighted | YVTAKKDKI | 6180 |
| weighted | GDSSPYNLK | 6181 |
| weighted | STSILQGKA | 6182 |
| weighted | VYLFNVMPP | 6183 |
| weighted | FRIHRGGRQ | 6184 |
| weighted | AEEGKSKDG | 6185 |
| weighted | VADLLCVLL | 6186 |
| weighted | PEKNETLYY | 6187 |
| weighted | LSPPGHLAL | 6188 |
| weighted | GVSRQPLSV | 6189 |
| weighted | SGKAHVNWR | 6190 |
| weighted | DNLVKGLDL | 6191 |
| weighted | NASSLTLAS | 6192 |
| weighted | IRAPLDDTF | 6193 |
| weighted | TYILCNLSD | 6194 |
| weighted | RQYERESEL | 6195 |
| weighted | CSPSEKYLN | 6196 |
| weighted | PHLKEALLP | 6197 |
| weighted | LKADVNDQR | 6198 |
| weighted | TKDQQWTGG | 6199 |
| weighted | HFRNHGSLS | 6200 |
| weighted | RQPVTFPNA | 6201 |
| weighted | VSLEKYSEL | 6202 |
| weighted | SAQNDLGTP | 6203 |
| weighted | SEEECGFFI | 6204 |
| weighted | FKSELGKPF | 6205 |
| weighted | LEEAHGEEG | 6206 |
| weighted | HAGDYLKVV | 6207 |
| weighted | RHQVRSLTY | 6208 |
| weighted | PEGQDSTPN | 6209 |
| weighted | PSGAPTARH | 6210 |
| weighted | SLGNSTDTL | 6211 |
| weighted | FASDCNWPK | 6212 |
| weighted | TKEEHLSLE | 6213 |
| weighted | EVLSDFALD | 6214 |
| weighted | VANDFIILC | 6215 |
| weighted | QGFKANIAA | 6216 |
| weighted | AILSQIRWD | 6217 |
| weighted | GKFNTTEFY | 6218 |
| weighted | NNGDTRAGM | 6219 |
| weighted | MKLIAVRKI | 6220 |
| weighted | PVCRDKGTA | 6221 |
| weighted | TYRAPQYRL | 6222 |
| weighted | QKSRFQPYE | 6223 |
| weighted | RGTEFNGQS | 6224 |
| weighted | LAIQIPQVE | 6225 |
| weighted | IGRIIVQDA | 6226 |
| weighted | VAVVLYAPY | 6227 |
| weighted | MELRPALSG | 6228 |
| weighted | FRDKDLEGI | 6229 |
| weighted | GYYQPIPHF | 6230 |
| weighted | DKMADQGVP | 6231 |
| weighted | KELQYFMGL | 6232 |
| weighted | GQVSSDSHV | 6233 |
| weighted | LLKGPCHLP | 6234 |
| weighted | INKTGISEG | 6235 |
| weighted | KEEELQVVP | 6236 |
| weighted | AYHNPDVAY | 6237 |
| weighted | KKLYSVLVI | 6238 |
| weighted | PMQESGVWQ | 6239 |
| weighted | KEPPEFAVL | 6240 |
| weighted | GDGGIDPVG | 6241 |
| weighted | LSLSVPGNP | 6242 |
| weighted | EGIQFEPQT | 6243 |
| weighted | GGEKVGGSS | 6244 |
| weighted | QGPLKMTSS | 6245 |
| weighted | ESHIPPYPL | 6246 |
| weighted | PHNEEDNLI | 6247 |
| weighted | GWFPQVSVL | 6248 |
| weighted | DAVNEREEK | 6249 |
| weighted | RLITLVVPT | 6250 |
| weighted | KAIARSRGE | 6251 |
| weighted | DVRAQINTA | 6252 |
| weighted | GPVVGEKEN | 6253 |
| weighted | EDVVLGLVV | 6254 |
| weighted | VGEQKASKL | 6255 |
| weighted | ELGKKENVL | 6256 |
| weighted | LRNSNMRMM | 6257 |
| weighted | EICGFDIAL | 6258 |
| weighted | TVLAVQNFG | 6259 |
| weighted | NSIVRTICA | 6260 |
| weighted | RIRLDEDEM | 6261 |
| weighted | PLYEVWEQQ | 6262 |
| weighted | VSEFWCPCW | 6263 |
| weighted | DVSRAEKPI | 6264 |
| weighted | TPIAKGTLQ | 6265 |
| weighted | KNFQQQADV | 6266 |
| weighted | GSAQCKLKD | 6267 |
| weighted | HDEPAQINL | 6268 |
| weighted | GPNIPHQGV | 6269 |
| weighted | SESKIFSAP | 6270 |
| weighted | AKYPNRKRL | 6271 |
| weighted | LKCVLVRFL | 6272 |
| weighted | GPLCPKRLL | 6273 |
| weighted | CHLLTYLQR | 6274 |
| weighted | VGYPTSQPE | 6275 |
| weighted | TKVWDRTMM | 6276 |
| weighted | HPLAPVLRN | 6277 |
| weighted | MSLEHSNPL | 6278 |
| weighted | LNLNLVKQL | 6279 |
| weighted | RTKASQLGA | 6280 |
| weighted | AAFDDSPLD | 6281 |
| weighted | IEHSSLVGC | 6282 |
| weighted | RQHKTELLT | 6283 |
| weighted | IVFDSSEPD | 6284 |
| weighted | GVTRIMPTQ | 6285 |
| weighted | PTGFLSPSV | 6286 |
| weighted | VRFQTLAAF | 6287 |
| weighted | HLKKAIMLL | 6288 |
| weighted | IVETSVNAE | 6289 |
| weighted | DSKCGYQRD | 6290 |
| weighted | QESRAMHTE | 6291 |
| weighted | ALSGVLTLM | 6292 |
| weighted | MPFDSDPNA | 6293 |
| weighted | AKSERECNH | 6294 |
| weighted | QSHLEDLYG | 6295 |
| weighted | FSDDHETVP | 6296 |
| weighted | PTRRYPQPH | 6297 |
| weighted | TSLDTQKGS | 6298 |
| weighted | PLMTPKLRT | 6299 |
| weighted | KFMKKLCKL | 6300 |
| weighted | PSPRTHDTV | 6301 |
| weighted | KWREQPSHE | 6302 |
| weighted | TPITAPSRY | 6303 |
| weighted | PGAAELQTP | 6304 |
| weighted | YKMSPFVGL | 6305 |
| weighted | STWGLFVVS | 6306 |
| weighted | TLTSGSLLT | 6307 |
| weighted | TPRAPRLRF | 6308 |
| weighted | LPEREQFSN | 6309 |
| weighted | LVATENAYL | 6310 |
| weighted | DTLNDGIYQ | 6311 |
| weighted | LGSVNTLEP | 6312 |
| weighted | VMGQEVTAT | 6313 |
| weighted | TPGLTENDG | 6314 |
| weighted | KTQDSEYST | 6315 |
| weighted | YQYVRTFAQ | 6316 |
| weighted | VQAITELGF | 6317 |
| weighted | RSRKHTFVN | 6318 |
| weighted | TIKEAVGLS | 6319 |
| weighted | TLLLKSEIW | 6320 |
| weighted | GVSNLFVTI | 6321 |
| weighted | YNNDHTDCI | 6322 |
| weighted | SVKILHEAE | 6323 |
| weighted | ILTGSGVRL | 6324 |
| weighted | LLTIFTMLH | 6325 |
| weighted | TVELRIPTD | 6326 |
| weighted | MQARLVADS | 6327 |
| weighted | VASYVPTLS | 6328 |
| weighted | TPVPFNMGE | 6329 |
| weighted | LTKAPACFQ | 6330 |
| weighted | VSQLLQFLP | 6331 |
| weighted | FAPAAVALV | 6332 |
| weighted | ENARDEDLG | 6333 |
| weighted | LRIRNTHSV | 6334 |
| weighted | GEKPVKNKE | 6335 |
| weighted | QVATGIKRT | 6336 |
| weighted | SDVQQFGLV | 6337 |
| weighted | RQELRDNPG | 6338 |
| weighted | DISEKFDRE | 6339 |
| weighted | PSTAESPPV | 6340 |
| weighted | EKPIPYWVE | 6341 |
| weighted | TLQEESIYY | 6342 |
| weighted | RTTEIIRCN | 6343 |
| weighted | AACKSPAFD | 6344 |
| weighted | LGLRRNGSS | 6345 |
| weighted | DALRARPWS | 6346 |
| weighted | PQAEILSRE | 6347 |
| weighted | GTCKGTFLC | 6348 |
| weighted | AEEFVKKAG | 6349 |
| weighted | FKSKKRQVA | 6350 |
| weighted | KVEESHNGL | 6351 |
| weighted | LMLPRIMAR | 6352 |
| weighted | KEVIFAPIW | 6353 |
| weighted | EGSHEALTA | 6354 |
| weighted | VTKRADGYL | 6355 |
| weighted | EQSNLEVKI | 6356 |
| weighted | LVYDGRQPP | 6357 |
| weighted | IMQEGVKNW | 6358 |
| weighted | FPPSDGFRT | 6359 |
| weighted | YSLKLPKCA | 6360 |
| weighted | KERETHIVE | 6361 |
| weighted | LASGSRDDL | 6362 |
| weighted | SLQVGPGGD | 6363 |
| weighted | YQMLLGLVL | 6364 |
| weighted | DTTSLVGGS | 6365 |
| weighted | TLEQDSNKV | 6366 |
| weighted | WGKSKSLEA | 6367 |
| weighted | RNYLDGKQK | 6368 |
| weighted | MSAAPPEQI | 6369 |
| weighted | RLRLSVPVF | 6370 |
| weighted | SKPLKKNIQ | 6371 |
| weighted | APHTTLPAT | 6372 |
| weighted | ANQSKEEQV | 6373 |
| weighted | IVASTVLWI | 6374 |
| weighted | HKSETKQHK | 6375 |
| weighted | AAEEIKLEE | 6376 |
| weighted | EECREMTVE | 6377 |
| weighted | RHWFPFLKL | 6378 |
| weighted | LYGSLLRDI | 6379 |
| weighted | PTIGFRRNS | 6380 |
| weighted | HPTYRNDRS | 6381 |
| weighted | RYIAAVWAF | 6382 |
| weighted | SRANLHPRV | 6383 |
| weighted | DTKKLKEAC | 6384 |
| weighted | CGAVPFSAR | 6385 |
| weighted | LPATKVQRT | 6386 |
| weighted | YKLLDDKED | 6387 |
| weighted | CDSRACKSY | 6388 |
| weighted | QDTTQLREM | 6389 |
| weighted | PFHDDLSMN | 6390 |
| weighted | RLAFNLRPG | 6391 |
| weighted | LYKIVHLRW | 6392 |
| weighted | PLPDPLVWK | 6393 |
| weighted | REKTKGSAH | 6394 |
| weighted | GAPLEVVSA | 6395 |
| weighted | YQVSRENLS | 6396 |
| weighted | PPMVAISKL | 6397 |
| weighted | KFERGEQAG | 6398 |
| weighted | VNQLYKLLA | 6399 |
| weighted | ITTIALEAQ | 6400 |
| weighted | ESYQPSCSE | 6401 |
| weighted | KHADQLQLA | 6402 |
| weighted | QARNAAAPS | 6403 |
| weighted | LCQSLDHEH | 6404 |
| weighted | KAGTPLMMK | 6405 |
| weighted | TPDWESDVW | 6406 |
| weighted | CADFTLKIH | 6407 |
| weighted | QRVSRTYPG | 6408 |
| weighted | LYCGLENIQ | 6409 |
| weighted | SARLAQPMQ | 6410 |
| weighted | QLPSRQNQL | 6411 |
| weighted | DAKPEFELA | 6412 |
| weighted | AHKEYHLSP | 6413 |
| weighted | DERIKTRLI | 6414 |
| weighted | VTAFRISEG | 6415 |
| weighted | DRGPGLFYS | 6416 |
| weighted | VDCEDPSLQ | 6417 |
| weighted | PSTEQRRGS | 6418 |
| weighted | KSTRQHEGI | 6419 |
| weighted | APADLSQRP | 6420 |
| weighted | TDAGGVVSG | 6421 |
| weighted | RVKLATSPA | 6422 |
| weighted | VGSNGANLL | 6423 |
| weighted | DAYLIQSLV | 6424 |
| weighted | GQSCIATRI | 6425 |
| weighted | PQERTDGEL | 6426 |
| weighted | QQFLAEAQG | 6427 |
| weighted | CIILFEIST | 6428 |
| weighted | PDHSTSESE | 6429 |
| weighted | KFAEKSRLM | 6430 |
| weighted | CIEAETTIF | 6431 |
| weighted | RQDTQMWAW | 6432 |
| weighted | LLLIVVIGV | 6433 |
| weighted | ELSIELEPP | 6434 |
| weighted | NRGIVALKL | 6435 |
| weighted | PASVQVWCL | 6436 |
| weighted | SNSRTPKMN | 6437 |
| weighted | GSGMAVMLR | 6438 |
| weighted | HPTVGAPHV | 6439 |
| weighted | HEENQADAV | 6440 |
| weighted | AVGLHITRE | 6441 |
| weighted | YKFLSEYLQ | 6442 |
| weighted | RILIDGKQE | 6443 |
| weighted | LALQMQQDE | 6444 |
| weighted | TDLASSLGK | 6445 |
| weighted | PVPQQVFKK | 6446 |
| weighted | THPRRNASQ | 6447 |
| weighted | HDGQITGPC | 6448 |
| weighted | ALKDGGAGL | 6449 |
| weighted | RVMVVQDVP | 6450 |
| weighted | RMSCKDLDG | 6451 |
| weighted | NAMASEIAI | 6452 |
| weighted | GFEEAFTLA | 6453 |
| weighted | KTVSESNNA | 6454 |
| weighted | CVSDNSIEI | 6455 |
| weighted | LAPLQGVIE | 6456 |
| weighted | YEFGRVGAT | 6457 |
| weighted | PKRYERTSI | 6458 |
| weighted | DEVAGVHTD | 6459 |
| weighted | IYARSKSVA | 6460 |
| weighted | EGKTQVISD | 6461 |
| weighted | TGKLSAGIR | 6462 |
| weighted | SRLAFGVVT | 6463 |
| weighted | QFTAKRDFD | 6464 |
| weighted | PLEAKGLYK | 6465 |
| weighted | RPVLWTDLK | 6466 |
| weighted | FVFILNQPP | 6467 |
| weighted | SSALLQSPV | 6468 |
| weighted | AVSTNIALS | 6469 |
| weighted | ELTWLQPAS | 6470 |
| weighted | NIVILNWGC | 6471 |
| weighted | LIYRPLEAW | 6472 |
| weighted | LNSAETRGK | 6473 |
| weighted | AGGVLDLAS | 6474 |
| weighted | TCPPNSGSH | 6475 |
| weighted | KGVCIETES | 6476 |
| weighted | YENLRILLA | 6477 |
| weighted | CAVSDDWPQ | 6478 |
| weighted | RIEKGAPEE | 6479 |
| weighted | YSKAGRKNR | 6480 |
| weighted | LTPEMMKRA | 6481 |
| weighted | RLNIVVSAN | 6482 |
| weighted | VRGQTEAIE | 6483 |
| weighted | DPVPELETE | 6484 |
| weighted | ETESVRQRP | 6485 |
| weighted | QTSTSRFSV | 6486 |
| weighted | TPFYATWSK | 6487 |
| weighted | LLNIAPLRD | 6488 |
| weighted | WFLGRGYVR | 6489 |
| weighted | QSEELSNSQ | 6490 |
| weighted | QLPNFRYRG | 6491 |
| weighted | VYTRDVAQY | 6492 |
| weighted | PRGWKRVAH | 6493 |
| weighted | YSQRVRKSQ | 6494 |
| weighted | NSIKDKSLP | 6495 |
| weighted | DGEYNFETE | 6496 |
| weighted | NAVKDANGE | 6497 |
| weighted | KNSQLRPHT | 6498 |
| weighted | KASLSLLTL | 6499 |
| weighted | KWSDTLLLD | 6500 |
| weighted | TSLQGTYPQ | 6501 |
| weighted | PSNSEIERA | 6502 |
| weighted | IKTTPVDRM | 6503 |
| weighted | QYPLKLAAC | 6504 |
| weighted | ADKVRWEEG | 6505 |
| weighted | MRGRWFFQE | 6506 |
| weighted | KDGGPQGSR | 6507 |
| weighted | TLESFYDSI | 6508 |
| weighted | LGPMSYVSE | 6509 |
| weighted | LFMSWQSVP | 6510 |
| weighted | VMKPMFIAV | 6511 |
| weighted | PVFSLLKAR | 6512 |
| weighted | KFENWKFDH | 6513 |
| weighted | ESQFLQCDE | 6514 |
| weighted | RYGATSLLT | 6515 |
| weighted | VNLKAASRE | 6516 |
| weighted | KYSHKLTVN | 6517 |
| weighted | YLAGSSEDI | 6518 |
| weighted | LVEGGQTTT | 6519 |
| weighted | SRRQALAAI | 6520 |
| weighted | RIGMHEQYK | 6521 |
| weighted | SYLDRPIHC | 6522 |
| weighted | LLLGASDSK | 6523 |
| weighted | DGEADRESL | 6524 |
| weighted | LPCALVCGA | 6525 |
| weighted | DINSSDYCV | 6526 |
| weighted | SSGPQKMLM | 6527 |
| weighted | LIQANDNKK | 6528 |
| weighted | QDTIREDNL | 6529 |
| weighted | PARLLLLCQ | 6530 |
| weighted | MRLCAEVLR | 6531 |
| weighted | RREPKEWCL | 6532 |
| weighted | LAKPGHLGN | 6533 |
| weighted | VVGNNNRLA | 6534 |
| weighted | KQSDTWFNA | 6535 |
| weighted | SIAERSGVS | 6536 |
| weighted | SQQMIRLTG | 6537 |
| weighted | PGLTLGTLQ | 6538 |
| weighted | RLTPLETRE | 6539 |
| weighted | LRYANDPRG | 6540 |
| weighted | SLLRMRLAP | 6541 |
| weighted | AQLLPNLYT | 6542 |
| weighted | TGHTQSFRR | 6543 |
| weighted | DRQLSAGQV | 6544 |
| weighted | LPGWYLGTF | 6545 |
| weighted | PGQPRDRQD | 6546 |
| weighted | VFKRLIDFT | 6547 |
| weighted | LETLYGRDG | 6548 |
| weighted | KFKHRRHLP | 6549 |
| weighted | TAPDDAVPP | 6550 |
| weighted | GANRKPLAC | 6551 |
| weighted | ETPLRYSLI | 6552 |
| weighted | YPTSIEPGS | 6553 |
| weighted | KVAPGDLTS | 6554 |
| weighted | ILEQLAKRN | 6555 |
| weighted | TRGYVLNRP | 6556 |
| weighted | EQSPLSMIS | 6557 |
| weighted | PSPENRHHV | 6558 |
| weighted | SEAAVNEIT | 6559 |
| weighted | LDRKDNFNQ | 6560 |
| weighted | CNFVSATEG | 6561 |
| weighted | VALCGHHTP | 6562 |
| weighted | WLLVLSFKK | 6563 |
| weighted | LDFPSLLAN | 6564 |
| weighted | LKAYPATDA | 6565 |
| weighted | QPDHKVLQG | 6566 |
| weighted | QPVVLKMYT | 6567 |
| weighted | VLLIRFKAN | 6568 |
| weighted | NPGALVQPL | 6569 |
| weighted | MFVSPNSAL | 6570 |
| weighted | HYYRSGFSG | 6571 |
| weighted | PNSQMVLRS | 6572 |
| weighted | ILPAVEQAG | 6573 |
| weighted | SVQEDPSSD | 6574 |
| weighted | ATSFKSKAY | 6575 |
| weighted | AMGQWIALC | 6576 |
| weighted | ATQPPLDVP | 6577 |
| weighted | TDNGLENFS | 6578 |
| weighted | GLKQQIKIL | 6579 |
| weighted | SSQIDRVRK | 6580 |
| weighted | KVGSDGLQG | 6581 |
| weighted | GNRTYVLDL | 6582 |
| weighted | KLSLTGHIL | 6583 |
| weighted | ALARFTSVH | 6584 |
| weighted | DAFIMSKFT | 6585 |
| weighted | EGSTSLDLV | 6586 |
| weighted | GGLEGLASG | 6587 |
| weighted | NLTADLHLS | 6588 |
| weighted | RQSSDTKNE | 6589 |
| weighted | SGGASARQI | 6590 |
| weighted | VAQLVHGVG | 6591 |
| weighted | SGDARRIKF | 6592 |
| weighted | RNSDGKSKY | 6593 |
| weighted | RQSEARGTV | 6594 |
| weighted | LSQASVGQK | 6595 |
| weighted | LIAGAGNAV | 6596 |
| weighted | LLRLTCAFK | 6597 |
| weighted | SPGVIKMAQ | 6598 |
| weighted | SAFNTESSS | 6599 |
| weighted | LSSAKEEQG | 6600 |
| weighted | NSSMESSKV | 6601 |
| weighted | APPKPLFQH | 6602 |
| weighted | FLCTLISSG | 6603 |
| weighted | FPLSVHGFG | 6604 |
| weighted | VKRAGCHPK | 6605 |
| weighted | VDESEESMK | 6606 |
| weighted | GIEYPRYEI | 6607 |
| weighted | KRQLYSSVG | 6608 |
| weighted | QLAIRYFKL | 6609 |
| weighted | EIGGPIEDQ | 6610 |
| weighted | GPSLLGQES | 6611 |
| weighted | VVYLSELQR | 6612 |
| weighted | GQLSESVAT | 6613 |
| weighted | LTSQIHDGL | 6614 |
| weighted | DAFDCDVPG | 6615 |
| weighted | AFVADTAHS | 6616 |
| weighted | LNSHLQPKH | 6617 |
| weighted | EVIEICLKD | 6618 |
| weighted | RRKLPRERC | 6619 |
| weighted | TGKKDPGGS | 6620 |
| weighted | QLALLGFAH | 6621 |
| weighted | WPAREFDQS | 6622 |
| weighted | LVFADFATQ | 6623 |
| weighted | HPAPLKSLT | 6624 |
| weighted | PSLFSMGVM | 6625 |
| weighted | SKYRGEPVW | 6626 |
| weighted | YAHWWPFDT | 6627 |
| weighted | TRFLRRQHT | 6628 |
| weighted | EGTVHFQVH | 6629 |
| weighted | LVKLTWRVP | 6630 |
| weighted | HGELTLHDQ | 6631 |
| weighted | FSSVELLAL | 6632 |
| weighted | LRLKNLLTC | 6633 |
| weighted | SQPLVVLSA | 6634 |
| weighted | YLRSTDGQG | 6635 |
| weighted | FHLAKLLLK | 6636 |
| weighted | ETLQHPDPV | 6637 |
| weighted | SRRAILGYL | 6638 |
| weighted | YEDIYSLRY | 6639 |
| weighted | AYILWQVAW | 6640 |
| weighted | LIEKMVFVW | 6641 |
| weighted | LWKNSVAKD | 6642 |
| weighted | CRRGCKQTK | 6643 |
| weighted | ERFEASVEA | 6644 |
| weighted | NYSKKTKLG | 6645 |
| weighted | LTYILRQTG | 6646 |
| weighted | VPLELSASS | 6647 |
| weighted | TSAPVLHPQ | 6648 |
| weighted | KLLCIGERQ | 6649 |
| weighted | TPFLQMYAT | 6650 |
| weighted | PQPKSHSCL | 6651 |
| weighted | SHWEPKEFW | 6652 |
| weighted | DNSPSSGLL | 6653 |
| weighted | NPELIPHIE | 6654 |
| weighted | IALSYMRGG | 6655 |
| weighted | SIVGARKGV | 6656 |
| weighted | PTLSQPAAA | 6657 |
| weighted | RHETAYCTL | 6658 |
| weighted | KGTTKIPIF | 6659 |
| weighted | NGAKADPAI | 6660 |
| weighted | LEVVLNETN | 6661 |
| weighted | SMGTTIHFL | 6662 |
| weighted | DQVELLYVL | 6663 |
| weighted | NLGVGRILG | 6664 |
| weighted | KEKPAEGLR | 6665 |
| weighted | FVEALTVDC | 6666 |
| weighted | RTGGEYATQ | 6667 |
| weighted | ELPELEDEE | 6668 |
| weighted | RSAASHCEA | 6669 |
| weighted | VQKSLLRPL | 6670 |
| weighted | AYKPMLNDP | 6671 |
| weighted | QACTPIFLF | 6672 |
| weighted | PADYTSPPN | 6673 |
| weighted | LDEQQGAFQ | 6674 |
| weighted | DGGAPSAGT | 6675 |
| weighted | DTKDEPPGQ | 6676 |
| weighted | CAITRREDY | 6677 |
| weighted | AVFDSPGKK | 6678 |
| weighted | EAVNGESIL | 6679 |
| weighted | SPMKVFQSL | 6680 |
| weighted | ISLAHLIMG | 6681 |
| weighted | NKVWRPKAI | 6682 |
| weighted | VNNTERLPK | 6683 |
| weighted | LHSVVLGIS | 6684 |
| weighted | DNSDKLELS | 6685 |
| weighted | DNENVEVQS | 6686 |
| weighted | LDVLEAYTT | 6687 |
| weighted | YVKSHKDKR | 6688 |
| weighted | DDCATLDFA | 6689 |
| weighted | EGLLAQMRS | 6690 |
| weighted | RGQPFVQQV | 6691 |
| weighted | VIVSRPRND | 6692 |
| weighted | SSRYGIWRV | 6693 |
| weighted | GKDYTTLRE | 6694 |
| weighted | KWRLVYGML | 6695 |
| weighted | WANKDEARQ | 6696 |
| weighted | SVFFRQDYT | 6697 |
| weighted | ATAGSPGEK | 6698 |
| weighted | QFENYEGRE | 6699 |
| weighted | SPIEAECTH | 6700 |
| weighted | PESSINPNS | 6701 |
| weighted | TEDSPEAVP | 6702 |
| weighted | GLEQINYTS | 6703 |
| weighted | GKLKSSEND | 6704 |
| weighted | APRVLPRPS | 6705 |
| weighted | NSSKDDGND | 6706 |
| weighted | RNVKWVCAF | 6707 |
| weighted | QALLFYSVA | 6708 |
| weighted | LGSLPEFLT | 6709 |
| weighted | VAYLLRAAS | 6710 |
| weighted | IIVRESRFK | 6711 |
| weighted | DEVFGQMGA | 6712 |
| weighted | SIKAPRLDN | 6713 |
| weighted | NGAADSRRE | 6714 |
| weighted | SPFILSQGV | 6715 |
| weighted | HDSGCPEQA | 6716 |
| weighted | HPLFRPTRL | 6717 |
| weighted | FGDTPPNIA | 6718 |
| weighted | TPRLYVHSP | 6719 |
| weighted | ESTPLSAVN | 6720 |
| weighted | IVLEEVEST | 6721 |
| weighted | REVRFDGER | 6722 |
| weighted | NRLDQFMSP | 6723 |
| weighted | PLMMALWRD | 6724 |
| weighted | RKFQSNYQS | 6725 |
| weighted | SHVAQICDF | 6726 |
| weighted | LHASNIVAV | 6727 |
| weighted | PKNPPYFQS | 6728 |
| weighted | SSEQGRPAI | 6729 |
| weighted | ELKRESSAH | 6730 |
| weighted | PTDYEAALD | 6731 |
| weighted | LIFGPLTSQ | 6732 |
| weighted | EAKQLSSMT | 6733 |
| weighted | AIRWLQAQS | 6734 |
| weighted | NNPRSFRVV | 6735 |
| weighted | SSESDTQLG | 6736 |
| weighted | DSFPKKLRV | 6737 |
| weighted | FTGPLSKGP | 6738 |
| weighted | HRMSTQPAP | 6739 |
| weighted | LCKTVERIY | 6740 |
| weighted | LLVREAQHK | 6741 |
| weighted | RNQARTRYS | 6742 |
| weighted | VFVGGGTSW | 6743 |
| weighted | ILMGQKINE | 6744 |
| weighted | VWGEPIGRR | 6745 |
| weighted | QIQLTNYIA | 6746 |
| weighted | GNHSRLGEE | 6747 |
| weighted | HFFGDDGCK | 6748 |
| weighted | QEYSDNLWP | 6749 |
| weighted | SKGTPARDA | 6750 |
| weighted | GLHQAIENF | 6751 |
| weighted | NCFFLKDTV | 6752 |
| weighted | RKVINAAIQ | 6753 |
| weighted | SIESGKKCA | 6754 |
| weighted | LRSFLRNKG | 6755 |
| weighted | YTPSKCSSG | 6756 |
| weighted | HTLQCQPHG | 6757 |
| weighted | MNARTPPQN | 6758 |
| weighted | RLRPLNDST | 6759 |
| weighted | SFDWRGLSV | 6760 |
| weighted | HASAEPMEM | 6761 |
| weighted | SLLGSWRLF | 6762 |
| weighted | LFCYEIGAL | 6763 |
| weighted | PHPYSASDR | 6764 |
| weighted | ESVFKKCQQ | 6765 |
| weighted | TGGLAAREN | 6766 |
| weighted | DGYQIEVYS | 6767 |
| weighted | LTVGCVNIR | 6768 |
| weighted | KFDEIFMQL | 6769 |
| weighted | MPPKDHDPK | 6770 |
| weighted | PLLFWIKHE | 6771 |
| weighted | SAAVPDYQV | 6772 |
| weighted | HLYAIFVDQ | 6773 |
| weighted | PNEDSAGLY | 6774 |
| weighted | DERTRVGIK | 6775 |
| weighted | ATLRFSINF | 6776 |
| weighted | QLQVNVGLA | 6777 |
| weighted | IPTTPISDL | 6778 |
| weighted | VESIMLRLL | 6779 |
| weighted | LIGDIVDQL | 6780 |
| weighted | LNIFLISWA | 6781 |
| weighted | GATNKIKRY | 6782 |
| weighted | KPAGTDRIV | 6783 |
| weighted | VSTEGICQT | 6784 |
| weighted | TMMPLGFSL | 6785 |
| weighted | TAFYLGLNE | 6786 |
| weighted | VLTLLKDQV | 6787 |
| weighted | SLSRSSISR | 6788 |
| weighted | NPFRSEPAG | 6789 |
| weighted | ERAAYSAVA | 6790 |
| weighted | LADTHPERP | 6791 |
| weighted | PLGLYEASP | 6792 |
| weighted | YQCGATELS | 6793 |
| weighted | AIPPNKEKQ | 6794 |
| weighted | THSSKISSM | 6795 |
| weighted | SYTRLYCRV | 6796 |
| weighted | CDKSRYLKR | 6797 |
| weighted | AEAHKTEAV | 6798 |
| weighted | IELGSLYTL | 6799 |
| weighted | KHNRYQKES | 6800 |
| weighted | KSSRIGSFY | 6801 |
| weighted | INDCLRKFPH | 6802 |
| weighted | NFLCHSSGV | 6803 |
| weighted | YKLFHWPVW | 6804 |
| weighted | FPERRWQVS | 6805 |
| weighted | KSMQYKVMS | 6806 |
| weighted | QFYTVEADF | 6807 |
| weighted | FGDPASQLH | 6808 |
| weighted | ECEVSGKTQ | 6809 |
| weighted | PKGAISRNY | 6810 |
| weighted | WNPSFSLHL | 6811 |
| weighted | CYFLEMLQF | 6812 |
| weighted | ADRSGGDAP | 6813 |
| weighted | SKQTAHEIQ | 6814 |
| weighted | CISDEDMYF | 6815 |
| weighted | NYSFFDSLM | 6816 |
| weighted | RIPGNLLHN | 6817 |
| weighted | GTGFRPVVL | 6818 |
| weighted | TDSLAVDQL | 6819 |
| weighted | LLDQMIGIV | 6820 |
| weighted | MNRFRGERT | 6821 |
| weighted | VGGRSHKKE | 6822 |
| weighted | PLRKSLGLD | 6823 |
| weighted | TKTTDNNDN | 6824 |
| weighted | STDIPKLKI | 6825 |
| weighted | AWEAPNTLP | 6826 |
| weighted | SVSISLREN | 6827 |
| weighted | FQFLSHLLE | 6828 |
| weighted | LKAVYQFPV | 6829 |
| weighted | FVSSIDEKG | 6830 |
| weighted | GWEAENYFA | 6831 |
| weighted | ALKMDSGHR | 6832 |
| weighted | DPRTRLELS | 6833 |
| weighted | RGMSEEQLS | 6834 |
| weighted | LSRGLGHDP | 6835 |
| weighted | VINSEMDGE | 6836 |
| weighted | RWDTLHINP | 6837 |
| weighted | YEAGSFIAQ | 6838 |
| weighted | SSRRGGKDI | 6839 |
| weighted | GASMRLEGL | 6840 |
| weighted | EFNKAKIPI | 6841 |
| weighted | IVQGSVPNL | 6842 |
| weighted | LRMKNELGL | 6843 |
| weighted | GPFDLVFAS | 6844 |
| weighted | SAGANFRWN | 6845 |
| weighted | CESDASKLG | 6846 |
| weighted | EHRKRSNND | 6847 |
| weighted | GMSNKETSS | 6848 |
| weighted | TPDETLSSL | 6849 |
| weighted | AQSQRRSCV | 6850 |
| weighted | SERNMDAQV | 6851 |
| weighted | LIPRLGKFQ | 6852 |
| weighted | LRNYCTILG | 6853 |
| weighted | FENLDLLTS | 6854 |
| weighted | PQEASFDHV | 6855 |
| weighted | CVGFLINEN | 6856 |
| weighted | IPASWLKLI | 6857 |
| weighted | QMTFPTGFF | 6858 |
| weighted | SFSQLKASL | 6859 |
| weighted | AGFAEDSAF | 6860 |
| weighted | AQQESCRLH | 6861 |
| weighted | CDGFEATVP | 6862 |
| weighted | AMELLLKKN | 6863 |
| weighted | PASGTKCLQ | 6864 |
| weighted | GAKTGEEPP | 6865 |
| weighted | PFIKIPALE | 6866 |
| weighted | KLERLLYDG | 6867 |
| weighted | QSELTKFSE | 6868 |
| weighted | IPSSPFQEL | 6869 |
| weighted | PERGTLRKS | 6870 |
| weighted | ERMQLTAQQ | 6871 |
| weighted | DQMSFKNMR | 6872 |
| weighted | DPAGSSEQQ | 6873 |
| weighted | VVNDYKRFQ | 6874 |
| weighted | EETAYLPRL | 6875 |
| weighted | HEEESSGSH | 6876 |
| weighted | DDFEVDKIS | 6877 |
| weighted | GALFAKSHN | 6878 |
| weighted | ETGPETSPV | 6879 |
| weighted | MPSGICRTL | 6880 |
| weighted | LDLSQDQFR | 6881 |
| weighted | DYLYSSFRK | 6882 |
| weighted | LDLRGNFGL | 6883 |
| weighted | KRQAGCNSV | 6884 |
| weighted | GGVMCELLI | 6885 |
| weighted | SKLVYKRFF | 6886 |
| weighted | KTPKPLVTQ | 6887 |
| weighted | PEYVAKVLG | 6888 |
| weighted | ATDRECCNP | 6889 |
| weighted | LFLKPQPTT | 6890 |
| weighted | KVLLLAKQN | 689 |
| weighted | NSERKFRLK | 6892 |
| weighted | GHAVPPEEV | 6893 |
| weighted | VLSTYGELP | 6894 |
| weighted | FDPSTDLYT | 6895 |
| weighted | WQLQILVIP | 6896 |
| weighted | SETASGPWE | 6897 |
| weighted | YKREFFDLE | 6898 |
| weighted | ATVFLDREC | 6899 |
| weighted | SREEHLKTE | 6900 |
| weighted | AHALSREVV | 6901 |
| weighted | SSIQEMKCA | 6902 |
| weighted | RHSIYELQS | 6903 |
| weighted | PETSDLPGT | 6904 |
| weighted | SIQKFQLDI | 6905 |
| weighted | FPVFAKASL | 6906 |
| weighted | DAQALPPLE | 6907 |
| weighted | FQLLRQWAF | 6908 |
| weighted | HLLEFLGTT | 6909 |
| weighted | LGGFIEPEL | 6910 |
| weighted | DELSPIEGT | 6911 |
| weighted | SSDEARTNL | 6912 |
| weighted | PPNLDQPSV | 6913 |
| weighted | LWKAESIKP | 6914 |
| weighted | PEVLFTIVD | 6915 |
| weighted | QTQDSQGQV | 6916 |
| weighted | SDGASSLHL | 6917 |
| weighted | VLAVPIVNN | 6918 |
| weighted | RPIGASKVG | 6919 |
| weighted | CDVPWRQVV | 6920 |
| weighted | IKSKGAANS | 6921 |
| weighted | TNNNTFKLG | 6922 |
| weighted | LPGMTNLGV | 6923 |
| weighted | SFSSVKYKI | 6924 |
| weighted | YGFTGYIIT | 6925 |
| weighted | FSSNKAYPQ | 6926 |
| weighted | RPSLIEKTT | 6927 |
| weighted | LDCMPGFID | 6928 |
| weighted | LACAFPNEG | 6929 |
| weighted | NQATRRHPV | 6930 |
| weighted | MHQSRIPEE | 6931 |
| weighted | GAQPFRDRP | 6932 |
| weighted | FTLPMVWLN | 6933 |
| weighted | ANRVMEVLT | 6934 |
| weighted | EFRLPTLSS | 6935 |
| weighted | PDLAAKVVE | 6936 |
| weighted | RNDISVEGM | 6937 |
| weighted | YENGWLNDV | 6938 |
| weighted | TDLVRPTGE | 6939 |
| weighted | AMQAKDDLD | 6940 |
| weighted | LATMQTVSV | 6941 |
| weighted | SEVHYDKES | 6942 |
| weighted | AKELCNPDL | 6943 |
| weighted | AEQKVSSLL | 6944 |
| weighted | ERGPHEGHG | 6945 |
| weighted | HGEKPLEIK | 6946 |
| weighted | TPGNCRKLL | 6947 |
| weighted | ALVLETTEA | 6948 |
| weighted | HNAGARDLS | 6949 |
| weighted | FPEKTAESY | 6950 |
| weighted | ACILGAIDR | 6951 |
| weighted | PYPGCVQKI | 6952 |
| weighted | NKDISSFSR | 6953 |
| weighted | LKAYEQPVE | 6954 |
| weighted | GSNGRYGKQ | 6955 |
| weighted | PTGAELSAH | 6956 |
| weighted | IEILDVSHS | 6957 |
| weighted | LLSRALDVM | 6958 |
| weighted | GRQPCLSVS | 6959 |
| weighted | GTVIAISYP | 6960 |
| weighted | RPEFSRQDL | 6961 |
| weighted | STGDNRLEI | 6962 |
| weighted | GEFVLKDPA | 6963 |
| weighted | KSVSVDYRH | 6964 |
| weighted | KQARIESRG | 6965 |
| weighted | RAWLSPVMP | 6966 |
| weighted | HNGCCDMQL | 6967 |
| weighted | STSMTIIPP | 6968 |
| weighted | ANTEVSTQL | 6969 |
| weighted | SPFAKLTPE | 6970 |
| weighted | SKVPGMLFP | 6971 |
| weighted | AHPKPLLEC | 6972 |
| weighted | PATQMLHEC | 6973 |
| weighted | ACTPVHTNT | 6974 |
| weighted | PFTFREERK | 6975 |
| weighted | KRGYECTDC | 6976 |
| weighted | PASNTASPM | 6977 |
| weighted | ANTLLYTSE | 6978 |
| weighted | CLTFPAAQS | 6979 |
| weighted | RKDKKGSAP | 6980 |
| weighted | HELFHSEQE | 6981 |
| weighted | DFDLAGGIA | 6982 |
| weighted | ASAAMLMVP | 6983 |
| weighted | AFEARRDAI | 6984 |
| weighted | TLAWNPKNC | 6985 |
| weighted | YALSFGQKC | 6986 |
| weighted | VDAKYSARY | 6987 |
| weighted | LCARVTAFR | 6988 |
| weighted | GLPALSQAL | 6989 |
| weighted | RGISKPSQV | 6990 |
| weighted | FDPKGAETK | 6991 |
| weighted | ANFKENHKD | 6992 |
| weighted | VPFCETPVA | 6993 |
| weighted | GIYLGPLFF | 6994 |
| weighted | AITDKKLMP | 6995 |
| weighted | KAGGLDLGG | 6996 |
| weighted | THRAKTSDI | 6997 |
| weighted | INNEELSATY | 6998 |
| weighted | YEAAHERSP | 6999 |
| weighted | DQSPVDGTL | 7000 |
| weighted | VPNYCDSAT | 7001 |
| weighted | GYSHNRDTN | 7002 |
| weighted | LLAAEGGRE | 7003 |
| weighted | VRDMKRTMA | 7004 |
| weighted | PGFELEQCA | 7005 |
| weighted | DTKGEFDVGV | 7006 |
| weighted | VYGVTPADAL | 7007 |
| weighted | YSAHPKMADE | 7008 |
| weighted | GSMSAPLPWM | 7009 |
| weighted | SPNDPQEMQN | 7010 |
| weighted | YAEEGLARKD | 7011 |
| weighted | IDGSEDLLFR | 7012 |
| weighted | VAADQLFAME | 7013 |
| weighted | AIALVDPVPP | 7014 |
| weighted | WDGPYREVAK | 7015 |
| weighted | SLQLWLRKTE | 7016 |
| weighted | LVLLRHFCFL | 7017 |
| weighted | SEYNSISIAT | 7018 |
| weighted | DLMPKYDTGD | 7019 |
| weighted | SEPADLQLVI | 7020 |
| weighted | SLLEEKEDRW | 7021 |
| weighted | CNDSNLSAEF | 7022 |
| weighted | DSNSFFSSAG | 7023 |
| weighted | GQDPEKVAHG | 7024 |
| weighted | KGIQLQISAC | 7025 |
| weighted | KFHDFDITPV | 7026 |
| weighted | DWEQKVRESA | 7027 |
| weighted | VLMGDGKTGG | 7028 |
| weighted | SGVSKLCPDC | 7029 |
| weighted | TNASARDREP | 7030 |
| weighted | KGHGNLGEVL | 7031 |
| weighted | PASATTAAIL | 7032 |
| weighted | LYLNLNLAIL | 7033 |
| weighted | LKKINLAVTH | 7034 |
| weighted | KLALAPCYFG | 7035 |
| weighted | TSSQQLIEGV | 7036 |
| weighted | TRVLVQLGAE | 7037 |
| weighted | AMFEAKLLVL | 7038 |
| weighted | CARHGMPGVL | 7039 |
| weighted | AVIMIRGIKL | 7040 |
| weighted | KHAKLVCSLD | 7041 |
| weighted | SSMLVQLSRK | 7042 |
| weighted | VDGARNLYYS | 7043 |
| weighted | RSNCHLLTIC | 7044 |
| weighted | LAEAKTSRLF | 7045 |
| weighted | PLDNIDIPNT | 7046 |
| weighted | MKLMVFLSES | 7047 |
| weighted | TFDDGQPDLH | 7048 |
| weighted | RSDDFPFLEV | 7049 |
| weighted | KPNSDGHHEP | 7050 |
| weighted | EVGDAPQALF | 7051 |
| weighted | AVPVKITGAA | 7052 |
| weighted | MLTPSFHRLI | 7053 |
| weighted | AIEIGAKNCI | 7054 |
| weighted | TGESTFAASE | 7055 |
| weighted | LGIFGDKGLL | 7056 |
| weighted | CTRHYQRKEA | 7057 |
| weighted | SAVIEAKLAA | 7058 |
| weighted | LLLFRSRIPG | 7059 |
| weighted | FNPLQSAYLV | 7060 |
| weighted | DEGVEQSRCA | 7061 |
| weighted | TVSKAALNVE | 7062 |
| weighted | PRGRAAFKSP | 7063 |
| weighted | ANQIGRLSIK | 7064 |
| weighted | SRYKRNSCHT | 7065 |
| weighted | VPASKGQPGK | 7066 |
| weighted | SIKAFRKDWR | 7067 |
| weighted | LEKDVPNENA | 7068 |
| weighted | VLKVLGVLQS | 7069 |
| weighted | ADRGSLPPGR | 7070 |
| weighted | GELTAHNFAK | 7071 |
| weighted | GVVLMLSFNI | 7072 |
| weighted | AISPFKEASV | 7073 |
| weighted | ERYPKLFEAH | 7074 |
| weighted | SPHFKHMTAK | 7075 |
| weighted | ALIEQTDGQA | 7076 |
| weighted | EPEVRSEADA | 7077 |
| weighted | GVYVQRHGPS | 7078 |
| weighted | VYYLRGYITK | 7079 |
| weighted | NEPIVEDKSH | 7080 |
| weighted | GDSKAKGGVS | 7081 |
| weighted | DHDEALEGEW | 7082 |
| weighted | PFPLIIPFNV | 7083 |
| weighted | SVLEESVTMK | 7084 |
| weighted | DPRDPRSPKP | 7085 |
| weighted | NTSFQQMQIP | 7086 |
| weighted | LTAQKLSSEI | 7087 |
| weighted | RMQIVDLQLN | 7088 |
| weighted | VLAAASADAS | 7089 |
| weighted | DLKILGKGAF | 7090 |
| weighted | LPAIAVDKGI | 7091 |
| weighted | TLRAGSYCLI | 7092 |
| weighted | DRVHRSVPDK | 7093 |
| weighted | IVALELVMIL | 7094 |
| weighted | QLSNAGVGRH | 7095 |
| weighted | LPSFCTFSTQ | 7096 |
| weighted | NTRSALFQGQ | 7097 |
| weighted | RRFLQGIRIG | 7098 |
| weighted | GEQQTKRLAG | 7099 |
| weighted | VIQLYEIHNA | 7100 |
| weighted | INPFNRFGPC | 7101 |
| weighted | VHVNEASDDY | 7102 |
| weighted | ILVDRNQLCL | 7103 |
| weighted | KRGPDGQCFG | 7104 |
| weighted | KHIEPSIPPA | 7105 |
| weighted | VEPLRLDPDE | 7106 |
| weighted | ILYGGRGLRK | 7107 |
| weighted | AMSTVGVDVA | 7108 |
| weighted | TAWVGRVTGQ | 7109 |
| weighted | PTIALVHFHD | 7110 |
| weighted | GLRNIGNLAE | 7111 |
| weighted | SEFHIQVSPP | 7112 |
| weighted | VGPQAVLKHE | 7113 |
| weighted | VSRNTVDIVL | 7114 |
| weighted | SAVTHSLINP | 7115 |
| weighted | VFCKLWCREI | 7116 |
| weighted | NDSVALSGFV | 7117 |
| weighted | GSAESSCKIQ | 7118 |
| weighted | LLMAGTTASI | 7119 |
| weighted | SSKTPQSAGE | 7120 |
| weighted | RSMVKHIKER | 7121 |
| weighted | YQARNSVVME | 7122 |
| weighted | DKCTLMAGFL | 7123 |
| weighted | QLSTEKLSLR | 7124 |
| weighted | TRSRKLQVKR | 7125 |
| weighted | DGEYDSELDR | 7126 |
| weighted | GLPATGSSFG | 7127 |
| weighted | PQSGEHSYFV | 7128 |
| weighted | KLIDSEKARY | 7129 |
| weighted | RDGALRHSLK | 7130 |
| weighted | YIALESNYGM | 7131 |
| weighted | LSSRKCDSYS | 7132 |
| weighted | ESSFWHCSLA | 7133 |
| weighted | NAYYATPRQT | 7134 |
| weighted | YLEDKGCCQY | 7135 |
| weighted | GDGSDAAAIV | 7136 |
| weighted | LLLRNLGLQE | 7137 |
| weighted | WSKQLEVVAQ | 7138 |
| weighted | SRALIKTIYT | 7139 |
| weighted | ACMKNRGQIP | 7140 |
| weighted | NPLFWPSIAR | 7141 |
| weighted | HSYCVLKTSE | 7142 |
| weighted | GYNLSLESDV | 7143 |
| weighted | LDSIEFYGSA | 7144 |
| weighted | HSIIFNYPVK | 7145 |
| weighted | ELQVTIGCLQ | 7146 |
| weighted | QQPASPRLIF | 7147 |
| weighted | EREDSAEQID | 7148 |
| weighted | ENFDLILNEA | 7149 |
| weighted | ILRINTPAEE | 7150 |
| weighted | EHKRLLLGDE | 7151 |
| weighted | TSKFEYEQED | 7152 |
| weighted | SEEAKGQPMA | 7153 |
| weighted | HCGTLGSNIN | 7154 |
| weighted | ASQPRLLDIY | 7155 |
| weighted | LALHGYTTVR | 7156 |
| weighted | CLMFSFGFHL | 7157 |
| weighted | IRSYQEENYC | 7158 |
| weighted | IEADPVQETA | 7159 |
| weighted | ELPNTPDAVS | 7160 |
| weighted | DSTHSIGEQC | 7161 |
| weighted | SWRAKWGKTY | 7162 |
| weighted | PLIKRLDILL | 7163 |
| weighted | TEFQEKPGES | 7164 |
| weighted | PKDEKRDSYY | 7165 |
| weighted | GICRLSAPTL | 7166 |
| weighted | SGLDSNNVRE | 7167 |
| weighted | QDPTALESFI | 7168 |
| weighted | SITDLPIRPS | 7169 |
| weighted | TGGQRRTLKK | 7170 |
| weighted | RLELPRTIVV | 7171 |
| weighted | YRVHCDSNTQ | 7172 |
| weighted | SDMLSAVRKS | 7173 |
| weighted | VRSSLSAVPT | 7174 |
| weighted | WDNQAGQYPD | 7175 |
| weighted | PQQDESTALL | 7176 |
| weighted | QTKKDKPSLM | 7177 |
| weighted | TSEGEENNPK | 7178 |
| weighted | LRFSTELKVE | 7179 |
| weighted | YLVWCCLREE | 7180 |
| weighted | WLARSTKKEK | 7181 |
| weighted | GLKWHEQKDR | 7182 |
| weighted | SRDEYLSSMR | 7183 |
| weighted | YDSMESGDDK | 7184 |
| weighted | TPYPKVVEKP | 7185 |
| weighted | EWMRCPVNAA | 7186 |
| weighted | SYWNKLRTQE | 7187 |
| weighted | PRGTGVGCEQ | 7188 |
| weighted | RLPQFIVVGP | 7189 |
| weighted | TSIAGIYLAI | 7190 |
| weighted | RLEKCLCYSF | 7191 |
| weighted | LLNFPAQLYH | 7192 |
| weighted | GAFEIEEPAL | 7193 |
| weighted | TSQEQSQTMS | 7194 |
| weighted | KNALVAIAPD | 7195 |
| weighted | IRFATGIVIN | 7196 |
| weighted | KEYIRGSCII | 7197 |
| weighted | LGVEPSEHVA | 7198 |
| weighted | LSRRVNFGEF | 7199 |
| weighted | ATKGMASIKN | 7200 |
| weighted | VESQLVEQNS | 7201 |
| weighted | PSTGQCQEIL | 7202 |
| weighted | VFQQTDKEWC | 7203 |
| weighted | KVLRILEFIR | 7204 |
| weighted | ERYIGLVHRG | 7205 |
| weighted | EPRAAKQVPL | 7206 |
| weighted | PISRVFMMGC | 7207 |
| weighted | LSDEAERKSA | 7208 |
| weighted | KPVFGHYTRK | 7209 |
| weighted | RALHYKQGQE | 7210 |
| weighted | DLLKGSYCAM | 7211 |
| weighted | LAVNSTLNST | 7212 |
| weighted | TVAWACYSEL | 7213 |
| weighted | PRKCTDTMWS | 7214 |
| weighted | WHVDTPERMN | 7215 |
| weighted | LLLLNFDRDP | 7216 |
| weighted | EDDRAAIAIH | 7217 |
| weighted | ISDCIISYLM | 7218 |
| weighted | YLRPVLKGGY | 7219 |
| weighted | GQRAHGPRTL | 7220 |
| weighted | WDRLAFLDSG | 7221 |
| weighted | LECVGCADVA | 7222 |
| weighted | AEKNVLYYAR | 7223 |
| weighted | SMLDKSVDVP | 7224 |
| weighted | YAILTKQLFQ | 7225 |
| weighted | KPPALVDIVK | 7226 |
| weighted | TSDWNTAASV | 7227 |
| weighted | SEECTASLER | 7228 |
| weighted | LPKILNSGTS | 7229 |
| weighted | PLQMNLDIAP | 7230 |
| weighted | TNSTGLVVKA | 7231 |
| weighted | FDERRGLKEP | 7232 |
| weighted | EKPFSMNLNV | 7233 |
| weighted | MIDDLSPDWV | 7234 |
| weighted | FGMDRSSKDF | 7235 |
| weighted | TPSPPEATIL | 7236 |
| weighted | NATRELQRGT | 7237 |
| weighted | CGATSARRTS | 7238 |
| weighted | KVAKRLPVNV | 7239 |
| weighted | AGDRAPKLWL | 7240 |
| weighted | RYGNGGKTLG | 7241 |
| weighted | YHFTHCNWIT | 7242 |
| weighted | IKSADFGHFM | 7243 |
| weighted | KLDRLDCRQS | 7244 |
| weighted | KTQQAGYGVL | 7245 |
| weighted | HVDVDTECAM | 7246 |
| weighted | LQLTSDCSIG | 7247 |
| weighted | SELNILVMIE | 7248 |
| weighted | MLLFFGLKAG | 7249 |
| weighted | EERLACPPKF | 7250 |
| weighted | DADMLRFDSR | 7251 |
| weighted | GNSRMRKAPP | 7252 |
| weighted | PALPSVKTVP | 7253 |
| weighted | IYGDMDQRAN | 7254 |
| weighted | TAQAALIEVK | 7255 |
| weighted | LAGRDRGLGL | 7256 |
| weighted | SYGEFVYKDD | 7257 |
| weighted | PPASEMESPI | 7258 |
| weighted | EDKHTAAGAG | 7259 |
| weighted | NHRMTKRMNV | 7260 |
| weighted | NTCRAASRFE | 7261 |
| weighted | LSFSTFGVVI | 7262 |
| weighted | KGSAATKHPL | 7263 |
| weighted | YPPVSSVGFQ | 7264 |
| weighted | GHGSEITSAE | 7265 |
| weighted | AMDQHDKSVS | 7266 |
| weighted | GFPQIGHLAL | 7267 |
| weighted | LGYIKLKPLI | 7268 |
| weighted | RPVKDGMYGR | 7269 |
| weighted | SRMCLPFQDH | 7270 |
| weighted | EERAFALEPS | 7271 |
| weighted | KLEKDKSFLP | 7272 |
| weighted | LWFSLSSQRL | 7273 |
| weighted | PESREESPFK | 7274 |
| weighted | SSKTLGFSGS | 7275 |
| weighted | PLTEETFLPE | 7276 |
| weighted | FLFDNPRRED | 7277 |
| weighted | MALESPADQL | 7278 |
| weighted | VQVSRDPNEM | 7279 |
| weighted | SFGVPSLTRL | 7280 |
| weighted | GHPSDRMMPQ | 7281 |
| weighted | NEQNLSPEEV | 7282 |
| weighted | RPDPLPIEDS | 7283 |
| weighted | SMSAGQYFIL | 7284 |
| weighted | TVGLMSQKNI | 7285 |
| weighted | MKDDEISVGR | 7286 |
| weighted | ARAAWFLPEI | 7287 |
| weighted | YATAVETFRA | 7288 |
| weighted | ERGIAIGKCL | 7289 |
| weighted | QPTPAPEGVS | 7290 |
| weighted | LQENPISVHR | 7291 |
| weighted | YLANVFYYTI | 7292 |
| weighted | ILGELSGLPAM | 7293 |
| weighted | STKYNESKIH | 7294 |
| weighted | LPHGHRSGSR | 7295 |
| weighted | SAVWEEQIGN | 7296 |
| weighted | SLESHGLRPL | 7297 |
| weighted | LIYLKRDSQF | 7298 |
| weighted | VLWPLSVVCT | 7299 |
| weighted | RQSTHVELQT | 7300 |
| weighted | KLDQEGTLWA | 7301 |
| weighted | QDVLKITIPK | 7302 |
| weighted | ITHTHQKLGS | 7303 |
| weighted | ISRKPSIYLF | 7304 |
| weighted | ALILQVLHIF | 7305 |
| weighted | GIYYLADALQ | 7306 |
| weighted | QSSSEALLAG | 7307 |
| weighted | GTKGELKLER | 7308 |
| weighted | EPSLFSCAYP | 7309 |
| weighted | DYNYSSPQTI | 7310 |
| weighted | ELSDEKPPTR | 7311 |
| weighted | EGYMRTLGKK | 7312 |
| weighted | QRLHSCGPNK | 7313 |
| weighted | LRVGIGVLFL | 7314 |
| weighted | HMIHSSTTRT | 7315 |
| weighted | TLHRARIEFA | 7316 |
| weighted | EPSDGAFPPD | 7317 |
| weighted | HVQDYAKRLG | 7318 |
| weighted | SPPGHKVQAR | 7319 |
| weighted | KTTLGPACYR | 7320 |
| weighted | LLCFTSYLAL | 7321 |
| weighted | NPKGSLLEVV | 7322 |
| weighted | ELKNCITWPI | 7323 |
| weighted | IRDVTSGQGL | 7324 |
| weighted | PLWLLSLRTI | 7325 |
| weighted | VRGGTGDPNA | 7326 |
| weighted | KLGKCYADDS | 7327 |
| weighted | DAAKDSAHMA | 7328 |
| weighted | TKILEYLCKP | 7329 |
| weighted | RIQERECPVP | 7330 |
| weighted | QLRQMDSLSL | 7331 |
| weighted | RMYPSTAFGF | 7332 |
| weighted | GGFMVITEPK | 7333 |
| weighted | KQASRFQVWD | 7334 |
| weighted | IEWSVLIYLR | 7335 |
| weighted | MSQATPAAAE | 7336 |
| weighted | KWSPLPEIVQ | 7337 |
| weighted | PTEPFHPLCG | 7338 |
| weighted | FPYIAIKPDE | 7339 |
| weighted | QNYKETRCCA | 7340 |
| weighted | VFNAGIPTVS | 7341 |
| weighted | TNDQYHPNNK | 7342 |
| weighted | CTLCQMGGVS | 7343 |
| weighted | YSLYRSLSEC | 7344 |
| weighted | AKILYNAELA | 7345 |
| weighted | PALYMAIEEE | 7346 |
| weighted | IWNDLDQNWL | 7347 |
| weighted | LSHVEEICLS | 7348 |
| weighted | AVLMVQSRIM | 7349 |
| weighted | FFFFNNPEAN | 7350 |
| weighted | RLGIFDLKLF | 7351 |
| weighted | RTVHIATAEI | 7352 |
| weighted | KGHQGETCQN | 7353 |
| weighted | LEYAMTSSVW | 7354 |
| weighted | FESEPNLIRK | 7355 |
| weighted | QVLKVLWRQE | 7356 |
| weighted | DQVTLCLLYP | 7357 |
| weighted | LSNPVVQHER | 7358 |
| weighted | EAKDFTRSVQ | 7359 |
| weighted | TLYCSVAHSM | 7360 |
| weighted | WSLNSHTGGP | 7361 |
| weighted | IDVRRVSTPA | 7362 |
| weighted | LEVSASSAFY | 7363 |
| weighted | AGGLLSSHIA | 7364 |
| weighted | LPFELRRQNV | 7365 |
| weighted | INNSVKEANIS | 7366 |
| weighted | ARPLVLLGSS | 7367 |
| weighted | ESGAGLKNPI | 7368 |
| weighted | LFEPAKLDHM | 7369 |
| weighted | CGYQQLRFHN | 7370 |
| weighted | TPRERLIADE | 7371 |
| weighted | AFVHAGPHEA | 7372 |
| weighted | EQQAGKVIQV | 7373 |
| weighted | FLQLPPRGSI | 7374 |
| weighted | CIRPPESLCY | 7375 |
| weighted | NELATKANLS | 7376 |
| weighted | DSLLVEMLAQ | 7377 |
| weighted | SGVFIVHRAR | 7378 |
| weighted | VELRTLSLIH | 7379 |
| weighted | LRLRAGILIV | 7380 |
| weighted | GCQLTVHEHP | 7381 |
| weighted | RVELANSPPH | 7382 |
| weighted | ETAAWKSTKT | 7383 |
| weighted | AYSTYKVVRH | 7384 |
| weighted | RRRKTEVRPW | 7385 |
| weighted | TPPAVQYRGA | 7386 |
| weighted | ATATSGNACL | 7387 |
| weighted | RHAASLHEGA | 7388 |
| weighted | MLSIEGAPDD | 7389 |
| weighted | GSMASDAIQR | 7390 |
| weighted | YSDRCDGVKN | 7391 |
| weighted | SGFFSGGMNE | 7392 |
| weighted | REYIHIAAAQ | 7393 |
| weighted | FLMIHDKVRL | 7394 |
| weighted | FHHLETQGLV | 7395 |
| weighted | LGPNRVTKSN | 7396 |
| weighted | IAPEPVVENS | 7397 |
| weighted | HSITEARRLS | 7398 |
| weighted | SVCAQRTNVG | 7399 |
| weighted | SARRLIIDHR | 7400 |
| weighted | YKLDKEVLPN | 7401 |
| weighted | SAQVEGHKNY | 7402 |
| weighted | VSLRILALDP | 7403 |
| weighted | KIQIPQMDPP | 7404 |
| weighted | SFGNLGRAAG | 7405 |
| weighted | GLAKAPSDEG | 7406 |
| weighted | CGGGREQRSG | 7407 |
| weighted | GCRMCTSTVH | 7408 |
| weighted | KKEDDVVVSY | 7409 |
| weighted | RESDDSASTK | 7410 |
| weighted | EVLSSTIYPP | 7411 |
| weighted | LVIRDILPVM | 7412 |
| weighted | KCLLIKANLY | 7413 |
| weighted | IVLKLRKPFY | 7414 |
| weighted | VMWSVDEDKL | 7415 |
| weighted | NLYANQAEAQ | 7416 |
| weighted | QMIEGAMTTS | 7417 |
| weighted | SSIKEGAQFG | 7418 |
| weighted | QSDTKTMEND | 7419 |
| weighted | DVVTYQFVQV | 7420 |
| weighted | PVRDHYEREC | 7421 |
| weighted | LELEDENKQD | 7422 |
| weighted | SDRKYSAELQ | 7423 |
| weighted | RSPLYALRQT | 7424 |
| weighted | KGSILYNKKK | 7425 |
| weighted | DLIVKSFTGY | 7426 |
| weighted | PISPSICADL | 7427 |
| weighted | SDLTLTISKE | 7428 |
| weighted | FSLLNSIEDP | 7429 |
| weighted | VSALWAPVKA | 7430 |
| weighted | FQRRVGPATM | 7431 |
| weighted | GELSEWLSPV | 7432 |
| weighted | FHLVNLNAIE | 7433 |
| weighted | DQAVDPEWGQ | 7434 |
| weighted | YPHKPTKFGK | 7435 |
| weighted | QHCQEHDLLM | 7436 |
| weighted | GLDPSIQQGL | 7437 |
| weighted | HSRHASSAPL | 7438 |
| weighted | VRKTHILKSI | 7439 |
| weighted | EQYNGDNCQK | 7440 |
| weighted | SDSMAPPSDE | 7441 |
| weighted | SGSGMLLFQA | 7442 |
| weighted | KAPSDTPALA | 7443 |
| weighted | EPSKVTITEL | 7444 |
| weighted | APLIASPQIG | 7445 |
| weighted | AGCFQVCVLV | 7446 |
| weighted | EWMGMVEQDH | 7447 |
| weighted | FLNVELSGYL | 7448 |
| weighted | DDKFPLSMRA | 7449 |
| weighted | ARTPISLCSK | 7450 |
| weighted | VHDEDISSPG | 7451 |
| weighted | PCNGQTLTEK | 7452 |
| weighted | VQHFRHMQSL | 7453 |
| weighted | GVRLKPLHLP | 7454 |
| weighted | EVQQTLPESL | 7455 |
| weighted | KNQEPPRCFP | 7456 |
| weighted | VDKQDLCPLK | 7457 |
| weighted | NNIERNPNLL | 7458 |
| weighted | YALKCLHLAH | 7459 |
| weighted | ALERADECFV | 7460 |
| weighted | LLPMSATEIT | 7461 |
| weighted | TQMRAKVESN | 7462 |
| weighted | NISVKWSLKS | 7463 |
| weighted | VFEMPFGGTY | 7464 |
| weighted | YDVDPAYLDG | 7465 |
| weighted | EWGSLYVKIL | 7466 |
| weighted | SKTRLSVVAF | 7467 |
| weighted | WSSGLEKREL | 7468 |
| weighted | YKGKVSHTTY | 7469 |
| weighted | DENELPQALA | 7470 |
| weighted | FHVICFEEKC | 7471 |
| weighted | LHPAENSKIP | 7472 |
| weighted | KVGPRGKGPH | 7473 |
| weighted | ELITKLGALD | 7474 |
| weighted | VIQYTTLVYE | 7475 |
| weighted | EITPPVSIRL | 7476 |
| weighted | VKGEMSCQFV | 7477 |
| weighted | IEGGEPTEKI | 7478 |
| weighted | HAHARLSHII | 7479 |
| weighted | RTTFFESQVR | 7480 |
| weighted | NGGAMLLRKP | 7481 |
| weighted | AHRLDMLLAI | 7482 |
| weighted | PSWVLDQLDI | 7483 |
| weighted | TLPLLSWSYV | 7484 |
| weighted | AIAICILFAK | 7485 |
| weighted | AQYRNECLNF | 7486 |
| weighted | SEEATFPVSG | 7487 |
| weighted | SFRDLQLLSE | 7488 |
| weighted | TRTEKEETFL | 7489 |
| weighted | LEDDVFKTQD | 7490 |
| weighted | RAADGVCLCL | 7491 |
| weighted | GLHVKRLESM | 7492 |
| weighted | ARPHRLWAEG | 7493 |
| weighted | MAMEWEEMTK | 7494 |
| weighted | KVFYFFRLDP | 7495 |
| weighted | PNIQQSDRRE | 7496 |
| weighted | CSSMRQARKL | 7497 |
| weighted | SQLPITVLEK | 7498 |
| weighted | ELQSIKREPT | 7499 |
| weighted | QNFVPPPRHE | 7500 |
| weighted | TDRARSCSAR | 7501 |
| weighted | LVSQAGYESS | 7502 |
| weighted | TGLEGHSPYV | 7503 |
| weighted | GSHLQLVTPL | 7504 |
| weighted | GEEKEKQYDF | 7505 |
| weighted | QVDKLPLEAR | 7506 |
| weighted | AERSYRTQAN | 7507 |
| weighted | PRPLTGEHTT | 7508 |
| weighted | QPHKLPKPGF | 7509 |
| weighted | LMLSIKSTVQ | 7510 |
| weighted | NLKVESSLTE | 7511 |
| weighted | ELEMLKAQKI | 7512 |
| weighted | VVQVRFMVTA | 7513 |
| weighted | KSDLVAERTP | 7514 |
| weighted | CVELGSTLED | 7515 |
| weighted | GQEIRKGKAR | 7516 |
| weighted | LSLAFEEYVD | 7517 |
| weighted | GQLCKQSSYK | 7518 |
| weighted | LLQSCASHWS | 7519 |
| weighted | TERLSPGALT | 7520 |
| weighted | ELYEPAAKAY | 7521 |
| weighted | EEYDGCNGFP | 7522 |
| weighted | LDSAPSASER | 7523 |
| weighted | KTSQTLQPGN | 7524 |
| weighted | EAEPLLHLPV | 7525 |
| weighted | NVLKWATGNK | 7526 |
| weighted | MELQMPSTTN | 7527 |
| weighted | RLNSFDTKLS | 7528 |
| weighted | MNATSPKRRA | 7529 |
| weighted | LMRLVGKGAS | 7530 |
| weighted | PWIMKTQDYS | 7531 |
| weighted | EEACHAVQAL | 7532 |
| weighted | NTTLDSVILC | 7533 |
| weighted | YGMPYNLSGR | 7534 |
| weighted | HVHATQSDMA | 7535 |
| weighted | GDRAKFNRVL | 7536 |
| weighted | DKVRWPLEND | 7537 |
| weighted | LRDQPVMPVK | 7538 |
| weighted | GIKEVKPTMF | 7539 |
| weighted | YNNLETMKEY | 7540 |
| weighted | QFQVFATCQG | 7541 |
| weighted | TVCDHFPKEY | 7542 |
| weighted | HEWGKAPVGT | 754 |
| weighted | MKATSDAPEW | 7544 |
| weighted | TGRILHGSCE | 7545 |
| weighted | DIAEVALNAY | 7546 |
| weighted | LPVFRAYSIH | 7547 |
| weighted | QNSEEEYYTH | 7548 |
| weighted | LWPRQARIQE | 7549 |
| weighted | THESEERKPQ | 7550 |
| weighted | PELPLPELEV | 7551 |
| weighted | FIPHESGISC | 7552 |
| weighted | KHVGLKLDAL | 7553 |
| weighted | QVSLMLRLLP | 7554 |
| weighted | LLVPTNYCDK | 7555 |
| weighted | FCSMRVSYDK | 7556 |
| weighted | RLLLAACFGH | 7557 |
| weighted | TEGVGNPEPS | 7558 |
| weighted | HALTAGWPEG | 7559 |
| weighted | NALKVVFELL | 7560 |
| weighted | GMSSVRPKLL | 7561 |
| weighted | CERTFKYYTS | 7562 |
| weighted | ELAEAGQEYW | 7563 |
| weighted | SQADDVTPVL | 7564 |
| weighted | GGTFRLPVEV | 7565 |
| weighted | KIEKVLNFKH | 7566 |
| weighted | GDFSHGKVVI | 7567 |
| weighted | FPSLVIQPIL | 7568 |
| weighted | FRTLNTEAKN | 7569 |
| weighted | SKEDKLIKPI | 7570 |
| weighted | LVNRCLTTNF | 7571 |
| weighted | ETLALAERAP | 7572 |
| weighted | AQKQENLTQL | 7573 |
| weighted | VWFKQQQATA | 7574 |
| weighted | FAENIGLPRT | 7575 |
| weighted | EGQSTNVELS | 7576 |
| weighted | EDSWGSTNED | 7577 |
| weighted | SSELWEAHER | 7578 |
| weighted | EDHDPKQGGY | 7579 |
| weighted | EFHEYLSAQP | 7580 |
| weighted | GREKLGGGPV | 7581 |
| weighted | EVLGLLSGVV | 7582 |
| weighted | LVYVDMNFGN | 7583 |
| weighted | NDADFPEESE | 7584 |
| weighted | ATPIQPTMQK | 7585 |
| weighted | DVASVDFLSF | 7586 |
| weighted | SDANRAGLVA | 7587 |
| weighted | PRGDLDSAEP | 7588 |
| weighted | ICNFLDIQRL | 7589 |
| weighted | EPCPRPELYT | 7590 |
| weighted | YDIAHAQVDE | 7591 |
| weighted | LVDSTLADYV | 7592 |
| weighted | ILKIQLGLPFC | 7593 |
| weighted | RCSVKASTKN | 7594 |
| weighted | TASICPPGVK | 7595 |
| weighted | RLQSADEHFP | 7596 |
| weighted | FIDIKDWVGK | 7597 |
| weighted | LSIELSPRLE | 7598 |
| weighted | GLELQPGFPT | 7599 |
| weighted | ESQKALQHLF | 7600 |
| weighted | VTGTRDSNYQ | 7601 |
| weighted | LSVLLPPQLR | 7602 |
| weighted | SQATGLPPNT | 7603 |
| weighted | PSTEFCARRN | 7604 |
| weighted | VDPLVGYTEA | 7605 |
| weighted | SQVRAMREQS | 7606 |
| weighted | NGQFDVLAGL | 7607 |
| weighted | ISNRAQLVVL | 7608 |
| weighted | DEYPETGDED | 7609 |
| weighted | ACLLDQSYAT | 7610 |
| weighted | KADEHPAFAF | 7611 |
| weighted | KAVAERVYKI | 7612 |
| weighted | IPMPGPLWHG | 7613 |
| weighted | LGDTVQPTTP | 7614 |
| weighted | SSLSCSVPEL | 7615 |
| weighted | LYRDGIRDSC | 7616 |
| weighted | TSAYTNSCQT | 7617 |
| weighted | PREIITIATM | 7618 |
| weighted | DVPYRSFMGI | 7619 |
| weighted | GHSIKSSPKI | 7620 |
| weighted | SSPFPAAQTF | 7621 |
| weighted | FKEAQWDKPN | 7622 |
| weighted | HVVEYSEQNT | 7623 |
| weighted | LRQIRFLSTA | 7624 |
| weighted | STVTTEGLEY | 7625 |
| weighted | FCVFVKKPAL | 7626 |
| weighted | MKQIAGVSRV | 7627 |
| weighted | NEPSTLRSTK | 7628 |
| weighted | ANGQETLSQS | 7629 |
| weighted | SLSADINESY | 7630 |
| weighted | KAVSSAAKDL | 7631 |
| weighted | SAVMKNWYKD | 7632 |
| weighted | KMRPGLNQEE | 7633 |
| weighted | CIFSRMQADE | 7634 |
| weighted | NQPLTYFYEK | 7635 |
| weighted | LSEIIEENPL | 7636 |
| weighted | KNNTSKNYDQ | 7637 |
| weighted | SLLLWLPRSS | 7638 |
| weighted | FSGIPDMSSI | 7639 |
| weighted | ESSVHPLMLL | 7640 |
| weighted | HDESGASPKR | 7641 |
| weighted | PTSDRDATLC | 7642 |
| weighted | RKNIPPGSTV | 7643 |
| weighted | FVLVDGEVSD | 7644 |
| weighted | RLDQFVLGKR | 7645 |
| weighted | ALTPSFFCSI | 7646 |
| weighted | ASRDGPAVSK | 7647 |
| weighted | PPAKGVMKDI | 7648 |
| weighted | QAAHVSDCET | 7649 |
| weighted | CPLSDLSMSV | 7650 |
| weighted | REKPMIINLP | 7651 |
| weighted | KKAVIKPFGT | 7652 |
| weighted | ECGELHVVEK | 7653 |
| weighted | SAIVGVLMNQ | 7654 |
| weighted | DKMYFAEELF | 7655 |
| weighted | NFVLLTIMQE | 7656 |
| weighted | RLNKGACNCV | 7657 |
| weighted | LLSADQPPSN | 7658 |
| weighted | PNFVSESFRY | 7659 |
| weighted | VFLVAAPSFY | 7660 |
| weighted | LLECKSLYPM | 7661 |
| weighted | WTHRVDLSRQ | 7662 |
| weighted | KSLLVAPVKY | 7663 |
| weighted | PPKLLKDDAG | 7664 |
| weighted | PASVQQGCSK | 7665 |
| weighted | MALGDSICHS | 7666 |
| weighted | RAPPCVLVEL | 7667 |
| weighted | SLPRNHQGGL | 7668 |
| weighted | ARAVLKPDSA | 7669 |
| weighted | GFAEVMISID | 7670 |
| weighted | TLFSWKHCYT | 7671 |
| weighted | CLPYKILSVD | 7672 |
| weighted | VLKVGEGLRS | 7673 |
| weighted | VKYGYRNFEG | 7674 |
| weighted | REGKAVTSDF | 7675 |
| weighted | ALPLSFMTQM | 7676 |
| weighted | PGRVEGCPQM | 7677 |
| weighted | GEQKYKIRQP | 7678 |
| weighted | FLGKNLREKG | 7679 |
| weighted | KEGDSGLGLC | 7680 |
| weighted | VPVTAYARTS | 7681 |
| weighted | NDKGYERKSR | 7682 |
| weighted | WGGWRDMEAA | 7683 |
| weighted | RTLVTMGKPM | 7684 |
| weighted | NPAAWHATKS | 7685 |
| weighted | ICKANHKTTY | 7686 |
| weighted | PNSIPEPPDA | 7687 |
| weighted | GPSKGQCPLL | 7688 |
| weighted | TVESFVEKTR | 7689 |
| weighted | KPWLMGVLSF | 7690 |
| weighted | SFSLPCDQEC | 7691 |
| weighted | VRVEKREGCN | 7692 |
| weighted | TSGWVVVPRK | 7693 |
| weighted | PPNECFPVLE | 7694 |
| weighted | REQKCEGTCA | 7695 |
| weighted | SHTPHRSGSK | 7696 |
| weighted | RKVKSTLCLC | 7697 |
| weighted | SSHKPELPVK | 7698 |
| weighted | QYAFGPSELG | 7699 |
| weighted | QQVPGGDKFL | 7700 |
| weighted | GRLGRDLKAV | 7701 |
| weighted | SHCPARNSDH | 7702 |
| weighted | GVNTIAGHHL | 7703 |
| weighted | GIRIIQDQDF | 7704 |
| weighted | NSRSEAKCRA | 7705 |
| weighted | YPDELCQQEL | 7706 |
| weighted | DVLIRNPPGG | 7707 |
| weighted | CDSRMVEQFA | 7708 |
| weighted | RGLAGPKFYM | 7709 |
| weighted | ISPSRIQVDV | 7710 |
| weighted | PLTINRGCTL | 7711 |
| weighted | NRHAPPEVLL | 7712 |
| weighted | VTYSQDDSCG | 7713 |
| weighted | WNNNTREVKT | 7714 |
| weighted | SESNYYASVL | 7715 |
| weighted | PILKYVNSGL | 7716 |
| weighted | MGLAILSPGA | 7717 |
| weighted | TLSRSSKMSC | 7718 |
| weighted | SRQEELAEEN | 7719 |
| weighted | LRKHEHQRMQ | 7720 |
| weighted | AQPLPRTQDV | 7721 |
| weighted | SAELSLFFLS | 7722 |
| weighted | NLGRPLDGGR | 7723 |
| weighted | PMEGLAFSGI | 7724 |
| weighted | LSKWCTKGAE | 7725 |
| weighted | GAETILKISL | 7726 |
| weighted | AAYNSEVLMN | 7727 |
| weighted | KGREGQYCLC | 7728 |
| weighted | GLWPTLARPA | 7729 |
| weighted | VGSICPQYKA | 7730 |
| weighted | KAVFTLAKDP | 7731 |
| weighted | GGEPLIIKSS | 7732 |
| weighted | HPPSQETDRQ | 7733 |
| weighted | YWSIFTRWAL | 7734 |
| weighted | GHTHGALVQF | 7735 |
| weighted | CRSGFAHPFI | 7736 |
| weighted | IGPERLRLAK | 7737 |
| weighted | IFDPKGCQCS | 7738 |
| weighted | LQVDPPLSDK | 7739 |
| weighted | VHRLDESVIH | 7740 |
| weighted | ISNVTTGTDV | 7741 |
| weighted | TKQLSFAQEV | 7742 |
| weighted | CYKFLNRPES | 7743 |
| weighted | SMTTAPFRPV | 7744 |
| weighted | LALDIRPIVV | 7745 |
| weighted | FQRSIRKHNT | 7746 |
| weighted | PKFTGALGKF | 7747 |
| weighted | IAADLESGGG | 7748 |
| weighted | DPFLIKLKGS | 7749 |
| weighted | PFISAAAGST | 7750 |
| weighted | DDKTLEICSS | 7751 |
| weighted | FPKRVLTREL | 7752 |
| weighted | WAYRFKGDSV | 7753 |
| weighted | GSYACGASNE | 7754 |
| weighted | KELALACVEF | 7755 |
| weighted | LTFDFPDALR | 7756 |
| weighted | GDRLSTLTLR | 7757 |
| weighted | EHEDPFGQAM | 7758 |
| weighted | LPSNLEQEQV | 7759 |
| weighted | PAVFNDKLTT | 7760 |
| weighted | QRLKDQPGIK | 7761 |
| weighted | SETRILVWTP | 7762 |
| weighted | ASFTCSETYY | 7763 |
| weighted | YVPLSNLEEV | 7764 |
| weighted | ATSKVKQAWN | 7765 |
| weighted | EPWLAALLLY | 7766 |
| weighted | LAPRRSLPYM | 7767 |
| weighted | FCGQSSDWTV | 7768 |
| weighted | CPTKGDNSPL | 7769 |
| weighted | FVGFTMIPWQ | 7770 |
| weighted | NDSFNECLKM | 7771 |
| weighted | MRGIAKSWVS | 7772 |
| weighted | EFSCAYRTKK | 7773 |
| weighted | SLKVYRVFLA | 7774 |
| weighted | PRGGTLYSRN | 7775 |
| weighted | FPFAPREGTK | 7776 |
| weighted | ERKTILSYAI | 7777 |
| weighted | LQLYFGINGP | 7778 |
| weighted | AKSQFAQSPI | 7779 |
| weighted | EDFMLSLSDE | 7780 |
| weighted | KLQAPPKGER | 7781 |
| weighted | VMPSFERVKA | 7782 |
| weighted | QIYSMKDVEK | 7783 |
| weighted | LQPDEYDLFG | 7784 |
| weighted | VPFLHGKSQT | 7785 |
| weighted | GIYCILKIWG | 7786 |
| weighted | RLQRFIIMCI | 7787 |
| weighted | ILEESTQMLS | 7788 |
| weighted | EWASPISGCS | 7789 |
| weighted | SSEKLACELV | 7790 |
| weighted | RKQTQQTLPL | 7791 |
| weighted | IEFFAILFPA | 7792 |
| weighted | PVARLEFRSR | 7793 |
| weighted | IECHIPQVVA | 7794 |
| weighted | WSRAGSSTLA | 7795 |
| weighted | DFGFAQLRWP | 7796 |
| weighted | SFPNAPMTSC | 7797 |
| weighted | DKEMQELKMT | 7798 |
| weighted | LPVDALPKCV | 7799 |
| weighted | LLVPEHTRLP | 7800 |
| weighted | LCLMADHDIE | 7801 |
| weighted | YKNSHSRIIA | 7802 |
| weighted | QIVVTNPEFL | 7803 |
| weighted | AEDAFRASAG | 7804 |
| weighted | SQISDEIWQV | 7805 |
| weighted | HALTKFSQLP | 7806 |
| weighted | ADSARHAGQK | 7807 |
| weighted | AVPKAKLLGF | 7808 |
| weighted | APAEIDLKTF | 7809 |
| weighted | DRMSFATWLK | 7810 |
| weighted | MNSELHPGEL | 7811 |
| weighted | MFLCDLGWCM | 7812 |
| weighted | RDFDAEGTGL | 7813 |
| weighted | YFVSAACATH | 7814 |
| weighted | RSQHASLYSP | 7815 |
| weighted | GMEGDCEDGK | 7816 |
| weighted | RFPANGCIMT | 7817 |
| weighted | AKYHIKRGGN | 7818 |
| weighted | LRVLTKANRA | 7819 |
| weighted | RPRIYVSFAE | 7820 |
| weighted | YFKVYPENEP | 7821 |
| weighted | VVSLLPYNGG | 7822 |
| weighted | ETGRIDCCYE | 7823 |
| weighted | NVGGLDRSGG | 7824 |
| weighted | ISSQSEVKQE | 7825 |
| weighted | PGIYENTVLS | 7826 |
| weighted | QCKHDVGAPL | 7827 |
| weighted | GSLNSPFPVG | 7828 |
| weighted | PEADECPEQP | 7829 |
| weighted | EKETSLSFLW | 7830 |
| weighted | QVPVGTNDNM | 7831 |
| weighted | TQLLSLPPVI | 7832 |
| weighted | VLKSDNLLGI | 7833 |
| weighted | AAPDLPLAKR | 7834 |
| weighted | FTIINNVREV | 7835 |
| weighted | QFQWLVGSFN | 7836 |
| weighted | LNEASYSPRG | 7837 |
| weighted | SIKNGCDLFK | 7838 |
| weighted | LERRYPQRRL | 7839 |
| weighted | PGKWPIACDA | 7840 |
| weighted | ELVAVDVLHY | 7841 |
| weighted | VMVLHRLSFK | 7842 |
| weighted | VRDPTKQGTC | 7843 |
| weighted | WKNTPDDKQP | 7844 |
| weighted | THQDLPTLLF | 7845 |
| weighted | GIFSDERNMQ | 7846 |
| weighted | SSTVLIEPCA | 7847 |
| weighted | TNLSTLGLSV | 7848 |
| weighted | ILKPAKRLSP | 7849 |
| weighted | QGIHFLMDVN | 7850 |
| weighted | AGDNNKDRQV | 7851 |
| weighted | NLTKQLKPGS | 7852 |
| weighted | IVVFIEVNEP | 7853 |
| weighted | YQKSLTKQHP | 7854 |
| weighted | GGVNKLMPGP | 7855 |
| weighted | IFALSSGSTL | 7856 |
| weighted | TEVAPSEKHT | 7857 |
| weighted | SSSFLLSGRP | 7858 |
| weighted | TICRPDVWEG | 7859 |
| weighted | SAFPEKLSIA | 7860 |
| weighted | NQTRLSRVTE | 7861 |
| weighted | KGCQDPDQPP | 7862 |
| weighted | SGAQTYSVSL | 7863 |
| weighted | PFWKPSLCSQ | 7864 |
| weighted | GGAKVACRQM | 7865 |
| weighted | YISPPQVACK | 7866 |
| weighted | RPIKFVPFSE | 7867 |
| weighted | EYDIGLPKMD | 7868 |
| weighted | CKAGLAASLA | 7869 |
| weighted | DTNGRRAAKL | 7870 |
| weighted | TLRGIFMGIQ | 7871 |
| weighted | TERTQGGKFH | 7872 |
| weighted | LFVRENSTNL | 7873 |
| weighted | RLTDTEHERI | 7874 |
| weighted | RSTESWLFTI | 7875 |
| weighted | DNVPEEGEWR | 7876 |
| weighted | HRNYARFLTR | 7877 |
| weighted | NGLSQVELLY | 7878 |
| weighted | ESRASIARRK | 7879 |
| weighted | RPCKIMLHFN | 7880 |
| weighted | WFRRSLAMHA | 7881 |
| weighted | SLLAFTGRPV | 7882 |
| weighted | GQDLAYSTDR | 7883 |
| weighted | TSKLGSFLIK | 7884 |
| weighted | SMANAAQLSK | 7885 |
| weighted | GGEYSLLTYL | 7886 |
| weighted | TFGPWLNLGR | 7887 |
| weighted | WPMRNRYFNG | 7888 |
| weighted | DSNRWFAQVP | 7889 |
| weighted | CLNALGEREY | 7890 |
| weighted | LATVGSVCLM | 7891 |
| weighted | LKGEGRPVPP | 7892 |
| weighted | EKLKEEGPKH | 7893 |
| weighted | RKTVDPCPKT | 7894 |
| weighted | ECCVSTVLVG | 7895 |
| weighted | NWRYVGKQEP | 7896 |
| weighted | FRFFFEKAPR | 7897 |
| weighted | JEEPPSMGISS | 7898 |
| weighted | SSKSDTDLYH | 7899 |
| weighted | RMLWNKINLG | 7900 |
| weighted | QKMIIEKHVT | 7901 |
| weighted | SLRETTMLVL | 7902 |
| weighted | KLSLHYILAH | 7903 |
| weighted | EHVVFGSIIV | 7904 |
| weighted | KIPTVCTVRM | 7905 |
| weighted | SVKAGHQTFK | 7906 |
| weighted | HPAPLDTRLQ | 7907 |
| weighted | TDGAAQTEKS | 7908 |
| weighted | MLCTGHVIGP | 7909 |
| weighted | VCQERKADLC | 7910 |
| weighted | GPMSPVLPIK | 7911 |
| weighted | QCRALSVVTP | 7912 |
| weighted | PHSGEDYWAA | 7913 |
| weighted | RETPRHGANR | 7914 |
| weighted | IGLVKFNGKK | 7915 |
| weighted | IKVLSPRLGK | 7916 |
| weighted | IKFKGPAARR | 7917 |
| weighted | QTVMVYRHIQ | 7918 |
| weighted | EGAQNSNCAF | 7919 |
| weighted | KLSFAPDEMS | 7920 |
| weighted | QEFFLWVMGY | 7921 |
| weighted | SQTQATSTHK | 7922 |
| weighted | KTFTNEPKKV | 7923 |
| weighted | AVCPGQFKPD | 7924 |
| weighted | MSSERRSQQI | 7925 |
| weighted | RGGPQHYEHP | 7926 |
| weighted | GQTLAEVLGA | 7927 |
| weighted | KHLLGLGTWA | 7928 |
| weighted | KPHTVAPLNN | 7929 |
| weighted | PFEGLPIFPP | 7930 |
| weighted | YHHLNRAIDI | 7931 |
| weighted | GHNQVQDTQL | 7932 |
| weighted | SLSSLSLGEV | 7933 |
| weighted | PFEPRRAIVM | 7934 |
| weighted | ALPCADESLN | 7935 |
| weighted | FHLGGERVNK | 7936 |
| weighted | MDAPFKSPQR | 7937 |
| weighted | YLTAAAKDDQ | 7938 |
| weighted | AHLGVSEQFS | 7939 |
| weighted | QGTSISFWPK | 7940 |
| weighted | SDPAAKPAVL | 7941 |
| weighted | LFDLFEYIDL | 7942 |
| weighted | SVDNLPNTPE | 7943 |
| weighted | PNFVNLARAT | 7944 |
| weighted | YYKTDTAIPL | 7945 |
| weighted | APANSDRTYA | 7946 |
| weighted | RVKLVFPDFD | 7947 |
| weighted | LLPASAIIPE | 7948 |
| weighted | HEDKKAGCDQ | 7949 |
| weighted | HLQRRIPPKR | 7950 |
| weighted | DSHKCNQEFL | 7951 |
| weighted | EIQCTGASSV | 7952 |
| weighted | ITSVVAVSPN | 7953 |
| weighted | EICLRVPEGR | 7954 |
| weighted | RSNAKVLGDL | 7955 |
| weighted | SEHHGHLKKA | 7956 |
| weighted | TMDSRCDMRN | 7957 |
| weighted | AVWIAIVSKD | 7958 |
| weighted | VKLAEMQLWD | 7959 |
| weighted | PKLKIRGLDQ | 7960 |
| weighted | APKCTFLQSS | 7961 |
| weighted | MAATIKMTVP | 7962 |
| weighted | RMDLDGQQEG | 7963 |
| weighted | LQQSGNMAWV | 7964 |
| weighted | HQYQLETDPV | 7965 |
| weighted | WTKELQLVLK | 7966 |
| weighted | GVEINTSELY | 7967 |
| weighted | RYENQQMGHS | 7968 |
| weighted | TFFFQAMEQV | 7969 |
| weighted | DVAYELELEF | 7970 |
| weighted | MKPSKTRDLV | 7971 |
| weighted | AGPVESVNEA | 7972 |
| weighted | MEHEQEYECY | 7973 |
| weighted | PARHDSQESA | 7974 |
| weighted | IQQQPCFPGD | 7975 |
| weighted | IAIGPSAIVG | 7976 |
| weighted | QGPAKKLYET | 7977 |
| weighted | WWVQNWFTLE | 7978 |
| weighted | AQDLCKGAVD | 7979 |
| weighted | SHDENAQEPF | 7980 |
| weighted | SATGLTITPT | 7981 |
| weighted | CPHESPQETT | 7982 |
| weighted | DEGIRAAILS | 7983 |
| weighted | MGKLKQGQRD | 7984 |
| weighted | GHRHSRLHLY | 7985 |
| weighted | SKYDRLWFLE | 7986 |
| weighted | GDRYYKAAKK | 7987 |
| weighted | GESLRTINSR | 7988 |
| weighted | QTNTEMNKSI | 7989 |
| weighted | GPHVQKKDLL | 7990 |
| weighted | PQEWLTTLPN | 7991 |
| weighted | RPWVEPKSGH | 7992 |
| weighted | SRYQDLDTKR | 7993 |
| weighted | LMECSSQCFD | 7994 |
| weighted | GEWETFSPPQ | 7995 |
| weighted | CPILELGNVL | 7996 |
| weighted | LRLYYENLRV | 7997 |
| weighted | PRGRNQSGST | 7998 |
| weighted | GEQNAWKLKN | 7999 |
| weighted | AKRYPIKGRG | 8000 |
| weighted | IGEAQGEISD | 8001 |
| weighted | PLAFQGFKKV | 8002 |
| weighted | SLNLLDMKSQ | 8003 |
| weighted | ELTFVLLEDE | 8004 |
| weighted | FVLADTPSLE | 8005 |
| weighted | GGGMGLDDHSR | 8006 |
| weighted | LLARLSLCESN | 8007 |
| weighted | RLILEEIQANE | 8008 |
| weighted | FQAPGRYAAKR | 8009 |
| weighted | EDLGDSRGIRI | 8010 |
| weighted | VALEFKPESFN | 8011 |
| weighted | GHRGFHPKSST | 8012 |
| weighted | LSMAFRLVVLI | 8013 |
| weighted | SHIRDKYSERK | 8014 |
| weighted | GVHAVGQFLHA | 8015 |
| weighted | KILQFTFRYEF | 8016 |
| weighted | SATPGLECAEN | 8017 |
| weighted | FPHEYIHAKWK | 8018 |
| weighted | GTQLSYPDLRE | 8019 |
| weighted | GSKWESNHMTA | 8020 |
| weighted | TGTMCISVEVL | 8021 |
| weighted | GPITKKGTKNL | 8022 |
| weighted | EPDSTHGYNLD | 8023 |
| weighted | YGATGSHRIKA | 8024 |
| weighted | DEKVAILLCNE | 8025 |
| weighted | LFLTLLNLPSF | 8026 |
| weighted | TLYASEKHCLN | 8027 |
| weighted | QAMESFQPLSP | 8028 |
| weighted | SEAEDVRPERG | 8029 |
| weighted | MPMCVSNGDAT | 8030 |
| weighted | FPDVKIHCSEA | 8031 |
| weighted | KTQAGFGRTVC | 8032 |
| weighted | HEFVTCCFLDT | 8033 |
| weighted | PKGCAREDIQD | 8034 |
| weighted | RAGERTQYLSR | 8035 |
| weighted | TVDTALCCLVT | 8036 |
| weighted | CNKWGALPRTT | 8037 |
| weighted | QSYATFDAIRL | 8038 |
| weighted | NQDASKVVSRR | 8039 |
| weighted | DDIPFHGPPPR | 8040 |
| weighted | NKQSEYLRTLD | 8041 |
| weighted | YLCKMLPIKAL | 8042 |
| weighted | EMDIMFQTTFT | 8043 |
| weighted | PCSRDVTNDHE | 8044 |
| weighted | SLKMACDNEGG | 8045 |
| weighted | LNSWKIEVRMD | 8046 |
| weighted | QQSQSIPANVL | 8047 |
| weighted | VFAFPHRKLFR | 8048 |
| weighted | MESSHAMPSLF | 8049 |
| weighted | RLGAEEIDEPA | 8050 |
| weighted | AFYVRARIFLL | 8051 |
| weighted | LEVQTEVWKDT | 8052 |
| weighted | SCIDSSSLAAG | 8053 |
| weighted | TVSGLRNYARY | 8054 |
| weighted | NESYRAGAHPE | 8055 |
| weighted | LQMHYDALSDY | 8056 |
| weighted | EMRQGLMMSSL | 8057 |
| weighted | SSYQPEQGLTL | 8058 |
| weighted | GFTTSTHEFGP | 8059 |
| weighted | GMESLSGGKVV | 8060 |
| weighted | NSMLVPALTWF | 8061 |
| weighted | ARGANQGRIKW | 8062 |
| weighted | EPKTQNVQDRV | 8063 |
| weighted | ELVSFPNSPLK | 8064 |
| weighted | ALDSLFGVIYT | 8065 |
| weighted | LMTNGAPLPPA | 8066 |
| weighted | TRAQGKKYKFS | 8067 |
| weighted | SWMIAQGIPHN | 8068 |
| weighted | KCPALNPSPDT | 8069 |
| weighted | SLGPYGALEGL | 8070 |
| weighted | GLVTSDSINIL | 8071 |
| weighted | RSWLGQVKDLH | 8072 |
| weighted | VRSESSYYQYR | 8073 |
| weighted | ESIRELITVFV | 8074 |
| weighted | SRQPGIEPECF | 8075 |
| weighted | EDVLTEEQRSR | 8076 |
| weighted | QIPQSPANGKQ | 8077 |
| weighted | TCELCRCVPLS | 8078 |
| weighted | EDRGGNLQMDT | 8079 |
| weighted | AALTCPQLAQS | 8080 |
| weighted | MSLFIDLSAVH | 8081 |
| weighted | LANMFVQFPCN | 8082 |
| weighted | FPVHGQVKPIP | 8083 |
| weighted | IPQQPSPTVVL | 8084 |
| weighted | DTAELNIRQDP | 8085 |
| weighted | QCSGRLMVGLH | 8086 |
| weighted | ELEGISLSWEP | 8087 |
| weighted | WQMQLTSLKYA | 8088 |
| weighted | EPSENCRGMGL | 8089 |
| weighted | DLSSDRSKSFL | 8090 |
| weighted | RVLRCTALAQA | 8091 |
| weighted | EEKEEDCRNCA | 8092 |
| weighted | HGRDTRQSDYY | 8093 |
| weighted | NPPNLASSGAE | 8094 |
| weighted | QLPSNPDIDSS | 8095 |
| weighted | VSFAPSSVILK | 8096 |
| weighted | GEDVTNDPHTD | 8097 |
| weighted | EPSQFQSLPCD | 8098 |
| weighted | DIKDAINKTQV | 8099 |
| weighted | SQVPSTTVGEI | 8100 |
| weighted | EPKLFLYPATH | 8101 |
| weighted | LPGVVQATCSL | 8102 |
| weighted | VSLPHLDKFIY | 8103 |
| weighted | IEGMSGLVLKV | 8104 |
| weighted | FNLLEVVFGTI | 8105 |
| weighted | QLLARTVPLAL | 8106 |
| weighted | PKNQSAKNSPN | 8107 |
| weighted | YADEVAPAICR | 8108 |
| weighted | RSSTCVASPLL | 8109 |
| weighted | NRAGSADDNIS | 8110 |
| weighted | MPLKYCTGELF | 8111 |
| weighted | QLTDFLGEMQS | 8112 |
| weighted | RQLYFECVKQV | 8113 |
| weighted | DIKMTWLSTSL | 8114 |
| weighted | EKPEKFSKSLV | 8115 |
| weighted | KMSQSEEEASF | 8116 |
| weighted | LFLFQFEFGGA | 8117 |
| weighted | PHVKSNRLCNN | 8118 |
| weighted | SGRGLMVCDGQ | 8119 |
| weighted | VNTSHDTPLAY | 8120 |
| weighted | PTQRAKLNLFV | 8121 |
| weighted | GIEAISMEDWA | 8122 |
| weighted | EDMSQSEFCER | 8123 |
| weighted | SLAEVNAFPDN | 8124 |
| weighted | JEKLRFQGEGFS | 8125 |
| weighted | SWWFPVILNNS | 8126 |
| weighted | SELDDQWGEGL | 8127 |
| weighted | IHSRGSVTPSN | 8128 |
| weighted | IFKSLATPPPC | 8129 |
| weighted | TDGQFPPRVKL | 8130 |
| weighted | FRALKDAGNIL | 8131 |
| weighted | PQKGRGFSIEG | 8132 |
| weighted | KGTAFNRSSNP | 8133 |
| weighted | VWHVTFPGSSL | 8134 |
| weighted | VKNEEGIDNQL | 8135 |
| weighted | STYSSALRPGL | 8136 |
| weighted | DVMCLQIVRMC | 8137 |
| weighted | GGNAFTLRSFN | 8138 |
| weighted | FSNPPACETLF | 8139 |
| weighted | HTMMPKDFPPA | 8140 |
| weighted | GLERQRTACNK | 8141 |
| weighted | NMYPVGFWPEL | 8142 |
| weighted | FADVNKVLLDP | 8143 |
| weighted | ASHDMAQRPVG | 8144 |
| weighted | RQQGYQPSLLF | 8145 |
| weighted | KEMAPIFLHQS | 8146 |
| weighted | PTTKFWEGKEY | 8147 |
| weighted | SGLQAPIPSPL | 8148 |
| weighted | QEGYDGLSLKE | 8149 |
| weighted | IYSLWVRESNP | 8150 |
| weighted | ADQRSLTEKAG | 8151 |
| weighted | ASLPRAKQYTC | 8152 |
| weighted | LNQRFKEDDPD | 8153 |
| weighted | VDEDEKSLASR | 8154 |
| weighted | AAHLLMTRWPI | 8155 |
| weighted | PEGARNTTDAK | 8156 |
| weighted | VRGRVENKALI | 8157 |
| weighted | TATNSFTLFLI | 8158 |
| weighted | NISRPTSAVPE | 8159 |
| weighted | LLARQWVFQEL | 8160 |
| weighted | MQTQILGSEEE | 8161 |
| weighted | YYEAPDDHEMG | 8162 |
| weighted | LGTIEPRSSVS | 8163 |
| weighted | ICALHDFSDES | 8164 |
| weighted | QATLCDNSDSK | 8165 |
| weighted | SEQKLIQSCTK | 8166 |
| weighted | AHNPSHQCYSA | 8167 |
| weighted | RSTTGTLAVVC | 8168 |
| weighted | ADLIRRQQDYR | 8169 |
| weighted | FVNKLLESLKL | 8170 |
| weighted | SIIPVYWYKDP | 8171 |
| weighted | DASDAEIESYV | 8172 |
| weighted | STVLGLPVGLP | 8173 |
| weighted | FRSAPVILVPM | 8174 |
| weighted | DVVTAGNTALK | 8175 |
| weighted | YTGTLILHTIS | 8176 |
| weighted | GSFPPMSSEAG | 8177 |
| weighted | VARAAVISVNS | 8178 |
| weighted | PPFTLKYVDSH | 8179 |
| weighted | HRLPTKSEGMQ | 8180 |
| weighted | ESPYVEIKNVH | 8181 |
| weighted | EEILLVILTAF | 8182 |
| weighted | PILSALLHAER | 8183 |
| weighted | RLEAGHSGEAN | 8184 |
| weighted | RPTTSSLKDAA | 8185 |
| weighted | ERDREESHYSE | 8186 |
| weighted | MTSSVKVDGSS | 8187 |
| weighted | LVVDPANQQDT | 8188 |
| weighted | YRKSRSAGPKL | 8189 |
| weighted | SPAMPMRNAAG | 8190 |
| weighted | SKRELLNAVVS | 8191 |
| weighted | ECAIFVIYVQQ | 8192 |
| weighted | SFGFTSASFVT | 8193 |
| weighted | PRARLKRSNDI | 8194 |
| weighted | VLNKFIGWAQK | 8195 |
| weighted | ADAMCLSMHLD | 8196 |
| weighted | QKNRECISRAR | 8197 |
| weighted | PLEDTDAGSEG | 8198 |
| weighted | NFYDQLKLDSL | 8199 |
| weighted | VPQGINCLNIG | 8200 |
| weighted | PLVRTKLNKTL | 8201 |
| weighted | LCEWLLGKGGM | 8202 |
| weighted | KISVFASRLHN | 8203 |
| weighted | NVQGPRKVDYQ | 8204 |
| weighted | ELGFMNGKGDL | 8205 |
| weighted | DATTREGRPEW | 8206 |
| weighted | LIGKPSRAVKM | 8207 |
| weighted | TLDLLEPHQRL | 8208 |
| weighted | SKSPVKSVVLA | 8209 |
| weighted | SVGKFHHAGPE | 8210 |
| weighted | RLLSYDRSDIP | 8211 |
| weighted | CQLHSFTVSCS | 8212 |
| weighted | ASHYRNCALPV | 8213 |
| weighted | NESQGRDPPDE | 8214 |
| weighted | SPAQPKRARIM | 8215 |
| weighted | KYFPSASRVTA | 8216 |
| weighted | CSIASKVRIRF | 8217 |
| weighted | STLHLAEGNSP | 8218 |
| weighted | SWYESEFAGFR | 8219 |
| weighted | VKHEDRFKTKK | 8220 |
| weighted | SLNRGDTEIEA | 8221 |
| weighted | KTRKSQSTHIS | 8222 |
| weighted | PRQPAQVQVRS | 8223 |
| weighted | MATMVKFNRQQ | 8224 |
| weighted | LICHQERNPHL | 8225 |
| weighted | IKGVRSQGFEE | 8226 |
| weighted | TVNMGFLYLPN | 8227 |
| weighted | LGPPYVFAGAK | 8228 |
| weighted | PGDSDCVPIWC | 8229 |
| weighted | ACLTSTSKKGP | 8230 |
| weighted | IVQMIQRGAQK | 8231 |
| weighted | VALAHLKGTEP | 8232 |
| weighted | SVDTQNALQLS | 8233 |
| weighted | QRLFPPKEHIV | 8234 |
| weighted | PDALPIALNDE | 8235 |
| weighted | SENAIGGLLTC | 8236 |
| weighted | GQLLKTCGHCD | 8237 |
| weighted | SLIHFEYEAPP | 8238 |
| weighted | EASEVARSPDV | 8239 |
| weighted | VGDLDAVEIVY | 8240 |
| weighted | LTTGKLEPAMP | 8241 |
| weighted | VTPVPSQNQRL | 8242 |
| weighted | IAEADFRACVN | 8243 |
| weighted | SRDAQNRNLSG | 8244 |
| weighted | ILGLKKALLPAN | 8245 |
| weighted | IPENYWEYSRI | 8246 |
| weighted | EVQRQNYRVRG | 8247 |
| weighted | SNDDALPTQIV | 8248 |
| weighted | DMAAALIGKEG | 8249 |
| weighted | HQLHDESQRTD | 8250 |
| weighted | SRGKLNRFLRF | 8251 |
| weighted | MSTCDMMVAYG | 8252 |
| weighted | MYLVTPGAYQE | 8253 |
| weighted | KSFSTKVDDLS | 8254 |
| weighted | VVGMVKCEQRF | 8255 |
| weighted | SRGSTADVAEM | 8256 |
| weighted | LAFKQRSSDCK | 8257 |
| weighted | HATLNQVTQFS | 8258 |
| weighted | SVTKNLEFRGP | 8259 |
| weighted | LRVKAQDLWAY | 8260 |
| weighted | SMNCKGMKLKE | 8261 |
| weighted | VPVALAMEPTI | 8262 |
| weighted | VRPPDASKEDL | 8263 |
| weighted | KTSDQYYWLNG | 8264 |
| weighted | LSVKVETVKHY | 8265 |
| weighted | WAKENDCFDPF | 8266 |
| weighted | RALITWNRRSS | 8267 |
| weighted | VFCTMVGQCNL | 8268 |
| weighted | SDLLWFLGRLT | 8269 |
| weighted | LRDYISLPATV | 8270 |
| weighted | DVQKYVLNILC | 8271 |
| weighted | FKIPHNLDLKD | 8272 |
| weighted | QPPAGVFSGCK | 8273 |
| weighted | TGSARFVLLDR | 8274 |
| weighted | HKLQHRGVFPL | 8275 |
| weighted | IIPLCGPAEST | 8276 |
| weighted | CKAGQNETSRR | 8277 |
| weighted | QGASAQRRRSE | 8278 |
| weighted | MSLSTEKPMEA | 8279 |
| weighted | QLRSFPAATSK | 8280 |
| weighted | TKESHTLKTPT | 8281 |
| weighted | PQHFTFGGRLP | 8282 |
| weighted | CPADMGAMSQC | 8283 |
| weighted | PMEIERNSQKS | 8284 |
| weighted | DEAIEATLNDL | 8285 |
| weighted | KRYQSSLKYRY | 8286 |
| weighted | CVGLYPLDVSK | 8287 |
| weighted | ALFSNVPLKLA | 8288 |
| weighted | DLGLAPLAADS | 8289 |
| weighted | VLRVEHKEKAD | 8290 |
| weighted | KQCSVISCMLE | 8291 |
| weighted | SRSSARQQPEC | 8292 |
| weighted | GDEAIVPITFE | 8293 |
| weighted | YAHECCEETCI | 8294 |
| weighted | DGLETEGLGYE | 8295 |
| weighted | DIKSRLKLKAT | 8296 |
| weighted | AASGRHRMSLK | 8297 |
| weighted | LTCELPVNGFF | 8298 |
| weighted | TFPKGLASEKL | 8299 |
| weighted | TCQPQSLELVT | 8300 |
| weighted | LMVIWKSRKRI | 8301 |
| weighted | AAKSTCIVSEE | 8302 |
| weighted | DCDKDPLKEMA | 8303 |
| weighted | NNGAAAGDFPA | 8304 |
| weighted | ATIIRNIAASG | 8305 |
| weighted | ELSDTFMVSLR | 8306 |
| weighted | SNSYFCKSDDD | 8307 |
| weighted | SHMYPWEWFSM | 8308 |
| weighted | KDLRLTPATRS | 8309 |
| weighted | GNAGLKECCEC | 8310 |
| weighted | YSSKGDLQAMG | 8311 |
| weighted | TTPEDCRLEIF | 8312 |
| weighted | RDGDFGVGRNT | 8313 |
| weighted | SHLLPGFSFVA | 8314 |
| weighted | LRTAAAGKQGI | 8315 |
| weighted | WIREFKLFDTL | 8316 |
| weighted | WLAPEVGVKKA | 8317 |
| weighted | RKRGGGTSRAI | 8318 |
| weighted | IAAYDILQWEV | 8319 |
| weighted | CFEWHSNSKTE | 8320 |
| weighted | AQAFAWIPHVV | 8321 |
| weighted | SLGFIAGYIKA | 8322 |
| weighted | RWQGTAKASAS | 8323 |
| weighted | MQCNVQDSEDT | 8324 |
| weighted | RQQFDLRVQEV | 8325 |
| weighted | AQVDMTDAASG | 8326 |
| weighted | KIPKCNSNTAA | 8327 |
| weighted | LKIIGKSPYLE | 8328 |
| weighted | EKAKEDSGERV | 8329 |
| weighted | LQYRQEFPKLV | 8330 |
| weighted | TFLITPLILAL | 8331 |
| weighted | PGTATQTRCTK | 8332 |
| weighted | DEQFQQRCGSQ | 8333 |
| weighted | TEQDIEGRSRD | 8334 |
| weighted | RFFCNRETMIT | 8335 |
| weighted | NKKYSHPMPHA | 8336 |
| weighted | ESTPLASLKKV | 8337 |
| weighted | PGKDDLQKRAK | 8338 |
| weighted | QLTEPLSDFRL | 8339 |
| weighted | AELTGHEGRTS | 8340 |
| weighted | CTDIESATIEY | 8341 |
| weighted | KELPHKIVEVF | 8342 |
| weighted | NVKKVLPVITR | 8343 |
| weighted | SVSEPCLARSH | 8344 |
| weighted | DFVGVHDMLEK | 8345 |
| weighted | RLCSRPPSICT | 8346 |
| weighted | ILLVLTLIRVP | 8347 |
| weighted | FLDGSKKLRAM | 8348 |
| weighted | QRNLLFVGLSV | 8349 |
| weighted | PQDDTLLSRVS | 8350 |
| weighted | AYCLIGSSSPR | 8351 |
| weighted | MNVELPGDTKS | 8352 |
| weighted | AGTVQQMTYVG | 8353 |
| weighted | PPEYFALEMAG | 8354 |
| weighted | MKLKIMDSNRF | 8355 |
| weighted | VIYNLIGQGRK | 8356 |
| weighted | QSLLPFSCDMV | 8357 |
| weighted | RCEQKPGPIAA | 8358 |
| weighted | KSDEKHERNLT | 8359 |
| weighted | PNAPDALDGFQ | 8360 |
| weighted | GESLHSPARSP | 8361 |
| weighted | LIPASLTRDNL | 8362 |
| weighted | RNSDSFLAGDK | 8363 |
| weighted | NSIARSLPSPR | 8364 |
| weighted | GHKEHLPEMGP | 8365 |
| weighted | VCNALSIKARV | 8366 |
| weighted | GDFDHPASSLL | 8367 |
| weighted | TWGNITRSPIP | 8368 |
| weighted | SGRILDSLEQI | 8369 |
| weighted | GNRLRHEDVGF | 8370 |
| weighted | PDLTRLYGRSH | 8371 |
| weighted | KPLANKGIENR | 8372 |
| weighted | PNEDVQRDGLA | 8373 |
| weighted | ISLFGVGDKEG | 8374 |
| weighted | TIRVSAYDVLT | 8375 |
| weighted | EAHIFTNIVEA | 8376 |
| weighted | EWDNYSRRAGS | 8377 |
| weighted | GEEGCRVRRIG | 8378 |
| weighted | LFSYFIHYYIA | 8379 |
| weighted | SLALENTTPYL | 8380 |
| weighted | EKKPERQGSML | 8381 |
| weighted | VLPTIPESEKQ | 8382 |
| weighted | NHSLPFAYLKS | 8383 |
| weighted | QAVGLNDTVKL | 8384 |
| weighted | QLDPKQESIEL | 8385 |
| weighted | GFARKSAVDEN | 8386 |
| weighted | ILRMPQIICKL | 8387 |
| weighted | SGQSVQLHSAL | 8388 |
| weighted | FECPLGPRWAL | 8389 |
| weighted | LLAFQTPTGDG | 8390 |
| weighted | KGLIQKLKRLI | 8391 |
| weighted | YGVNLNLGSPR | 8392 |
| weighted | QCMDMPMMGDC | 8393 |
| weighted | SAVTKRRIIDR | 8394 |
| weighted | LKLSAVASAPL | 8395 |
| weighted | ARRLEDYIPHK | 8396 |
| weighted | INCLQGLNTVP | 8397 |
| weighted | CRLPCINSRLP | 8398 |
| weighted | PSICNCAFNAA | 8399 |
| weighted | GAPCRMVLNRP | 8400 |
| weighted | ISASKWRPNNT | 8401 |
| weighted | APSMEVGDMER | 8402 |
| weighted | PLQGNAAKDDT | 8403 |
| weighted | PHLEVSLLPTR | 8404 |
| weighted | AVWSIQDGNRM | 8405 |
| weighted | KGTDFAIKNSL | 8406 |
| weighted | WDLRLSQDIQA | 8407 |
| weighted | TAPKGPSWTEG | 8408 |
| weighted | VICVGDPTAPL | 8409 |
| weighted | DGELDRYMGTA | 8410 |
| weighted | PTAGKELKASR | 8411 |
| weighted | MNFLGHRKPAG | 8412 |
| weighted | VFGILHFCSSV | 8413 |
| weighted | INGRSPSEQRSL | 8414 |
| weighted | SEETILLFSGL | 8415 |
| weighted | HEDLRTEPTLE | 8416 |
| weighted | TGLPQEPTEFP | 8417 |
| weighted | VDASVYRLKKT | 8418 |
| weighted | FASRSLEFQDS | 8419 |
| weighted | FIIVARVISFF | 8420 |
| weighted | QEYNGLPPMRS | 8421 |
| weighted | SVMAGDKLNSV | 8422 |
| weighted | TQELRRVNVLA | 8423 |
| weighted | AEPQTLYGCGV | 8424 |
| weighted | KSTNGPLLGIR | 8425 |
| weighted | GSNTGEARYEG | 8426 |
| weighted | VVLTKIEQTAL | 8427 |
| weighted | VIKLDLPTAKL | 8428 |
| weighted | KLGYGVNRNMS | 8429 |
| weighted | RSIVGQLSPES | 8430 |
| weighted | SLGSFRLVYMP | 8431 |
| weighted | PQLSRNDITDI | 8432 |
| weighted | SQTRELNSMPC | 8433 |
| weighted | PQDHEIPGVAR | 8434 |
| weighted | ELQKTPSRMAT | 8435 |
| weighted | QLMGSQNDWLG | 8436 |
| weighted | SQGLIHVADIP | 8437 |
| weighted | YRRELPESHSC | 8438 |
| weighted | APLYLRCSGQA | 8439 |
| weighted | ATEWLVIPDKY | 8440 |
| weighted | ENHSQKMTMQP | 8441 |
| weighted | FDSIQEVSTQQ | 8442 |
| weighted | GDHFKGIIELL | 8443 |
| weighted | SGAEGGSNVGS | 8444 |
| weighted | WQPFTELDYAK | 8445 |
| weighted | LRHPCKNCFGL | 8446 |
| weighted | NAGCMQNRGLG | 8447 |
| weighted | PAVINWPSGLA | 8448 |
| weighted | LLYFLFIPIRS | 8449 |
| weighted | SRNGVLLKSHA | 8450 |
| weighted | YNPPKLENPMA | 8451 |
| weighted | LEVRYHHTYQM | 8452 |
| weighted | ECPASYVLKNC | 8453 |
| weighted | QGAAIYDDVDT | 8454 |
| weighted | VLCRTYAISIF | 8455 |
| weighted | THRFGLVNCEE | 8456 |
| weighted | ILVAYQEKSTV | 8457 |
| weighted | EWLVGEPRDAA | 8458 |
| weighted | QTLKKSLVLEF | 8459 |
| weighted | QSSKRPEGSSL | 8460 |
| weighted | EDSEHFPYKAM | 8461 |
| weighted | KEACLGLSKRH | 8462 |
| weighted | QAETSVPIGVR | 8463 |
| weighted | VSAELEMLHSL | 8464 |
| weighted | QVVPRSELPVR | 8465 |
| weighted | TRFVDQLHVFL | 8466 |
| weighted | CVIDSQDTRTL | 8467 |
| weighted | TGLLGSTHGVS | 8468 |
| weighted | LSLKGRSQSMH | 8469 |
| weighted | EGYNVAERLSH | 8470 |
| weighted | VPVVVYHELKR | 8471 |
| weighted | CKLSSIRKMSV | 8472 |
| weighted | SGTQCRCRITP | 8473 |
| weighted | TSCLFFAEAIF | 8474 |
| weighted | QKNSEGSLTAM | 8475 |
| weighted | KCLTRHGVNFA | 8476 |
| weighted | DCGAERCQRKA | 8477 |
| weighted | SLGNHGAVRRP | 8478 |
| weighted | FLVQLLSVTPL | 8479 |
| weighted | KIKVNSAVKLG | 8480 |
| weighted | EAVEVMVLRLF | 8481 |
| weighted | PVLVAGTSQLV | 8482 |
| weighted | EAAQQAQSGFT | 8483 |
| weighted | SVQGGRKKTKH | 8484 |
| weighted | DDTCVNMNQTH | 8485 |
| weighted | NNVPVASNEGQ | 8486 |
| weighted | PLIQVSFGTQK | 8487 |
| weighted | THPNRGYESNP | 8488 |
| weighted | ESEIRPNGEGA | 8489 |
| weighted | SNLRKRAHKIE | 8490 |
| weighted | LSFLATATYRV | 8491 |
| weighted | YESQHYRVRVC | 8492 |
| weighted | AGGSSPMNLEE | 8493 |
| weighted | GGLSWSVRDYQ | 8494 |
| weighted | LERPQQQKCKA | 8495 |
| weighted | REPPGMLLTHE | 8496 |
| weighted | LEPRATDLQPK | 8497 |
| weighted | LFAVAHLTQLW | 8498 |
| weighted | TTLKQKLGSYI | 8499 |
| weighted | TIFRRCRFGKN | 8500 |
| weighted | LAKFEMIIHPV | 8501 |
| weighted | DEVLSASKSWV | 8502 |
| weighted | INRECPLRQFM | 8503 |
| weighted | SPFSHTSSFNS | 8504 |
| weighted | LEFITRPPMYV | 8505 |
| weighted | VNPQELECEKV | 8506 |
| weighted | IARPLQPGEDP | 8507 |
| weighted | LVRLVLITSVE | 8508 |
| weighted | PICLQPPKNQE | 8509 |
| weighted | DENASHADYAR | 8510 |
| weighted | SLQARSADELV | 8511 |
| weighted | GDCVKCQLRPT | 8512 |
| weighted | EKVPAHLQLPK | 8513 |
| weighted | EPQSDGMFSDA | 8514 |
| weighted | EGSEVDYTEGE | 8515 |
| weighted | GLCAPYSPQQP | 8516 |
| weighted | KGVPYEEDGVR | 8517 |
| weighted | KTSQSKQQSRD | 8518 |
| weighted | ASNHYATEITT | 8519 |
| weighted | PGHYYQILGHG | 8520 |
| weighted | LTWLTVGIDTC | 8521 |
| weighted | KKVGEMFYCRA | 8522 |
| weighted | RLIYPVVLAQD | 8523 |
| weighted | KTGRHVKDIHS | 8524 |
| weighted | GDQAGQACKQV | 8525 |
| weighted | SPKKKKFEQFD | 8526 |
| weighted | GQVGAQQVSSI | 8527 |
| weighted | TKRPNPVLLKN | 8528 |
| weighted | SVETEDNFAWV | 8529 |
| weighted | SVDAGNLNTQL | 8530 |
| weighted | AKAPGCHSNNY | 8531 |
| weighted | APRVGKQSKSL | 8532 |
| weighted | CRDAERLLSDP | 8533 |
| weighted | SDESAADEISS | 8534 |
| weighted | SPGVIMWEVEY | 8535 |
| weighted | SGGAGLAWVQT | 8536 |
| weighted | AKCEEPPLSPQ | 8537 |
| weighted | SVQVHYGKGRE | 8538 |
| weighted | YPPILLAKYLI | 8539 |
| weighted | MPYTRVPGIAS | 8540 |
| weighted | IYFFEVRDYAP | 8541 |
| weighted | SVIQPEGRTGS | 8542 |
| weighted | GLQPSRRPTQI | 8543 |
| weighted | ELPFRGGKRVY | 8544 |
| weighted | SKSTARRLYRI | 8545 |
| weighted | KGGYFKTCKCS | 8546 |
| weighted | ACCQPASLWVH | 8547 |
| weighted | LLSCNSVLVAI | 8548 |
| weighted | NIKRTVSVVTE | 8549 |
| weighted | SVMATLLAKVY | 8550 |
| weighted | CEQWVPKPAES | 8551 |
| weighted | TAAGQNSQMAQ | 8552 |
| weighted | EGGHDSVQGGN | 8553 |
| weighted | ICMQWATREFP | 8554 |
| weighted | KRLNIEPQRKE | 8555 |
| weighted | SPARHSEEQDP | 8556 |
| weighted | RGSWSLGVHFM | 8557 |
| weighted | PVLGLEHYFAI | 8558 |
| weighted | HKIPAPGSVNM | 8559 |
| weighted | YDEPAESVNLE | 8560 |
| weighted | LILALIETNLG | 8561 |
| weighted | MTKHSSEDVLS | 8562 |
| weighted | AADEIYSHPTF | 8563 |
| weighted | YAGFSALVTLN | 8564 |
| weighted | AMIVRLGFQPL | 8565 |
| weighted | PVRKCSQEREK | 8566 |
| weighted | FDTNTIKLFRI | 8567 |
| weighted | SPNKVICARYP | 8568 |
| weighted | PNHALVWPHGS | 8569 |
| weighted | RRDEPKVSGGA | 8570 |
| weighted | VCQSVSEFLAQ | 8571 |
| weighted | LRRGLNYSQIQ | 8572 |
| weighted | VGSTPLTELTS | 8573 |
| weighted | PPLAVPCAELE | 8574 |
| weighted | KLLGSKGKIRS | 8575 |
| weighted | GMKPMKDDEID | 8576 |
| weighted | VAWSTIFTHLI | 8577 |
| weighted | INCKLSNSKHL | 8578 |
| weighted | SGPTDRMIREN | 8579 |
| weighted | LAYVVKRLVTQ | 8580 |
| weighted | NGRLLVARKAY | 8581 |
| weighted | LLTGPDPAWGD | 8582 |
| weighted | PQPSFRALSSL | 8583 |
| weighted | SDKSRALGCHA | 8584 |
| weighted | VSDGREPMSER | 8585 |
| weighted | LLNYKTDDTKP | 8586 |
| weighted | FAVRSQEEITP | 8587 |
| weighted | PVANTGKPAVE | 8588 |
| weighted | PWSPKMAIVCG | 8589 |
| weighted | CLCSHVPIWDR | 8590 |
| weighted | KTYSPPLTTYL | 8591 |
| weighted | HLLGIEGCKLQ | 8592 |
| weighted | CLPGQCGAPAA | 8593 |
| weighted | ANKPGRNPGRR | 8594 |
| weighted | LKDQSIPELDD | 8595 |
| weighted | QAIPWPNPIKL | 8596 |
| weighted | RMEIKASDVMQ | 8597 |
| weighted | SMHIYSMQEGL | 8598 |
| weighted | LCGVANEDSEK | 8599 |
| weighted | KKKEEGSPTKW | 8600 |
| weighted | SGPTKTTIYLR | 8601 |
| weighted | AFLVNEAFMVT | 8602 |
| weighted | QTHTGTHSMAN | 8603 |
| weighted | HDAFGSCRVCL | 8604 |
| weighted | PVYTSSRILPP | 8605 |
| weighted | PVIGIGKFGVW | 8606 |
| weighted | SGQNRLRSHSE | 8607 |
| weighted | TQYVKDPACHW | 8608 |
| weighted | HLSLLGDELLN | 8609 |
| weighted | RQSLSIEEFSR | 8610 |
| weighted | ARAPDDVCKDR | 8611 |
| weighted | LFSRDQIREES | 8612 |
| weighted | RRLIWDILIGS | 8613 |
| weighted | ALAVERCFDER | 8614 |
| weighted | RLEVSILGMRS | 8615 |
| weighted | SSEQRQGKEQN | 8616 |
| weighted | TSQLLYAEESI | 8617 |
| weighted | QLLKAHPLASY | 8618 |
| weighted | SQVILKGAVRD | 8619 |
| weighted | ACQNKDSMEVL | 8620 |
| weighted | LNLLFLIIEEC | 8621 |
| weighted | SSVFNYAEEES | 8622 |
| weighted | EEVTGPAISVV | 8623 |
| weighted | SGFEYPRSKDF | 8624 |
| weighted | KYASPPVSLQM | 8625 |
| weighted | SLNDLASTIWN | 8626 |
| weighted | IQLYDGPEVPL | 8627 |
| weighted | QRCDVMNDSTV | 8628 |
| weighted | LTGPSREYHVI | 8629 |
| weighted | LRPAMERDQSF | 8630 |
| weighted | IKEESFKATGS | 8631 |
| weighted | QLSGGEPSTAY | 8632 |
| weighted | KTSQKEVTITL | 8633 |
| weighted | QFNFDSKEKRY | 8634 |
| weighted | VGRVFHSKLPA | 8635 |
| weighted | TPLILKEYDDR | 8636 |
| weighted | KIGRSPEVLVD | 8637 |
| weighted | LAAHFELAAQA | 8638 |
| weighted | AAGCELSIREI | 8639 |
| weighted | PRNYKPSQIYD | 8640 |
| weighted | ILGVNVMGDCG | 8641 |
| weighted | PHSNAEKLLGF | 8642 |
| weighted | FNTDPVPAVLQ | 8643 |
| weighted | LIFVKFLPPSA | 8644 |
| weighted | QFESMNSYASS | 8645 |
| weighted | NLIMSDQQGVL | 8646 |
| weighted | QRQPLCIALNM | 8647 |
| weighted | JEDARTDLKPPA | 8648 |
| weighted | VFLIGDEDVRK | 8649 |
| weighted | ASTRSFQQLYE | 8650 |
| weighted | IIIAETQLPPS | 8651 |
| weighted | AGNTESESYLQ | 8652 |
| weighted | TVELLSSNTNL | 8653 |
| weighted | LGTHFLKCGAW | 8654 |
| weighted | SPPRCFQAEPG | 8655 |
| weighted | ELVFEPEPLGA | 8656 |
| weighted | SAPEEHEYLTK | 8657 |
| weighted | RGLYITCLYAA | 8658 |
| weighted | VPQPIRSRNVI | 8659 |
| weighted | PSSYMLGMAQI | 8660 |
| weighted | QDHDKIPNPFS | 8661 |
| weighted | YMTGLWLLVPW | 8662 |
| weighted | QASFISRTQSL | 8663 |
| weighted | DTWGSWTIEFQ | 8664 |
| weighted | EASAVRSEFQA | 8665 |
| weighted | HVKFVKPELYA | 8666 |
| weighted | RNPIMEQQGSL | 8667 |
| weighted | PDWVVPHSSET | 8668 |
| weighted | TRLAKGHIMGQ | 8669 |
| weighted | YVSSPSERDWA | 8670 |
| weighted | IQNCKDTATVL | 8671 |
| weighted | FTHWSLEGIKT | 8672 |
| weighted | LEIVELFRSVG | 8673 |
| weighted | LVQNAGIMSRN | 8674 |
| weighted | AERRGLRNAEE | 8675 |
| weighted | WKGIFGAEWVQ | 8676 |
| weighted | GEGCNPMEAQL | 8677 |
| weighted | IHLSGDRKAVL | 8678 |
| weighted | CSVAGMGVLQQ | 8679 |
| weighted | KGQTYLARVQK | 8680 |
| weighted | YSSTNEDPHSR | 8681 |
| weighted | TLTSQVDQQHE | 8682 |
| weighted | IPGTMKYEQRA | 8683 |
| weighted | ENVQGKIRRPE | 8684 |
| weighted | RESRPGLRGQD | 8685 |
| weighted | ADRLPVLVKSI | 8686 |
| weighted | NFSRIAEEIHA | 8687 |
| weighted | AGESNSNVQWD | 8688 |
| weighted | ELTRVVAKSCP | 8689 |
| weighted | TLGTKKKILDL | 8690 |
| weighted | VKLMMETAGQV | 8691 |
| weighted | GIKANRNKMYI | 8692 |
| weighted | PESGTRKLPKP | 8693 |
| weighted | NKTACKSNVRR | 8694 |
| weighted | QMTRKSVRSDD | 8695 |
| weighted | FGKQWYASGKA | 8696 |
| weighted | IESAKYDIAGM | 8697 |
| weighted | SETREKLENSH | 8698 |
| weighted | DHGVRRPEEKI | 8699 |
| weighted | WPDLTWTPSKY | 8700 |
| weighted | EATGRTSVSVV | 8701 |
| weighted | DGKVSVLPLGN | 8702 |
| weighted | YRRVEKSQAPH | 8703 |
| weighted | EHIFTKGAPAF | 8704 |
| weighted | ALKTTVELPDP | 8705 |
| weighted | HTSAASQAPER | 8706 |
| weighted | DIELVEDVKRK | 8707 |
| weighted | GGNITSESETL | 8708 |
| weighted | PARRTSGPLIR | 8709 |
| weighted | LLLMCFCAPVL | 8710 |
| weighted | FTEDPANSEMQ | 8711 |
| weighted | SMSGARVERGV | 8712 |
| weighted | THSLGPELSQI | 8713 |
| weighted | EEELAIKAFTH | 8714 |
| weighted | SEVVYFPQKGG | 8715 |
| weighted | NACYSPRSEVT | 8716 |
| weighted | SELPLQPGSEF | 8717 |
| weighted | GGNMQREPWAE | 8718 |
| weighted | KFAELVRCAYS | 8719 |
| weighted | ICGDAEQSVVE | 8720 |
| weighted | LHDGQASIDFE | 8721 |
| weighted | HKLQAEKTEAD | 8722 |
| weighted | GTTTIMMRKFE | 8723 |
| weighted | LSEQLDSPGPL | 8724 |
| weighted | NIGTSSENTPN | 8725 |
| weighted | TYILALMGQTF | 8726 |
| weighted | YEFRSFCYGVQ | 8727 |
| weighted | EPTELASLFTA | 8728 |
| weighted | RPLSGEVYVAN | 8729 |
| weighted | AATLCLCDSGS | 8730 |
| weighted | YWYVTEAFTVQ | 8731 |
| weighted | ILRVEYPLLMFL | 8732 |
| weighted | SALIHEGTLMF | 8733 |
| weighted | ESYYSGPTSDQ | 8734 |
| weighted | VKASMDGQAFW | 8735 |
| weighted | RVVLEPRSRVG | 8736 |
| weighted | PVGNLKYTVRE | 8737 |
| weighted | AQLNINIETQP | 8738 |
| weighted | LLQLDGDITGV | 8739 |
| weighted | GIGADRCPGTH | 8740 |
| weighted | RAETNALLRFA | 8741 |
| weighted | KIPSVTVVHLG | 8742 |
| weighted | LGLELGLTCTY | 8743 |
| weighted | VQDAQSASKKK | 8744 |
| weighted | WSDIFAARQQI | 8745 |
| weighted | MEGPRIQPLLG | 8746 |
| weighted | LTTTKATGQQK | 8747 |
| weighted | MQMVNRSFKHK | 8748 |
| weighted | ARINQLRLGIV | 8749 |
| weighted | IKEEYTPPEAA | 8750 |
| weighted | QPIPYNAQKGG | 8751 |
| weighted | ISPMHPASIAH | 8752 |
| weighted | NIVVSCHTAFL | 8753 |
| weighted | TEVFKQAFPLV | 8754 |
| weighted | SVPLTGCSEAI | 8755 |
| weighted | EDGTTRYGHMP | 8756 |
| weighted | WYVVCQLSMDN | 8757 |
| weighted | KEFGEGAIIMQ | 8758 |
| weighted | LRFREVLLNFG | 8759 |
| weighted | LKEQVPASGWK | 8760 |
| weighted | DNLLGTYSRNL | 8761 |
| weighted | TSFSGFQSLGL | 8762 |
| weighted | YSVVVRKAMYR | 8763 |
| weighted | LVFKGCFPGSH | 8764 |
| weighted | GLMVEKTYIHF | 8765 |
| weighted | VGQGRRFLRLK | 8766 |
| weighted | NALVIHLPTIC | 8767 |
| weighted | LATGALEGLYA | 8768 |
| weighted | QDRMKTGLGTP | 8769 |
| weighted | LSWNNSEQLSK | 8770 |
| weighted | IPLGPRGVREL | 8771 |
| weighted | LSLQPPMELLK | 8772 |
| weighted | VKVLVSMMWVL | 8773 |
| weighted | RMCDLWEVLGT | 8774 |
| weighted | RFFVVEIAPSG | 8775 |
| weighted | HRSDWEGGHEA | 8776 |
| weighted | WEFFDGGSVDG | 8777 |
| weighted | PFEKLAIAPIN | 8778 |
| weighted | YSRSEEIGGQS | 8779 |
| weighted | LAPVSSELVSE | 8780 |
| weighted | EDPGYKTSRTF | 8781 |
| weighted | LQKSQIEKDKS | 8782 |
| weighted | TCCILDKRKDT | 8783 |
| weighted | KQHELGLIFEW | 8784 |
| weighted | HEPAIIDVAPF | 8785 |
| weighted | LKVGGPEQEKF | 8786 |
| weighted | LVRILILNILS | 8787 |
| weighted | GQAVAPQSQAP | 8788 |
| weighted | NRAWWTSIQRT | 8789 |
| weighted | ESENDQPLDGF | 8790 |
| weighted | VHCNYDSLGSN | 8791 |
| weighted | THREESIHLSG | 8792 |
| weighted | KVTYILEVSHF | 8793 |
| weighted | RLPPVITQNSD | 8794 |
| weighted | EVKVTTPLQTL | 8795 |
| weighted | MTLLCPDAVRA | 8796 |
| weighted | AGTTEPTGEDK | 8797 |
| weighted | CTTYTGLQSLI | 8798 |
| weighted | ARTGCLKPKAY | 8799 |
| weighted | TSPLLRNILFL | 8800 |
| weighted | AEALTRFKGSW | 8801 |
| weighted | AICQSPGARYA | 8802 |
| weighted | TVRVKKHTNST | 8803 |
| weighted | LMDYGLSLPPS | 8804 |
| weighted | GTKMEHPCGRS | 8805 |
| weighted | PKLYCSPRCQE | 8806 |
| weighted | LQKWLGYCFGE | 8807 |
| weighted | EHYQNEATLLI | 8808 |
| weighted | VEVVTNAQKHK | 8809 |
| weighted | HTRMLRVKPQR | 8810 |
| weighted | GSEPDEYDDLS | 8811 |
| weighted | FADFGFLRGLY | 8812 |
| weighted | ICKGFTVLHYP | 8813 |
| weighted | SICTIPGPNIV | 8814 |
| weighted | AFQARKEKKRK | 8815 |
| weighted | FMPSTRHVQPA | 8816 |
| weighted | GLGPAPGWLIE | 8817 |
| weighted | LQGTLLPYGLV | 8818 |
| weighted | GCSACDSALEP | 8819 |
| weighted | ILTTLCLDSGR | 8820 |
| weighted | KVLPFVGWEVK | 8821 |
| weighted | MLLPDDLSYHL | 8822 |
| weighted | RSSELAHCVRA | 8823 |
| weighted | SLPRDLIVPIY | 8824 |
| weighted | AALAEEIRFYS | 8825 |
| weighted | NTLKLWPDTKA | 8826 |
| weighted | EDGARHPYGSP | 8827 |
| weighted | MQFPEQVGLLK | 8828 |
| weighted | NPFVLEHLVIE | 8829 |
| weighted | EPPKEGGVVLL | 8830 |
| weighted | RNECKCVTIVL | 8831 |
| weighted | GGLPSPLGDYL | 8832 |
| weighted | ESNQRIFEVLH | 8833 |
| weighted | SRLGLEILPYV | 8834 |
| weighted | ESTLLASTLRE | 8835 |
| weighted | LQHASTLPFLL | 8836 |
| weighted | VTKATPPHGGE | 8837 |
| weighted | CQYRSEALDPL | 8838 |
| weighted | YIPGRKIELST | 8839 |
| weighted | QEGKDGRASEA | 8840 |
| weighted | RVRRKNATELP | 8841 |
| weighted | EGWQRSKESKH | 8842 |
| weighted | WLSYPGLNRSM | 8843 |
| weighted | SESTLTLEKLG | 8844 |
| weighted | CVYFNPEYCNG | 8845 |
| weighted | SGPTLTKGGDD | 8846 |
| weighted | KGNKSLSATEL | 8847 |
| weighted | RKPVLRGKPHR | 8848 |
| weighted | AEKLSTLGKGD | 8849 |
| weighted | GLYPRSKIICW | 8850 |
| weighted | ISLARAFAIVA | 8851 |
| weighted | GGCHKTFILST | 8852 |
| weighted | AQHAGNHLLVK | 8853 |
| weighted | GIGNFIDCFFA | 8854 |
| weighted | IAPEKLELKPL | 8855 |
| weighted | AVRAPKASWKN | 8856 |
| weighted | QSSRYMIEHNP | 8857 |
| weighted | LEEITAGSDHW | 8858 |
| weighted | VQFDLQRARVG | 8859 |
| weighted | PNAYTENFLLV | 8860 |
| weighted | LEPVGHFKDLL | 8861 |
| weighted | ITSELLLLYIF | 8862 |
| weighted | GCIAITRQASP | 8863 |
| weighted | SANWSGRVKDH | 8864 |
| weighted | AEMISFIQAWK | 8865 |
| weighted | LYYLCTIIVSA | 8866 |
| weighted | KTENLPITRAL | 8867 |
| weighted | GQSIYAFQAIK | 8868 |
| weighted | IQENERRRRRV | 8869 |
| weighted | YLEGLPVAEAT | 8870 |
| weighted | EGFPRDLDDAT | 8871 |
| weighted | EQSANELQLWS | 8872 |
| weighted | DGPTNRCFKPK | 8873 |
| weighted | PCLKDAIVPTS | 8874 |
| weighted | PLREQLKKRVS | 8875 |
| weighted | IRGSDCLYIMV | 8876 |
| weighted | GGKLEFLALNR | 8877 |
| weighted | GGFEESRDEEN | 8878 |
| weighted | LYSASFWVVHE | 8879 |
| weighted | GLPGALDHDDF | 8880 |
| weighted | EQSSEGHPNET | 8881 |
| weighted | PWTTKARIQFS | 8882 |
| weighted | IVMRFSKTPFV | 8883 |
| weighted | GDCKESSISCP | 8884 |
| weighted | SQEVTNSLDFG | 8885 |
| weighted | DDHKFCSPMFL | 8886 |
| weighted | PQLEILELVQF | 8887 |
| weighted | ALQVRIPGMTK | 8888 |
| weighted | KIGMGNGISNI | 8889 |
| weighted | GSLVVVNKGLD | 8890 |
| weighted | VNTKSIQSLSA | 8891 |
| weighted | LKLGHSSPVCR | 8892 |
| weighted | KPFTKHAAVMV | 8893 |
| weighted | MAGDLGNRMAM | 8894 |
| weighted | IDERIAPHSSP | 8895 |
| weighted | SYCHYLQVPAN | 8896 |
| weighted | LEDAGIAVSKK | 8897 |
| weighted | DIVSNLRPALG | 8898 |
| weighted | QLFDSGVGPDG | 8899 |
| weighted | HSYQGTPLNGS | 8900 |
| weighted | RAFFPSISIQA | 8901 |
| weighted | HCAAVQKLASA | 8902 |
| weighted | ESKKIIASEAC | 8903 |
| weighted | LYGTALSGRAT | 8904 |
| weighted | SDEEAEPAIAD | 8905 |
| weighted | QDPTSEAQLFE | 8906 |
| weighted | GDNMAAGYAEV | 8907 |
| weighted | ITIEREGSWPT | 8908 |
| weighted | FNYLHPMGSLE | 8909 |
| weighted | EELVYWAQTDR | 8910 |
| weighted | LLKGGESSEME | 8911 |
| weighted | LQKSYVEASVE | 8912 |
| weighted | DLVQRVNFLRC | 8913 |
| weighted | EKINLKRLVKT | 8914 |
| weighted | ARLGDTSLLKS | 8915 |
| weighted | VYGSIPLGGSR | 8916 |
| weighted | KIAITPSRANG | 8917 |
| weighted | VEPHPNMGYAI | 8918 |
| weighted | HPEEDRSPGPI | 8919 |
| weighted | IVRQQGVELTL | 8920 |
| weighted | DMIVVALPYGL | 8921 |
| weighted | ALTLCSLQKGP | 8922 |
| weighted | GYENDPKPSEK | 8923 |
| weighted | SVGCGAVLSLG | 8924 |
| weighted | PRKAFVIRKHT | 8925 |
| weighted | RPNFKLGLNYE | 8926 |
| weighted | PGENGRTIIIG | 8927 |
| weighted | WKEVHVELKIP | 8928 |
| weighted | KALANQIGLSM | 8929 |
| weighted | FSRQSIEKHET | 8930 |
| weighted | QANSHYVQLPH | 8931 |
| weighted | LKMSKTSKSIA | 8932 |
| weighted | YGPQADSPWMC | 8933 |
| weighted | LGIAMVRGFFP | 8934 |
| weighted | VGHEGVAPSLF | 8935 |
| weighted | ELAKGTPANVK | 8936 |
| weighted | KLIQTWNIKLQ | 8937 |
| weighted | HRFCEGLFDKV | 8938 |
| weighted | SNYTFSGDHTL | 8939 |
| weighted | WELRLDHQNVK | 8940 |
| weighted | NFTPTSLQDDY | 8941 |
| weighted | LSCHRHYQQMP | 8942 |
| weighted | MAELELMILLT | 8943 |
| weighted | MCQQLKELPYC | 8944 |
| weighted | LLEQIAGFGSI | 8945 |
| weighted | TDGRAHATLRI | 8946 |
| weighted | QIGVQSQSVQL | 8947 |
| weighted | EGTKFTSDNTE | 8948 |
| weighted | KFVKKSLSLSV | 8949 |
| weighted | AQYMKEDLTNN | 8950 |
| weighted | DPKCYLTSGEN | 8951 |
| weighted | VFSYILCRSGR | 8952 |
| weighted | LGLFGLPGYGM | 8953 |
| weighted | ANPLVKGSVIF | 8954 |
| weighted | ERDSSFWPVVD | 8955 |
| weighted | DSYYPGEPAYR | 8956 |
| weighted | SKGDAAVIAKN | 8957 |
| weighted | IWNTTDLLQLG | 8958 |
| weighted | EAPPSREEPLS | 8959 |
| weighted | MAPTTHGQTWG | 8960 |
| weighted | DVSNGPIEIRF | 8961 |
| weighted | KSTSMKFDHIS | 8962 |
| weighted | LPSAKGVVGGP | 8963 |
| weighted | PSQDPGYIPKT | 8964 |
| weighted | CCLIWVRLCSA | 8965 |
| weighted | VLELGNKAEVL | 8966 |
| weighted | AKYSQDQYVKR | 8967 |
| weighted | PGESAPVLKSA | 8968 |
| weighted | IPLVAKLKATA | 8969 |
| weighted | PPDTLRGDYSP | 8970 |
| weighted | HHPDQGQLTTC | 8971 |
| weighted | RNVVHELWVQP | 8972 |
| weighted | DEPQGHASIFL | 8973 |
| weighted | IVLQMADTGPV | 8974 |
| weighted | AKLMLRDQAMY | 8975 |
| weighted | VVEIGTKTKDP | 8976 |
| weighted | PDVELVSVICS | 8977 |
| weighted | PTQVMPAGLRG | 8978 |
| weighted | PTPGVSYSATA | 8979 |
| weighted | SSVGEELALAI | 8980 |
| weighted | AQELPSEQAEK | 8981 |
| weighted | HRERLERCGVV | 8982 |
| weighted | NTRRGGRRGQQ | 8983 |
| weighted | WPLVNIEGTKE | 8984 |
| weighted | YNDTGRRSETE | 8985 |
| weighted | NDPVQTETVKA | 8986 |
| weighted | EVYCLSPEFGM | 8987 |
| weighted | DYNEGSSLQLV | 8988 |
| weighted | FQNPSVKAALK | 8989 |
| weighted | EAAGPGLGLWS | 8990 |
| weighted | SLAFHALALAF | 8991 |
| weighted | LEASRVGQPRV | 8992 |
| weighted | SGMPPQQADQN | 8993 |
| weighted | EWACQYSYIRT | 8994 |
| weighted | HSFKQEKVYND | 8995 |
| weighted | PCFGGQGLTNQ | 8996 |
| weighted | AYKKVSASPNL | 8997 |
| weighted | NRETLVPVRNL | 8998 |
| weighted | MQFLRMAEETG | 8999 |
| weighted | RLSKFSKKVNV | 9000 |
| weighted | RVLELAPSDGS | 9001 |
| weighted | ETAMVALYVKY | 9002 |
| weighted | DGQRILLEAAV | 9003 |
| weighted | LFDCASDKSTL | 9004 |
| weighted | JEAMAQSREQLT | 9005 |
| weighted | QELLSRSASCIV | 9006 |
| weighted | CDIPRYDLRGGH | 9007 |
| weighted | EPVQDKLFWAKE | 9008 |
| weighted | DPALLWKLYWHT | 9009 |
| weighted | GSFEEKRVLAGQ | 9010 |
| weighted | NTADFAQPLLFS | 9011 |
| weighted | SQSRMFKKTNSQ | 9012 |
| weighted | PKECAATLFKGS | 9013 |
| weighted | CSYDDTKSEVPI | 9014 |
| weighted | NNFLSLDIESKP | 9015 |
| weighted | LCEVGREHEHLP | 9016 |
| weighted | LNLSLCVETRGG | 9017 |
| weighted | KGYYELYTLLFP | 9018 |
| weighted | QRFAQGFLSTFS | 9019 |
| weighted | LWGVCWFRSDSL | 9020 |
| weighted | WQTKLPKEVQTS | 9021 |
| weighted | TFQQPEQATVFT | 9022 |
| weighted | RCFGGQVWHIGQ | 9023 |
| weighted | SGEPNLDAHPGL | 9024 |
| weighted | SKNFTNEPQPLE | 9025 |
| weighted | NGSPSIRVMGIQ | 9026 |
| weighted | ISLLFEGSLLLG | 9027 |
| weighted | GCEWSNLSGGRG | 9028 |
| weighted | GGPTRLPSPNTR | 9029 |
| weighted | ECFQLLTLQKSQ | 9030 |
| weighted | ADRLGVPVWRSG | 9031 |
| weighted | YIVLGSQDKMPS | 9032 |
| weighted | ELVGFYPLLQQR | 9033 |
| weighted | SVKYNDKAHRLF | 9034 |
| weighted | KFVPIGGDEAPT | 9035 |
| weighted | RKSHIFTVQLFC | 9036 |
| weighted | ITTNQAGSMEPV | 9037 |
| weighted | VDKSLYPDTPQQ | 9038 |
| weighted | EIMVKNIVDEDW | 9039 |
| weighted | DQKTVAGPAFKS | 9040 |
| weighted | SSPDSQEMTLPT | 9041 |
| weighted | PNHAAATEVNQL | 9042 |
| weighted | KRDCGYFLRCPW | 9043 |
| weighted | CPLFYAIELPTL | 9044 |
| weighted | LFYADPWSAKNA | 9045 |
| weighted | IQGSNCLPDSTH | 9046 |
| weighted | NNTSITLWQRFA | 9047 |
| weighted | YPVSKETPVPAF | 9048 |
| weighted | INTFIECIAADI | 9049 |
| weighted | GPGRYSADKING | 9050 |
| weighted | CNPRRRLRVETA | 9051 |
| weighted | VMLDYIGPAVYA | 9052 |
| weighted | EESQSPIGLQSK | 9053 |
| weighted | QKASVKLLNLEL | 9054 |
| weighted | ALRDMVRILSPY | 9055 |
| weighted | QELTNLTGYENM | 9056 |
| weighted | RLKSAIRVYERG | 9057 |
| weighted | AFDEVFWVHGID | 9058 |
| weighted | DLPQLYIPRGSS | 9059 |
| weighted | ARADPHKFQNGD | 9060 |
| weighted | KCHPLQNQPLFV | 9061 |
| weighted | QGAGNMPGQAKE | 9062 |
| weighted | PGNWCLGRVVFP | 9063 |
| weighted | EHMNVWSPIYTP | 9064 |
| weighted | DYLPRSEILIRP | 9065 |
| weighted | IPGKHERSTIRL | 9066 |
| weighted | SLIAGICRHQPD | 9067 |
| weighted | PYGKILYKIRNM | 9068 |
| weighted | FQQALLKVGKVG | 9069 |
| weighted | DSYALTTSLDKL | 9070 |
| weighted | VANLEGLEWFLL | 9071 |
| weighted | FSVAVGSPFCRP | 9072 |
| weighted | SVPDLPSKKTPI | 9073 |
| weighted | GRRLMISLLYRH | 9074 |
| weighted | CNDNEKSIWGSD | 9075 |
| weighted | IETTIVGYGGGL | 9076 |
| weighted | IEQFLKTREMYE | 9077 |
| weighted | TLFPLNHHGDAV | 9078 |
| weighted | RDEADFGASLYF | 9079 |
| weighted | CFITPNRGLIFC | 9080 |
| weighted | YELGKGNSAREM | 9081 |
| weighted | SGFHEPGIEKWE | 9082 |
| weighted | KVATMAENVVKA | 9083 |
| weighted | EMLSCGKQTCVP | 9084 |
| weighted | LGFAHAREPERV | 9085 |
| weighted | KSCEKKCDAKNI | 9086 |
| weighted | QLEAYETKRPRP | 9087 |
| weighted | RKYVDRCDQSSC | 9088 |
| weighted | IESPGDKDKAMS | 9089 |
| weighted | HYAQLLLRNQST | 9090 |
| weighted | ADALPLDENRKK | 9091 |
| weighted | PLKRNKAIAKSS | 9092 |
| weighted | JEAMWGIVASHSF | 9093 |
| weighted | PQELALKLHVMC | 9094 |
| weighted | GRDEQPVHLLFP | 9095 |
| weighted | MVVRSTAQNGFL | 9096 |
| weighted | SCDYDYVQALGP | 9097 |
| weighted | LQPELQTMYPDT | 9098 |
| weighted | NLFNSELACQSC | 9099 |
| weighted | RSQTSGRAVEES | 9100 |
| weighted | AIQGLSPEHELD | 9101 |
| weighted | YATEALPEAQSV | 9102 |
| weighted | CGSWIRESLDLR | 9103 |
| weighted | SRTAFQGGATDQ | 9104 |
| weighted | QPKGSERHLKAV | 9105 |
| weighted | AAGVGELFKPSP | 9106 |
| weighted | LAVNQHKQADFR | 9107 |
| weighted | LNQREPPWKLTS | 9108 |
| weighted | ILPGKTAVSLDNW | 9109 |
| weighted | YLMGAPLSVSNG | 9110 |
| weighted | KKERIVSPYDER | 9111 |
| weighted | LIGTNIMLQPIL | 9112 |
| weighted | ALEPIHFLNAVM | 9113 |
| weighted | RLWSGRQSQGSH | 9114 |
| weighted | IYLSHHTMYSRP | 9115 |
| weighted | GEMSLEDRKEQL | 9116 |
| weighted | TLAAEVWLYGAT | 9117 |
| weighted | SDGTLQQWMEHL | 9118 |
| weighted | ATTQNNSQQSAK | 9119 |
| weighted | APATRYCGRKYE | 9120 |
| weighted | DAALAILSRAQS | 9121 |
| weighted | MPQVTGQSSDQG | 9122 |
| weighted | KSDFQLLIVVLF | 9123 |
| weighted | LEQSRLLTLDTR | 9124 |
| weighted | QAKLQGSLWIIQ | 9125 |
| weighted | WDDQSFALFRSE | 9126 |
| weighted | LGVPKLISLCSV | 9127 |
| weighted | YSQADLRENSPK | 9128 |
| weighted | QECEALESASTL | 9129 |
| weighted | DTGIFLRSSKRQ | 9130 |
| weighted | SGWTGKPIAPII | 9131 |
| weighted | FIFGTASRLLDG | 9132 |
| weighted | FDSVSILPISRA | 9133 |
| weighted | LHQLFWRDSSVK | 9134 |
| weighted | DHQELMENLIAD | 9135 |
| weighted | DGMNPMLRLMRD | 9136 |
| weighted | ASIIPLRDRFET | 9137 |
| weighted | PKALPSPFDKVP | 9138 |
| weighted | KHTETSQAVFSM | 9139 |
| weighted | EFLRNEQPTLGA | 9140 |
| weighted | IFEKLDYHVSIV | 9141 |
| weighted | QPFIDIADSIIA | 9142 |
| weighted | EDTMLHVELDQS | 9143 |
| weighted | LDRLPFTHFIGR | 9144 |
| weighted | FVYLTHLEDLKN | 9145 |
| weighted | CCVNQVTYLDMS | 9146 |
| weighted | PSVDESFNFGVH | 9147 |
| weighted | WMEFPELPTTGG | 9148 |
| weighted | TMNPVIRLGMRD | 9149 |
| weighted | EAEEAKIQSMIL | 9150 |
| weighted | EFLCLFFGQEDV | 9151 |
| weighted | KAMLLPEGTAEA | 9152 |
| weighted | KSWLLGKILDRL | 9153 |
| weighted | MLKVPCLHEFPG | 9154 |
| weighted | TVDIAVLKIPVL | 9155 |
| weighted | EGGLLQLFDCMS | 9156 |
| weighted | KEASGCEMQSAA | 9157 |
| weighted | RPSSEPVVTMPL | 9158 |
| weighted | RSKLSLQGVDKL | 9159 |
| weighted | DHAKLESSKIAA | 9160 |
| weighted | KREKADKVFGDG | 9161 |
| weighted | IIKLEVWGTMPL | 9162 |
| weighted | GRDRFLQSGSRV | 9163 |
| weighted | PGIVVSQAITDP | 9164 |
| weighted | KNELGRIIVPTD | 9165 |
| weighted | FQDVNPIVRTLK | 9166 |
| weighted | TYSSTALLYTFS | 9167 |
| weighted | LRSLPYLTNMSG | 9168 |
| weighted | ENLQSLPFGWTS | 9169 |
| weighted | SKLAVKLMLDCL | 9170 |
| weighted | SLKLLNELAFKR | 9171 |
| weighted | RIEAKQESVRLG | 9172 |
| weighted | VMAYIPMSEITF | 9173 |
| weighted | DKPQHQDAAHGH | 9174 |
| weighted | FCPLQSQLRDSQ | 9175 |
| weighted | GDQIGEVETEPK | 9176 |
| weighted | QPLHQGPIELNA | 9177 |
| weighted | WDENAHSDSTFG | 9178 |
| weighted | RPLEYGECILQN | 9179 |
| weighted | NMDFQASSLENA | 9180 |
| weighted | IAIWIVPRNYGR | 9181 |
| weighted | PEPSDQTIETNH | 9182 |
| weighted | YRQNLGKISLLV | 9183 |
| weighted | ASSISRVWEAGP | 9184 |
| weighted | AIFSPLSNPNVT | 9185 |
| weighted | RRKMSKQQAGLH | 9186 |
| weighted | GCGVINLGIPIR | 9187 |
| weighted | KSNTRQLGNFSP | 9188 |
| weighted | DKILCIDLIKAP | 9189 |
| weighted | QSIYTGTSFACS | 9190 |
| weighted | NTEEQWPARFED | 9191 |
| weighted | RLWANEVVPYRE | 9192 |
| weighted | EIDAIFEFGAKE | 9193 |
| weighted | RARYAHKERYLS | 9194 |
| weighted | KKSHQNLCASLP | 9195 |
| weighted | GAPSAKFGLMWE | 9196 |
| weighted | RHQWNSHNSRLT | 9197 |
| weighted | QVPIDAFSQFVD | 9198 |
| weighted | KNSLCLAQKAAQ | 9199 |
| weighted | FCEYDHNRVSLA | 9200 |
| weighted | DPTNVYINAEER | 9201 |
| weighted | KSKTGRFYFHKE | 9202 |
| weighted | YDTLTSLQKGNR | 9203 |
| weighted | GQSLQWMFIYEL | 9204 |
| weighted | VKSLLSVLPSCN | 9205 |
| weighted | EASARTGNGMEP | 9206 |
| weighted | TGLNESVVSEVG | 9207 |
| weighted | NKKCYDANPPAR | 9208 |
| weighted | RRTKSASSVKLV | 9209 |
| weighted | AIGMRGLGMCGS | 9210 |
| weighted | YNPQALTCGPKG | 9211 |
| weighted | SGGLPRNTQQWH | 9212 |
| weighted | HTRQEGLPEKRA | 9213 |
| weighted | CVKDWTPRPPDS | 9214 |
| weighted | DNLQGVFPSKGK | 9215 |
| weighted | HNPELVSELLIY | 9216 |
| weighted | EQPLNTLLAYDA | 9217 |
| weighted | QLQMYKYMRAKA | 9218 |
| weighted | EDYVRVKCAALP | 9219 |
| weighted | LAHHMADSRILR | 9220 |
| weighted | EDRTFDFLAITL | 9221 |
| weighted | HYALGQKPSDCT | 9222 |
| weighted | LSGKKADSAFHL | 9223 |
| weighted | VIARAVHIRTTL | 9224 |
| weighted | WVVAAEYSLPFR | 9225 |
| weighted | TSKGKSIREMPN | 9226 |
| weighted | QQDQVRGPGLLL | 9227 |
| weighted | LRSECQTKFLQH | 9228 |
| weighted | QTESHYQLHYSI | 9229 |
| weighted | GNTKYQARKHKE | 9230 |
| weighted | DRKIGVLDPPKA | 9231 |
| weighted | PSEIQSVGVMEE | 9232 |
| weighted | TPLISQGPRRFS | 9233 |
| weighted | SEGSALEMFRSN | 9234 |
| weighted | MVEGHLSEKSRA | 9235 |
| weighted | LINPESGEVDPQ | 9236 |
| weighted | PRPIVLFCETKI | 9237 |
| weighted | PSQDKGMAGPDW | 9238 |
| weighted | AFGLKEQVRPIL | 9239 |
| weighted | CATNEYDQPRAK | 9240 |
| weighted | TPSAARSFAIES | 9241 |
| weighted | FEPSFRLLLDLR | 9242 |
| weighted | GETSMVSYVTRA | 9243 |
| weighted | PACHMGMCADEA | 9244 |
| weighted | HCPKFETKLLSP | 9245 |
| weighted | RVTGTKSEADIA | 9246 |
| weighted | CPVAGGPALGLT | 9247 |
| weighted | PDQGDGYSVFSI | 9248 |
| weighted | QKQCLELVDVPL | 9249 |
| weighted | LGELLSDNNNGM | 9250 |
| weighted | ASDEQVIVTFEK | 9251 |
| weighted | HGPGALCAKVAT | 9252 |
| weighted | SLVVGESKPSSD | 9253 |
| weighted | AEKPRNSSDDEG | 9254 |
| weighted | ALASSQVGPVMS | 9255 |
| weighted | QKGLAVSLTQDM | 9256 |
| weighted | HGCAGEGDSSDE | 9257 |
| weighted | RQAAPVHLLELL | 9258 |
| weighted | GSTSVGVMSNMS | 9259 |
| weighted | IHGIDDNSASCT | 9260 |
| weighted | NSAPELDILMLI | 9261 |
| weighted | YKGKLAITGMNA | 9262 |
| weighted | DIKLPPIYDSAC | 9263 |
| weighted | DDRCQKQHRHCS | 9264 |
| weighted | PQRLCLQEEIIP | 9265 |
| weighted | RKEIIEDDDDSD | 9266 |
| weighted | KLSYLSKDSILV | 9267 |
| weighted | GFFTDNGISDLN | 9268 |
| weighted | VQSPFLMSFLEA | 9269 |
| weighted | AVYASFPAVARP | 9270 |
| weighted | SGRLGPHICLGR | 9271 |
| weighted | VPKSCKTDSPEF | 9272 |
| weighted | IKPSVLKYGDRV | 9273 |
| weighted | GGVRRECETMDT | 9274 |
| weighted | GMLFRMSNPNHS | 9275 |
| weighted | FPFCPVVWCTCA | 9276 |
| weighted | VHVCEYPLKRGA | 9277 |
| weighted | DTKPGVFGDEER | 9278 |
| weighted | MLEPRDYTYMAC | 9279 |
| weighted | PIKESALRKQPD | 9280 |
| weighted | GVFPPQSLTTTD | 9281 |
| weighted | AESSLLESMTTV | 9282 |
| weighted | NAHRSKRQVIDS | 9283 |
| weighted | HVLKASSCARHS | 9284 |
| weighted | ESAFARGLNRLV | 9285 |
| weighted | KNVRQDTDIWHP | 9286 |
| weighted | GFGGAKVKGCEG | 9287 |
| weighted | SFGQRCSRDQND | 9288 |
| weighted | EVLVTRRTCTES | 9289 |
| weighted | ANEETALTLSTV | 9290 |
| weighted | SIQRVTMFAKLQ | 9291 |
| weighted | LTGLARPVRVDL | 9292 |
| weighted | AAIQPPESASQR | 9293 |
| weighted | SAQAPPPQAAGV | 9294 |
| weighted | ATAKSCMVAPPP | 9295 |
| weighted | SFQRSVNNSQGV | 9296 |
| weighted | SEEIHDCHNCSS | 19297 |
| weighted | EELMDSRSDKAA | 9298 |
| weighted | EAGAYFQELLYE | 9299 |
| weighted | GQDGTGEFRKTF | 9300 |
| weighted | GHGRTRAYQSTT | 9301 |
| weighted | ERTILAAEVASD | 9302 |
| weighted | STSVNLESVLWL | 9303 |
| weighted | YDGFLDEERRDN | 9304 |
| weighted | WACTPLNNIVRR | 9305 |
| weighted | NSPSVSEDRCRG | 9306 |
| weighted | SKIEEKRKPSFF | 9307 |
| weighted | RIDRMMVELRMQ | 9308 |
| weighted | PYIHQLPSRDAD | 9309 |
| weighted | EIELVMSVPPTK | 9310 |
| weighted | SLRQCNPSPIEG | 9311 |
| weighted | MLKMHRLNPTSR | 9312 |
| weighted | LTHLPGVCFAIR | 9313 |
| weighted | WKGFPAEAHAEQ | 9314 |
| weighted | QAVCLYTSGGNL | 9315 |
| weighted | MQEKPQQIKKHQ | 9316 |
| weighted | VPHAKGKDSNPI | 9317 |
| weighted | IALFRECTCHFV | 9318 |
| weighted | TLDALPVIRSKP | 9319 |
| weighted | ASGAPPCAENME | 9320 |
| weighted | LDLLTEHEGTDP | 9321 |
| weighted | LPAFRPVGKGLP | 9322 |
| weighted | VQFRKLGGAWCG | 9323 |
| weighted | NYEPAANDVLRF | 9324 |
| weighted | AFFNYAVMADGV | 9325 |
| weighted | TNDLTTMQKVRK | 9326 |
| weighted | PERGVEGDRSLH | 9327 |
| weighted | LIDRECMGTHIS | 9328 |
| weighted | KYKDVSVIGHDS | 9329 |
| weighted | VEFYSIVSKKQL | 9330 |
| weighted | LTDPGIRARTGS | 9331 |
| weighted | QLKLLPSQSSPL | 9332 |
| weighted | EQACQYVEESLS | 9333 |
| weighted | TGCCQQIMNQLE | 9334 |
| weighted | DVASLNFGAEKP | 9335 |
| weighted | FASPSEPSDLNI | 9336 |
| weighted | RDNRPPLKHKAK | 9337 |
| weighted | RNWGQGNQFHQL | 9338 |
| weighted | PNSNVGTCLQTL | 9339 |
| weighted | LKDETDLMKGMP | 9340 |
| weighted | AALDLSLKRKES | 9341 |
| weighted | FGGTVTIAGSYD | 9342 |
| weighted | SLEEKLTLRRSA | 9343 |
| weighted | DSPTHYYVETLA | 9344 |
| weighted | NLPYDKGHTHDV | 9345 |
| weighted | PVGHDGLGLYMN | 9346 |
| weighted | EPASPPLGPGML | 9347 |
| weighted | QPELISEAEFSE | 9348 |
| weighted | PHVWLPLCKSRL | 9349 |
| weighted | CLDDPVPEQGAE | 9350 |
| weighted | SEKPFTHFDRIK | 9351 |
| weighted | RQAEGSEKSFSE | 9352 |
| weighted | WLCGAFSIGFDF | 9353 |
| weighted | PHLCIRNLFHQF | 9354 |
| weighted | SGVPPMKKVTPT | 9355 |
| weighted | KTHMGRGEASCQ | 9356 |
| weighted | PKPIKELGFWEP | 9357 |
| weighted | VQPYELPEPERT | 9358 |
| weighted | LDTARGHAALTL | 9359 |
| weighted | PKFDVVGSRVSQ | 9360 |
| weighted | AYGLEDPKANDI | 9361 |
| weighted | FAKDCQWDEGNG | 9362 |
| weighted | VIFVTSPVHPTK | 9363 |
| weighted | DLVLCWAFAKNK | 9364 |
| weighted | FRSSFAKSDKLG | 9365 |
| weighted | SDLPDCSVELTV | 9366 |
| weighted | SVSPQYGYGGVA | 9367 |
| weighted | TPIEAGVINEDE | 9368 |
| weighted | SHVDLKSHLYVC | 9369 |
| weighted | RSTVEWIIANPM | 9370 |
| weighted | IKHIMAGISASA | 9371 |
| weighted | SKAEEPKGPKDA | 9372 |
| weighted | GHTVRVIPLTVY | 9373 |
| weighted | SLKRGDKSPEFR | 9374 |
| weighted | GIDLDIQGDTRY | 9375 |
| weighted | NTLPTCELLIVL | 9376 |
| weighted | SATYSSNDLDLH | 9377 |
| weighted | YKRLATATGRQL | 9378 |
| weighted | RFQKGDNNSSKQ | 9379 |
| weighted | SEQAGDVAARLP | 9380 |
| weighted | LISPYRKGSGNL | 9381 |
| weighted | DNACDEHLVRQP | 9382 |
| weighted | FDLQPIRAHHEI | 9383 |
| weighted | QHPEDVIHINDN | 9384 |
| weighted | GCGKPDENNVSR | 9385 |
| weighted | RELTDIHCSLSV | 9386 |
| weighted | LLTLDRLECQLK | 9387 |
| weighted | PIQLIIIPRGVH | 9388 |
| weighted | ETYNAIFDLPCF | 9389 |
| weighted | YTTGEWSRGFNL | 9390 |
| weighted | QGQSKGIRQPKL | 9391 |
| weighted | TSTEYVHGLTEC | 9392 |
| weighted | PLLIRPGIKAVA | 9393 |
| weighted | LMQEQYPVNHSS | 9394 |
| weighted | GDAISSLYTPSQ | 9395 |
| weighted | LDMHGQQASKSD | 9396 |
| weighted | ELRSLGTTISLP | 9397 |
| weighted | LLELGMCRFGSE | 9398 |
| weighted | VGFSPKTRFKEA | 9399 |
| weighted | LAEIVNACGSGK | 9400 |
| weighted | EKDSVENNWPSQ | 9401 |
| weighted | TTATTVTTGQPG | 9402 |
| weighted | ASCRSPRKELEL | 9403 |
| weighted | IHDNGAIFAALN | 9404 |
| weighted | FQWPHAMRVSNS | 9405 |
| weighted | SNGKNRPAAPRW | 9406 |
| weighted | PVITEEKLNKQI | 9407 |
| weighted | CYHCLECCVAAN | 9408 |
| weighted | ERFGDLTYQLKA | 9409 |
| weighted | WPDLESAQEYVQ | 9410 |
| weighted | LNAKSFNMFSSV | 9411 |
| weighted | GLEVINYSPAQA | 9412 |
| weighted | ACYFVLCAAPML | 9413 |
| weighted | DRCGVWTNSREL | 9414 |
| weighted | FAPEAKFCNVRH | 9415 |
| weighted | QEASQSPLDVEI | 9416 |
| weighted | PLWAVPSLWPPD | 9417 |
| weighted | LDRFLMQNKCNP | 9418 |
| weighted | WIVFQGERGTTQ | 9419 |
| weighted | SQCPGRAGPRKE | 9420 |
| weighted | AFVVYCVISRER | 9421 |
| weighted | LALRDNVHPFNQ | 9422 |
| weighted | VLNDTWTCFCIL | 9423 |
| weighted | VQTRITFGDGDS | 9424 |
| weighted | QSSQLMLDGCSI | 9425 |
| weighted | WEGPRPMRMNFS | 9426 |
| weighted | VTIFSKELIWAI | 9427 |
| weighted | ASDDLENGAGGR | 9428 |
| weighted | LNGALQVTIDPE | 9429 |
| weighted | GKVVAMPLLGGR | 9430 |
| weighted | SKQDSVDEAREV | 9431 |
| weighted | EPISAGGCGSLF | 9432 |
| weighted | LPKNLAIKVSSV | 9433 |
| weighted | ETGCDLSMSFFC | 9434 |
| weighted | QPYSTFSDCYVP | 9435 |
| weighted | ETPGHEASTRKS | 9436 |
| weighted | RHQRSVTAKDCL | 9437 |
| weighted | PEVERTKFQLKA | 9438 |
| weighted | NQAVLHVKDKVG | 9439 |
| weighted | AAGALLLSTCSI | 9440 |
| weighted | DYMSNSVSAHSL | 9441 |
| weighted | HEPLLKTGLLQP | 9442 |
| weighted | CKTFIYVNTLKF | 9443 |
| weighted | VSIFDTSDADCL | 9444 |
| weighted | SNISSQAHVNVI | 9445 |
| weighted | ISPELHPTSGGM | 9446 |
| weighted | HRVRQVQKWVSS | 9447 |
| weighted | FVQGMSVWFLKP | 9448 |
| weighted | VSVLADVEDLAR | 9449 |
| weighted | ILLGKVTPLGPVP | 9450 |
| weighted | FVIWNDCEGPPR | 9451 |
| weighted | DHGVPINSMKQC | 9452 |
| weighted | MQCFRTDSGKMH | 9453 |
| weighted | KVGLPSLSMDQN | 9454 |
| weighted | SELIVRVINEFA | 9455 |
| weighted | KVGSMNLYVERS | 9456 |
| weighted | LPVIANFALQSE | 9457 |
| weighted | VDALHSVCCLVC | 9458 |
| weighted | ESNVESMTCFVQ | 9459 |
| weighted | TAELQDDEVGLQ | 9460 |
| weighted | ARALMPSGDALF | 9461 |
| weighted | CKFLSGIMDVDW | 19462 |
| weighted | YSLISLSSDWRH | 9463 |
| weighted | VLSGAQFSREQT | 9464 |
| weighted | ILALLTAVVFEL | 9465 |
| weighted | PELHDVQYKIPM | 9466 |
| weighted | PPHLVFYTNDVT | 9467 |
| weighted | PVPRVRMIDTCI | 9468 |
| weighted | VVMAQAVGQAHC | 9469 |
| weighted | ILTPFQVLLAMKT | 9470 |
| weighted | PVKHKKRDSSKS | 9471 |
| weighted | GTASGAWRWEVF | 9472 |
| weighted | GVQLNSKIHYRL | 9473 |
| weighted | VKALYWKPPVGI | 9474 |
| weighted | VTDRSSKNRFGC | 9475 |
| weighted | DVMVGSNDEKVP | 9476 |
| weighted | DDPTRVVNRGPK | 9477 |
| weighted | KLGDLGGTNPEA | 9478 |
| weighted | ADRPNPIPERGG | 9479 |
| weighted | KKLASQRQQTKV | 9480 |
| weighted | LTNDVQSTESKP | 9481 |
| weighted | DTFGYQACLHDC | 9482 |
| weighted | LPSLQGPVSRFA | 9483 |
| weighted | EECGEFFFLEVF | 9484 |
| weighted | KALNHGGMSFLN | 9485 |
| weighted | TFKDDQVSEYQK | 9486 |
| weighted | DVNPEDGLCSLA | 9487 |
| weighted | ELINLNYLEELD | 9488 |
| weighted | LGQRIKQLAREL | 9489 |
| weighted | RREPIQPQEDRD | 9490 |
| weighted | DLYVFWPSGGGV | 9491 |
| weighted | HCDVSVQLCSPK | 9492 |
| weighted | SSYQQTMDGEKT | 9493 |
| weighted | SYRIMFGQEILD | 9494 |
| weighted | LKLTRGIEPSYH | 9495 |
| weighted | IFLRKKAIKGGS | 9496 |
| weighted | STSADDLFGTAL | 9497 |
| weighted | DTYFKAGVVYKI | 9498 |
| weighted | VIRKGGGQFTPC | 9499 |
| weighted | TMLLPKCLLLKA | 9500 |
| weighted | CRAEAYALKEVN | 9501 |
| weighted | PIGTSTAANDSD | 9502 |
| weighted | AEYPQTINVKGK | 9503 |
| weighted | NEVKSLETALHR | 9504 |
| weighted | PPRPSVNQNNLL | 9505 |
| weighted | LLGNKASFGAVK | 9506 |
| weighted | GSGATSFLYGEP | 9507 |
| weighted | VLVEAGPVQIYD | 9508 |
| weighted | NLRTPTEQIKPK | 9509 |
| weighted | TYFKAGKTSEAR | 9510 |
| weighted | NTRAFLGVHVET | 9511 |
| weighted | KANAGLGGDFDF | 9512 |
| weighted | SRFRSQLLPILK | 9513 |
| weighted | QEVAPTKAAIKQ | 9514 |
| weighted | RIGQEPLTQKVV | 9515 |
| weighted | SLVRYWACELEE | 9516 |
| weighted | LTEKSSEQLTSQ | 9517 |
| weighted | AYLLAETCLSER | 9518 |
| weighted | FEQSTKGITRRG | 9519 |
| weighted | RPRLPQATLSLS | 9520 |
| weighted | GLSGTNLKQCSH | 9521 |
| weighted | NNSSEVNLTKED | 9522 |
| weighted | QKTGKKFKYPEK | 9523 |
| weighted | TRQPFDKGTEYA | 9524 |
| weighted | QKSLDHLLFALD | 9525 |
| weighted | ESLFATADQSIG | 9526 |
| weighted | DQPKVESPVIDG | 9527 |
| weighted | KMKFVFKEVRTL | 9528 |
| weighted | VQQLYLSPSLDT | 9529 |
| weighted | MCGHHGLDGARR | 9530 |
| weighted | CAEELAKAFNWE | 9531 |
| weighted | KELMRYDPLRLI | 9532 |
| weighted | KESDRAKYQSTV | 9533 |
| weighted | AVANRVIHPQGF | 9534 |
| weighted | RIKHIKEEPPKT | 9535 |
| weighted | TSESSIFGIHEL | 9536 |
| weighted | TSSVVSNSEKKA | 9537 |
| weighted | LATMVPGLEIVI | 9538 |
| weighted | HTSHTKTYGSAF | 9539 |
| weighted | TAFEGYGAERPE | 9540 |
| weighted | IVGKTISRCKDW | 9541 |
| weighted | GTSPIAPAFQDN | 9542 |
| weighted | LLCSLFRTVHAV | 9543 |
| weighted | DEIQERGEPLNA | 9544 |
| weighted | SLGYERDRTPLL | 9545 |
| weighted | PGDMVLNMIVCG | 9546 |
| weighted | VCHLQKRCAPTR | 9547 |
| weighted | HKEATTATKQIN | 9548 |
| weighted | LMSIALPLLRIE | 9549 |
| weighted | PQPCRLEDCFLV | 9550 |
| weighted | PESTSPTITLHG | 9551 |
| weighted | LEQNRGSNLIHF | 9552 |
| weighted | QNSCSVKTEEHD | 9553 |
| weighted | NLLPSLDALVMN | 9554 |
| weighted | PMKWLYGFLFPL | 9555 |
| weighted | KRARFRLPTGVP | 9556 |
| weighted | HAHVSVGPAGCD | 9557 |
| weighted | DEHEIQFRSYLY | 9558 |
| weighted | TMTTKEVRKLTN | 9559 |
| weighted | RNKLHIKIARLT | 9560 |
| weighted | IRTGEPIAKAPP | 9561 |
| weighted | GSVVSHKDGPAV | 9562 |
| weighted | QEAGYPHDIARS | 9563 |
| weighted | SLAEQAWHKAPT | 9564 |
| weighted | PPKHPVLMCSYK | 9565 |
| weighted | SQKFPTQMIERV | 9566 |
| weighted | QITPKSYLITIR | 9567 |
| weighted | LPPVRGDCLNQN | 9568 |
| weighted | PRTYGKPEGSHH | 19569 |
| weighted | AIKRVADFVLLV | 9570 |
| weighted | AMVCVVKLDYRP | 9571 |
| weighted | LTELYDLKRGPI | 9572 |
| weighted | DKPIPFQMRLIA | 9573 |
| weighted | HSGVSLQVKDME | 9574 |
| weighted | HGYMIEAKAPSK | 9575 |
| weighted | DMTLGKQLMIQE | 9576 |
| weighted | VKEILMCPMGDF | 9577 |
| weighted | GNKIYKMRCMST | 9578 |
| weighted | DLSFLLTLVAGI | 9579 |
| weighted | RMDYKAADSIID | 9580 |
| weighted | TSELLPAPQNLC | 9581 |
| weighted | GSHLVAMQVQYR | 9582 |
| weighted | IRINFHAAGPES | 9583 |
| weighted | FFKKPDLGLKPE | 9584 |
| weighted | ENVLNHSSDQLS | 9585 |
| weighted | RLYYQELALMFR | 9586 |
| weighted | KKLFLGVHHIVV | 9587 |
| weighted | AAPHQSHQQSSL | 9588 |
| weighted | LHQAENKRQEFP | 9589 |
| weighted | LDLGSAYPDGSR | 9590 |
| weighted | QMHGTLAKLYWD | 9591 |
| weighted | SMCQGRDCERIQ | 9592 |
| weighted | NTSQPNKQSGDK | 9593 |
| weighted | PRREPVPSTSPV | 9594 |
| weighted | VYYEDNSEAGSF | 9595 |
| weighted | FCFEAANLLLPF | 9596 |
| weighted | LRKLNMEEIQHQ | 9597 |
| weighted | ETRAQLEYLLFF | 9598 |
| weighted | QQLKYLYPAVAL | 9599 |
| weighted | LSKTLQKVEVSV | 9600 |
| weighted | IATVVNQQGMAD | 9601 |
| weighted | WEALKSWDEQSL | 9602 |
| weighted | KKSILTGKINDS | 9603 |
| weighted | GVEVEKTTDWIP | 9604 |
| weighted | DAVGHLDEAGQN | 9605 |
| weighted | SIGHSPTDSRLN | 9606 |
| weighted | EDGDPLYRVNQF | 9607 |
| weighted | RFYLLECWQEHK | 9608 |
| weighted | ESHLDMEYVEVY | 9609 |
| weighted | EHRSEKPDRFDR | 9610 |
| weighted | RLQGSTFFGFSL | 9611 |
| weighted | ALALDYEFKKSA | 9612 |
| weighted | ILTPADRFYPGV | 9613 |
| weighted | SPGNNPIRGLHP | 9614 |
| weighted | EPDTHLSASGGS | 9615 |
| weighted | RPASRVYSNTWK | 9616 |
| weighted | GFKCFTQLLRFL | 9617 |
| weighted | RALFMGRHKEGN | 9618 |
| weighted | YSKLARGSLESH | 9619 |
| weighted | RCVPLEEKQREI | 9620 |
| weighted | QILQQGNEALVR | 9621 |
| weighted | HGDGKEPQSKDN | 9622 |
| weighted | LTPCLGSADPKN | 9623 |
| weighted | RLDLQTSIDVFK | 9624 |
| weighted | TERESHMAAISG | 9625 |
| weighted | VNSAAVFESCMF | 9626 |
| weighted | DDAKLLRYLRST | 9627 |
| weighted | GMTQAARLNITG | 9628 |
| weighted | QVSPETVEMEAA | 19629 |
| weighted | EVCRAYGGLKAA | 9630 |
| weighted | VLPAQSHFLATN | 9631 |
| weighted | GPADEFLSVHLG | 9632 |
| weighted | IFTKRESVSTLG | 9633 |
| weighted | INDSNFTILANPS | 9634 |
| weighted | FDHGVKVEKPPS | 9635 |
| weighted | LTSGVCSVGVFS | 9636 |
| weighted | PLLTFLRRWTQK | 9637 |
| weighted | GPVDSIFGKTWQ | 9638 |
| weighted | INGADVVGVDAI | 9639 |
| weighted | REDKRFDQTSVT | 9640 |
| weighted | PGFQSTESLAFQ | 9641 |
| weighted | IPSHHFPFRILN | 9642 |
| weighted | FQVLIGSVLTHG | 9643 |
| weighted | VPYVPGPPGEDI | 9644 |
| weighted | PTFPCYFLEQGS | 9645 |
| weighted | VEPCNPVGKTFV | 9646 |
| weighted | RDAARTGQLKKQ | 9647 |
| weighted | LLKYNPDCDSQA | 9648 |
| weighted | LDADMSDVDICY | 9649 |
| weighted | LAVMTRGAELDS | 9650 |
| weighted | ERPSSFLQIFHS | 9651 |
| weighted | GIVLGTALNEER | 9652 |
| weighted | RPKPEWTLVLRR | 9653 |
| weighted | HTDLLLLTNVDI | 9654 |
| weighted | VHVLGLRVSSDE | 9655 |
| weighted | TCKENVPGHCET | 9656 |
| weighted | ESLNMEATLNHI | 9657 |
| weighted | ESKSLGLLDKKP | 19658 |
| weighted | RNSTRTECPQAK | 9659 |
| weighted | LSHGALQPEVLL | 9660 |
| weighted | YILVSDSTAPVM | 9661 |
| weighted | LIRAKRSADHAA | 9662 |
| weighted | RSGRGHGLHVML | 9663 |
| weighted | PKKYPLTFSPVD | 9664 |
| weighted | EQGFLQLCFPRQ | 9665 |
| weighted | TTTLNRPFCGNT | 9666 |
| weighted | EHVLEFKGLQTN | 9667 |
| weighted | MESSSMLQVNRR | 9668 |
| weighted | PEIPVPELFKYE | 9669 |
| weighted | FGKLFIRMRYAM | 9670 |
| weighted | PYLVDTRLSTQH | 9671 |
| weighted | CFFVRNCLRTPA | 19672 |
| weighted | GNALSLNFLTGL | 9673 |
| weighted | SCPIKFLLREVV | 9674 |
| weighted | WPLRSKKPRFSD | 9675 |
| weighted | YTIVCNRAQWVV | 9676 |
| weighted | IQSKRDPPADTI | 9677 |
| weighted | ARIYLDLELVPV | 9678 |
| weighted | LEGASQGTSCDG | 9679 |
| weighted | RETRLIGPMCLL | 9680 |
| weighted | TGKLSTNNWDLG | 9681 |
| weighted | KEWSLVKTHPTQ | 9682 |
| weighted | QWISVGLSGAVL | 9683 |
| weighted | HTLCRLSALGKD | 9684 |
| weighted | DFNQFDHLTQSL | 9685 |
| weighted | ASSALLAPDKVR | 9686 |
| weighted | AVLVEAGTILKD | 9687 |
| weighted | GSHLVPTMLAST | 9688 |
| weighted | EYVYACTRTLGL | 9689 |
| weighted | GFGVGMRSPYLI | 9690 |
| weighted | WYKAFFEVTQMR | 9691 |
| weighted | AHITDSALNNPP | 19692 |
| weighted | HSREPETRSALL | 9693 |
| weighted | EPHLLATSHDKI | 9694 |
| weighted | KLNYDIGVSEMV | 9695 |
| weighted | DIKYVQSQNSES | 9696 |
| weighted | YIEVQRPVKTSK | 9697 |
| weighted | QHQSRIWPIVYL | 9698 |
| weighted | LPFDRRTPALGT | 9699 |
| weighted | ANTPDNTSFSRD | 9700 |
| weighted | YGPHLKKDQVLI | 9701 |
| weighted | IALTKFLESKSL | 9702 |
| weighted | KPSVSEETAFRQ | 9703 |
| weighted | SRAASLSLIAEE | 9704 |
| weighted | TCARRAAMSWYS | 9705 |
| weighted | RHKNLAWGVNVF | 9706 |
| weighted | LQDENTSLGIKS | 9707 |
| weighted | VKGSHANEENKC | 9708 |
| weighted | GAVDFKWSVSDH | 9709 |
| weighted | LKPLALVLAKAT | 9710 |
| weighted | SQPQQALINPKK | 9711 |
| weighted | ITYELHIKNSTL | 9712 |
| weighted | GFIQYMRLIEQE | 9713 |
| weighted | TQKLVKPGKTLK | 9714 |
| weighted | GVLLVTNFMVVE | 9715 |
| weighted | KAPVCSKCEEET | 9716 |
| weighted | KKQLNKQPHALK | 9717 |
| weighted | EINQVKRPTILN | 9718 |
| weighted | DSFRQNSGQKES | 9719 |
| weighted | TLDKLQEPCSLG | 9720 |
| weighted | GRYPTPFDAQSA | 9721 |
| weighted | KREELLRAKERD | 9722 |
| weighted | EIGVCLERKAPF | 9723 |
| weighted | VGGSKIAITAFR | 9724 |
| weighted | LTGFRVNIVFQP | 9725 |
| weighted | GNQQGVQLETLE | 9726 |
| weighted | AEGSSKKRLLFL | 9727 |
| weighted | EAYEKEYPSDVL | 9728 |
| weighted | YKLAVQREIAAA | 9729 |
| weighted | REEFADIGAEDA | 9730 |
| weighted | FPHAALGDGKFD | 9731 |
| weighted | IAKMTLEVPEPE | 9732 |
| weighted | ECEGLVVQLPSR | 9733 |
| weighted | EYVCWGCHMKAE | 9734 |
| weighted | CFKKPLKSALDR | 9735 |
| weighted | TPAALYSTSVHL | 9736 |
| weighted | VPVPKLHEPPPR | 9737 |
| weighted | ALQPSLPEKAMR | 9738 |
| weighted | YPTAKPLVIAGE | 9739 |
| weighted | LRLCDQVEDCGS | 9740 |
| weighted | SDCAETVLLISF | 9741 |
| weighted | SFDCMELLETCT | 9742 |
| weighted | GLLDEACMPRAH | 9743 |
| weighted | GSDYTWNTLRST | 9744 |
| weighted | RSEKTIRGEYTR | 9745 |
| weighted | SKMRWLRPHPCV | 9746 |
| weighted | HELQNGIPRDNH | 9747 |
| weighted | AILLHTLPNVIQ | 9748 |
| weighted | KSPVISKKEQIF | 9749 |
| weighted | AAPLLHEPDKPI | 9750 |
| weighted | GENLSLDTGDTI | 9751 |
| weighted | SDSESKETNAPL | 9752 |
| weighted | MIQFVHRLILSY | 9753 |
| weighted | TVFIVHDLPRQP | 9754 |
| weighted | LPKQDMKLRNVL | 9755 |
| weighted | VVKKPLSSDFNQ | 9756 |
| weighted | GSQNMNYAYHVN | 9757 |
| weighted | KHGRTLASTQDL | 9758 |
| weighted | CVRRLWSEVAET | 9759 |
| weighted | LEYIRNQLVGSM | 9760 |
| weighted | LTVDIYTEQTID | 9761 |
| weighted | GRVSCEGWLNTA | 9762 |
| weighted | RACKLDMVRFVV | 9763 |
| weighted | DEGFMDPPPAYE | 9764 |
| weighted | MRNFTGRMCMLG | 9765 |
| weighted | EDRVSGITSPIM | 9766 |
| weighted | TGQSLDSNLFYG | 9767 |
| weighted | EAHLDRIEYNSL | 9768 |
| weighted | ALLVMRALQQDL | 9769 |
| weighted | LCDPPGESHQGL | 9770 |
| weighted | TCHLTVSSLQPS | 9771 |
| weighted | EVWFEWGVRHVI | 9772 |
| weighted | VELTPPIPLQGA | 9773 |
| weighted | DYELTLIIDFRS | 9774 |
| weighted | KGGRGEPKNDRL | 9775 |
| weighted | QRSEKSTFSRHP | 9776 |
| weighted | KPIVITSLGAPR | 9777 |
| weighted | DVFVDESACKVF | 9778 |
| weighted | SIGAYCSVINTI | 9779 |
| weighted | SESSIEMGSLVW | 9780 |
| weighted | QEHPVAKAIGIG | 9781 |
| weighted | SRLRNANPFDSI | 9782 |
| weighted | DSQPDKLPPERE | 9783 |
| weighted | GVSTENDVTWNK | 9784 |
| weighted | QDNPQKGELRPY | 9785 |
| weighted | TDLTKLQICFSS | 9786 |
| weighted | DQCPAGQSPSLR | 9787 |
| weighted | STTGNLQLSAVE | 19788 |
| weighted | RRTLATLYKLLK | 9789 |
| weighted | FTCFVGETQGAE | 9790 |
| weighted | CAQDHPQRSGES | 9791 |
| weighted | INEGKFKSAVQV | 9792 |
| weighted | SVRYTCLAKNEE | 9793 |
| weighted | KESDKEWLIDGG | 9794 |
| weighted | IAKVCTSLALPT | 9795 |
| weighted | ANVTPHQMQSRP | 9796 |
| weighted | HPQPARSCNALL | 9797 |
| weighted | HSKENIVPGADV | 9798 |
| weighted | LRHEPVGTNGSG | 9799 |
| weighted | RELDESKWAPPY | 9800 |
| weighted | GELLGDALEEAK | 9801 |
| weighted | DRLGKATKTYCN | 9802 |
| weighted | GSKSNECWYMFQ | 9803 |
| weighted | FHAHRLIHRAPS | 9804 |
| weighted | NAERGPLRTSTL | 9805 |
| weighted | DRSPTREGILGS | 9806 |
| weighted | FTDNTQPDLRGK | 9807 |
| weighted | NQVREGALKYMR | 9808 |
| weighted | INGNRALHAPVT | 9809 |
| weighted | LRKLAWSTLAAW | 9810 |
| weighted | CITEESYALAAN | 9811 |
| weighted | NNDLENTNYSFY | 9812 |
| weighted | AEANSSGLGAEE | 9813 |
| weighted | GLQQWGAAFDFC | 9814 |
| weighted | QVKEFVPHCGLL | 9815 |
| weighted | RMAFTVESLEGM | 9816 |
| weighted | SDYPIAVTQPLG | 9817 |
| weighted | DTPHTKALDPSD | 9818 |
| weighted | ATVFLSMSLNAP | 9819 |
| weighted | LQAGSCPDLLRV | 9820 |
| weighted | EKHLLSYHVALN | 9821 |
| weighted | PERSATLPCMHP | 9822 |
| weighted | VKKRSEPNLYDH | 9823 |
| weighted | PEDTTILYQNDL | 9824 |
| weighted | ILKKLSMGKPPPR | 9825 |
| weighted | GLCPYQSDGQDH | 9826 |
| weighted | YLGVLCAHVVCA | 9827 |
| weighted | DPQMETTRITWG | 9828 |
| weighted | RPNNFVNPSHYL | 9829 |
| weighted | CEPARKGLEKWS | 9830 |
| weighted | RVLLDNYSPAQI | 9831 |
| weighted | RYHKISKEASAL | 9832 |
| weighted | LAKSVEFTLQSP | 9833 |
| weighted | ILMRYIFPVLLR | 9834 |
| weighted | PSKQRSQKSEED | 9835 |
| weighted | VEQLRLKPVLLD | 9836 |
| weighted | DAGYQVVAAKVI | 9837 |
| weighted | EHLMALGTSCGK | 9838 |
| weighted | NNLSSRQKEAQQ | 9839 |
| weighted | NLDYGSHEVITS | 9840 |
| weighted | VSTSLEVVRSAE | 9841 |
| weighted | RAIIHLPSTRDY | 9842 |
| weighted | DQSNSVHDNTST | 9843 |
| weighted | PDVDVAVETEIF | 9844 |
| weighted | EESARKGGYSPR | 9845 |
| weighted | KEVAPTLEGFEL | 9846 |
| weighted | PWDSNAPIKALL | 9847 |
| weighted | TNFYGVHGIGES | 9848 |
| weighted | LKSTHEVHQLSQ | 9849 |
| weighted | YQFHAAEATRAK | 9850 |
| weighted | ECMTLTLDVCLD | 9851 |
| weighted | GIGIEGVKLFYS | 9852 |
| weighted | DSLEGTLQGVDR | 9853 |
| weighted | LEIDHLYRLVGV | 9854 |
| weighted | VKAQAKEKLNSD | 9855 |
| weighted | MEQNLEFAKNTV | 9856 |
| weighted | FDDTWKEQHLVI | 9857 |
| weighted | DPPIEDDGSKRG | 9858 |
| weighted | QIFSAKGLFGCG | 9859 |
| weighted | LLSLSRTQLLPL | 9860 |
| weighted | LCDEQKQVGPKA | 9861 |
| weighted | GIYVQKHYKDTS | 9862 |
| weighted | GEWIAKAGELKN | 9863 |
| weighted | AHELMVIKVDAQ | 9864 |
| weighted | TLRTGDELIQLA | 9865 |
| weighted | EPCPPWRIMAES | 9866 |
| weighted | GEKVKKPLPMGG | 9867 |
| weighted | KDVYNEAPGSSK | 9868 |
| weighted | GSIEADRENGFS | 9869 |
| weighted | DYPFILSINPRE | 9870 |
| weighted | AGAFRPKIQSTL | 9871 |
| weighted | ACEQLEEGRLAI | 9872 |
| weighted | RIENGYVSSQRC | 9873 |
| weighted | AAQSKRFAYGGT | 9874 |
| weighted | HLSMFASQMMFG | 9875 |
| weighted | IRIFKTRKVLKN | 9876 |
| weighted | SLKNHCKCMLTI | 9877 |
| weighted | SKITQLRQLHKA | 9878 |
| weighted | FPGLNSAHNTVY | 9879 |
| weighted | AVKSMQRNPPEQ | 9880 |
| weighted | PKKPELEDKGMS | 9881 |
| weighted | ASVAGYPPRKIT | 9882 |
| weighted | LPLNKQLPSSST | 9883 |
| weighted | VSDRHGERAKKD | 9884 |
| weighted | IDVMSGRSLRLAD | 9885 |
| weighted | LQPGASLNSDKV | 9886 |
| weighted | FCHTPLQNHIAP | 9887 |
| weighted | PNAELLNLAQEQ | 9888 |
| weighted | LLKRLAYSRKSS | 9889 |
| weighted | EAQLIPQFQILI | 9890 |
| weighted | LQMYKQGIAPPW | 9891 |
| weighted | QTTTPRSDMIEA | 9892 |
| weighted | HAVQCETEKTFT | 9893 |
| weighted | GIELVQFMLVSE | 9894 |
| weighted | FELPSSPCQFLM | 9895 |
| weighted | LVLQGERQAMEE | 9896 |
| weighted | PRRKKKQRNVRR | 9897 |
| weighted | VCMPPNTDHTVC | 9898 |
| weighted | WAGFATMAVIEA | 9899 |
| weighted | VDVTAKMLTGSP | 9900 |
| weighted | TMTGESRVVVSK | 9901 |
| weighted | GIYCHLTPVVLS | 9902 |
| weighted | TCTMAADLNHFG | 9903 |
| weighted | YKQNHKVMEPTP | 9904 |
| weighted | APFAARPKVAET | 9905 |
| weighted | QIALSQLRTNKE | 9906 |
| weighted | KRDPPKNDNGTC | 9907 |
| weighted | LPVKYAVSIKVH | 9908 |
| weighted | SPHNVSLGLLFK | 9909 |
| weighted | EIFALLDPTGSA | 9910 |
| weighted | LKTRCFATILIK | 9911 |
| weighted | VKHLAGQIGSGS | 9912 |
| weighted | VMLVLETVREAY | 9913 |
| weighted | RDIEVLNKMHKY | 9914 |
| weighted | SHVDGNSLGIKS | 9915 |
| weighted | AKVHRQTVTAAV | 9916 |
| weighted | MEPPKNLGWSVR | 9917 |
| weighted | PDLHRIVSILLS | 9918 |
| weighted | GLPPDIPPSVRG | 9919 |
| weighted | LGQGKEWMIGHL | 9920 |
| weighted | YEEIEMVMVRLA | 9921 |
| weighted | EEQLHDYWGQKC | 9922 |
| weighted | CPDAVMSLTPVC | 9923 |
| weighted | YGVGLLGKVYDL | 9924 |
| weighted | SPHYTKLGGIRY | 9925 |
| weighted | IGRFSRLLDTSY | 9926 |
| weighted | PPFPIAGESALG | 9927 |
| weighted | LQALDLIAEDFI | 9928 |
| weighted | SLVAQRIGAFAH | 9929 |
| weighted | MGLTLSTKVVIL | 9930 |
| weighted | RALNCILSQRVK | 9931 |
| weighted | KEKNLLKERDGS | 9932 |
| weighted | SVVSKALVTSDE | 9933 |
| weighted | RNGASDLECRTD | 9934 |
| weighted | SFRGQEISQAEF | 9935 |
| weighted | REQEMQYRHMKR | 9936 |
| weighted | FVSEGGSGDGKG | 9937 |
| weighted | AFEIKYKSYTSQ | 9938 |
| weighted | AFWCYDAFPHEK | 9939 |
| weighted | EVRYLASVNKMH | 9940 |
| weighted | SGSKVVREYFAV | 9941 |
| weighted | LGLLAALHQEVE | 9942 |
| weighted | SSGFTGSGRQGP | 9943 |
| weighted | KQDKKRIGPTTP | 9944 |
| weighted | PHCQQQALPSVM | 9945 |
| weighted | RYNLQCNRDSFV | 9946 |
| weighted | RGVKTLRAEPRT | 9947 |
| weighted | SHTNNVKYTLTK | 9948 |
| weighted | HYEIVKVRESPM | 19949 |
| weighted | EWASPLMLEPHF | 9950 |
| weighted | AGRPASTFECYS | 9951 |
| weighted | LSLVAECRDSCT | 9952 |
| weighted | CTGGRLHFKDYP | 9953 |
| weighted | LKLLARKKGTRG | 9954 |
| weighted | QWNLGKGLETDA | 9955 |
| weighted | RKRLYARAGPWK | 9956 |
| weighted | GTIAMIVGGHAR | 9957 |
| weighted | GPYPIHIKVARE | 9958 |
| weighted | SSCPRYYPEQDD | 9959 |
| weighted | GTQSCYSEYLGT | 9960 |
| weighted | GSFSQIFFSQRR | 9961 |
| weighted | PTHEVPKFGEWG | 9962 |
| weighted | VGTTSTASPRFP | 9963 |
| weighted | LYRSLGALIPGD | 9964 |
| weighted | DAYLNAIEPSNF | 9965 |
| weighted | LAPKLTHKIGYM | 9966 |
| weighted | IIPVPEDLDLAV | 9967 |
| weighted | IDLVSAVTSRRF | 9968 |
| weighted | PENLFQLKLMAG | 9969 |
| weighted | LEILFAQGLAPG | 9970 |
| weighted | DAAQPKQSTKVW | 9971 |
| weighted | ARASKKDKTHVP | 9972 |
| weighted | GQLGKSSTTHVE | 9973 |
| weighted | AKFGLENNLARK | 9974 |
| weighted | RDATAAILFVYN | 19975 |
| weighted | KKPTMLCHFGVK | 9976 |
| weighted | GFQPLSTNMLVY | 9977 |
| weighted | IDEDYCLEERVM | 9978 |
| weighted | TEPRFLKLPFLK | 9979 |
| weighted | GQAVLSTPRIQS | 9980 |
| weighted | GPYKLAAKEVVE | 9981 |
| weighted | TDGNKQDNTGDF | 9982 |
| weighted | YPEKVQENLETR | 9983 |
| weighted | GRTCDGILEWDH | 9984 |
| weighted | TGNTKQSWEPAT | 9985 |
| weighted | LDCKIEIFVHVA | 9986 |
| weighted | RAYLYKRLHIPQ | 9987 |
| weighted | MKIQIYILTLEV | 9988 |
| weighted | PSGGQLNKSERN | 9989 |
| weighted | EPQPIALLVEGP | 9990 |
| weighted | ILTLEFVRSLQNS | 9991 |
| weighted | AARSAVTSPLRI | 9992 |
| weighted | NWLERINKKIRA | 9993 |
| weighted | IFDPKSKTKPGT | 9994 |
| weighted | TGHQDRTILGIP | 9995 |
| weighted | AGKLRRVQSGVK | 9996 |
| weighted | HSAQRLSGGLIL | 9997 |
| weighted | PTFVLDEDHLSG | 9998 |
| weighted | KVKNGHPLHNTP | 9999 |
| weighted | FINSQKDTLTVK | 10000 |
| weighted | TMAFVSTGTLYA | 10001 |
| weighted | FRGFFSDGGAQQ | 10002 |
| weighted | PFVGFPVVIYGI | 10003 |
| weighted | FGPNSCTIGAPY | 10004 |
| weighted | QPFVKLRCVEEV | 10005 |

We claim:

1. A method of determining a putative source of a peptide sequence of a peptide, the method comprising:
receiving the peptide sequence; and
determining, based at least in part on one or more searches of the peptide sequence within one or more databases, the putative source associated with the peptide sequence,
wherein each respective search of the one or more searches has a random hit rate that is based at least in part on a number of random sequences found by the respective search, and
wherein the one or more searches are performed in order of increasing random hit rates until the putative source is determined.

2. The method of claim 1,
wherein the one or more databases comprises an expanded human proteome database, and
wherein the expanded human proteome database comprises computer-readable representations of translations from messenger ribonucleic acids (RNAs) and non-coding RNAs.

3. The method of claim 2, wherein the expanded human proteome database comprises computer-readable representations of translations from micro RNAs.

4. The method of claim 2, wherein the expanded human proteome database comprises computer-readable representations of translations from long non-coding RNAs.

5. The method of claim 2, wherein the expanded human proteome database comprises computer-readable representations of translations of human endogenous retroviruses.

6. The method of claim 2,
wherein the one or more searches comprises a linear human proteome search for the peptide sequence within the expanded human proteome database, and
wherein the putative source is a linear expanded human proteome source when the linear human proteome search for the peptide sequence within the expanded human proteome database finds the peptide sequence within the expanded human proteome database.

7. The method of claim 6, further comprising:
identifying, when the source is the linear expanded human proteome source, whether the peptide is putatively translated from messenger RNA or non-coding RNA.

8. The method of claim 2,
wherein the one or more databases comprises a human genome database,
wherein the one or more searches comprises a linear human genome search of translations of the human genome database, and
wherein the putative source is a linear genome source when the linear human genome search finds human genome sequence from which the peptide is putatively synthesized.

9. The method of claim 8,
wherein the linear human genome search excludes portions of the human genome from which the messenger RNA and the non-coding RNA of the expanded human proteome database are transcribed and includes remaining portions of the human genome, and
wherein the linear human genome search comprises a search of six frame translations of the human genome.

10. The method of claim 2,
wherein the one or more searches comprises a linear mismatch search for peptides having a mismatch to the peptide sequence within the expanded human proteome database, and
wherein the putative source is a linear mismatch of the expanded human proteome when the linear mismatch search finds a peptide sequence having a mismatch to the peptide sequence within expanded human proteome database.

11. The method of claim 10, wherein the linear mismatch search is a search for peptide sequences having only a single mismatch to the peptide sequence.

12. The method of claim 1,
wherein the one or more databases comprises a non-endogenous proteome database comprising computer-readable representations of proteins translated from RNA from non-endogenous organisms and/or proteins synthesized by non-endogenous organisms,
wherein the one or more searches comprises a linear non-endogenous search for the peptide sequence within the non-endogenous proteome database, and
wherein the putative source is a linear non-endogenous proteome source when the linear non-endogenous search finds the peptide sequence within the non-endogenous proteome database.

13. The method of claim 12, wherein the non-endogenous proteome database comprises a Basic Local Alignment Search Tool (BLAST) database.

14. The method of claim 2,
wherein the one or more searches comprises a cis-spliced search, within the expanded human proteome database, for peptide fragments that can be cis-spliced to match the peptide sequence, and
wherein the source is a cis-spliced human proteome source when the cis-spliced search finds, within the expanded human proteome database, peptide fragments that can be cis-spliced to match the peptide sequence.

15. The method of claim 2,
wherein the one or more searches comprises a trans-spliced search, within the expanded human proteome database, for computer-readable representations of peptide fragments that can be trans-spliced to match the peptide sequence, and
wherein the source is a trans-spliced human proteome source when the trans-spliced search finds, within the expanded human proteome database, computer-readable representations of peptide fragments that can be trans-spliced to match the peptide sequence.

16. The method of claim 15, wherein the putative source is determined to be unidentified when the trans-spiced search does not find computer-readable representations of peptide fragments that can be trans-spliced to match the peptide sequence.

17. The method of claim 2,
wherein the one or more databases comprises a human genome database, and
wherein the one or more searches comprise the following searches ordered sequentially in a workflow as follows:
a linear human proteome search for the peptide sequence within the expanded human proteome database;
a linear human genome search of translations of the human genome database;
a linear mismatch search for peptides having a mismatch to the peptide sequence within the expanded human proteome database; and a cis-spliced search, within the expanded human proteome database, for peptide fragments that can be cis-spliced to match the peptide sequence.

18. The method of claim 17,
wherein the one or more databases comprises a non-endogenous proteome database comprising computer-readable representations of proteins translated from RNA from non-endogenous organisms and/or proteins synthesized by non-endogenous organisms,
wherein the one or more searches further comprises a linear non-endogenous search for the peptide sequence within the non-endogenous proteome database, and
wherein the linear non-endogenous search is ordered sequentially in the workflow after the linear mismatch search and before the cis-spliced search.

19. The method of claim 17,
wherein the one or more searches further comprises a trans-spliced search, within the expanded human proteome database, for peptide fragments that can be trans-spliced to match the peptide sequence, and
wherein the trans-spliced search is ordered sequentially in the workflow after the cis-spliced search.

20. The method of claim 17, further comprising:
halting advancement of the workflow to a subsequent search of the one or more searches when the putative source is determined for the peptide sequence.

21. The method of claim 1, wherein the peptide sequence comprises at least one ambiguous residue, the method further comprising:
generating a plurality of permutated peptide sequences each comprising a potential residue for each of the at least one ambiguous residue;
determining, for each of the plurality of permutated peptide sequences, a respective potential source; and
determining the putative source of the peptide sequence such that the putative source is a respective potential source.

22. The method of claim 21, wherein the potential residue for each of the at least one ambiguous residue comprises leucine and isoleucine.

23. The method of claim 21, further comprising:
determining a respective random hit rate for each of the respective potential sources such that the random hit rate increases as a number of random sequences are found by a respective search of the one or more searches; and
determining the putative source such that the respective random hit rate of the putative source is the lowest of the respective random hit rates for each of the potential sources.

24. The method of claim 21, further comprising:
identifying one or more likely permutated peptide sequences of the plurality of permutated peptide sequences such that each of the one or more likely permutated peptide sequences are associated with the putative source.

25. The method of claim 1, wherein the peptide sequence is a de novo peptide sequence determined via mass spectrometry.

26. Non-transitory computer-readable medium configured to communicate with one or more processor(s) of a computational device, the non-transitory computer-readable medium including instructions thereon, that when executed by the processor(s), cause the computational device to:
receive, as an input, a peptide sequence;
determine, based at least in part on one or more searches of the peptide sequence within one or more databases, a putative source associated with the peptide sequence,
wherein each respective search of the one or more searches has a random hit rate that is based at least in part on a number of random sequences found by the respective search, and
wherein the one or more searches are performed in order of increasing random hit rates until the putative source is determined; and
provide, as an output, the putative source.

27. The non-transitory computer readable medium of claim 26,
wherein the one or more databases comprises an expanded human proteome database, and
wherein the expanded human proteome database comprises computer-readable representations of translations from messenger ribonucleic acids (RNAs) and non-coding RNAs.

28. The non-transitory computer readable medium of claim 27, wherein the expanded human proteome database comprises computer-readable representations of translations from micro RNAs.

29. The non-transitory computer readable medium of claim 27, wherein the expanded human proteome database comprises computer-readable representations of translations from long non-coding RNAs.

30. The non-transitory computer readable medium of claim 27, wherein the expanded human proteome database comprises computer-readable representations of translations of human endogenous retroviruses.

31. The non-transitory computer readable medium of claim 27,
wherein the one or more searches comprises a linear human proteome search for the peptide sequence within the expanded human proteome database, and
wherein the putative source is a linear expanded human proteome source when the linear human proteome search for the peptide sequence within the expanded human proteome database finds the peptide sequence within the expanded human proteome database.

32. The non-transitory computer readable medium of claim 31, wherein, the instructions,
when executed by the processor(s), cause the computational device to:
identify, when the source is the linear expanded human proteome source, whether the peptide is putatively translated from messenger RNA or non-coding RNA.

33. The non-transitory computer readable medium of claim 27,
wherein the one or more databases comprises a human genome database,
wherein the one or more searches comprises a linear human genome search of translations of the human genome database, and
wherein the putative source is a linear genome source when the linear human genome search finds human genome sequence from which the peptide is putatively synthesized.

34. The non-transitory computer readable medium of claim 33, wherein the linear human genome search excludes portions of the human genome from which the messenger RNA and the non-coding RNA of the expanded human proteome database are transcribed and includes remaining portions of the human genome.

35. The non-transitory computer readable medium of claim 27,
wherein the one or more searches comprises a linear mismatch search for peptides having a mismatch to the peptide sequence within the expanded human proteome database, and
wherein the putative source is a linear mismatch of the expanded human proteome when the linear mismatch search finds a peptide sequence having a mismatch to the peptide sequence within expanded human proteome database.

36. The non-transitory computer readable medium of claim 35, wherein the linear mismatch search is a search for peptide sequences having only a single mismatch to the peptide sequence.

37. The non-transitory computer readable medium of claim 26,
wherein the one or more databases comprises a non-endogenous proteome database comprising computer-readable representations of proteins translated from RNA from non-endogenous organisms and/or proteins synthesized by non-endogenous organisms,
wherein the one or more searches comprises a linear non-endogenous search for the peptide sequence within the non-endogenous proteome database, and
wherein the putative source is a linear non-endogenous proteome source when the linear non-endogenous search finds the peptide sequence within the non-endogenous proteome database.

38. The non-transitory computer readable medium of claim 37, wherein the non-endogenous proteome database comprises a Basic Local Alignment Search Tool (BLAST) database.

39. The non-transitory computer readable medium of claim 27,
wherein the one or more searches comprises a cis-spliced search, within the expanded human proteome database, for peptide fragments that can be cis-spliced to match the peptide sequence, and
wherein the source is a cis-spliced human proteome source when the cis-spliced search finds, within the expanded human proteome database, peptide fragments that can be cis-spliced to match the peptide sequence.

40. The non-transitory computer readable medium of claim 27,
wherein the one or more searches comprises a trans-spliced search, within the expanded human proteome database, for computer-readable representations of peptide fragments that can be trans-spliced to match the peptide sequence, and
wherein the source is a trans-spliced human proteome source when the trans-spliced search finds, within the expanded human proteome database, computer-readable representations of peptide fragments that can be trans-spliced to match the peptide sequence.

41. The non-transitory computer readable medium of claim 40, wherein the putative source is determined to be unidentified when the trans-spiced search does not find computer-readable representations of peptide fragments that can be trans-spliced to match the peptide sequence.

42. The non-transitory computer readable medium of claim 27,
wherein the one or more databases comprises a human genome database, and
wherein the one or more searches comprise the following searches ordered sequentially in a workflow as follows:
a linear human proteome search for the peptide sequence within the expanded human proteome database;
a linear human genome search of translations of the human genome database;
a linear mismatch search for peptides having a mismatch to the peptide sequence within the expanded human proteome database; and
a cis-spliced search, within the expanded human proteome database, for peptide fragments that can be cis-spliced to match the peptide sequence.

43. The non-transitory computer readable medium of claim 42,
wherein the one or more databases comprises a non-endogenous proteome database comprising computer-readable representations of proteins translated from RNA from non-endogenous organisms and/or proteins synthesized by non-endogenous organisms,
wherein the one or more searches further comprises a linear non-endogenous search for the peptide sequence within the non-endogenous proteome database, and
wherein the linear non-endogenous search is ordered sequentially in the workflow after the linear mismatch search and before the cis-spliced search.

44. The method of claim 42,
wherein the one or more searches further comprises a trans-spliced search, within the expanded human proteome database, for peptide fragments that can be trans-spliced to match the peptide sequence, and
wherein the trans-spliced search is ordered sequentially in the workflow after the cis-spliced search.

45. The non-transitory computer readable medium of claim 42, wherein, the instructions,
when executed by the processor(s), cause the computational device to:
halt advancement of the workflow to a subsequent search of the one or more searches when the putative source is determined for the peptide sequence.

46. The non-transitory computer readable medium of claim 26,
wherein the peptide sequence comprises at least one ambiguous residue, and
wherein, the instructions, when executed by the processor(s), cause the computational device to:
generate a plurality of permutated peptide sequences each comprising a potential residue for each of the at least one ambiguous residue;
determine, for each of the plurality of permutated peptide sequences, a respective potential source; and
determine the putative source of the peptide sequence such that the putative source is a respective potential source.

47. The non-transitory computer readable medium of claim 46, wherein the potential residue for each of the at least one ambiguous residue comprises leucine and isoleucine.

48. The non-transitory computer readable medium of claim 46, wherein, the instructions,
when executed by the processor(s), cause the computational device to:
determine a respective random hit rate for each of the respective potential sources such that the random hit rate increases as a number of random sequences are found by a respective search of the one or more searches; and
determine the putative source such that the respective random hit rate of the putative source is the lowest of the respective random hit rates for each of the potential sources.

49. The non-transitory computer readable medium of claim 46, wherein, the instructions,
when executed by the processor(s), cause the computational device to:
identify one or more likely permutated peptide sequences of the plurality of permutated peptide sequences such that each of the one or more likely permutated peptide sequences are associated with the putative source.

50. The non-transitory computer readable medium of claim 26, wherein the peptide sequence is a de novo peptide sequence determined via mass spectrometry.

51. A method of ordering a peptide source assignment workflow, the method comprising:
generating a plurality of random peptide sequences;
determining a plurality of peptide source search steps;
searching for each of the plurality of random peptide sequences by each of the plurality of peptide source search steps;
determining, for each of the plurality of peptide source search steps, a random hit rate for a respective search step of the plurality of peptide source search steps based at least in part on a number of the plurality of random peptide sequences found by the respective search step; and
ordering the peptide source search steps in the peptide source assignment workflow from lowest random hit rate to highest random hit rate.

52. The method of claim 51, wherein the random peptide sequences comprise random sequences uniformly sampling all amino acids.

53. The method of claim 51, wherein the random peptide sequences comprise sequences with frequencies of amino acids matching those found in vertebrates.

54. The method of claim 51, wherein each peptide of the random peptide sequences comprises a length of eight to fourteen amino acids.

55. The method of claim 51, wherein each peptide of the random peptide sequences comprises a length of nine to fourteen amino acids, ten to fourteen amino acids, eleven to fourteen amino acids, twelve to fourteen amino acids, thirteen to fourteen amino acids, eight to thirteen amino acids, eight to twelve amino acids, eight to eleven amino acids, eight to ten amino acids, eight to nine amino acids, nine to thirteen amino acids, nine to twelve amino acids, nine to eleven amino acids, nine to ten amino acids, ten to thirteen amino acids, ten to twelve amino acids, ten to eleven amino acids, eleven to thirteen amino acids, elven to twelve amino acids, or twelve to thirteen amino acids.

56. The method of claim 51,
wherein the plurality of peptide source search steps comprises a linear human proteome search for a peptide sequence within an expanded human proteome database, and
wherein the expanded human proteome database comprises computer-readable representations of translations from messenger ribonucleic acids (RNAs) and non-coding RNAs.

57. The method of claim 56, wherein the expanded human proteome database comprises computer-readable representations of translations from micro RNAs.

58. The method of claim 56, wherein the expanded human proteome database comprises computer-readable representations of translations from long non-coding RNAs.

59. The method of claim 56, wherein the expanded human proteome database comprises computer-readable representations of translations of human endogenous retroviruses.

60. The method of claim 56, wherein the plurality of peptide source search steps comprises a linear human genome search of translations of a human genome database.

61. The method of claim 60, wherein the linear human genome search excludes portions of the human genome from which the messenger RNA and the non-coding RNA of the expanded human proteome database are transcribed and includes remaining portions of the human genome.

62. The method of claim 51,
wherein the plurality of peptide source search steps comprises a linear mismatch search for peptides having a mismatch to the peptide sequence within an expanded human proteome database, and
wherein the expanded human proteome database comprises computer-readable representations of translations from messenger ribonucleic acids (RNAs) and non-coding RNAs.

63. The method of claim 51,
wherein the plurality of peptide source search steps comprises a linear non-endogenous search for the peptide sequence within a non-endogenous proteome database, and
wherein the non-endogenous proteome database comprises computer-readable representations of proteins translated from RNA from non-endogenous organisms and/or proteins synthesized by non-endogenous organisms.

64. The method of claim 63, wherein the non-endogenous proteome database comprises a Basic Local Alignment Search Tool (BLAST) database.

65. The method of claim 51,
wherein the plurality of peptide source search steps comprises a cis-spliced search, within an expanded human proteome database, for peptide fragments that can be cis-spliced to match the peptide sequence, and
wherein the expanded human proteome database comprises computer-readable representations of translations from messenger ribonucleic acids (RNAs) and non-coding RNAs.

66. The method of claim 51,
wherein the plurality of peptide source search steps comprises a trans-spliced search, within an expanded human proteome database, for peptide fragments that can be trans-spliced to match the peptide sequence, and
wherein the expanded human proteome database comprises computer-readable representations of translations from messenger ribonucleic acids (RNAs) and non-coding RNAs.

67. The method of claim 51, wherein the peptide source assignment workflow terminates with a peptide being not assigned when the peptide is not assigned a peptide source by any of the plurality of peptide source search steps.

68. The method of claim 51, wherein the peptide source assignment workflow comprises the following searches ordered sequentially as follows:
a linear human proteome search for the peptide sequence within the expanded human proteome database;
a linear human genome search of translations of a human genome database;
a linear mismatch search for peptides having a mismatch to the peptide sequence within the expanded human proteome database; and
a cis-spliced search, within the expanded human proteome database, for peptide fragments that can be cis-spliced to match the peptide sequence.

69. The method of claim 68,
wherein the peptide source assignment workflow comprises a linear non-endogenous search for the peptide sequence within a non-endogenous proteome database, and
wherein the linear non-endogenous search is ordered sequentially within the peptide assignment workflow after the linear mismatch search and before the cis-spliced search.

70. The method of claim 68,
wherein the peptide source assignment workflow comprises a trans-spliced search,
within the expanded human proteome database, for peptide fragments that can be trans-spliced to match the peptide sequence, and
wherein the trans-spliced search is ordered sequentially within the peptide assignment workflow after the cis-spliced search.

71. Non-transitory computer-readable medium configured to communicate with one or more processor(s) of a computational device, the non-transitory computer-readable medium including instructions thereon, that when executed by the processor(s), cause the computational device to:
receive, as an input, a plurality of peptide source search steps;
generate a plurality of random peptide sequences;
search for each of the plurality of random peptide sequences by each of the plurality of peptide source search steps;
determine, for each of the plurality of peptide source search steps, a random hit rate for a respective search step of the plurality of peptide source search steps based at least in part on a number of the plurality of random peptide sequences found by the respective search step;
order the peptide source search steps in a peptide source assignment workflow from lowest random hit rate to highest random hit rate; and
provide, as an output, the peptide source assignment workflow.

72. The non-transitory computer readable medium of claim 71, wherein the random peptide sequences comprise random sequences uniformly sampling all amino acids.

73. The non-transitory computer readable medium of claim 71, wherein the random peptide sequences comprise sequences with frequencies of amino acids matching those found in vertebrates.

74. The non-transitory computer readable medium of claim 71, wherein each peptide of the random peptide sequences comprises a length of eight to fourteen amino acids.

75. The non-transitory computer readable medium of claim 71, wherein each peptide of the random peptide sequences comprises a length of nine to fourteen amino acids, ten to fourteen amino acids, eleven to fourteen amino acids, twelve to fourteen amino acids, thirteen to fourteen amino acids, eight to thirteen amino acids, eight to twelve amino acids, eight to eleven amino acids, eight to ten amino acids, eight to nine amino acids, nine to thirteen amino acids, nine to twelve amino acids, nine to eleven amino acids, nine to ten amino acids, ten to thirteen amino acids, ten to twelve amino acids, ten to eleven amino acids, eleven to thirteen amino acids, elven to twelve amino acids, or twelve to thirteen amino acids.

76. The non-transitory computer readable medium of claim 71,
wherein the plurality of peptide source search steps comprises a linear human proteome search for a peptide sequence within an expanded human proteome database, and
wherein the expanded human proteome database comprises computer-readable representations of translations from messenger ribonucleic acids (RNAs) and non-coding RNAs.

77. The non-transitory computer readable medium of claim 76, wherein the expanded human proteome database comprises computer-readable representations of translations from micro RNAs.

78. The non-transitory computer readable medium of claim 76, wherein the expanded human proteome database comprises computer-readable representations of translations from long non-coding RNAs.

79. The non-transitory computer readable medium of claim 76, wherein the expanded human proteome database comprises computer-readable representations of translations of human endogenous retroviruses.

80. The non-transitory computer readable medium of claim 76, wherein the plurality of peptide source search steps comprises a linear human genome search of translations of a human genome database.

81. The non-transitory computer readable medium of claim 80, wherein the linear human genome search excludes portions of the human genome from which the messenger RNA and the non-coding RNA of the expanded human proteome database are transcribed and includes remaining portions of the human genome.

82. The non-transitory computer readable medium of claim 71,
wherein the plurality of peptide source search steps comprises a linear mismatch search for peptides having a mismatch to the peptide sequence within an expanded human proteome database, and
wherein the expanded human proteome database comprises computer-readable representations of translations from messenger ribonucleic acids (RNAs) and non-coding RNAs.

83. The non-transitory computer readable medium of claim 71,
wherein the plurality of peptide source search steps comprises a linear non-endogenous search for the peptide sequence within a non-endogenous proteome database, and
wherein the non-endogenous proteome database comprises computer-readable representations of proteins translated from RNA from non-endogenous organisms and/or proteins synthesized by non-endogenous organisms.

84. The non-transitory computer readable medium of claim 83, wherein the non-endogenous proteome database comprises a Basic Local Alignment Search Tool (BLAST) database.

85. The non-transitory computer readable medium of claim 71,
wherein the plurality of peptide source search steps comprises a cis-spliced search, within an expanded human proteome database, for peptide fragments that can be cis-spliced to match the peptide sequence, and
wherein the expanded human proteome database comprises computer-readable representations of translations from messenger ribonucleic acids (RNAs) and non-coding RNAs.

86. The non-transitory computer readable medium of claim 71,
wherein the plurality of peptide source search steps comprises a trans-spliced search, within an expanded human proteome database, for peptide fragments that can be trans-spliced to match the peptide sequence, and
wherein the expanded human proteome database comprises computer-readable representations of translations from messenger ribonucleic acids (RNAs) and non-coding RNAs.

87. The non-transitory computer readable medium of claim 71, wherein the peptide source assignment workflow terminates with a peptide being not assigned when the peptide is not assigned a peptide source by any of the plurality of peptide source search steps.

88. The non-transitory computer readable medium of claim 71, wherein the peptide source assignment workflow comprises the following searches ordered sequentially as follows:
a linear human proteome search for the peptide sequence within the expanded human proteome database;
a linear human genome search of translations of a human genome database;
a linear mismatch search for peptides having a mismatch to the peptide sequence within the expanded human proteome database; and
a cis-spliced search, within the expanded human proteome database, for peptide fragments that can be cis-spliced to match the peptide sequence.

89. The non-transitory computer readable medium of claim 88,
wherein the peptide source assignment workflow comprises a linear non-endogenous search for the peptide sequence within a non-endogenous proteome database, and
wherein the linear non-endogenous search is ordered sequentially within the peptide assignment workflow after the linear mismatch search and before the cis-spliced search.

90. The non-transitory computer readable medium of claim 88,
wherein the peptide source assignment workflow comprises a trans-spliced search, within the expanded human proteome database, for peptide fragments that can be trans-spliced to match the peptide sequence, and
wherein the trans-spliced search is ordered sequentially within the peptide assignment workflow after the cis-spliced search.

91. A method comprising:
generating a plurality of simulated random queries;
determining, based on applying the plurality of simulated random queries to each source of a plurality of sources, a number of matches associated with each source;
determining, based on the numbers of matches associated with each source, a false discovery rate associated with each source; and
generating, based on the false discovery rates, a query support data structure configured to facilitate application of a new query to the plurality of sources.

92. The method of claim 91, wherein generating the plurality of simulated random queries comprises at least one of:
generating a plurality of uniform random queries; or
generating a plurality of weighted random queries.

93. The method of claim 91, wherein the plurality of simulated random queries comprises a plurality of simulated random text strings.

94. The method of claim 91, wherein the plurality of simulated random queries comprises a plurality of simulated random peptide sequences.

95. The method of claim 91, wherein determining, based on the numbers of matches associated with each source, the false discovery rate associated with each source comprises a function of the number of matches and a number of the plurality of simulated random queries.

96. The method of claim 91, wherein determining, based on the numbers of matches associated with each source, the false discovery rate associated with each source comprises dividing the number of matches by a number of the plurality of simulated random queries.

97. An apparatus comprising:
one or more processors; and
a memory storing processor-executable instructions that, when executed by the one or more processors, cause the apparatus to:
generate a plurality of simulated random queries;
determine, based on applying the plurality of simulated random queries to each source of a plurality of sources, a number of matches associated with each source;
determine, based on the numbers of matches associated with each source, a false discovery rate associated with each source; and
generate, based on the false discovery rates, a query support data structure configured to facilitate application of a new query to the plurality of sources.

98. The apparatus of claim 97, wherein the processor-executable instructions that cause the apparatus to generate the plurality of simulated random queries further cause the apparatus to at least one of:
generate a plurality of uniform random queries; or
generate a plurality of weighted random queries.

99. The apparatus of claim 97, wherein the plurality of simulated random queries comprises a plurality of simulated random text strings.

100. The apparatus of claim 97, wherein the plurality of simulated random queries comprises a plurality of simulated random peptide sequences.

101. The apparatus of claim 97, wherein the processor-executable instructions that cause the apparatus to determine, based on the numbers of matches associated with each source, the false discovery rate associated with each source further cause the apparatus to determine the false discovery rate as a function of the number of matches and a number of the plurality of simulated random queries.

102. The apparatus of claim 97, wherein the processor-executable instructions further cause the apparatus to determine the false discovery rate by dividing the number of matches by a number of the plurality of simulated random queries.

103. One or more non-transitory computer-readable media storing processor-executable instructions thereon that, when executed by a processor, cause the processor to:
generate a plurality of simulated random queries;
determine, based on applying the plurality of simulated random queries to each source of a plurality of sources, a number of matches associated with each source;
determine, based on the numbers of matches associated with each source, a false discovery rate associated with each source; and
generate, based on the false discovery rates, a query support data structure configured to facilitate application of a new query to the plurality of sources.

104. The one or more non-transitory computer-readable media of claim 103, wherein the processor-executable instructions that cause the processor to generate the plurality of simulated random queries further cause the processor to at least one of:
generate a plurality of uniform random queries; or
generate a plurality of weighted random queries.

105. The one or more non-transitory computer-readable media of claim 103, wherein the plurality of simulated random queries comprises a plurality of simulated random text strings.

106. The one or more non-transitory computer-readable media of claim 103, wherein the plurality of simulated random queries comprises a plurality of simulated random peptide sequences.

107. The one or more non-transitory computer-readable media of claim 103, wherein the processor-executable instructions that cause the processor to determine, based on the numbers of matches associated with each source, the false discovery rate associated with each source further cause the processor to determine the false discovery rate as a function of the number of matches and a number of the plurality of simulated random queries.

108. The one or more non-transitory computer-readable media of claim 103, wherein the processor-executable instructions that cause the processor to determine, based on the numbers of matches associated with each source, the false discovery rate associated with each source further cause the processor to determine the false discovery rate by dividing the number of matches by a number of the plurality of simulated random queries.

109. A system comprising:
a computing device configured to:
generate a plurality of simulated random queries,
determine, based on applying the plurality of simulated random queries to each source of a plurality of sources, a number of matches associated with each source,
determine, based on the numbers of matches associated with each source, a false discovery rate associated with each source, and
generate, based on the false discovery rates, a query support data structure configured to facilitate application of a new query to the plurality of sources; and
the plurality of sources configured to:
receive the plurality of simulated random queries,
determine if a number of matches for the plurality of simulated random queries exists, and
output a result indicating the number of matches.

110. The system of claim 109, wherein the computing device configured to generate the plurality of simulated random queries is further configured to cause the processor to at least one of:
generate a plurality of uniform random queries; or
generate a plurality of weighted random queries.

111. The system of claim 109, wherein the plurality of simulated random queries comprises a plurality of simulated random text strings.

112. The system of claim 109, wherein the plurality of simulated random queries comprises a plurality of simulated random peptide sequences.

113. The system of claim 109, wherein the computing device configured to determine,
based on the numbers of matches associated with each source, the false discovery rate associated with each source is further configured to determine the false discovery rate as a function of the number of matches and a number of the plurality of simulated random queries.

114. The system of claim 109, wherein the computing device configured to determine,
based on the numbers of matches associated with each source, the false discovery rate associated with each source is further configured to determine the false discovery rate by dividing the number of matches by a number of the plurality of simulated random queries.

115. A method comprising:
receiving a query;
applying, based on a query support data structure, the query to one or more sources of a plurality of sources;
determining, based on a query result, a label associated with a source of the plurality of sources associated with the query result; and
applying the label to the query.

116. The method of claim 115, wherein the query comprises a text string.

117. The method of claim 115, wherein the query comprises a peptide sequence.

118. The method of claim 117, wherein receiving the query comprises receiving the peptide sequence from a mass spectrometer system.

119. The method of claim 115, further comprising determining, via the mass spectrometer system, one or more amino acids of the peptide sequence.

120. The method of claim 115, wherein the query support data structure indicates an order of the plurality of sources to apply the query, wherein the order is based on a false discovery rate associated with each source of the plurality of sources.

121. The method of claim 115, further comprising determining one or more permutations of the query.

122. The method of claim 121, wherein applying, based on the query support data structure, the query to the one or more sources of the plurality of sources comprises:
applying each permutation of the one or more permutations of the query to the one or more sources of the plurality of sources;
if an identical match to the one or more permutations of the query is found in a first source of the plurality of sources, discontinuing additional searches and applying a linear label to the one or more permutations of the query associated with the identical match; and
assigning the one or more permutations of the query associated with the identical match as a correct query.

123. The method of claim 115, wherein applying the query to one or more sources of a plurality of sources comprises:
searching for an identical match to the query in a first source of the plurality of sources; and
if an identical match to the query is found in the first source of the plurality of sources, discontinuing additional searches.

124. The method of claim 123, wherein the query result comprises the identical match and wherein the label associated with a source of the plurality of sources associated with the query result comprises a linear label.

125. The method of claim 115, wherein applying the query to one or more sources of a plurality of sources comprises:
searching for an identical match to the query in a first source of the plurality of sources; and
if an identical match to the one or more permutations of the query is found in the first source of the plurality of sources, discontinuing additional searches.

126. The method of claim 125, wherein the query result comprises the identical match and wherein the label associated with a source of the plurality of sources associated with the query result comprises a linear label.

127. The method of claim 125, wherein applying the query to one or more sources of a plurality of sources comprises:
searching for an identical match to the query in any frame of a plurality of frames of a second source of the plurality of sources; and
if an identical match to the query is found in any frame of a plurality of frames of the second source of the plurality of sources, discontinuing additional searches.

128. The method of claim 127, wherein the query result comprises the identical match and wherein the label associated with a source of the plurality of sources associated with the query result comprises a linear label.

129. The method of claim 127, wherein applying the query to one or more sources of a plurality of sources comprises:
searching for a non-identical match to the query in a third source of the plurality of sources; and
if a non-identical match to the query is found in the third source of the plurality of sources, discontinuing additional searches.

130. The method of claim 129, wherein the query result comprises the non-identical match and wherein the label associated with a source of the plurality of sources associated with the query result comprises a mismatch label.

131. The method of claim 129, wherein applying the query to one or more sources of a plurality of sources comprises:
searching for a homologous match to the query in a fourth source of the plurality of sources; and
if a homologous match to the query is found in the fourth source of the plurality of sources, discontinuing additional searches.

132. The method of claim 131, wherein the query result comprises the homologous match and wherein the label associated with a source of the plurality of sources associated with the query result comprises a homologous label.

133. The method of claim 131, wherein applying the query to one or more sources of a plurality of sources comprises:
splitting the query into a plurality of sets of fragments;
searching for each set of fragments in a fifth source of the plurality of sources;
if a match for a set of fragments is found in the fifth source of the plurality of sources, discontinuing additional searches; and
if a first match for a first fragment of the set of fragments and a second match for a second fragment of the set of fragments is found in the fifth source of the plurality of sources, discontinuing additional searches.

134. The method of claim 133, wherein the query result comprises the match for the set of fragments and wherein the label associated with a source of the plurality of sources associated with the query result comprises a cis-spliced label.

135. The method of claim 133, wherein the query result comprises the first match for the first fragment of the set of fragments and the second match for the second fragment of the set of fragments and wherein the label associated with a source of the plurality of sources associated with the query result comprises a trans-spliced label.

136. The method of claim 115, further comprising determining, based on the label, a source of the query.

137. The method of claim 136, further comprising, validating output of a mass spectrometer system based on the source of the query.

138. An apparatus comprising:
one or more processors; and
a memory storing processor-executable instructions that, when executed by the one or more processors, cause the apparatus to:
receive a query;
apply, based on a query support data structure, the query to one or more sources of a plurality of sources;
determine, based on a query result, a label associated with a source of the plurality of sources associated with the query result; and
apply the label to the query.

139. The apparatus of claim 138, wherein the query comprises a text string.

140. The apparatus of claim 138, wherein the query comprises a peptide sequence.

141. The apparatus of claim 138, wherein the processor-executable instructions that cause the apparatus to receive the query further cause the apparatus to receive the peptide sequence from a mass spectrometer system.

142. The apparatus of claim 138, wherein the processor-executable instructions further cause the apparatus to determine, via the mass spectrometer system, one or more amino acids of the peptide sequence.

143. The apparatus of claim 138, wherein the query support data structure indicates an order of the plurality of sources to apply the query, wherein the order is based on a false discovery rate associated with each source of the plurality of sources.

144. The apparatus of claim 138, wherein the processor-executable instructions further cause the apparatus to determine one or more permutations of the query.

145. The apparatus of claim 144, wherein the processor-executable instructions that cause the apparatus to apply, based on the query support data structure, the query to the one or more sources of the plurality of sources further cause the apparatus to:
apply each permutation of the one or more permutations of the query to the one or more sources of the plurality of sources;
if an identical match to the one or more permutations of the query is found in a first source of the plurality of sources, discontinue additional searches and applying a linear label to the one or more permutations of the query associated with the identical match; and
assign the one or more permutations of the query associated with the identical match as a correct query.

146. The apparatus of claim 138, wherein the processor-executable instructions that cause the apparatus to apply the query to one or more sources of a plurality of sources further cause the apparatus to:
search for an identical match to the query in a first source of the plurality of sources; and
if an identical match to the query is found in the first source of the plurality of sources, discontinue additional searches.

147. The apparatus of claim 146, wherein the query result comprises the identical match and wherein the label associated with a source of the plurality of sources associated with the query result comprises a linear label.

148. The apparatus of claim 138, wherein the processor-executable instructions that cause the apparatus to apply the query to one or more sources of a plurality of sources further cause the apparatus to:
search for an identical match to the query in a first source of the plurality of sources; and
if an identical match to the one or more permutations of the query is found in the first source of the plurality of sources, discontinue additional searches.

149. The apparatus of claim 148, wherein the query result comprises the identical match and wherein the label associated with a source of the plurality of sources associated with the query result comprises a linear label.

150. The apparatus of claim 148, wherein the processor-executable instructions that cause the apparatus to apply the query to one or more sources of a plurality of sources further cause the apparatus to:
search for an identical match to the query in any frame of a plurality of frames of a second source of the plurality of sources; and
if an identical match to the query is found in any frame of a plurality of frames of the second source of the plurality of sources, discontinue additional searches.

151. The apparatus of claim 150, wherein the query result comprises the identical match and wherein the label associated with a source of the plurality of sources associated with the query result comprises a linear label.

152. The apparatus of claim 150, wherein the processor-executable instructions that cause the apparatus to apply the query to one or more sources of a plurality of sources further cause the apparatus to:
search for a non-identical match to the query in a third source of the plurality of sources; and
if a non-identical match to the query is found in the third source of the plurality of sources, discontinue additional searches.

153. The apparatus of claim 152, wherein the query result comprises the non-identical match and wherein the label associated with a source of the plurality of sources associated with the query result comprises a mismatch label.

154. The apparatus of claim 152, wherein the processor-executable instructions that cause the apparatus to apply the query to one or more sources of a plurality of sources further cause the apparatus:
search for a homologous match to the query in a fourth source of the plurality of sources; and
if a homologous match to the query is found in the fourth source of the plurality of sources, discontinue additional searches.

155. The apparatus of claim 154, wherein the query result comprises the homologous match and wherein the label associated with a source of the plurality of sources associated with the query result comprises a homologous label.

156. The apparatus of claim 154, wherein the processor-executable instructions that cause the apparatus to apply the query to one or more sources of a plurality of sources further cause the apparatus to:
split the query into a plurality of sets of fragments;
search for each set of fragments in a fifth source of the plurality of sources;
if a match for a set of fragments is found in the fifth source of the plurality of sources, discontinue additional searches; and
if a first match for a first fragment of the set of fragments and a second match for a second fragment of the set of fragments is found in the fifth source of the plurality of sources, discontinue additional searches.

157. The apparatus of claim 156, wherein the query result comprises the match for the set of fragments and wherein the label associated with a source of the plurality of sources associated with the query result comprises a cis-spliced label.

158. The apparatus of claim 156, wherein the query result comprises the first match for the first fragment of the set of fragments and the second match for the second fragment of the set of fragments and wherein the label associated with a source of the plurality of sources associated with the query result comprises a trans-spliced label.

159. The apparatus of claim 138 wherein the processor-executable instructions further cause the apparatus to determine, based on the label, a source of the query.

160. The apparatus of claim 159 wherein the processor-executable instructions further cause the apparatus to validate output of a mass spectrometer system based on the source of the query.

161. One or more non-transitory computer-readable media storing processor-executable instructions thereon that, when executed by a processor, cause the processor to:
receive a query;
apply, based on a query support data structure, the query to one or more sources of a plurality of sources;
determine, based on a query result, a label associated with a source of the plurality of sources associated with the query result; and
apply the label to the query.

162. The one or more non-transitory computer-readable media of claim 161, wherein the query comprises a text string.

163. The one or more non-transitory computer-readable media of claim 161, wherein the query comprises a peptide sequence.

164. The one or more non-transitory computer-readable media of claim 161, wherein the processor-executable instructions that cause the processor to receive the query further cause the processor to receive the peptide sequence from a mass spectrometer system.

165. The one or more non-transitory computer-readable media of claim 161, wherein the processor-executable instructions further cause the processor to determine, via the mass spectrometer system, one or more amino acids of the peptide sequence.

166. The one or more non-transitory computer-readable media of claim 161, wherein the query support data structure indicates an order of the plurality of sources to apply the query, wherein the order is based on a false discovery rate associated with each source of the plurality of sources.

167. The one or more non-transitory computer-readable media of claim 161, wherein the processor-executable instructions further cause the processor to determine one or more permutations of the query.

168. The one or more non-transitory computer-readable media of claim 167, wherein the processor-executable instructions that cause the processor to apply, based on the query support data structure, the query to the one or more sources of the plurality of sources further cause the processor to:
apply each permutation of the one or more permutations of the query to the one or more sources of the plurality of sources;
if an identical match to the one or more permutations of the query is found in a first source of the plurality of sources, discontinue additional searches and applying a linear label to the one or more permutations of the query associated with the identical match; and
assign the one or more permutations of the query associated with the identical match as a correct query.

169. The one or more non-transitory computer-readable media of claim 161, wherein the processor-executable instructions that cause the processor to apply the query to one or more sources of a plurality of sources further cause the processor to:
search for an identical match to the query in a first source of the plurality of sources; and
if an identical match to the query is found in the first source of the plurality of sources, discontinue additional searches.

170. The one or more non-transitory computer-readable media of claim 169, wherein the query result comprises the identical match and wherein the label associated with a source of the plurality of sources associated with the query result comprises a linear label.

171. The one or more non-transitory computer-readable media of claim 161, wherein the processor-executable instructions that cause the processor to apply the query to one or more sources of a plurality of sources further cause the processor to:
search for an identical match to the query in a first source of the plurality of sources; and
if an identical match to the one or more permutations of the query is found in the first source of the plurality of sources, discontinue additional searches.

172. The one or more non-transitory computer-readable media of claim 171, wherein the query result comprises the identical match and wherein the label associated with a source of the plurality of sources associated with the query result comprises a linear label.

173. The one or more non-transitory computer-readable media of claim 171, wherein the processor-executable instructions that cause the processor to apply the query to one or more sources of a plurality of sources further cause the processor to:
search for an identical match to the query in any frame of a plurality of frames of a second source of the plurality of sources; and
if an identical match to the query is found in any frame of a plurality of frames of the second source of the plurality of sources, discontinue additional searches.

174. The one or more non-transitory computer-readable media of claim 173, wherein the query result comprises the identical match and wherein the label associated with a source of the plurality of sources associated with the query result comprises a linear label.

175. The one or more non-transitory computer-readable media of claim 173, wherein the processor-executable instructions that cause the processor to apply the query to one or more sources of a plurality of sources further cause the processor to:
search for a non-identical match to the query in a third source of the plurality of sources; and
if a non-identical match to the query is found in the third source of the plurality of sources, discontinue additional searches.

176. The one or more non-transitory computer-readable media of claim 175, wherein the query result comprises the non-identical match and wherein the label associated with a source of the plurality of sources associated with the query result comprises a mismatch label.

177. The one or more non-transitory computer-readable media of claim 176, wherein the processor-executable instructions that cause the processor to apply the query to one or more sources of a plurality of sources further cause the processor to:
search for a homologous match to the query in a fourth source of the plurality of sources; and
if a homologous match to the query is found in the fourth source of the plurality of sources, discontinue additional searches.

178. The one or more non-transitory computer-readable media of claim 177, wherein the query result comprises the homologous match and wherein the label associated with a source of the plurality of sources associated with the query result comprises a homologous label.

179. The one or more non-transitory computer-readable media of claim 177, wherein the processor-executable instructions that cause the processor to apply the query to one or more sources of a plurality of sources further cause the processor to:
split the query into a plurality of sets of fragments;
search for each set of fragments in a fifth source of the plurality of sources;
if a match for a set of fragments is found in the fifth source of the plurality of sources, discontinue additional searches; and
if a first match for a first fragment of the set of fragments and a second match for a second fragment of the set of fragments is found in the fifth source of the plurality of sources, discontinue additional searches.

180. The one or more non-transitory computer-readable media of claim 179, wherein the query result comprises the match for the set of fragments and wherein the label associated with a source of the plurality of sources associated with the query result comprises a cis-spliced label.

181. The one or more non-transitory computer-readable media of claim 179, wherein the query result comprises the first match for the first fragment of the set of fragments and the second match for the second fragment of the set of fragments and wherein the label associated with a source of the plurality of sources associated with the query result comprises a trans-spliced label.

182. The one or more non-transitory computer-readable media of claim 161, wherein the processor-executable instructions further cause the processor to determine, based on the label, a source of the query.

183. The one or more non-transitory computer-readable media of claim 182, wherein the processor-executable instructions further cause the processor to validate output of a mass spectrometer system based on the source of the query.

184. A system comprising:
a computing device configured to:
receive a query,
apply, based on a query support data structure, the query to one or more sources of a plurality of sources,
determine, based on a query result, a label associated with a source of the plurality of sources associated with the query result, and
apply the label to the query; and
the one or more sources of the plurality of sources configured to:
receive the query, and
determine the query result.

185. The system of claim 184, wherein the query comprises a text string.

186. The system of claim 184, wherein the query comprises a peptide sequence.

187. The system of claim 184, wherein the computing device configured to receive the query is further configured to receive the peptide sequence from a mass spectrometer system.

188. The system of claim 184, wherein the computing device is further configured to cause the processor to determine, via the mass spectrometer system, one or more amino acids of the peptide sequence.

189. The system of claim 184, wherein the query support data structure indicates an order of the plurality of sources to apply the query, wherein the order is based on a false discovery rate associated with each source of the plurality of sources.

190. The system of claim 184, wherein the computing device is further configured to determine one or more permutations of the query.

191. The system of claim 184, wherein the computing device configured to apply, based on the query support data structure, the query to the one or more sources of the plurality of sources is further configured to:
apply each permutation of the one or more permutations of the query to the one or more sources of the plurality of sources;
if an identical match to the one or more permutations of the query is found in a first source of the plurality of sources, discontinue additional searches and applying a linear label to the one or more permutations of the query associated with the identical match; and
assign the one or more permutations of the query associated with the identical match as a correct query.

192. The system of claim 184, wherein the computing device configured to cause the processor to apply the query to one or more sources of a plurality of sources is further configured to:
search for an identical match to the query in a first source of the plurality of sources; and
if an identical match to the query is found in the first source of the plurality of sources, discontinue additional searches.

193. The system of claim 192, wherein the query result comprises the identical match and wherein the label associated with a source of the plurality of sources associated with the query result comprises a linear label.

194. The system of claim 184, wherein the computing device configured to apply the query to one or more sources of a plurality of sources is further configured to:
search for an identical match to the query in a first source of the plurality of sources; and
if an identical match to the one or more permutations of the query is found in the first source of the plurality of sources, discontinue additional searches.

195. The system of claim 194, wherein the query result comprises the identical match and wherein the label associated with a source of the plurality of sources associated with the query result comprises a linear label.

196. The system of claim 194, wherein the computing device configured to apply the query to one or more sources of a plurality of sources is further configured to:
search for an identical match to the query in any frame of a plurality of frames of a second source of the plurality of sources; and
if an identical match to the query is found in any frame of a plurality of frames of the second source of the plurality of sources, discontinue additional searches.

197. The system of claim 196, wherein the query result comprises the identical match and wherein the label associated with a source of the plurality of sources associated with the query result comprises a linear label.

198. The system of claim 196, wherein the computing device configured to apply the query to one or more sources of a plurality of sources is further configured to:
search for a non-identical match to the query in a third source of the plurality of sources; and
if a non-identical match to the query is found in the third source of the plurality of sources, discontinue additional searches.

199. The system of claim 198, wherein the query result comprises the non-identical match and wherein the label associated with a source of the plurality of sources associated with the query result comprises a mismatch label.

200. The system of claim 198, wherein the computing device configured to apply the query to one or more sources of a plurality of sources is further configured to:
search for a homologous match to the query in a fourth source of the plurality of sources; and
if a homologous match to the query is found in the fourth source of the plurality of sources, discontinue additional searches.

201. The system of claim 200, wherein the query result comprises the homologous match and wherein the label associated with a source of the plurality of sources associated with the query result comprises a homologous label.

202. The system of claim 200, wherein the computing device configured to apply the query to one or more sources of a plurality of sources is further configured to:
split the query into a plurality of sets of fragments;
search for each set of fragments in a fifth source of the plurality of sources;
if a match for a set of fragments is found in the fifth source of the plurality of sources, discontinue additional searches; and
if a first match for a first fragment of the set of fragments and a second match for a second fragment of the set of fragments is found in the fifth source of the plurality of sources, discontinue additional searches.

203. The system of claim 202, wherein the query result comprises the match for the set of fragments and wherein the label associated with a source of the plurality of sources associated with the query result comprises a cis-spliced label.

204. The system of claim 202, wherein the query result comprises the first match for the first fragment of the set of fragments and the second match for the second fragment of the set of fragments and wherein the label associated with a source of the plurality of sources associated with the query result comprises a trans-spliced label.

205. The system of claim 184, wherein the processor-executable instructions further cause the processor to determine, based on the label, a source of the query.

206. The system of claim 205, wherein the computing device is further configured to validate output of a mass spectrometer system based on the source of the query.
